(19) United States
(12) Patent Application Publication
(10) Pub. No.: US 2024/0262905 A1
(43) Pub. Date: Aug. 8, 2024

(54) SKIN TARGETED IMMUNOTOLERANCE

(71) Applicant: PANDION OPERATIONS, INC., Watertown, MA (US)

(72) Inventors: Purvi MANDE, Watertown, MA (US); Susmita BORTHAKUR, Watertown, MA (US); Daniel RIOS, Boston, MA (US); Nathan HIGGINSON-SCOTT, Hineham, MA (US); Kevin Lewis OTIPOBY, Ashland, MA (US); Joanne L. VINEY, Belmont, MA (US)

(73) Assignee: PANDION OPERATIONS, INC., Watertown, MA (US)

(21) Appl. No.: 18/248,401

(22) PCT Filed: Oct. 15, 2021
(86) PCT No.: PCT/US2021/055239

§ 371 Date: Apr. 10, 2023

Related U.S. Application Data
(60) Provisional application No. 63/092,756, filed on Oct. 16, 2020.
(60) Provisional application No. 63/175,780, filed on Apr. 16, 2021.

(51) Int. Cl. C07K 16/28; C07K 14/55; C07K 14/705

(52) U.S. Cl. CPC C07K 16/28; C07K 14/55; C07K 14/70596; C07K 2317/31; C07K 2317/622; C07K 2317/64; C07K 2317/75; C07K 2317/92; C07K 2319/01; C07K 2319/74

(57) ABSTRACT

Methods and compounds for conferring skin-specific immune privilege.

### CROSS-REFERENCE TO RELATED APPLICATIONS

This application claims priority to U.S. Provisional Application No. 63/175,780, filed Apr. 16, 2021, U.S. Provisional Application No. 63/092,756, filed Oct. 16, 2020, each of which is hereby incorporated by reference in its entirety.

### FIELD

The embodiments provided herein relate to, for example, methods and compositions for skin-targeted immune-privilege.

### BACKGROUND

Instances of unwanted immune responses, e.g., as in the rejection of transplanted tissue or in autoimmune disorders, constitute a major health problem for millions of people across the world. Long-term outcomes for organ transplantation are frequently characterized by chronic rejection, and eventual failure of the transplanted organ. More than twenty autoimmune disorders are known, affecting essentially every organ of the body, and affecting over fifty million people in North America alone. The broadly active immunosuppressive medications used to combat the pathogenic immune response in both scenarios have serious side effects.

### SUMMARY

Disclosed herein are methods, compositions, polypeptides, and compounds that provide skin-specific immune privilege. Embodiments disclosed herein are incorporated by reference into this section.

In some embodiments, a polypeptide comprising a skin targeting moiety that binds to a target cell and an effector binding/modulating moiety, wherein the effector binding/modulating moiety is a PD-1 agonist, CD39 Effector Domain, or an IL-2 mutein polypeptide (IL-2 mutein).

In some embodiments, the targeting moiety comprises an antibody that binds to a target protein on the surface of the skin cell. In some embodiments, the antibody is an antibody that binds to a desmoglein protein. In some embodiments, the IL-2 mutein binds to a receptor expressed by an immune cell. In some embodiments, the immune cell contributes to an unwanted immune response. In some embodiments, the immune cell causes a disease pathology.

In some embodiments, the targeting moiety comprises an anti-desmoglein 1 antibody, an anti-desmoglein 2 antibody, an anti-desmoglein 3 antibody, or an anti-desmoglein 4 antibody.

In some embodiments, pharmaceutical compositions comprising a protein or polypeptide provided herein are provided.

In some embodiments, methods of treating a subject with inflammatory disorder, such as graft versus host disease (GVHD), or a skin disorder are provided. In some embodiments, the methods comprise administering a protein, an antibody, or pharmaceutical compositions as provided for herein. In some embodiments, the skin disorder is vitiligo or alopecia.

In some embodiments, methods of treating a transplant subject, such as a skin transplant, treating GVHD in a subject having transplanted donor tissue, treating a subject having, or at risk, or elevated risk, of having an autoimmune disorder are provided. In some embodiments, the methods comprising administering a protein, an antibody, or pharmaceutical compositions as provided for herein. In some embodiments, the subject has an autoimmune disorder and the therapeutic compound does not comprise an autoantigenic peptide or polypeptide characteristic of the autoimmune disorder, e.g., does not comprise a peptide or polypeptide against which the subject has autoantibodies.

### BRIEF DESCRIPTION OF THE DRAWINGS

FIG. 1 depicts human IgG staining of mouse skin tissue expressing desmoglein.

FIG. 2A depicts de-pigmentation scores in a mouse Vitiligo model.

FIG. 2B depicts de-pigmentation in a mouse Vitiligo model.

FIG. 3 depicts a non-limiting illustration of the therapeutic compounds provided herein.

FIG. 3A depicts a non-limiting illustration of the therapeutic compounds provided herein.

FIG. 4 depicts a non-limiting illustration of the therapeutic compounds provided herein.

FIG. 5 depicts a non-limiting illustration of the therapeutic compounds provided herein.

FIG. 6 depicts a non-limiting illustration of the therapeutic compounds provided herein.

FIG. 7 depicts a non-limiting illustration of the therapeutic compounds provided herein.

FIG. 8 depicts a non-limiting illustration of the therapeutic compounds provided herein.

FIG. 9 depicts a non-limiting illustration of the therapeutic compounds provided herein.

FIG. 10 depicts a non-limiting illustration of the therapeutic compounds provided herein.

FIG. 11 depicts a non-limiting illustration of the therapeutic compounds provided herein.

FIG. 12 depicts a non-limiting illustration of the therapeutic compounds provided herein.

FIG. 13 depicts a non-limiting illustration of the therapeutic compounds provided herein.

FIG. 14 depicts a non-limiting illustration of the therapeutic compounds provided herein.

FIG. 15 depicts a non-limiting illustration of the therapeutic compounds provided herein.

FIG. 16 depicts a non-limiting illustration of the therapeutic compounds provided herein.

FIG. 17 depicts a non-limiting illustration of the therapeutic compounds provided herein.

FIG. 18 depicts a non-limiting illustration of the therapeutic compounds provided herein.

FIG. 19 depicts a non-limiting illustration of the therapeutic compounds provided herein.

FIG. 20A depicts specificity of the anti-desmoglein 1 antibody tether.

FIG. 20B-C illustrate the effect of different tethered-effectors on inflammation in mice.

### DETAILED DESCRIPTION

This application incorporates by reference each of the following in its entirety: U.S. application Ser. No. 15/922,592 filed Mar. 15, 2018 and PCT Application No. PCT/US2018/022675, filed Mar. 15, 2018. This application also incorporate by reference, each of the following in their entirety: U.S. Provisional Application No. 62/721,644, filed Aug. 23, 2018, U.S. provisional Application No. 62/675,972 filed May 24, 2018, U.S. provisional Application No. 62/595,357 filed Dec. 6, 2017, U.S. Provisional Application No. 62/595,348, filed Dec. 6, 2017, U.S. Non-Provisional application Ser. No. 16/109,875, filed Aug. 23, 2018, U.S. Non-Provisional application Ser. No. 16/109,897, filed Aug. 23, 2018, U.S. Non-Provisional application Ser. No. 15/988,311, filed May 24, 2018, PCT Application No. PCT/US2018/034334, filed May 24, 2018, and, PCT/US2018/062780, filed Nov. 28, 2018.

As used herein and in the appended claims, the singular forms “a”, “an” and “the” include plural reference unless the context clearly dictates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±5% and remain within the scope of the disclosed embodiments.

As used herein, the term “animal” includes, but is not limited to, humans and non-human vertebrates such as wild, domestic, and farm animals. In some embodiments, the animal is a mammal. The term “mammal” means a rodent (i.e., a mouse, a rat, or a guinea pig), a monkey, a cat, a dog, a cow, a horse, a pig, or a human. In some embodiments, the mammal is a human.

As used herein, the term “contacting” means bringing together of two elements in an in vitro system or an in vivo system. For example, “contacting” a therapeutic compound with an individual or patient or cell includes the administration of the compound to an individual or patient, such as a human, as well as, for example, introducing a compound into a sample containing a cellular or purified preparation containing target.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

As used herein, the term “fused” or “linked” when used in reference to a protein having different domains or heterologous sequences means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another. In some embodiments, the various domains or proteins provided for herein are linked or fused directly to one another or via a linker sequence, such as the glycine/serine sequences described herein to link the two domains together.

As used herein, the term “individual,” “subject,” or “patient,” used interchangeably, means any animal, including mammals, such as mice, rats, other rodents, rabbits, dogs, cats, swine, cattle, sheep, horses, or primates, such as humans.

As used herein, the term “inhibit” refers to a result, symptom, or activity being reduced as compared to the activity or result in the absence of the compound that is inhibiting the result, symptom, or activity. In some embodiments, the result, symptom, or activity, is inhibited by about, or, at least, 10%, 20%, 30%, 40%, 50%, 60%, 70%, 80%, 90%, 95%, or 99%. A result, symptom, or activity can also be inhibited if it is completely elimination or extinguished.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, or condition is prevalent.

As used herein, the phrase “integer from X to Y” means any integer that includes the endpoints. For example, the phrase “integer from 1 to 5” means 1, 2, 3, 4, or 5.

In some embodiments, therapeutic compounds are provided herein. In some embodiments, the therapeutic compound is a protein or a polypeptide, that has multiple peptide chains that interact with one another. The polypeptides can interact with one another through non-covalent interactions or covalent interactions, such as through disulfide bonds or other covalent bonds. Therefore, if an embodiment refers to a therapeutic compound it can also be said to refer to a protein or polypeptide as provided for herein and vice versa as the context dictates.

As used herein, the phrase “ophthalmically acceptable” means having no persistent detrimental effect on the treated eye or the functioning thereof, or on the general health of the subject being treated. However, it will be recognized that transient effects such as minor irritation or a “stinging” sensation are common with topical ophthalmic administration of drugs and the existence of such transient effects is not inconsistent with the composition, formulation, or ingredient (e.g., excipient) in question being “ophthalmically acceptable” as herein defined. In some embodiments, the pharmaceutical compositions can be ophthalmically acceptable or suitable for ophthalmic administration.

“Specific binding” or “specifically binds to” or is “specific for” a particular antigen, target, or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen, target, or an epitope can be exhibited, for example, by an antibody having a K_{D} for an antigen or epitope of at least about 10^{-4M} at least about 10^{-5M}, at least about 10^{-6 M}, at least about 10^{-7M}, at least about 10^{-8M}, at least about 10^{-9M} alternatively at least about 10^{-10 M}, at least about 10^{-11M} at least about 10^{-12M}, or greater, where K_{D} refers to a dissociation rate of a particular antibody-target interaction. Typically, an antibody that specifically binds an antigen or target will have a K_{D} that is, or at least, 2-, 4-, 5-, 10-, 20-, 50-, 100-, 500-, 1000-, 5,000-, 10,000-, or more times greater for a control molecule relative to the antigen or epitope.

In some embodiments, specific binding for a particular antigen, target, or an epitope can be exhibited, for example, by an antibody having a K_{A} or Kₐ for a target, antigen, or epitope of at least 2-, 4-, 5-, 20-, 50-, 100-, 500-, 1000-, 5,000-, 10,000- or more times greater for the target, antigen, or epitope relative to a control, where K_{A} or Kₐ refers to an association rate of a particular antibody-antigen interaction.

As provided herein, the therapeutic compounds and compositions can be used in methods of treatment as provided herein.

### PD-1 Agonists

Provided herein are therapeutic compounds, e.g., therapeutic protein molecules, such as fusion proteins, including a targeting moiety and an effector binding/modulating moiety, typically as separate domains. Also provided are methods of using and making the therapeutic compounds. The targeting moiety serves to localize the therapeutic compound, and thus the effector binding/modulating moiety, to a site at which immune-privilege is desired. As used herein, “immune privilege” means lack of, or suppression of an inflammatory response. As a non-limiting example, immune privilege includes situations where a tissue or site in the body is able to tolerate the introduction of antigens without eliciting an inflammatory immune response (Forester J. V., Lambe H. Xu, Cornall R. Immune Previlege or privileged immunity? Mucosal Immunology, 1, 372-381 (2008)).

The present disclosure provides, for example, molecules that can act as PD-1 agonists. In some embodiments, the agonist is an antibody that binds to and agonizes PD-1. Without wishing to be bound to any particular theory, agonism of PD-1 inhibits T cell activation/signaling and can be accomplished by different mechanisms. For example cross-linking of bead-bound functional PD-1 agonists can lead to agonism. Functional PD-1 agonists have been described (Akkaya. Ph.D. Thesis: Modulation of the PD-1 pathway by inhibitory antibody superagonists. Christ Church College, Oxford, U K, 2012), which is hereby incorporated by reference. Crosslinking of PD-1 with two mAbs that bind non-overlapping epitopes induces PD-1 signaling (Davis, US 2011/0171220), which is hereby incorporated by reference. Another example is illustrated through the use of a goat anti-PD-1 antiserum (e.g. AF1086, R&D Systems) which is hereby incorporated by reference, which acts as an agonist when soluble (Said et al., 2010, Nat Med) which is hereby incorporated by reference. Non-limiting examples of PD-1 agonists that can be used in the present embodiments include, but are not limited to, UCB clone 19 or clone 10, PD1AB-1, PD1AB-2, PD1AB-3, PD1AB-4 and PD1AB-5, PD1AB-6 (Anaptys/Celgene), PD1-17, PD1-28, PD1-33 and PD1-35 (Collins et al, US 2008/0311117 A1), antibodies against PD-1 and uses therefor, which is hereby incorporated by reference, or can be a bispecific, monovalent anti-PD-1/anti-CD3 (Ono), and the like. In some embodiments, the PD-1 agonist antibodies can be antibodies that block binding of PD-L1 to PD-1. In some embodiments, the PD-1 agonist antibodies can be antibodies that do not block binding of PD-L1 to PD-1. In some embodiments, the antibody does not act as an antagonist of PD-1.

PD-1 agonism can be measured by any method, such as the methods described in the examples. For example, cells can be constructed that express, including stably express, constructs that include a human PD-1 polypeptide fused to a beta-galactosidase “Enzyme donor” and 2) a SHP-2 polypeptide fused to a beta-galactosidase “Enzyme acceptor.” Without being bound by any theory, when PD-1 is engaged, SHP-2 is recruited to PD-1. The enzyme acceptor and enzyme donor form a fully active beta-galactosidase enzyme that can be assayed. Although, the assay does not directly show PD-1 agonism, but shows activation of PD-1 signaling. PD-1 agonism can also be measured by measuring inhibition of T cell activation because, without being bound to any theory, PD-1 agonism inhibits anti-CD3-induced T cell activation. For example, PD-1 agonism can be measured by preactivating T cells with PHA (for human T cells) or ConA (for mouse T cells) so that they express PD-1. The cells can then be reactivated with anti-CD3 in the presence of anti-PD-1 (or PD-L1) for the PD-1 agonism assay. T cells that receive a PD-1 agonist signal in the presence of anti-CD3 will show decreased activation, relative to anti-CD3 stimulation alone. Activation can be readout by proliferation or cytokine production (IL-2, IFNg, IL-17) or other markers, such as CD69 activation marker. Thus, PD-1 agonism can be measured by either cytokine production or cell proliferation. Other methods can also be used to measure PD-1 agonism.

PD-1 is an Ig superfamily member expressed on activated T cells and other immune cells. The natural ligands for PD-1 appear to be PD-L1 and PD-L2. Without being bound to any particular theory, when PD-L1 or PD-L2 bind to PD-1 on an activated T cell, an inhibitory signaling cascade is initiated, resulting in attenuation of the activated T effector cell function. Thus, blocking the interaction between PD-1 on a T cell, and PD-L1/2 on another cell (e.g., tumor cell) with a PD-1 antagonist is known as checkpoint inhibition, and releases the T cells from inhibition. In contrast, PD-1 agonist antibodies can bind to PD-1 and send an inhibitory signal and attenuate the function of a T cell. Thus, PD-1 agonist antibodies can be incorporated into various embodiments described herein as an effector molecule binding/modulating moiety, (sometimes also referred to herein as an effector molecule) which can accomplish localized tissue-specific immunomodulation when paired with a targeting moiety.

The effector molecules, including the PD-1 agonist, can be linked to a targeting, moiety, such as one that binds to desmoglein-1, desmoglein-2, desmoglein-3, or desmoglein-4. As used herein, the term “desmoglein-1” refers to the protein desmoglein-1, which can also be referred to as cadherin family member 4, desmosomal glycoprotein 1, DSG1, DGI, DG1, or even pemphigus foliaceus antigen. As used herein, the term “desmoglein-2” refers to the protein desmoglein-2, which can also be referred to as cadherin family member 5, HDGC, or DSG2. As used herein, the term “desmoglein-3” refers to the protein desmoglein-3, which can also be referred to as cadherin family member 6, DSG3, PVA, or even 130 kDa pemphigus vulgaris antigen. As used herein, the term “desmoglein-4” refers to the protein desmoglein-4, which can also be referred to as cadherin family member 13, or DSG4.

In some embodiments, the targeting moiety (e.g., that binds to desmoglein-1, desmoglein-2, desmoglein-3, or desmoglein-4) and effector binding/modulating moiety (e.g. PD-1 agonist, CD39 effector domain, and/or IL-2 mutein) are physically tethered, covalently or non-covalently, directly or through a linker entity, to one another, e.g., as a member of the same protein molecule in a therapeutic protein molecule. In some embodiments, the targeting and effector moieties are provided in a therapeutic protein molecule, e.g., a fusion protein, typically as separate domains. In some embodiments, the targeting moiety, the effector binding/modulating moiety, or both each comprises a single domain antibody molecule, e.g., a camelid antibody VHH molecule or human soluble VH domain. It may also contain a single-chain fragment variable (scFv) or a Fab domain. In some embodiments, the therapeutic protein molecule, or a nucleic acid, e.g., an mRNA or DNA, encoding the therapeutic protein molecule, can be administered to a subject. In some embodiments, the targeting and effector molecule binding/modulating moieties are linked to a third entity, e.g., a carrier, e.g., a polymeric carrier, a dendrimer, or a particle, e.g., a nanoparticle. The therapeutic compounds can be used to down regulate an immune response at or in a tissue at a selected target or site while having no or substantially less immunosuppressive function systemically. The target or site can comprise donor tissue or autologous tissue.

Provided herein are methods of providing site-specific immune privilege for a transplanted donor tissue, e.g., an allograft tissue, e.g., a tissue described herein, e.g., an allograft liver, an allograft kidney, an allograft heart, an allograft pancreas, an allograft thymus or thymic tissue, an allograft skin, or an allograft lung, with therapeutic compounds disclosed herein. In embodiments the treatment minimizes rejection of, minimizes immune effector cell mediated damage to, prolongs acceptance of, or prolongs the functional life of, donor transplant tissue.

Also provided herein are methods of inhibiting graft versus host disease (GVHD) by minimizing the ability of donor immune cells, e.g., donor T cells, to mediate immune attack of recipient tissue, with therapeutic compounds disclosed herein.

Also provided herein are methods of treating, e.g., therapeutically treating or prophylactically treating (or preventing), an autoimmune disorder or autoimmune response in a subject by administration of a therapeutic compound disclosed herein, e.g., to provide site or tissue specific modulation of the immune system. In some embodiments, the method provides tolerance to, minimization of the rejection of, minimization of immune effector cell mediated damage to, or prolonging a function of, subject tissue. In some embodiments, the therapeutic compound includes a targeting moiety that targets, e.g., specifically targets, the tissue under, or at risk for, autoimmune attack. Non-limiting exemplary tissues include, but are not limited to, the pancreas, myelin, salivary glands, synoviocytes, and myocytes.

As used herein, the terms “treat,” “treated,” or “treating” in regards to therapeutic treatment wherein the object is to slow down (lessen) an undesired physiological condition, disorder or disease, or obtain beneficial or desired clinical results. For example, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment. Thus, “treatment of an autoimmune disease/disorder” means an activity that alleviates or ameliorates any of the primary phenomena or secondary symptoms associated with the autoimmune disease/disorder or other condition described herein. The various disease or conditions are provided herein. The therapeutic treatment can also be administered prophylactically to preventing or reduce the disease or condition before the onset.

In some embodiments, administration of an effective amount of the therapeutic compound begins after the disorder is apparent. In some embodiments, administration of the therapeutic compound begins prior to onset, or full onset, of the disorder, e.g., in a subject having the disorder, a high-risk subject, a subject having a biomarker for risk or presence of the disorder, a subject having a family history of the disorder, or other indicator of risk of, or asymptomatic presence of, the disorder. For example, in some embodiments, a subject having islet cell damage but which is not yet diabetic, is treated.

While not wishing to be bound by theory, it is believed that the targeting moiety functions to bind and accumulate the therapeutic compound to a target selectively or preferentially expressed at the anatomical site where immune privilege is desired. In some embodiments, e.g., in the context of donor tissue transplantation, the target moiety binds to a target, e.g., an allelic product, present in the donor tissue but not the recipient. For treatment of autoimmune disorders, the targeting moiety binds a target preferentially expressed at the anatomical site where immune privilege is desired, e.g., in the pancreas. For treatment of GVHID, the targeting moiety targets the host tissue, and protects the host against attack from transplanted immune effector cells derived from transplanted tissue.

Again, while not wishing to be bound by theory, it is believed that the effector binding/modulating moiety serves to deliver an immunosuppressive signal or otherwise create an immune privileged environment.

As used herein, effector, or effector moiety, refers to an entity, e.g., a cell or molecule, e.g., a soluble or cell surface molecule, which mediates an immune response. Non-limiting examples of effector molecules are PD-1 agonists, IL-2 muteins, and the CD39 domains and polypeptides, such as those provided for herein.

Effector ligand binding molecule, or effector ligand binding moiety, as used herein, refers to a polypeptide that has sufficient sequence from a naturally occurring counter ligand of an effector, that it can bind the effector with sufficient specificity that it can serve as an effector binding/modulating molecule. In some embodiments, an effector ligand binding molecule binds to effector with at least 10, 20, 30, 40, 50, 60, 70, 80, 90, or 95% of the affinity of the naturally occurring counter ligand. In some embodiments, an effector ligand binding molecule has at least 60, 70, 80, 90, 95, 99, or 100% sequence identity, or substantial sequence identity, with a naturally occurring counter ligand for the effector.

Effector specific binding polypeptide, or effector specific binding moiety, as used herein, refers to a polypeptide that can bind with sufficient specificity that it can serve as an effector binding/modulating moiety. In some embodiments, a specific binding polypeptide comprises a effector ligand binding molecule.

Unless otherwise defined, all technical and scientific terms used herein have the same meaning as commonly understood by one of ordinary skill in the art to which these embodiments belong. Although methods and materials similar or equivalent to those described herein can be used in the practice or testing of the present embodiments, suitable methods and materials are described below. All publications, patent applications, patents, and other references mentioned herein are incorporated by reference in their entirety. In addition, the materials, methods, and examples are illustrative only and not intended to be limiting. Headings, sub-headings or numbered or lettered elements, e.g., (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of the embodiments will be apparent from the description and drawings, and from the claims.

### Additional Definitions

Unless defined otherwise, all technical and scientific terms used herein have the same meaning as commonly understood by one of ordinary skill in the art to which the embodiments pertain. In describing and claiming the present embodiments, the following terminology and terminology otherwise referenced throughout the present application will be used according to how it is defined, where a definition is provided.

It is also to be understood that the terminology used herein is for the purpose of describing particular embodiments only, and is not intended to be limiting.

Antibody molecule, as that term is used herein, refers to a polypeptide, e.g., an immunoglobulin chain or fragment thereof, comprising at least one functional immunoglobulin variable domain sequence. An antibody molecule encompasses antibodies (e.g., full-length antibodies) and antibody fragments. In some embodiments, an antibody molecule comprises an antigen binding or functional fragment of a full-length antibody, or a full-length immunoglobulin chain. For example, a full-length antibody is an immunoglobulin (Ig) molecule (e.g., an IgG antibody) that is naturally occurring or formed by normal immunoglobulin gene fragment recombinatorial processes. In embodiments, an antibody molecule refers to an immunologically active, antigen binding portion of an immunoglobulin molecule, such as an antibody fragment. An antibody fragment, e.g., functional fragment, comprises a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full-length antibodies and antibody fragments, e.g., dAb (domain antibody), single chain, Fab, Fab′, and F(ab′)2 fragments, and single chain variable fragments (scFvs).

The term “antibody molecule” also encompasses whole or antigen binding fragments of domain, or single domain, antibodies, which can also be referred to as “sdAb” or “VHH.” Domain antibodies comprise either VH or VL that can act as stand-alone, antibody fragments. Additionally, domain antibodies include heavy-chain-only antibodies (HCAbs). Domain antibodies also include a CH2 domain of an IgG as the base scaffold into which CDR loops are grafted. It can also be generally defined as a polypeptide or protein comprising an amino acid sequence that is comprised of four framework regions interrupted by three complementarity determining regions. This is represented as FR1-CDR1-FR2-CDR2-FR3-CDR3-FR4. sdAbs can be produced in camelids such as llamas, but can also be synthetically generated using techniques that are well known in the art. The numbering of the amino acid residues of a sdAb or polypeptide is according to the general numbering for VH domains given by Kabat et al. (“Sequence of proteins of immunological interest,” US Public Health Services, NIH Bethesda, MD, Publication No. 91, which is hereby incorporated by reference). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113. Domain antibodies are also described in WO2004041862 and WO2016065323, each of which is hereby incorporated by reference. The domain antibodies can be a targeting moiety as described herein.

Antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, or hexavalent), or with higher orders of specificity (e.g., tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide.

Examples of formats for multispecific therapeutic compounds, e.g., bispecific antibody molecules are shown in the following non-limiting examples. Although illustrated with antibody molecules, they can be used as platforms for therapeutic molecules that include other non-antibody moieties as specific binding or effector moieties. In some embodiments, these non-limiting examples are based upon either a symmetrical or asymmetrical Fc formats.

For example, the figures illustrate non-limiting and varied symmetric homodimer approach. In some embodiments, the dimerization interface centers around human IgG1 CH2-CH3 domains, which dimerize via a contact interface spanning both CH2/CH2 and CH3/CH3. The resulting bispecific antibodies shown have a total valence comprised of four binding units with two identical binding units at the N-terminus on each side of the dimer and two identical units at the C-terminus on each side of the dimer. In each case the binding units at the N-terminus of the homodimer are different from those at the C-terminus of the homodimer. Using this type of bivalency for both an inhibitory T cell receptor at either terminus of the molecule and bivalency for a tissue tethering antigen can be achieved at either end of the molecule.

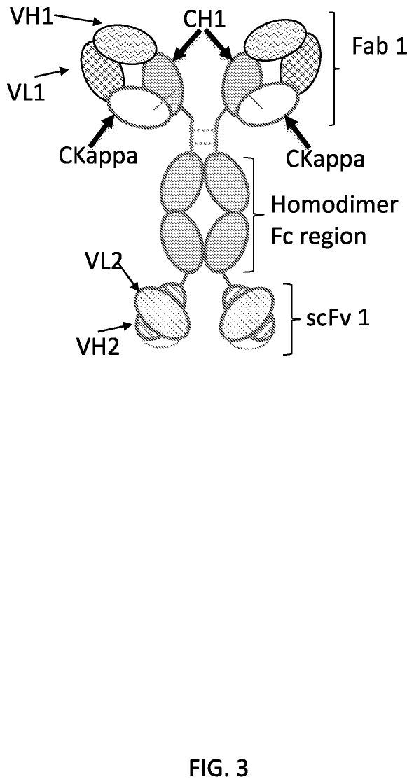

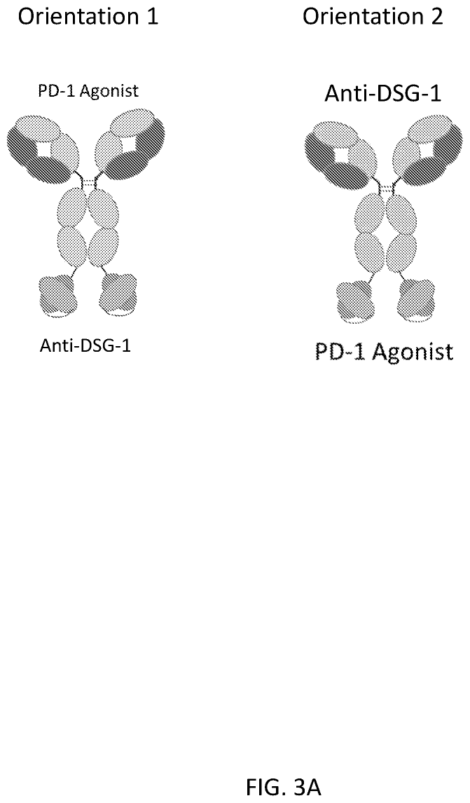

For example, in FIG. 3, a non-limiting embodiment is illustrated. The N-terminus of the homodimer contains two identical Fab domains comprised of two identical light chains, which are separate polypeptides, interfaced with the n-terminal VH-CH1 domains of each heavy chain via the VH/VL interaction and Ckappa or Clambda interaction with CH1. The native disulfide bond between the Ckappa or Clambda with CH1 provides a covalent anchor between the light and heavy chains. At the C-terminus of this design are two identical scFv units where by (in this example) the C-terminus of the CH3 domain of the Fc, is followed by a flexible, hydrophilic linker typically comprised of (but not limited to) serine, glycine, alanine, and/or threonine residues, which is followed by the VH domain of each scFv unit, which is followed by a glycine/serine rich linker, followed by a VL domain. These tandem VH and VL domains associate to form a single chain fragment variable (scFv) appended at the C-terminus of the Fc. Two such units exist at the C-terminus of this molecule owing to the homodimeric nature centered at the Fc. The domain order of scFvs may be configured to be from N- to C-terminus either VH-Linker-VL or VL-Linker-VH.

A non-limiting example of a molecule that has different binding regions on the different ends is where, one end is a PD-1 agonist and the antibody that provides target specificity is an anti-desmoglein 1 antibody, an anti-desmoglein 2 antibody, an anti-desmoglein 3 antibody, or an anti-desmoglein 4 antibody. This can be illustrated as shown, for example, in FIG. 3A, which illustrates the molecules in different orientations.

In some embodiments, the PD-1 agonist is replaced with an IL-2 mutein, such as, but not limited to, the ones described herein.

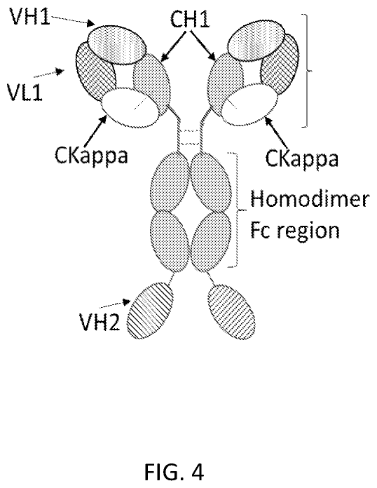

In another example, and as depicted in FIG. 4, the N-terminus of the homodimer contains two identical Fab domains comprised of two identical light chains, which are separate polypeptides, interfaced with the N-terminal VH-CH1 domains of each heavy chain via the VH/VL interaction and Ckappa or Clambda interaction with CH1. The native disulfide bond between the Ckappa or Clambda with CH1 provides a covalent anchor between the light and heavy chains. At the C-terminus of this design are two identical VH units (though non-antibody moieties could also be substituted here or at any of the four terminal attachment/fusion points) where by (in this example) the C-terminus of the CH3 domain of the Fc, is followed by a flexible, hydrophilic linker typically comprised of (but not limited to) serine, glycine, alanine, and/or threonine residues, which is followed by a soluble independent VH3 germline family based VH domain. Two such units exist at the C-terminus of this molecule owing to the homodimeric nature centered at the Fc.

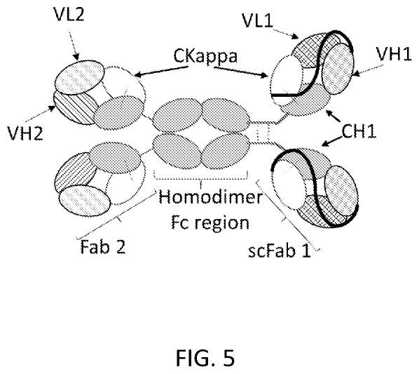

In another non-limiting example, as depicted in FIG. 5, the N-terminus of the homodimer contains two identical Fab domains comprised of two identical light chains, which, unlike FIG. 3 and FIG. 4, are physically conjoined with the heavy chain at the N-terminus via a linker between the C-terminus of Ckappa or Clambda and the N-terminus of the VH. The linker may be 36-80 amino acids in length and comprised of serine, glycine, alanine and threonine residues. The physically conjoined N-terminal light chains interface with the N-terminal VH-CH1 domains of each heavy chain via the VH/VL interaction and Ckappa or Clambda interaction with CH1. The native disulfide bond between the Ckappa or Clambda with CH1 provides additional stability between the light and heavy chains. At the C-terminus of this design are two identical Fab units where by (in this example) the C-terminus of the CH3 domain of the Fc, is followed by a flexible, hydrophilic linker typically comprised of (but not limited to) serine, glycine, alanine, and/or threonine residues, which is followed by a CH1 domain, followed by a VH domain at the C-terminus. The light chain that is designed to pair with the C-terminal CH1/VH domains is expressed as a separate polypeptide, unlike the N-terminal light chain which is conjoined to the N-terminal VH/CH1 domains as described. The C-terminal light chains form an interface at between VH/VL and Ckappa or Clambda with CH1. The native disulfide anchors this light chain to the heavy chain. Again, any of the antibody moieties at any of the four attachment/fusion points can be substituted with a non-antibody moiety, e.g., an effector binding/modulating moiety that does not comprise an antibody molecule.

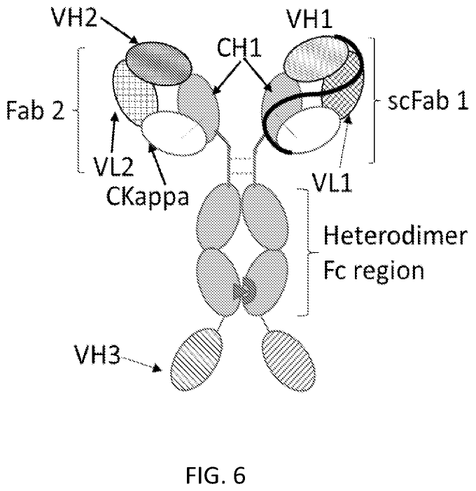

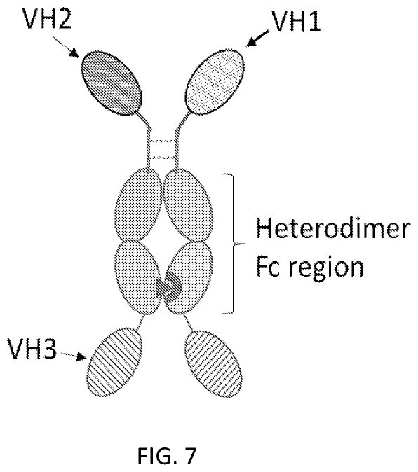

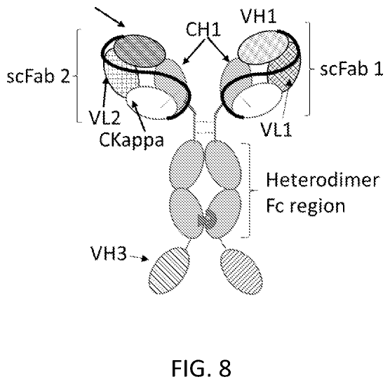

The bispecific antibodies can also be asymmetric as shown in the following non-limiting examples. Non-limiting example are also depicted in FIG. 6, FIG. 7, and FIG. 8, which illustrate an asymmetric/heterodimer approach. Again, in any of these formats, any of the antibody moieties at any of the four attachment/fusion points can be substituted with a non-antibody moiety, e.g., a effector binding/modulating moiety that does not comprise an antibody molecule. In some embodiments, the dimerization interface centers around the human IgG1 CH2-CH3 domains, which dimerize via a contact interface spanning both CH2/CH2 and CH3/CH3. However, in order to achieve heterodimerization instead of homodimerization of each heavy chain, mutations are introduced in each CH3 domain. The heterodimerizing mutations include T366W mutation (Kabat) in one CH3 domain and T366S, L368A, and Y407V (Kabat) mutations in the other CH3 domain. The heterodimerizing interface may be further stabilized with de novo disulfide bonds via mutation of native residues to cysteine residues such as S354 and Y349 on opposite sides of the CH3/CH3 interface. The resulting bispecific antibodies shown have a total valence comprised of four binding units. With this approach, the overall molecule can be designed to have bispecificity at just one terminus and monospecificity at the other terminus (trispecificity overall) or bispecificity at either terminus with an overall molecular specificity of 2 or 4. In the illustrative examples below, the C-terminus comprises two identical binding domains which could, for example, provide bivalent monospecificity for a tissue tethering target. At the N-terminus of all three of the illustrative examples, both binding domains comprise different recognition elements/paratopes, which could achieve recognition of two different epitopes on the same effector moiety target, or could recognize for example a T cell inhibitory receptor and CD3. In some embodiments, the N-terminal binding moieties may be interchanged with other single polypeptide formats such as scFv, single chain Fab, tandem scFv, VH or VHH domain antibody configurations for example. Other types of recognition element may be used also, such as linear or cyclic peptides.

An example of an asymmetric molecule is depicted in FIG. 6. Referring to FIG. 6, the N-terminus of the molecule is comprised of a first light chain paired with a first heavy chain via VH/VL and Ckappa or Clambda/CH1 interactions and a covalent tether comprised of the native heavy/light chain disulfide bond. On the opposite side of this heterodimeric molecule at the N-terminus is a second light chain and a second heavy chain which are physically conjoined via a linker between the C-terminus of Ckappa or Clambda and the N-terminus of the VH. The linker may be 36-80 amino acids in length and comprised of serine, glycine, alanine and threonine residues. The physically conjoined N-terminal light chains interface with the N-terminal VH-CH1 domains of each heavy chain via the VH/VL interaction and Ckappa or Clambda interaction with CH1. The native disulfide bond between the Ckappa or Clambda with CH1 provides additional stability between the light and heavy chains. At the C-terminus of the molecule are two identical soluble VH3 germline family VH domains joined via an N-terminal glycine/serine/alanine/threonine based linker to the C-terminus of the CH3 domain of both heavy chain 1 and heavy chain 2.

In some embodiments, an asymmetric molecule can be as illustrated as depicted in FIG. 7. For example, the N-terminus of the molecule is comprised of two different VH3 germlined based soluble VH domains linked to the human IgG1 hinge region via a glycine/serine/alanine/threonine based linker. The VH domain connected to the first heavy chain is different to the VH domain connected to the second heavy chain. At the C-terminus of each heavy chain is an additional soluble VH3 germline based VH domain, which is identical on each of the two heavy chains. The heavy chain heterodimerizes via the previously described knobs into holes mutations present at the CH3 interface of the Fc module.

In some embodiments, an asymmetric molecule can be as illustrated in FIG. 8. This example is similar to the molecule shown in FIG. 7, except both N-terminal Fab units are configured in a way that light chain 1 and light chain 2 are physically conjoined with heavy chain 1 and heavy chain 2 via a linker between the C-terminus of Ckappa or Clambda and the N-terminus of each respective VH. The linker in each case may be 36-80 amino acids in length and comprised of serine, glycine, alanine and threonine residues. The physically conjoined N-terminal light chains interface with the N-terminal VH-CH1 domains of each heavy chain via the VH/VL interaction and Ckappa or Clambda interaction with CH1. The native disulfide bond between the Ckappa or Clambda with CH1 provides additional stability between the light and heavy chains.

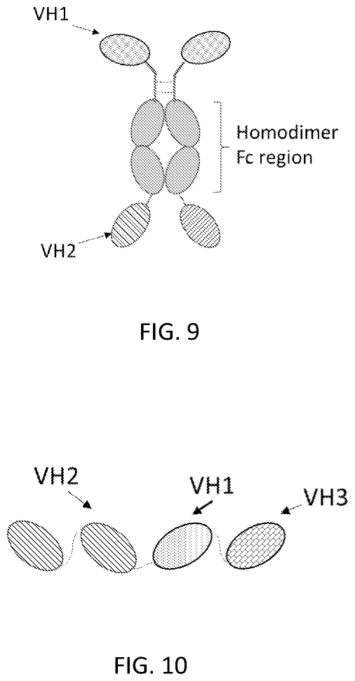

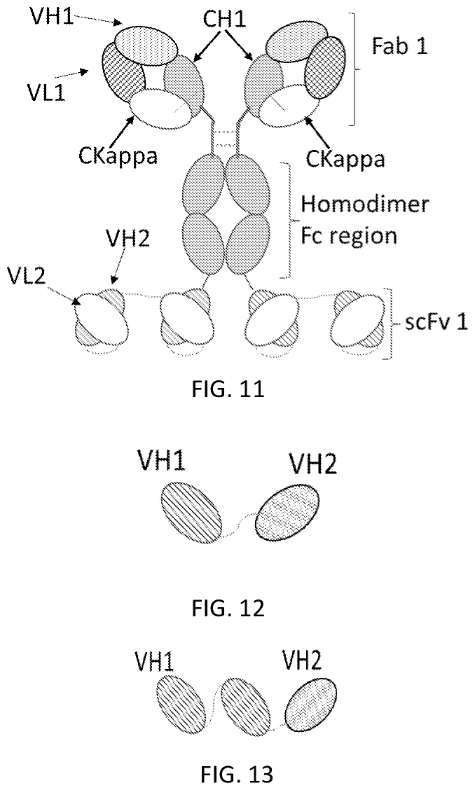

Bispecific molecules can also have a mixed format. This is illustrated, for example, in FIG. 9, FIG. 10, and FIG. 11.

For example, FIG. 9, illustrates a homodimer Fc based approach (see FIGS. 3, 4, and 5), combined with the moiety format selection of FIG. 7, whereby the total molecular valency is four, but specificity is restricted to two specificities. The N-terminus is comprised of two identical soluble VH3 germline based VH domains and the C-terminus is comprised of two identical soluble VH3 germlined based VH domains of different specificity to the N-terminal domains. Therefore, each specificity has a valence of two. Again, in this format, any of the antibody moieties at any of the four attachment/fusion points can be substituted with a non-antibody moiety, e.g., an effector binding/modulating moiety that does not comprise an antibody molecule.

FIG. 10 illustrates an example whereby the molecule is comprised of four VH3 germline based soluble VH domains. The first two domains have the same specificity (for example an inhibitory receptor), the 3rd domain from the N-terminus may have specificity for a tissue antigen and the fourth domain from the N-terminus may have specificity for human serum albumin (HSA), thereby granting the molecule extended half-life in the absence of an Ig Fc domain. Three glycine, serine, alanine and/or threonine rich linkers exists between domains 1 and 2, domains 2 and 3, and domains 3 and 4. This format may be configured with up to tetraspecificity, but monovalent in each case, or to have bispecificity with bivalency in each case. The order of domains can be changed. Again, in this format, any of the antibody moieties can be substituted with a non-antibody moiety, e.g., a effector binding/modulating moiety that does not comprise an antibody molecule.

FIG. 11 illustrates yet another approach. This example is similar to FIGS. 3 and 4, in that it is Fc homodimer based with two identical Fab units (bivalent monospecificity) at the N-terminus of the molecule. This example differs from FIGS. 3 and 4 in that the C-terminus of each heavy chain is appended with a tandem-scFv. Thus, in each case the C-terminus of the CH3 domain of the Fc is linked via a glycine/serine/alanine/threonine based linker to the N-terminus of a first VH domain, which is linked via the C-terminus by a 12-15 amino acid glycine/serine rich linker to the N-terminus of a first VL domain, which linked via a 25-35 amino acid glycine/serine/alanine/threonine based linker at the C-terminus to the N-terminus of a second VH domain, which is linked via the C-terminus with a 12-15 amino acid glycine/serine based linker to the N-terminus of a 2nd VL domain. In this Fc homodimer based molecule there are therefore two identical tandem scFvs at the C-terminus of the molecule offering either tetravalency for a single tissue antigen for example or bivalency to two different molecules. This format could also be adapted with a heterodimer Fc core allowing two different tandem-scFvs at the C-terminus of the Fc allowing for monovalent tetraspecificity at the C-terminus while retaining either bivalent monospecificity at the N-terminus or monovalent bispecificity at the N-terminal via usage of single chain Fab configurations as in FIGS. 5, 6, and 7. This molecule can therefore be configured to have 2, 3, 4, 5, or 6 specificities. The domain order of scFvs within the tandem-scFv units may be configured to be from N- to C-terminus either VH-Linker-VL or VL-Linker-VH. Again, in this format, any of the antibody moieties at any of the four attachment/fusion points can be substituted with a non-antibody moiety, e.g., an effector binding/modulating moiety that does not comprise an antibody molecule.

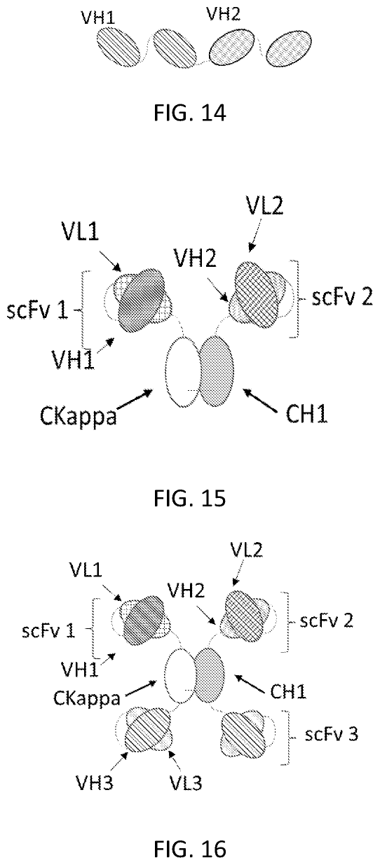

Bispecific antibodies can also be constructed to have, for example, shorter systemic PK while having increased tissue penetration. These types of antibodies can be based upon, for example, a human VH3 based domain antibody format. These are illustrated, for example, in FIGS. 12, 13, and 14. FIGS. 12, 13, and 14 each comprised a soluble VH3 germline family based VH domain modules. Each domain is approximately 12.5 kDa allowing for a small overall MW, which, without being bound to any particular theory, should be beneficial for enhanced tissue penetration. In these examples, none of the VH domains recognize any half-life extending targets such as FcRn or HSA. As illustrated in FIG. 12, the molecule is comprised of two VH domains joined with a flexible hydrophilic glycine/serine based linker between the C-terminus of the first domain and N-terminus of the second domain. In this example one domain may recognize a T cell costimulatory receptor and the second may recognize a tissue tethering antigen. As illustrated in FIG. 13, the molecule is comprised of three VH domains with N-C-terminal linkages of hydrophilic glycine/serine based linkers. The molecule may be configured to be trispecific but monovalent for each target. It may be bispecific with bivalency for one target and monovalency for another. As illustrated in FIG. 14, the molecule is comprised of four VH domains with N-C-terminal glycine/serine rich linkers between each domain. This molecule may be configured to be tetraspecific, trispecific, or bispecific with varying antigenic valencies in each case. Again, in this format, any of the antibody moieties at can be substituted with a non-antibody moiety, e.g., a effector binding/modulating moiety that does not comprise an antibody molecule.

Other embodiments of bispecific antibodies are illustrated in FIGS. 15 and 16. FIGS. 15 and 16 are comprised of the naturally heterodimerizing core of the human IgG CH1/Ckappa interface, including the C-terminal heavy/light disulfide bond which covalently anchors the interaction. This format does not contain an Fc or any moieties for half-life extension. As illustrated in FIG. 15, the molecule, at the N-terminus of the Ckappa domain is appended with an scFv fragment consisting of an N-terminal VH domain, linked at its C-terminus to the N-terminus of a VL domain via a 12-15 amino acid glycine/serine based linker, which is linked by its C-terminus to the N-terminus of the Ckappa domain via the native VL-Ckappa elbow sequence. The CH1 domain is appended at the N-terminus with an scFv fragment consisting of an N-terminal VL domain linked at its C-terminus via a 12-15 amino acid glycine/serine linker to the N-terminus of a VH domain, which is linked at its C-terminus to the N-terminus of the CH1 domains via the natural VH-CH1 elbow sequence. As illustrated in FIG. 16, the molecule has the same N-terminal configuration to Example 13. However the C-terminus of the Ckappa and CH1 domains are appended with scFv modules which may be in either the VH-VL or VL-VH configuration and may be either specific for the same antigen or specific for two different antigens. The VH/VL inter-domain linkers may be 12-15 amino acids in length and consisting of glycine/serine residues. The scFv binding sub-units may be swapped for soluble VH domains, or peptide recognition elements, or even tandem-scFv elements. This approach can also be configured to use Vlambda and/or Clambda domains. Again, in this format, any of the antibody moieties at any of the attachment/fusion points can be substituted with a non-antibody moiety, e.g., a effector binding/modulating moiety that does not comprise an antibody molecule.

FIG. 17 illustrates another embodiment. FIG. 17 represents a tandem scFv format consisting of a first N-terminal VL domain linked at its C-terminus to the N-terminus of a first VH domain with a 12-15 amino acid glycine/serine rich linker, followed at the first VH C-terminus by a 25-30 amino acid glycine/serine/alanine/threonine based linker to the N-terminus of a second VL domain. The second VL domain is linked at the C-terminus to the N-terminus of a 2nd VH domain by a 12-15 amino acid glycine/serine linker. Each scFv recognizes a different target antigen such as a costimulatory T cell molecule and a tissue tethering target. Again, in this format, any of the antibody moieties can be substituted with a non-antibody moiety, e.g., a effector binding/modulating moiety that does not comprise an antibody molecule.

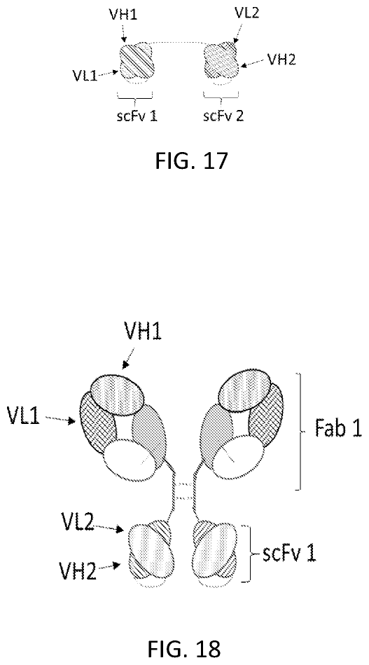

FIG. 18 illustrates another embodiment. FIG. 18 is a F(ab′)2 scFv fusion. This consists of two identical Fab components joined via two disulfide bonds in the native human IgG1 hinge region C-terminal of the human IgG CH1 domain. The human IgG1 CH2 and CH3 domains are absent. At the C-terminus of heavy chains 1 and 2 are two identical scFv fragments linked via a glycine/serine/alanine/threonine rich linker to the C-terminus of the huIgG1 hinge region. In the configuration shown, the VH is N-terminal in each scFv unit and linked via a 12-15 amino acid glycine/serine rich linker to the N-terminus of a VL domain. An alternative configuration would be N-term-VL-Linker-VH-C-term. In this design, the construct is bispecific with bivalency for reach target. Again, in this format, any of the antibody moieties at any of the four attachment/fusion points can be substituted with a non-antibody moiety, e.g., a effector binding/modulating moiety that does not comprise an antibody molecule.

As provided herein, the effector moiety that is linked or associate with can be a PD-1 agonist, a IL-2 mutein, or a CD39 molecule. A CD39 molecule, as that term is used herein, refers to a polypeptide having sufficient CD39 sequence that, as part of a therapeutic compound, phosphohydrolyzes ATP to AMP. In some embodiments, a CD39 molecule phosphohydrolizes ATP to AMP equivalent to, or at least, 10, 20, 30, 40, 50, 60, 70, 80, 90, or 95% of the rate of a naturally occurring CD39, e.g., the CD39 from which the CD39 molecule was derived. In some embodiments, a CD39 molecule has at least 60, 70, 80, 90, 95, 99, or 100% sequence identity, or substantial sequence identity, with a naturally occurring CD39.

Any functional isoform can be used (with CD39 or other proteins discussed herein). Exemplary CD39 sequence include Genbank accession #NP_001767.3 or a mature form from the following sequence:

(SEQ ID NO: 1)
MEDTKESNVKTFCSKNILAILGFSSIIAVIALLAVGLTQNKALPENVKYG
IVLDAGSSHTSLYIYKWPAEKENDTGVVHQVEECRVKGPGISKFVQKVNE
IGIYLTDCMERAREVIPRSQHQETPVYLGATAGMRLLRMESEELADRVLD
VVERSLSNYPFDFQGARIITGQEEGAYGWITINYLLGKFSQKTRWFSIVP
YETNNQETFGALDLGGASTQVTFVPQNQTIESPDNALQFRLYGKDYNVYT
HSFLCYGKDQALWQKLAKDIQVASNEILRDPCFHPGYKKVVNVSDLYKTP
CTKRFEMTLPFQQFEIQGIGNYQQCHQSILELFNTSYCPYSQCAFNGIFL
PPLQGDFGAFSAFYFVMKFLNLTSEKVSQEKVTEMMKKFCAQPWEEIKTS
YAGVKEKYLSEYCFSGTYILSLLLQGYHFTADSWEHIHFIGKIQGSDAGW
TLGYMLNLTNMIPAEQPLSTPLSHSTYVFLMVLFSLVLFTVAIIGLLIFH
KPSYFWKDMV.

In some embodiments, a CD39 molecule comprises a soluble catalytically active form of CD39 found to circulate in human or murine serum, see, e.g., Metabolism of circulating ADP in the bloodstream is mediated via integrated actions of soluble adenylate kinase-1 and NTPDase1/CD39 activities, Yegutkin et al. FASEB J. 2012 September; 26(9):3875-83. A soluble recombinant CD39 fragment is also described in Inhibition of platelet function by recombinant soluble ecto-ADPase/CD39, Gayle, et al., J Clin Invest. 1998 May 1; 101(9): 1851-1859.

In some embodiments, the CD39 effector domain comprises a sequence of:

(murine CD39, SEQ ID NO: 470)
TQNKPLPENVKYGIVLDAGSSHTNLYIYKWPAEKENDTGVVQQLEECQVK
GPGISKYAQKTDEIGAYLAECMELSTELIPTSKHHQTPVYLGATAGMRLL
RMESEQSADEVLAAVSTSLKGYPFDFQGAKIITGQEEGAYGWITINYLLG
RFTQEQSWLSLISDSQKQETFGALDLGGASTQITFVPQNSTIESPENSLQ
FRLYGEDYTVYTHSFLCYGKDQALWQKLAKDIQVSSGGVLKDPCFNPGYE
KVVNVSELYGTPCTERFEKKLPFDQFRIQGTGDYEQCHQSILELFNNSHC
PYSQCAFNGVFLPPLHGSFGAFSAFYFVMDFFKKVAKNSVISQEKMTEIT
KNFCSKSWEETKTSYPSVKEKYLSEYCFSGAYILSLLQGYNFTDSSWEQI
HFMGKIKDSNAGWTLGYMLNLTNMIPAEQPLSPPLPHSTYI;
or
(human CD39, SEQ ID NO: 471)
TQNKALPENVKYGIVLDAGSSHTSLYIYKWPAEKENDTGVVHQVEECRVK
GPGISKFVQKVNEIGIYLTDCMERAREVIPRSQHQETPVYLGATAGMRLL
RMESEELADRVLDVVERSLSNYPFDFQGARIITGQEEGAYGWITINYLLG
KFSQKTRWFSIVPYETNNQETFGALDLGGASTQVTFVPQNQTIESPDNAL
QFRLYGKDYNVYTHSFLCYGKDQALWQKLAKDIQVASNEILRDPCFHPGY
KKVVNVSDLYKTPCTKRFEMTLPFQQFEIQGIGNYQQCHQSILELFNTSY
CPYSQCAFNGIFLPPLQGDFGAFSAFYFVMKFLNLTSEKVSQEKVTEMMK
KFCAQPWEEIKTSYAGVKEKYLSEYCFSGTYILSLLLQGYHFTADSWEHI
HFIGKIQGSDAGWTLGYMLNLTNMIPAEQPLSTPLSHSTYV;
or
(human CD39 (N′d8), SEQ ID NO: 472)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLLQGYHFTADSWEHIHFIGKIQGSDAGWTLGYMLNLTNMIPAEQ
PLSTPLSHSTYV;
or
(human CD39 (E174A), SEQ ID NO: 473)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEAGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLLQGYHFTADSWEHIHFIGKIQGSDAGWTLGYMLNLTNMIPAEQ
PLSTPLSHSTYV;
or
(human CD39 (F305S), SEQ ID NO: 474)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRSEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLLQGYHFTADSWEHIHFIGKIQGSDAGWTLGYMLNLTNMIPAEQ
PLSTPLSHSTYV;
or
(human CD39 (L309E), SEQ ID NO: 475)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTEPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLLQGYHFTADSWEHIHFIGKIQGSDAGWTLGYMLNLTNMIPAEQ
PLSTPLSHSTYV;
or
(human CD39 (F314T), SEQ ID NO: 476)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTLPFQQTEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLLQGYHFTADSWEHIHFIGKIQGSDAGWTLGYMLNLTNMIPAEQ
PLSTPLSHSTYV;
or
(human CD39 (F414R), SEQ ID NO: 478)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCRSG
TYILSLLLQGYHFTADSWEHIHFIGKIQGSDAGWTLGYMLNLTNMIPAEQ
PLSTPLSHSTYV;
or
(human CD39 (L420S), SEQ ID NO: 479)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYISSLLLQGYHFTADSWEHIHFIGKIQGSDAGWTLGYMLNLTNMIPAEQ
PLSTPLSHSTYV;
or
(human CD39 (L424S, SEQ ID NO: 480)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLSQGYHFTADSWEHIHFIGKIQGSDAGWTLGYMLNLTNMIPAEQ
PLSTPLSHSTYV

In some embodiments, the CD39 sequence comprises mutations. In some embodiments, the mutations are insertions, deletions, or substitutions. In some embodiments, the mutations are insertions, deletions, or substitutions as compared to SEQ ID NO: 470 or 471. In some embodiments, the CD39 sequence comprises F305S, L309E, F314T, F314T, F414R, L420S, or L424S mutations. CD39 can also be referred to as ENTPD1. Other members of this gene family can also be used as an effector molecule, such as ENTPD1, ENTPD2, ENTPD3, ENTPD4, ENTPD5, ENTPD6, ENTPD7, ENTPD8, or ENTPD9.

In some embodiments, the effector molecule is an ENTPD2 polypeptide, which comprises the sequence of:

(SEQ ID NO: 481)
TRDVREPPALKYGIVLDAGSSHTSMFIYKWPADKENDTGIVGQHSSCDVP
GGGISSYADNPSGASQSLVGCLEQALQDVPKERHAGTPLYLGATAGMRLL
NLTNPEASTSVLMAVTHTLTQYPFDFRGARILSGQEEGVFGWVTANYLLE
NFIKYGWVGRWFRPRKGTLGAMDLGGASTQITFETTSPAEDRASEVQLHL
YGQHYRVYTHSFLCYGRDQVLQRLLASALQTHGFHPCWPRGFSTQVLLGD
VYQSPCTMAQRPQNFNSSARVSLSGSSDPHLCRDLVSGLFSFSSCPFSRC
SFNGVFQPPVAGNFVAFSAFFYTVDFLRTSMGLPVATLQQLEAAAVNVCN
QTWAQLQARVPGQRARLADYCAGAMFVQQLLSRGYGFDERAFGGVIFQKK
AADTAVGWALGYMLNLTNLIPADPPGLRKGTDFS

In some embodiments, the effector molecule is an ENTPD3 polypeptide, which comprises the sequence of:

(SEQ ID NO: 482)
QIHKQEVLPPGLKYGIVLDAGSSRTTVYVYQWPAEKENNTGVVSQTFKCS
VKGSGISSYGNNPQDVPRAFEECMQKVKGQVPSHLHGSTPIHLGATAGMR
LLRLQNETAANEVLESIQSYFKSQPFDFRGAQIISGQEEGVYGWITANYL
MGNFLEKNLWHMWVHPHGVETTGALDLGGASTQISFVAGEKMDLNTSDIM
QVSLYGYVYTLYTHSFQCYGRNEAEKKFLAMLLQNSPTKNHLTNPCYPRD
YSISFTMGHVFDSLCTVDQRPESYNPNDVITFEGTGDPSLCKEKVASIFD
FKACHDQETCSFDGVYQPKIKGPFVAFAGFYYTASALNLSGSFSLDTFNS
STWNFCSQNWSQLPLLLPKFDEVYARSYCFSANYIYHLFVNGYKFTEETW
PQIHFEKEVGNSSIAWSLGYMLSLINQIPAESPLIRLPIEPP

In some embodiments, the effector molecule is an ENTPD4 polypeptide, which comprises the sequence of:

(SEQ ID NO: 483)
KVEATSVLLPTDIKFGIVFDAGSSHTSLFLYQWLANKENGTGVVSQALAC
QVEGPGISSYTSNAAQAGESLQGCLEEALVLIPEAQHRKTPTFLGATAGM
RLLSRKNSSQARDIFAAVTQVLGRSPVDFWGAELLAGQAEGAFGWITVNY
GLGTLVKYSFTGEWIQPPEEMLVGALDMGGASTQITFVPGGPILDKSTQA
DFRLYGSDYSVYTHSYLCFGRDQMLSRLLVGLVQSRPAALLRHPCYLSGY
QTTLALGPLYESPCVHATPPLSLPQNLTVEGTGNPGACVSAIRELFNFSS
CQGQEDCAFDGVYQPPLRGQFYAFSNFYYTFHFLNLTSRQPLSTVNATIW
EFCQRPWKLVEASYPGQDRWLRDYCASGLYILTLLHEGYGFSEETWPSLE
FRKQAGGVDIGWTLGYMLNLTGMIPADAPAQWRAESYGVWVAK

The catalytic domains of these proteins can also be used in place of the sequences above. The catalytic domain of CD39 is referred to as the CD39 Effector Domain herein.

Elevated risk, as used herein, refers to the risk of a disorder in a subject, wherein the subject has one or more of (1) a medical history of the disorder or a symptom of the disorder, (2) a biomarker associated with the disorder or a symptom of the disorder, or (3) a family history of the disorder or a symptom of the disorder.

Sequence identity, percentage identity, and related terms, as those terms are used herein, refer to the relatedness of two sequences, e.g., two nucleic acid sequences or two amino acid or polypeptide sequences. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the naturally occurring sequence, or are encoded by a substantially identical nucleotide sequence, and are capable of having one or more activities of the naturally occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to for example any a nucleic acid sequence provided herein. BLAST protein searches can be performed with the XBLAST program, score=50, wordlength=3 to obtain amino acid sequences homologous to protein molecules provided herein. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules and compounds of the present embodiments may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally occurring amino acids. Exemplary amino acids include naturally occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some embodiments, the molecule comprises a CD39 molecule.

Specific targeting moiety, as that term is used herein, refers to donor specific targeting moiety or a tissue specific targeting moiety.

Subject, as that term is used herein, refers to a mammalian subject, e.g., a human subject. In some embodiments, the subject is a non-human mammal, e.g., a horse, dog, cat, cow, goat, or pig.

Target ligand binding molecule, as used herein, refers to a polypeptide that has sufficient sequence from a naturally occurring counter ligand of a target ligand that it can bind the target ligand on a target tissue (e.g., donor tissue or subject target tissue) with sufficient specificity that it can serve as a specific targeting moiety. In some embodiments, a target ligand binding molecule binds to target tissue or cells with at least 10, 20, 30, 40, 50, 60, 70, 80, 90, or 95% of the affinity of the naturally occurring counter ligand. In some embodiments, a target ligand binding molecule has at least 60, 70, 80, 90, 95, 99, or 100% sequence identity, or substantial sequence identity, with a naturally occurring counter ligand for the target ligand.

Target site, as that term is used herein, refers to a site which contains the entity, e.g., epitope, bound by a targeting moiety. In some embodiments, the target site is the site at which immune privilege is established.

Tissue specific targeting moiety, as that term is used herein, refers to a moiety, e.g., an antibody molecule, that as a component of a therapeutic molecule, localizes the therapeutic molecule preferentially to a target tissue, as opposed to other tissue of a subject. As a component of a therapeutic compound, the tissue specific targeting moiety provides site-specific immune privilege for a target tissue, e.g., an organ or tissue undergoing or at risk for autoimmune attack. In some embodiments, a tissue specific targeting moiety binds to a product, e.g., a polypeptide product, which is not present outside the target tissue, or is present at sufficiently low levels that, at therapeutic concentrations of therapeutic molecule, unacceptable levels of immune suppression are absent or substantially absent. In some embodiments, a tissue specific targeting moiety binds to an epitope, which epitope is not present outside, or not substantially present outside, the target site.

In some embodiments, a tissue specific targeting moiety, as a component of a therapeutic compound, preferentially binds to a target tissue or target tissue antigen, e.g., has a binding affinity for the target tissue or antigen that is greater for target antigen or tissue, e.g., at least 2, 4, 5, 10, 50, 100, 500, 1,000, 5,000, or 10,000 fold greater, than its affinity for non-target tissue or antigen present outside the target tissue. Affinity of a therapeutic compound of which the tissue specific moiety is a component, can be measured in a cell suspension, e.g., the affinity for suspended cells having the target antigen is compared with its affinity for suspended cells not having the target antigen. In some embodiments, the binding affinity for the target antigen bearing cells is below 10 nM.

In some embodiments, the binding affinity for the target antigen bearing cells is below 100 pM, 50 pM, or 10 pM. In some embodiments, the specificity for a target antigen is sufficient, that when the tissue specific targeting moiety is coupled to an immune down regulating effector: i) immune attack of the target tissue, e.g., as measured by histological inflammatory response, infiltrating T effector cells, or organ function, in the clinical setting, e.g., creatinine for kidney, is substantially reduced, e.g., as compared to what would be seen in an otherwise similar implant but lacking the tissue specific targeting moiety is coupled to an immune down regulating effector; and/or ii) immune function in the recipient, outside or away from the target tissue, is substantially maintained.

In some embodiments, one or more of the following is seen: at therapeutic levels of therapeutic compound, peripheral blood lymphocyte counts are not substantially impacted, e.g., the level of T cells is within 25, 50, 75, 85, 90, or 95% of normal, the level of B cells is within 25, 50, 75, 85, 90, or 95% of normal, and/or the level of granulocytes (PMN cells) is within 25, 50, 75, 85, 90, or 95% of normal, or the level of monocytes is within 25, 50, 75, 85, 90, or 95% of normal; at therapeutic levels of therapeutic compound, the ex vivo proliferative function of PBMCs against non-disease relevant antigens is substantially normal or is within 70, 80, or 90% of normal; at therapeutic levels of therapeutic compound, the incidence or risk of opportunistic infections and cancers associated with immunosuppression is not substantially increased over normal; or at therapeutic levels of therapeutic compound, the incidence or risk of opportunistic infections and cancers associated with immunosuppression is substantially less than would be seen with standard of care, or non-targeted, immunosuppression. In some embodiments, the tissue specific targeting moiety comprises an antibody molecule. In some embodiments, the donor specific targeting moiety comprises an antibody molecule, a target specific binding polypeptide, or a target ligand binding molecule. In some embodiments, the tissue specific targeting moiety binds a product, or a site on a product, that is present or expressed exclusively, or substantially exclusively, on target tissue.

### Inhibitory Immune Checkpoint Molecules

In some embodiments, the effector domain is an inhibitory immune checkpoint molecule ligand molecule. An “inhibitory immune checkpoint molecule ligand molecule”, as that term is used herein, refers to a polypeptide having sufficient inhibitory immune checkpoint molecule ligand sequence, e.g., in the case of a PD-L1 molecule, sufficient PD-L1 sequence, that when present as an ICIM binding/modulating moiety of a multimerized therapeutic compound, can bind and agonize its cognate inhibitory immune checkpoint molecule, e.g., again in the case of a PD-L1 molecule, PD-1.

In some embodiments, the inhibitory immune checkpoint molecule ligand molecule, e.g., a PD-L1 molecule, when binding as a monomer (or binding when the therapeutic compound is not multimerized), to its cognate ligand, e.g., PD-1, does not antagonize or substantially antagonize, or prevent binding, or prevent substantial binding, of an endogenous inhibitory immune checkpoint molecule ligand to the inhibitory immune checkpoint molecule. E.g., in the case of a PD-L1 molecule, the PD-L1 molecule does not antagonize binding of endogenous PD-L1 to PD-1.

In some embodiments, the inhibitory immune checkpoint molecule ligand when binding as a monomer, to its cognate inhibitory immune checkpoint molecule does not agonize or substantially agonize the inhibitory immune checkpoint molecule. By way of example, e.g., a PD-L1 molecule when binding to PD-1, does not agonize or substantially agonize PD-1.

In some embodiments, an inhibitory immune checkpoint molecule ligand molecule has at least 60, 70, 80, 90, 95, 99, or 100% sequence identity, or substantial sequence identity, with a naturally occurring inhibitory immune checkpoint molecule ligand.

Exemplary inhibitory immune checkpoint molecule ligand molecules include: a PD-L1 molecule, which binds to inhibitory immune checkpoint molecule PD-1, and in embodiments has at least 60, 70, 80, 90, 95, 99, or 100% sequence identity, or substantial sequence identity, with a naturally occurring PD-L1, e.g., the PD-L1 molecule comprising the sequence of MRIFAVFIFMTYWHLLNAFTVTVPKDLYVVEYGSNMTIECKFPVEKQLDLAALIVYWE MEDKNIIQFVHGEEDLKVQHSSYRQRARLLKDQLSLGNAALQITDVKLQDAGVYRCMI SYGGADYKRITVKVNAPYNKINQRILVVDPVTSEHELTCQAEGYPKAEVIWTSSDHQVL SGKTTTTNSKREEKLFNVTSTLRINTTTNEIFYCTFRRLDPEENHTAELVIPELPLAHPPNE RTHLVILGAILLCLGVALTFIFRLRKGRMMDVKKCGIQDTNSKKQSDTHLEET (SEQ ID NO: 3), or an active fragment thereof, in some embodiments, the active fragment comprises residues 19 to 290 of the PD-L1 sequence; a HLA-G molecule, which binds to any of inhibitory immune checkpoint molecules KIR2DL4, LILRB1, and LILRB2, and in embodiments has at least 60, 70, 80, 90, 95, 99, or 100% sequence identity, or substantial sequence identity, with a naturally occurring HLA-G. Exemplary HLA-G sequences include, e.g., a mature form found in the sequence at GenBank P17693.1 RecName: Full=HLA class I histocompatibility antigen, alpha chain G; AltName: Full=HLA G antigen; AltName: Full=MHC class I antigen G; Flags: Precursor, or in the sequence

(SEQ ID NO: 4)
MVVMAPRTLFLLLSGALTLTETWAGSHSMRYFSAAVSRPGRGEPRFIAMG
YVDDTQFVRFDSDSACPRMEPRAPWVEQEGPEYWEEETRNTKAHAQTDRM
NLQTLRGYYNQSEASSHTLQWMIGCDLGSDGRLLRGYEQYAYDGKDYLAL
NEDLRSWTAADTAAQISKRKCEAANVAEQRRAYLEGTCVEWLHRYLENGK
EMLQRADPPKTHVTHHPVFDYEATLRCWALGFYPAEIILTWQRDGEDQTQ
DVELVETRPAGDGTFQKWAAVVVPSGEEQRYTCHVQHEGLPEPLMLRWKQ
SSLPTIPIMGIVA.

In some embodiments, the anti-effector or inhibitory immune checkpoint molecule antibody molecule, when binding as a monomer (or binding when the therapeutic compound is not multimerized), to the effector or inhibitory immune checkpoint molecule, does not antagonize, substantially antagonize, prevent binding, or prevent substantial binding, of an endogenous counter ligand of the inhibitory immune checkpoint molecule to inhibitory immune checkpoint molecule. In some embodiments, the anti-effector or inhibitory immune checkpoint molecule antibody molecule when binding as a monomer (or binding when the therapeutic compound is not multimerized), to the inhibitory immune checkpoint molecule, does not agonize or substantially agonize, the effector or inhibitory molecule.

Exemplary inhibitory molecules (e.g., an inhibitory immune checkpoint molecule) (together with their counter ligands) can be found in Table 1. This table lists molecules to which exemplary ICIM binding moieties can bind.

TABLE 1
Cell surface inhibitory molecules, e.g., inhibitory
immune checkpoint molecules (column A), counter ligands
(column B) and cell types affected (column C).

| A | B | C |
|---|---|---|
| PD-1 | PD-L1, PD-L2 | T cells, B cells |
| Alkaline | | |
| phosphatase | | |
| B7-H3 | Unknown | T cells |
| B7-H4 | Neuropilin 1, | T cells |
| | Neuropilin 2, | |
| | Plexin4A | |
| BTLA | HVEM | T cells, B cells |
| CTLA-4 | CD80, CD86 | T cells |
| IDO1 | Tryptophan | Lymphocytes |
| IDO2 | Tryptophan | Lymphocytes |
| KIR2DL1, | HLA MHC class I | NK cells |
| KIR2DL2/3, | | |
| KIR3DL1, | | |
| KIR3DL2 | | |
| LAG3 | HLA MHC class II | T cells |
| TIM-3 | Galectin-9 | T cells |
| VISTA | Unknown | T cells, myeloid cells |
| TIGIT | CD155 | T cells |
| KIR2DL4 | HLA-G | NK cells |
| LILRB1 | HLA-G | T cells, NK cells, B cells, |
| | | monocytes, dendritic cells |
| LILRB2 | HLA-G | Monocytes, dendritic cells, |
| | | neutrophils, some tumor cells |
| NKG2A | Nonclassical MHC | T cells, NK cells |
| | Glycoproteins class I | |
| FCRL1-6 | FCRL1-2 not | B cells |
| | known | |
| | FCRL4 = IgA | |
| | FCRL5 = IgG | |
| | FCRL6 = MHC Class | |
| | II | |
| | BUTYROPHILINS, | Modulation of immune cells |
| | for example | |
| | BTN1A1, BTN2A2, | |
| | BTNL2, BTNL1, | |
| | BTNL8 | |

### The PD-L1/PD-1 Pathway

Programmed cell death protein 1, (often referred to as PD-1) is a cell surface receptor that belongs to the immunoglobulin superfamily. PD-1 is expressed on T cells and other cell types including, but not limited to, B cells, myeloid cells, dendritic cells, monocytes, T regulatory cells, iNK T cells. PD-1 binds two ligands, PD-L1 and PD-L2, and is an inhibitory immune checkpoint molecule. Engagement with a cognate ligand, PD-L1 or PD-L2, in the context of engagement of antigen loaded MHC with the T cell receptor on a T cell minimizes or prevents the activation and function of T cells. The inhibitory effect of PD-1 can include both promoting apoptosis (programmed cell death) in antigen specific T cells in lymph nodes and reducing apoptosis in regulatory T cells (suppressor T cells).

In some embodiments, a therapeutic compound comprises an ICIM binding/modulating moiety which agonizes PD-1 inhibition. An ICIM binding/modulating moiety can include an inhibitory molecule counter ligand molecule, e.g., comprising a fragment of a ligand of PD-1 (e.g., a fragment of PD-L1 or PD-L2) or another moiety, e.g., a functional antibody molecule, comprising, e.g., an scFv domain that binds PD-1.

In some embodiments, a therapeutic compound comprises a targeting moiety that is preferentially binds a donor antigen not present in, or present in substantially lower levels in the subject, e.g., a donor antigen from Table 2, and is localized to donor graft tissue in a subject. In some embodiments, it does not bind, or does not substantially bind, other tissues. In some embodiments, a therapeutic compound can include a targeting moiety that is specific for HLA-A2 and specifically binds donor allograft tissue but does not bind, or does not substantially bind, host tissues. In some embodiments, the therapeutic compound comprises an ICIM binding/modulating moiety, e.g., an inhibitory molecule counter ligand molecule, e.g., comprising a fragment of a ligand of PD-1 (e.g., a fragment of PD-L1 or PD-L2) or another moiety, e.g., a functional antibody molecule, comprising, e.g., an scFv domain that binds PD-1, such that the therapeutic compound, e.g., when bound to target, activates PD-1. The therapeutic compound targets an allograft and provides local immune privilege to the allograft.

In some embodiments, a therapeutic compound comprises a targeting moiety that is preferentially binds to an antigen of Table 3, and is localized to the target in a subject, e.g., a subject having an autoimmune disorder, e.g., an autoimmune disorder of Table 3. In some embodiments, it does not bind, or does not substantially bind, other tissues. In some embodiments, the therapeutic compound comprises an ICIM binding/modulating moiety, e.g., an inhibitory molecule counter ligand molecule, e.g., comprising a fragment of a ligand of PD-1 (e.g., a fragment of PD-L1 or PD-L2) or another moiety, e.g., a functional antibody molecule, comprising, e.g., an scFv domain that binds PD-1, such that the therapeutic compound, e.g., when bound to target, activates PD-1. The therapeutic compound targets a tissue subject to autoimmune attack and provides local immune privilege to the tissue.

PD-L1 and PDL2, or polypeptides derived therefrom, can provide candidate ICIM binding moieties. However, in monomer form, e.g., when the therapeutic compound is circulating in blood or lymph, this molecule could have an undesired effect of antagonizing the PD-L1/PD-1 pathway, and may only agonize the PD-1 pathway when clustered or multimerized on the surface of a target, e.g., a target organ. In some embodiments, a therapeutic compound comprises an ICIM binding/modulating moiety comprising a functional antibody molecule, e.g., a scFv domain, that is inert, or substantially inert, to the PD-1 pathway in a soluble form but which agonizes and drives an inhibitory signal when multimerized (by the targeting moiety) on the surface of a tissue.

IL-2 Mutein Molecules: IL-2 Receptor Binders that Activate Tregs

IL-2 mutein molecule, as that term is used herein, refers to an IL-2 variant that binds with high affinity to the CD25 (IL-2R alpha chain) and with low affinity to the other IL-2R signaling components CD122 (IL-2R beta) and CD132 (IL-2R gamma). Such an IL-2 mutein molecule preferentially activates Treg cells, In embodiments, either alone, or as a component of a therapeutic compound, an IL-2 mutein activates Tregs at least 2, 5, 10, or 100 fold more than cytotoxic or effector T cells. Exemplary IL-2 mutein molecules are described in WO2010085495, WO2016/164937, US2014/0286898A1, WO2014153111A2, WO2010/085495, cytotoxic WO2016014428A2, WO2016025385A1, and US20060269515. Muteins disclosed in these references that include additional domains, e.g., an Fc domain, or other domain for extension of half-life can be used in the therapeutic compounds and methods described herein without such additional domains. In another embodiment an IIC binding/modulating moiety comprises an IL-2 mutein, or active fragment thereof, coupled, e.g., fused, to another polypeptide, e.g., a polypeptide that extends in vivo half-life, e.g., an immunoglobulin constant region, or a multimer or dimer thereof, e.g., AMG 592. In an embodiment the therapeutic compound comprises the IL-2 portion of AMG 592. In an embodiment the therapeutic compound comprises the IL-2 portion but not the immunoglobulin portion of AMG 592. In some embodiments, the mutein does not comprise a Fc region. For some IL-2 muteins, the muteins are engineered to contain a Fc region because such region has been shown to increase the half-life of the mutein. In some embodiments, the extended half-life is not necessary for the methods described and embodied herein. In some embodiments, the Fc region that is fused with the IL-2 mutein comprises a N297 mutations, such as, but not limited to, N297A. In some embodiments, the Fc region that is fused with the IL-2 mutein does not comprise a N297 mutation, such as, but not limited to, N297A.

IL-2 mutein molecules that preferentially expand or stimulate Treg cells (over cytotoxic T cells) can be used as an IIC binding/modulating moiety.

In some embodiments, IIC binding/modulating moiety comprises a IL-2 mutein molecule. As used herein, the term “IL-2 mutein molecule” or “IL-2 mutein” refers to an IL-2 variant that preferentially activates Treg cells. In some embodiments, either alone, or as a component of a therapeutic compound, an IL-2 mutein molecule activates Tregs at least 2, 5, 10, or 100 fold more than cytotoxic T cells. A suitable assay for evaluating preferential activation of Treg cells can be found in U.S. Pat. No. 9,580,486 at, for example, Examples 2 and 3, or in WO2016014428 at, for example, Examples 3, 4, and 5, each of which is incorporated by reference in its entirety. The sequence of mature IL-2 is

(SEQ ID NO: 6)
APTSSSTKKTQLQLEHLLLDLQMILNGINNYKNPKLTRMLT
FKFYMPKKATELKHLQCLEEELKPLEEVLNLAQSKNFHLRP
RDLISNINVIVLELKGSETTFMCEYADETATIVEFLNRWITFC
QSIISTLT
(mature IL-2 sequence)

The immature sequence of IL-2 can be represented by

(SEQ ID NO: 15)
MYRMQLLSCIALSLALVTNSAPTSSSTKKTQLQLEHLLLDL
QMILNGINNYKNPKLTRMLTFKFYMPKKATELKHLQCLEE
ELKPLEEVLNLAQSKNFHLRPRDLISNINVIVLELKGSETTF
MCEYADETATIVEFLNRWITFCQSIISTLT.

In some embodiments, an IIC binding/modulating moiety comprises an IL-2 mutein, or active fragment thereof, coupled, e.g., fused, to another polypeptide, e.g., a polypeptide that extends in vivo half-life, e.g., an immunoglobulin constant region, or a multimer or dimer thereof.

An IL-2 mutein molecule can be prepared by mutating one or more of the residues of IL-2. Non-limiting examples of IL-2-muteins can be found in WO2016/164937, U.S. Pat. Nos. 9,580,486, 7,105,653, 9,616,105, 9,428,567, US2017/0051029, US2014/0286898A1, WO2014153111A2, WO2010/085495, WO2016014428A2, WO2016025385A1, and US20060269515, each of which are incorporated by reference in its entirety.

In some embodiments, the alanine at position 1 of the sequence above is deleted. In some embodiments, the IL-2 mutein molecule comprises a serine substituted for cysteine at position 125 of the mature IL-2 sequence. Other combinations of mutations and substitutions that are IL-2 mutein molecules are described in US20060269515, which is incorporated by reference in its entirety. In some embodiments, the cysteine at position 125 is also substituted with a valine or alanine. In some embodiments, the IL-2 mutein molecule comprises a V91K substitution. In some embodiments, the IL-2 mutein molecule comprises a N88D substitution. In some embodiments, the IL-2 mutein molecule comprises a N88R substitution. In some embodiments, the IL-2 mutein molecule comprises a substitution of H16E, D84K, V91N, N88D, V91K, or V91R, any combinations thereof. In some embodiments, these IL-2 mutein molecules also comprise a substitution at position 125 as described herein. In some embodiments, the IL-2 mutein molecule comprises one or more substitutions selected from the group consisting of: T3N, T3A, L12G, L12K, L12Q, L12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20H, D20I, D20Y, D20F, D20G, D20T, D20W, M23R, R81A, R81G, R81S, R81T, D84A, D84E, D84G, D84I, D84M, D84Q D84R, D84S, D84T, S87R, N88A, N88D, N88E, N88I, N88F, N88G, N88M, N88R, N88S, N88V, N88W, V91D, V91E, V91G, V91S, I92K, I92R, E95G, and Q126. In some embodiments, the amino acid sequence of the IL-2 mutein molecule differs from the amino acid sequence set forth in mature IL-2 sequence with a C125A or C125S substitution and with one substitution selected from T3N, T3A, L12G, L12K, L12Q L12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20F, D20G, D20T, D20W, M23R, R81A, R81G, R81S, R81T, D84A, D84E, D84G, D84I, D84M, D84Q, D84R, D84S, D84T, S87R, N88A, N88D, N88E, N88F, N88I, N88G, N88M, N88R, N88S, N88V, N88W, V91D, V91E, V91G, V91S, I92K, I92R, E95G, Q126I, Q126L, and Q126F. In some embodiments, the IL-2 mutein molecule differs from the amino acid sequence set forth in mature IL-2 sequence with a C125A or C125S substitution and with one substitution selected from D20H, D20I, D20Y, D20E, D20G, D20W, D84A, D84S, H16D, H16G, H16K, H16R, H16T, H16V, I92K, I92R, L12K, L19D, L19N, L19T, N88D, N88R, N88S, V91D, V91G, V91K, and V91S. In some embodiments, the IL-2 mutein comprises N88R and/or D20H mutations.

In some embodiments, the IL-2 mutein molecule comprises a mutation in the polypeptide sequence at a position selected from the group consisting of amino acid 30, amino acid 31, amino acid 35, amino acid 69, and amino acid 74. In some embodiments, the mutation at position 30 is N30S. In some embodiments, the mutation at position 31 is Y31H. In some embodiments, the mutation at position 35 is K35R. In some embodiments, the mutation at position 69 is V69A. In some embodiments, the mutation at position 74 is Q74P. In some embodiments, the mutein comprises a V69A mutation, a Q74P mutation, a N88D or N88R mutation, and one or more of L53I, L56I, L80I, or L118I mutations. In some embodiments, the mutein comprises a V69A mutation, a Q74P mutation, a N88D or N88R mutation, and a L to I mutation selected from the group consisting of: L53I, L56I, L80I, and L118I mutation. In some embodiments, the IL-2 mutein comprises a V69A, a Q74P, a N88D or N88R mutation, and a L53I mutation. In some embodiments, the IL-2 mutein comprises a V69A, a Q74P, a N88D or N88R mutation, and a L56I mutation. In some embodiments, the IL-2 mutein comprises a V69A, a Q74P, a N88D or N88R mutation, and a L80I mutation. In some embodiments, the IL-2 mutein comprises a V69A, a Q74P, a N88D or N88R mutation, and a L118I mutation. As provided for herein, the muteins can also comprise a C125A or C125S mutation.

In some embodiments, the IL-2 mutein molecule comprises a substitution selected from the group consisting of: N88R, N88I, N88G, D20H, D109 C, Q126L, Q126F, D84G, or D84I relative to mature human IL-2 sequence provided above. In some embodiments, the IL-2 mutein molecule comprises a substitution of D109C and one or both of a N88R substitution and a C125S substitution. In some embodiments, the cysteine that is in the IL-2 mutein molecule at position 109 is linked to a polyethylene glycol moiety, wherein the polyethylene glycol moiety has a molecular weight of between 5 and 40 kDa.

In some embodiments, any of the substitutions described herein are combined with a substitution at position 125. The substitution can be a C125S, C125A, or C125V substitution.

In addition to the substitutions or mutations described herein, in some embodiments, the IL-2 mutein has a substitution/mutation at one or more of positions 73, 76, 100, or 138 that correspond to SEQ ID NO: 15 or positions at one or more of positions 53, 56, 80, or 118 that correspond to SEQ ID NO: 6. In some embodiments, the IL-2 mutein comprises a mutation at positions 73 and 76; 73 and 100; 73 and 138; 76 and 100; 76 and 138; 100 and 138; 73, 76, and 100; 73, 76, and 138; 73, 100, and 138; 76, 100 and 138; or at each of 73, 76, 100, and 138 that correspond to SEQ ID NO: 15. In some embodiments, the IL-2 mutein comprises a mutation at positions 53 and 56; 53 and 80; 53 and 118; 56 and 80; 56 and 118; 80 and 118; 53, 56, and 80; 53, 56, and 118; 53, 80, and 118; 56, 80 and 118; or at each of 53, 56, 80, and 118 that correspond to SEQ ID NO: 6. As the IL-2 can be fused or tethered to other proteins, as used herein, the term corresponds to as reference to a SEQ ID NOs: 6 or 15 refer to how the sequences would align with default settings for alignment software, such as can be used with the NCBI website. In some embodiments, the mutation is leucine to isoleucine. Thus, the IL-2 mutein can comprise one more isoleucines at positions 73, 76, 100, or 138 that correspond to SEQ ID NO: 15 or positions at one or more of positions 53, 56, 80, or 118 that correspond to SEQ ID NO: 6. In some embodiments, the mutein comprises a mutation at L53 that correspond to SEQ ID NO: 6. In some embodiments, the mutein comprises a mutation at L56 that correspond to SEQ ID NO: 6. In some embodiments, the mutein comprises a mutation at L80 that correspond to SEQ ID NO: 6. In some embodiments, the mutein comprises a mutation at L118 that correspond to SEQ ID NO: 6. In some embodiments, the mutation is leucine to isoleucine. In some embodiments, the mutein also comprises a mutation as position 69, 74, 88, 125, or any combination thereof in these muteins that correspond to SEQ ID NO: 6. In some embodiments, the mutation is a V69A mutation. In some embodiments, the mutation is a Q74P mutation. In some embodiments, the mutation is a N88D or N88R mutation. In some embodiments, the mutation is a C125A or C125S mutation.

In some embodiments, the IL-2 mutein comprises a mutation at one or more of positions 49, 51, 55, 57, 68, 89, 91, 94, 108, and 145 that correspond to SEQ ID NO: 15 or one or more positions 29, 31, 35, 37, 48, 69, 71, 74, 88, and 125 that correspond to SEQ ID NO: 6. The substitutions can be used alone or in combination with one another. In some embodiments, the IL-2 mutein comprises substitutions at 2, 3, 4, 5, 6, 7, 8, 9, or each of positions 49, 51, 55, 57, 68, 89, 91, 94, 108, and 145. Non-limiting examples such combinations include, but are not limited to, a mutation at positions 49, 51, 55, 57, 68, 89, 91, 94, 108, and 145; 49, 51, 55, 57, 68, 89, 91, 94, and 108; 49, 51, 55, 57, 68, 89, 91, and 94; 49, 51, 55, 57, 68, 89, and 91; 49, 51, 55, 57, 68, and 89; 49, 51, 55, 57, and 68; 49, 51, 55, and 57; 49, 51, and 55; 49 and 51; 51, 55, 57, 68, 89, 91, 94, 108, and 145; 51, 55, 57, 68, 89, 91, 94, and 108; 51, 55, 57, 68, 89, 91, and 94; 51, 55, 57, 68, 89, and 91; 51, 55, 57, 68, and 89; 55, 57, and 68; 55 and 57; 55, 57, 68, 89, 91, 94, 108, and 145; 55, 57, 68, 89, 91, 94, and 108; 55, 57, 68, 89, 91, and 94; 55, 57, 68, 89, 91, and 94; 55, 57, 68, 89, and 91; 55, 57, 68, and 89; 55, 57, and 68; 55 and 57; 57, 68, 89, 91, 94, 108, and 145; 57, 68, 89, 91, 94, and 108; 57, 68, 89, 91, and 94; 57, 68, 89, and 91; 57, 68, and 89; 57 and 68; 68, 89, 91, 94, 108, and 145; 68, 89, 91, 94, and 108; 68, 89, 91, and 94; 68, 89, and 91; 68 and 89; 89, 91, 94, 108, and 145; 89, 91, 94, and 108; 89, 91, and 94; 89 and 91; 91, 94, 108, and 145; 91, 94, and 108; 91, and 94; or 94 and 108. Each mutation can be combined with one another. The same substitutions can be made in SEQ ID NO: 6, but the numbering would adjusted appropriately as is clear from the present disclosure (20 less than the numbering for SEQ ID NO: 15 corresponds to the positions in SEQ ID NO: 6).

In some embodiments, the IL-2 mutein comprises a mutation at one or more positions of 35, 36, 42, 104, 115, or 146 that correspond to SEQ ID NO: 15 or the equivalent positions at SEQ ID NO: 6 (e.g., positions 15, 16, 22, 84, 95, or 126). These mutations can be combined with the other leucine to isoleucine mutations described herein or the mutation at positions 73, 76, 100, or 138 that correspond to SEQ ID NO: 15 or at one or more of positions 53, 56, 80, or 118 that correspond to SEQ ID NO: 6. In some embodiments, the mutation is a E35Q, H36N, Q42E, D104N, E115Q, or Q146E, or any combination thereof. In some embodiments, one or more of these substitutions is wild-type. In some embodiments, the mutein comprises a wild-type residue at one or more of positions 35, 36, 42, 104, 115, or 146 that correspond to SEQ ID NO: 15 or the equivalent positions at SEQ ID NO: 6 (e.g., positions 15, 16, 22, 84, 95, and 126).

The mutations at these positions can be combined with any of the other mutations described herein, including, but not limited to substitutions at positions 73, 76, 100, or 138 that correspond to SEQ ID NO: 15 or positions at one or more of positions 53, 56, 80, or 118 that correspond to SEQ ID NO: 6 described herein and above. In some embodiments, the IL-2 mutein comprises a N49S mutation that corresponds to SEQ ID NO: 15. In some embodiments, the IL-2 mutein comprises a Y51S or a Y51H mutation that corresponds to SEQ ID NO: 15. In some embodiments, the IL-2 mutein comprises a K55R mutation that corresponds to SEQ ID NO: 15. In some embodiments, the IL-2 mutein comprises a T57A mutation that corresponds to SEQ ID NO: 15. In some embodiments, the IL-2 mutein comprises a K68E mutation that corresponds to SEQ ID NO: 15. In some embodiments, the IL-2 mutein comprises a V89A mutation that corresponds to SEQ ID NO: 15. In some embodiments, the IL-2 mutein comprises a N91R mutation that corresponds to SEQ ID NO: 15. In some embodiments, the IL-2 mutein comprises a Q94P mutation that corresponds to SEQ ID NO: 15. In some embodiments, the IL-2 mutein comprises a N108D or a N108R mutation that corresponds to SEQ ID NO: 15. In some embodiments, the IL-2 mutein comprises a C145A or C145S mutation that corresponds to SEQ ID NO: 15. These substitutions can be used alone or in combination with one another. In some embodiments, the mutein comprises each of these substitutions. In some embodiments, the mutein comprises 1, 2, 3, 4, 5, 6, 7, or 8 of these mutations. In some embodiments, the mutein comprises a wild-type residue at one or more of positions 35, 36, 42, 104, 115, or 146 that correspond to SEQ ID NO: 15 or the equivalent positions at SEQ ID NO: 6 (e.g. positions 15, 16, 22, 84, 95, and 126).

In some embodiments, the IL-2 mutein comprises a N29S mutation that corresponds to SEQ ID NO: 6. In some embodiments, the IL-2 mutein comprises a Y31S or a Y31H mutation that corresponds to SEQ ID NO: 6. In some embodiments, the IL-2 mutein comprises a K35R mutation that corresponds to SEQ ID NO: 6. In some embodiments, the IL-2 mutein comprises a T37A mutation that corresponds to SEQ ID NO: 6. In some embodiments, the IL-2 mutein comprises a K48E mutation that corresponds to SEQ ID NO: 6. In some embodiments, the IL-2 mutein comprises a V69A mutation that corresponds to SEQ ID NO: 6. In some embodiments, the IL-2 mutein comprises a N71R mutation that corresponds to SEQ ID NO: 6. In some embodiments, the IL-2 mutein comprises a Q74P mutation that corresponds to SEQ ID NO: 6. In some embodiments, the IL-2 mutein comprises a N88D or a N88R mutation that corresponds to SEQ ID NO: 6. In some embodiments, the IL-2 mutein comprises a C125A or C125S mutation that corresponds to SEQ ID NO: 6. These substitutions can be used alone or in combination with one another. In some embodiments, the mutein comprises 1, 2, 3, 4, 5, 6, 7, or 8 of these mutations. In some embodiments, the mutein comprises each of these substitutions. In some embodiments, the mutein comprises a wild-type residue at one or more of positions 35, 36, 42, 104, 115, or 146 that correspond to SEQ ID NO: 15 or the equivalent positions at SEQ ID NO: 6 (e.g., positions 15, 16, 22, 84, 95, and 126).

For any of the IL-2 muteins described herein, in some embodiments, one or more of positions 35, 36, 42, 104, 115, or 146 that correspond to SEQ ID NO: 15 or the equivalent positions at SEQ ID NO: 6 (e.g., positions 15, 16, 22, 84, 95, or 126) are wild-type (e.g., are as shown in SEQ ID NOs: 6 or 15). In some embodiments, 2, 3, 4, 5, 6, or each of positions 35, 36, 42, 104, 115, or 146 that correspond to SEQ ID NO: 15 or the equivalent positions at SEQ ID NO: 6 (e.g., positions 15, 16, 22, 84, 95, and 126) are wild-type.

In some embodiments, the IL-2 mutein comprises a sequence of:

(SEQ ID NO: 16)
MYRMQLLSCIALSLALVTNSAPTSSSTKKTQLQLEHLLLDL
QMILNGISNHKNPRLARMLTFKFYMPEKATEIKHLQCLEEE
LKPLEEALRLAPSKNFHLRPRDLISDINVIVLELKGSETTFMC
EYADETATIVEFLNRWITFSQSIISTLT

In some embodiments, the IL-2 mutein comprises a sequence of:

(SEQ ID NO: 17)
MYRMQLLSCIALSLALVTNSAPTSSSTKKTQLQLEHLLLDL
QMILNGISNHKNPRLARMLTFKFYMPEKATELKHIQCLEEE
LKPLEEALRLAPSKNFHLRPRDLISDINVIVLELKGSETTFMC
EYADETATIVEFLNRWITFSQSIISTLT

In some embodiments, the IL-2 mutein comprises a sequence of:

(SEQ ID NO: 18)
MYRMQLLSCIALSLALVTNSAPTSSSTKKTQLQLEHLLLDL
QMILNGISNHKNPRLARMLTFKFYMPEKATELKHLQCLEEE
LKPLEEALRLAPSKNFHIRPRDLISDINVIVLELKGSETTFMC
EYADETATIVEFLNRWITFSQSIISTLT

In some embodiments, the IL-2 mutein comprises a sequence of:

(SEQ ID NO: 19)
MYRMQLLSCIALSLALVTNSAPTSSSTKKTQLQLEHLLLDL
QMILNGISNHKNPRLARMLTFKFYMPEKATELKHLQCLEEE
LKPLEEALRLAPSKNFHLRPRDLISDINVIVLELKGSETTFMC
EYADETATIVEFINRWITFSQSIISTLT

In some embodiments, the IL-2 mutein sequences described herein do not comprise the IL-2 leader sequence. The IL-2 leader sequence can be represented by the sequence of MYRMQLLSCIALSLALVTNS (SEQ ID NO: 20). Therefore, in some embodiments, the sequences illustrated above can also encompass peptides without the leader sequence. Although SEQ ID NOs; 16-20 are illustrated with only mutation at one of positions 73, 76, 100, or 138 that correspond to SEQ ID NO: 15 or positions at one or more of positions 53, 56, 80, or 118 that correspond to SEQ ID NO: 6, the peptides can comprises one, two, three or 4 of the mutations at these positions. In some embodiments, the substitution at each position is isoleucine or other type of conservative amino acid substitution. In some embodiments, the leucine at the recited positions are substituted with, independently, isoleucine, valine, methionine, or phenylalanine.

In some embodiments, the IL-2 mutein molecule is fused to a Fc Region or other linker region as described herein. Examples of such fusion proteins can be found in U.S. Pat. Nos. 9,580,486, 7,105,653, 9,616,105, 9,428,567, US2017/0051029, WO2016/164937, US2014/0286898A1, WO2014153111A2, WO2010/085495, WO2016014428A2, WO2016025385A1, US2017/0037102, and US2006/0269515, each of which are incorporated by reference in its entirety.

In some embodiments, the Fc region comprises what is known as the LALA mutation. Using the Kabat numbering of the Fc region, this would correspond to L247A, L248A, and G250A. In some embodiments, using the EU numbering of the Fc region, the Fc region comprises a L234A mutation, a L235A mutation, and/or a G237A mutation. Regardless of the numbering system used, in some embodiments, the Fc portion can comprise mutations that correspond to these residues. In some embodiments, the Fc region comprises N297G or N297A (Kabat numbering) mutations. The Kabat numbering is based upon a full-length sequence, but would be used in a fragment based upon a traditional alignment used by one of skill in the art for the Fc region.

In some embodiments, the Fc region comprises a sequence of:

(SEQ ID NO: 21)
DKTHTCPPCPAPEAAGAPSVFLFPPKPKDTLMISRTPEVTCV
VVDVSHEDPEVKFNWYVDGVEVHNAKTKPREEQYNSTYR
VVSVLTVLHQDWLNGKEYKCKVSNKALPAPIEKTISKAKG
QPREPQVYTLPPSREEMTKNQVSLTCLVKGFYPSDIAVEWE
SNGQPENNYKTTPPVLDSDGSFFLYSKLTVDKSRWQQGNV
FSCSVMHEALHNHYTQKSLSLSPG.
or
(SEQ ID NO: 28)
DKTHTCPPCPAPELLGGPSVFLFPPKPKDTLMISRTPEVTCV
VVDVSHEDPEVKFNWYVDGVEVHNAKTKPREEQYNSTYR
VVSVLTVLHQDWLNGKEYKCKVSNKALPAPIEKTISKAKG
QPREPQVYTLPPSREEMTKNQVSLTCLVKGFYPSDIAVEWE
SNGQPENNYKTTPPVLDSDGSFFLYSKLTVDKSRWQQGNV
FSCSVMHEALHNHYTQKSLSLSPG.

In some embodiments, the IL-2 mutein is linked to the Fc region. Non-limiting examples of linkers are glycine/serine linkers. For example, a glycine/serine linkers can be a sequence of GGGGSGGGGSGGGGSGGGGS (SEQ ID NO: 22), GGGGSGGGGS (SEQ ID NO: 531), or GGGGSGGGGSGGGGS (SEQ ID NO: 30). This is simply a non-limiting example and the linker can have varying number of GGGGS (SEQ ID NO: 23) or GGGGA repeats (SEQ ID NO: 29). In some embodiments, the linker comprises 1, 2, 3, 4, 5, 6, 7, 8, 9, or 10 of the GGGGS (SEQ ID NO: 23) or GGGGA repeats (SEQ ID NO: 29) repeats.

Thus, the IL-2/Fc fusion can be represented by the formula of Z_{IL-2M}-L_{gs}-Z_{Fc}, wherein Z_{IL-2M} is a IL-2 mutein as described herein, L_{gs} is a linker sequence as described herein (e.g., glycine/serine linker) and Z_{Fc} is a Fc region described herein or known to one of skill in the art. In some embodiments, the formula can be in the reverse orientation Z_{Fc}-L_{gs}-Z_{IL-2M}.

In some embodiments, the IL-2/Fc fusion comprises a sequence of

(SEQ ID NO: 24)
MYRMQLLSCIALSLALVTNSAPTSSSTKKTQLQLEHLLLDL
QMILNGISNHKNPRLARMLTFKFYMPEKATEIKHLQCLEEE
LKPLEEALRLAPSKNFHLRPRDLISDINVIVLELKGSETTFMC
EYADETATIVEFLNRWITFSQSIISTLTGGGGSGGGGSGGGG
SGGGGSDKTHTCPPCPAPEAAGAPSVFLFPPKPKDTLMISRT
PEVTCVVVDVSHEDPEVKFNWYVDGVEVHNAKTKPREEQ
YNSTYRVVSVLTVLHQDWLNGKEYKCKVSNKALPAPIEKT
ISKAKGQPREPQVYTLPPSREEMTKNQVSLTCLVKGFYPSDI
AVEWESNGQPENNYKTTPPVLDSDGSFFLYSKLTVDKSRW
QQGNVFSCSVMHEALHNHYTQKSLSLSPG
(SEQ ID NO: 25)
MYRMQLLSCIALSLALVTNSAPTSSSTKKTQLQLEHLLLDL
QMILNGISNHKNPRLARMLTFKFYMPEKATELKHIQCLEEE
LKPLEEALRLAPSKNFHLRPRDLISDINVIVLELKGSETTFMC
EYADETATIVEFLNRWITFSQSIISTLTGGGGSGGGGSGGGG
SGGGGSDKTHTCPPCPAPEAAGAPSVFLFPPKPKDTLMISRT
PEVTCVVVDVSHEDPEVKFNWYVDGVEVHNAKTKPREEQ
YNSTYRVVSVLTVLHQDWLNGKEYKCKVSNKALPAPIEKT
ISKAKGQPREPQVYTLPPSREEMTKNQVSLTCLVKGFYPSDI
AVEWESNGQPENNYKTTPPVLDSDGSFFLYSKLTVDKSRW
QQGNVFSCSVMHEALHNHYTQKSLSLSPG
(SEQ ID NO: 26)
MYRMQLLSCIALSLALVTNSAPTSSSTKKTQLQLEHLLLDL
QMILNGISNHKNPRLARMLTFKFYMPEKATELKHLQCLEEE
LKPLEEALRLAPSKNFHIRPRDLISDINVIVLELKGSETTFMC
EYADETATIVEFLNRWITFSQSIISTLTGGGGSGGGGSGGGG
SGGGGSDKTHTCPPCPAPEAAGAPSVFLFPPKPKDTLMISRT
PEVTCVVVDVSHEDPEVKFNWYVDGVEVHNAKTKPREEQ
YNSTYRVVSVLTVLHQDWLNGKEYKCKVSNKALPAPIEKT
ISKAKGQPREPQVYTLPPSREEMTKNQVSLTCLVKGFYPSDI
AVEWESNGQPENNYKTTPPVLDSDGSFFLYSKLTVDKSRW
QQGNVFSCSVMHEALHNHYTQKSLSLSPG
(SEQ ID NO: 27)
MYRMQLLSCIALSLALVTNSAPTSSSTKKTQLQLEHLLLDL
QMILNGISNHKNPRLARMLTFKFYMPEKATELKHLQCLEEE
LKPLEEALRLAPSKNFHLRPRDLISDINVIVLELKGSETTFMC
EYADETATIVEFINRWITFSQSIISTLTGGGGSGGGGSGGGGS
GGGGSDKTHTCPPCPAPEAAGAPSVFLFPPKPKDTLMISRTP
EVTCVVVDVSHEDPEVKFNWYVDGVEVHNAKTKPREEQY
NSTYRVVSVLTVLHQDWLNGKEYKCKVSNKALPAPIEKTIS
KAKGQPREPQVYTLPPSREEMTKNQVSLTCLVKGFYPSDIA
VEWESNGQPENNYKTTPPVLDSDGSFFLYSKLTVDKSRWQ
QGNVFSCSVMHEALHNHYTQKSLSLSPG.

In some embodiments, the IL-2/Fc fusion comprises a sequence selected from the following table, Table 2:

TABLE 2
IL-2/Fc Fusion Protein Amino Acid Sequences

| Sequence | |
|---|---|
| Identi- | |
| fication | Sequence |
| SEQ ID | APTSSSTKKTQLQLEHLLLDLQMILNGINNYKNPKL |
| NO: 7 | TRMLTFKFYMPKKATELKHLQCLEEELKPLEEVLNL |
| | AQSKNFHLRPRDLISRINVIVLELKGSETTFMCEYA |
| | DETATIVEFLNRWITFSQSIISTLTGGGGAGGGGDK |
| | THTCPPCPAPELLGGPSVFLFPPKPKDTLMISRTPE |
| | VTCVVVDVSHEDPEVKFNWYVDGVEVHNAKTKPREE |
| | QYASTYRVVSVLTVLHQDWLNGKEYKCKVSNKALPA |
| | PIEKTISKAKGQPREPQVYTLPPSRDELTKNQVSLT |
| | CLVKGFYPSDIAVEWESNGQPENNYKTTPPVLDSDG |
| | SFFLYSKLTVDKSRWQQGNVFSCSVMHEALHNHYTQ |
| | KSLSLSPGK |
| SEQ ID | APTSSSTKKTQLQLEHLLLHLQMILNGINNYKNPKL |
| NO: 8 | TRMLTFKFYMPKKATELKHLQCLEEELKPLEEVLNL |
| | AQSKNFHLRPRDLISNINVIVLELKGSETTFMCEYA |
| | DETATIVEFLNRWITFSQSIISTLTVECPPCPAPPV |
| | AGPSVFLFPPKPKDTLMISRTPEVTCVVVDVSHEDP |
| | EVQFNWYVDGVEVHNAKTKPREEQFNSTFRVVSVLT |
| | VVHQDWLNGKEYKCKVSNKGLPAPIEKTISKTKGQP |
| | REPQVYTLPPSREEMTKNQVSLTCLVKGFYPSDIAV |
| | EWESNGQPENNYKTTPPMLDSDGSFFLYSKLTVDKS |
| | RWQQGNVFSCSVMHEALHNHYTQKSLSLSPGK |
| SEQ ID | APTSSSTKKTQLQLEHLLLDLQMILNGINNYKNPKL |
| NO: 9 | TRMLTFKFYMPKKATELKHLQCLEEELKPLEEVLNL |
| | AQSKNFHLRPRDLISRINVIVLELKGSETTFMCEYA |
| | DETATIVEFLNRWITFSQSIISTLTDKTHTCPPCPA |
| | PELLGGPSVFLFPPKPKDTLMISRTPEVTCVVVDVS |
| | HEDPEVKFNWYVDGVEVHNAKTKPREEQYASTYRVV |
| | SVLTVLHQDWLNGKEYKCKVSNKALPAPIEKTISKA |
| | KGQPREPQVYTLPPSRDELTKNQVSLTCLVKGFYPS |
| | DIAVEWESNGQPENNYKTTPPVLDSDGSFFLYSKLT |
| | VDKSRWQQGNVFSCSVMHEALHNHYTQKSLSLSPG |
| SEQ ID | APTSSSTKKTQLQLEHLLLDLQMILNGINNYKNPKL |
| NO: 10 | TRMLTFKFYMPKKATELKHLQCLEEELKPLEEVLNL |
| | AQSKNFHLRPRDLISRINVIVLELKGSETTFMCEYA |
| | DETATIVEFLNRWITFSQSIISTLTGGGGSDKTHTC |
| | PPCPAPELLGGPSVFLFPPKPKDTLMISRTPEVTCV |
| | VVDVSHEDPEVKFNWYVDGVEVHNAKTKPREEQYAS |
| | TYRVVSVLTVLHQDWLNGKEYKCKVSNKALPAPIEK |
| | TISKAKGQPREPQVYTLPPSRDELTKNQVSLTCLVK |
| | GFYPSDIAVEWESNGQPENNYKTTPPVLDSDGSFFL |
| | YSKLTVDKSRWQQGNVFSCSVMHEALHNHYTQKSLS |
| | LSPG |
| SEQ ID | APTSSSTKKTQLQLEHLLLDLQMILNGINNYKNPKL |
| NO: 11 | TRMLTFKFYMPKKATELKHLQCLEEELKPLEEVLNL |
| | AQSKNFHLRPRDLISRINVIVLELKGSETTFMCEYA |
| | DETATIVEFLNRWITFSQSIISTLTGGGGSGGGGSD |
| | KTHTCPPCPAPELLGGPSVFLFPPKPKDTLMISRTP |
| | EVTCVVVDVSHEDPEVKFNWYVDGVEVHNAKTKPRE |
| | EQYASTYRVVSVLTVLHQDWLNGKEYKCKVSNKALP |
| | APIEKTISKAKGQPREPQVYTLPPSRDELTKNQVSL |
| | TCLVKGFYPSDIAVEWESNGQPENNYKTTPPVLDSD |
| | GSFFLYSKLTVDKSRWQQGNVFSCSVMHEALHNHYT |
| | QKSLSLSPG |
| SEQ ID | APTSSSTKKTQLQLEHLLLDLQMILNGINNYKNPKL |
| NO: 12 | TRMLTFKFYMPKKATELKHLQCLEEELKPLEEVLNL |
| | AQSKNFHLRPRDLISRINVIVLELKGSETTFMCEYA |
| | DETATIVEFLNRWITFSQSIISTLTGGGGSGGGGSG |
| | KGGGSDTHTCPPCPAPELLGGPSVFLFPPKPKDTLM |
| | ISRTPEVTCVVVDVSHEDPEVKENWYVDGVEVHNAK |
| | TKPREEQYASTYRVVSVLTVLHQDWINGKEYKCKVS |
| | NKALPAPIEKTISKAKGQPREPQVYTLPPSRDELTK |
| | NQVSLTCLVKGFYPSDIAVEWESNGQPENNYKTTPP |
| | VLDSDGSFFLYSKLTVDKSRWQQGNVFSCSVMHEAL |
| | HNYHTQKSLSLSPG |
| SEQ ID | APTSSSTKKTQLQLEHLLLDLQMILNGINNYKNPKL |
| NO: 13 | TRMLTFKFYMPKKATELKHLQCLEEELKPLEEVLNL |
| | AQSKNFHLRPRDLISRINVIVLELKGSETTFMCEYA |
| | DETATIVEFLNRWITFSQSIISTLTGGGGSGGGGSG |
| | GGGSGGGGSDKTHTCPPCPAPELLGGPSVFLFPPKP |
| | KDTLMISRTPEVTCVVVDVSHEDPEVKFNWYVDGVE |
| | VHNAKTKPREEQYASTYRVVSVLTVLHQDWLNGKEY |
| | KCKVSNKALPAPIEKTISKAKGQPREPQVYTLPPSR |
| | DELTKNQVSLTCLVKGFYPSDIAVEWESNGQPENNY |
| | KTTPPVLDSDGSFFLYSKLTVDKSRWQQGNVFSCSV |
| | MHEALHNHYTQKSLSLSPG |
| SEQ ID | APTSSSTKKTQLQLEHLLLHLQMILNGINNYKNPKL |
| NO: 14 | TRMLTFKFYMPKKATELKHLQCLEEELKPLEEVLNL |
| | AQSKNFHLRPRDLISNINVIVLELKGSETTFMCEYA |
| | DETATIVEFLNRWITFSQSIISTLTGGGGSGGGGSG |
| | GGGSDKTHTCPPCPAPELLGGPSVFLFPPKPKDTLM |
| | ISRTPEVTCVVVDVSHEDPEVKFNWYVDGVEVHNAK |
| | TKPREEQYASTYPVVSVLTVLHQDWINGKEYKCKVS |
| | NKALPAPIEKTISKAKGQPREPQVYTLPPSRDELTK |
| | NQVSLTCLVKGFYPSDIAVEWESNGQPENNYKTTPP |
| | VLDSDGSFFLYSKLTVDKSRWQQGNVFSCSVMHEAL |
| | HNHYTQKSLSLSPG |

In some embodiments, the IL-2 muteins comprises one or more of the sequences provided in the following table, which, in some embodiments, shows the IL-2 mutein fused with other proteins or linkers. The table also provides sequences for a variety of Fc domains or variants that the IL-2 can be fused with:

| SEQ ID | Brief | |
|---|---|---|
| NO: | Description | Amino Acid Sequence |
| 31 | Human IL-2 | APTSSSTKKTQLQLEHLLLDLQMILNGINNYKNPKLTRMLTFKFYMPKKA |
| | with C125S | TELKHLQCLEEELKPLEEVLNLAQSKNFHLRPRDLISNINVIVLELKGSE |
| | mutation | TTFMCEYADETATIVEFLNRWITFSQSIISTLT |
| 32 | Human IL-2 | APASSSTKKTQLQLEHLLLDLQMILNGINNYKNPKLTRMLTFKFYMPKKA |
| | with C125S | TELKHLQCLEEELKPLEEVLNLAQSKNFHLRPRDLISNINVIVLELKGSE |
| | and T3A | TTFMCEYADETATIVEFLNRWITFSQSIISTLT |
| | mutations | |
| 33 | Human IL-2 | APTSSSTKKTQLQLEHLLLDLQMILNGINNYKNPKLTRMLTFKFYMPKKA |
| | with N88R and | TELKHLQCLEEELKPLEEVLNLAQSKNFHLRPRDLISRINVIVLELKGSE |
| | C125S | TTFMCEYADETATIVEFLNRWITFSQSIISTLT |
| 34 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISNINVIVLELKGSE |
| | Q74P and | TTFMCEYADETATIVEFLNRWITFSQSIISTLT |
| | C125S | |
| | mutations | |
| 35 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISDINVIVLELKGSE |
| | Q74P, N88D | TTFMCEYADETATIVEFLNRWITFSQSIISTLT |
| | and C125S | |
| | mutations | |
| 36 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISRINVIVLELKGSE |
| | Q74P, N88R | TTFMCEYADETATIVEFLNRWITFSQSIISTLT |
| | and C125S | |
| | mutations | |
| 37 | Human IL-2 | APTSSSTKKTQLQLEHLLLDLQMILNGINNYKNPKLTRMLTFKFYMPKKA |
| | with N88D and | TELKHLQCLEEELKPLEEVLNLAQSKNFHLRPRDLISDINVIVLELKGSE |
| | C125S | TTFMCEYADETATIVEFLNRWITFSQSIISTLT |
| 38 | Human IL-2 | APTSSSTKKTQLQLEHLLLDLQMILNGINNYKNPKLTRMLTFKFYMPKKA |
| | with L53I, | TEIKHLQCLEEELKPLEEALNLAPSKNFHLRPRDLISDINVIVLELKGSE |
| | V69A, Q74P, | TTFMCEYADETATIVEFLNRWITFSQSIISTLT |
| | N88D and | |
| | C125S | |
| | mutations | |
| 39 | Human IL-2 | APTSSSTKKTQLQLEHLLLDLQMILNGINNYKNPKLTRMLTFKFYMPKKA |
| | with L56I, | TELKHIQCLEEELKPLEEALNLAPSKNFHLRPRDLISDINVIVLELKGSE |
| | V69A, Q74P, | TTFMCEYADETATIVEFLNRWITFSQSIISTLT |
| | N88D and | |
| | C125S | |
| | mutations | |
| 40 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHIRPRDLISDINVIVLELKGSE |
| | Q74P, L80I, | TTFMCEYADETATIVEFLNRWITFSQSIISTLT |
| | N88D and | |
| | C125S | |
| | mutations | |
| 41 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISDINVIVLELKGSE |
| | Q74P, N88D, | TTFMCEYADx`ETATIVEFINRWITFSQSIISTLT |
| | L118I, and | |
| | C125S | |
| | mutations | |
| 42 | Human IgG1 Fc | DKTHTCPPCPAPEAAGAPSVFLFPPKPKDTLMISRTPEVTCVVVDVSHED |
| | (N-terminal | PEVKFNWYVDGVEVHNAKTKPREEQYNSTYRVVSVLTVLHQDWLNGKEYK |
| | fusions) with | CKVSNKALPAPIEKTISKAKGQPREPQVYTLPPSREEMTKNQVSLTCLVK |
| | L234A, L235A, | GFYPSDIAVEWESNGQPENNYKTTPPVLDSDGSFFLYSKLTVDKSRWQQG |
| | and G237A | NVFSCSVMHEALHNHYTQKSLSLSPG |
| | mutations | |
| 30 | GGGGSGGGGSGGG | GGGGSGGGGSGGGGS |
| | GS linker (15 | |
| | amino acids) | |
| 22 | GGGGSGGGGSGGG | GGGGSGGGGSGGGGSGGGGS |
| | GSGGGGS | |
| | linker (20 | |
| | amino acids) | |
| 23 | GGGGS linker | GGGGS |
| | (5 amino | |
| | acids) | |
| 43 | Human IgG1 Fc | DKTHTCPPCPAPELLGGPSVFLFPPKPKDTLMISRTPEVTCVVVDVSHED |
| | (truncated) | PEVKFNWYVDGVEVHNAKTKPREEQYGSTYRVVSVLTVLHQDWLNGKEYK |
| | with N297G | CKVSNKALPAPIEKTISKAKGQPREPQVYTLPPSREEMTKNQVSLTCLVK |
| | mutation | GFYPSDIAVEWESNGQPENNYKTTPPVLDSDGSFFLYSKLTVDKSRWQQG |
| | | NVFSCSVMHEALHNHYTQKSLSLSPG |
| 44 | Antibody | ASTKGPSVFPLAPSSKSTSGGTAALGCLVKDYFPEPVTVSWNSGALTSGV |
| | Heavy Chain | HTFPAVLQSSGLYSLSSVVTVPSSSLGTQTYICNVNHKPSNTKVDKKVEP |
| | CH1-CH2-CH3 | KSCDKTHTCPPCPAPEAAGAPSVELFPPKPKDTLMISRTPEVTCVVVDVS |
| | domains | HEDPEVKFNWYVDGVEVHNAKTKPREEQYNSTYRVVSVLTVLHQDWLNGK |
| | (human IgG1 | EYKCKVSNKALPAPIEKTISKAKGQPREPQVYTLPPSREEMTKNQVSLTC |
| | with L234A, | LVKGFYPSDIAVEWESNGQPENNYKTTPPVLDSDGSFFLYSKLTVDKSRW |
| | L235A, and | QQGNVFSCSVMHEALHNHYTQKSLSLSPG |
| | G237A) | |
| 45 | Antibody | RTVAAPSVFIFPPSDEQLKSGTASVVCLLNNFYPREAKVQWKVDNALQSG |
| | Kappa | NSQESVTEQDSKDSTYSLSSTLTLSKADYEKHKVYACEVTHQGLSSPVTK |
| | Constant | SFNRGEC |
| | Domain | |
| | (human) | |
| 46 | IL-2-G4Sx3-Fc | APTSSSTKKTQLQLEHLLLDLQMILNGINNYKNPKLTRMLTFKFYMPKKA |
| | | TELKHLQCLEEELKPLEEVLNLAQSKNFHLRPRDLISNINVIVLELKGSE |
| | | TTFMCEYADETATIVEFLNRWITFSQSIISTLTGGGGSGGGGSGGGGSDK |
| | | THTCPPCPAPEAAGAPSVELFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 47 | IL-2 T3A- | APASSSTKKTQLQLEHLLLDLQMILNGINNYKNPKLTRMLTFKFYMPKKA |
| | G4Sx3-Fc | TELKHLQCLEEELKPLEEVLNLAQSKNFHLRPRDLISNINVIVLELKGSE |
| | | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 48 | IL-2 N88R- | APTSSSTKKTQLQLEHLLLDLQMILNGINNYKNPKLTRMLTFKFYMPKKA |
| | G4Sx3-Fc | TELKHLQCLEEELKPLEEVLNLAQSKNFHLRPRDLISRINVIVLELKGSE |
| | | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 49 | IL-2 V69A, | APTSSSTKKTQLQLEHLLLDLQMILNGINNYKNPKLTRMLTFKFYMPKKA |
| | Q74P,-G4Sx3- | TELKHLQCLEEELKPLEEALNLAPSKNFHLRPRDLISNINVIVLELKGSE |
| | Fc | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 50 | IL-2 N88D | APTSSSTKKTQLQLEHLLLDLQMILNGINNYKNPKLTRMLTFKFYMPKKA |
| | V69A, Q74P- | TELKHLQCLEEELKPLEEALNLAPSKNFHLRPRDLISDINVIVLELKGSE |
| | G4Sx3-Fc | TTFMCEYADETATIVEFLNRWITFSQSIISTLTGGGGSGGGGSGGGGSDK |
| | | THTCPPCPAPEAAGAPSVELFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 51 | IL-2 N88R | APTSSSTKKTQLQLEHLLLDLQMILNGINNYKNPKLTRMLTFKFYMPKKA |
| | V69A, Q74P- | TELKHLQCLEEELKPLEEALNLAPSKNFHLRPRDLISRINVIVLELKGSE |
| | G4Sx3-Fc | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 52 | IL-2 N88D- | APTSSSTKKTQLQLEHLLLDLQMILNGINNYKNPKLTRMLTFKFYMPKKA |
| | G4Sx3-Fc | TELKHLQCLEEELKPLEEVLNLAQSKNFHLRPRDLISDINVIVLELKGSE |
| | | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 53 | IL-2 L53I | APTSSSTKKTQLQLEHLLLDLQMILNGINNYKNPKLTRMLTFKFYMPKKA |
| | N88D V69A, | TEIKHLQCLEEELKPLEEALNLAPSKNFHLRPRDLISDINVIVLELKGSE |
| | Q74P,C125S- | TTFMCEYADETATIVEFLNRWITFSQSIISTLTGGGGSGGGGSGGGGSGG |
| | G4Sx4-Fc | GGSDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 54 | IL-2 L56I | APTSSSTKKTQLQLEHLLLDLQMILNGINNYKNPKLTRMLTFKFYMPKKA |
| | N88D V69A, | TELKHIQCLEEELKPLEEALNLAPSKNFHLRPRDLISDINVIVLELKGSE |
| | Q74P, C125S- | TTFMCEYADETATIVEFLNRWITFSQSIISTLTGGGGSGGGGSGGGGSGG |
| | G4Sx4-Fc | GGSDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 55 | IL-2 L80I | APTSSSTKKTQLQLEHLLLDLQMILNGINNYKNPKLTRMLTFKFYMPKKA |
| | N88D V69A, | TELKHLQCLEEELKPLEEALNLAPSKNFHIRPRDLISDINVIVLELKGSE |
| | C125S Q74P- | TTFMCEYADETATIVEFLNRWITFSQSIISTLTGGGGSGGGGSGGGGSGG |
| | G4Sx4-Fc | GGSDKTHTCPPCPAPEAAGAPSVELFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 56 | IL-2 L118I | APTSSSTKKTQLQLEHLLLDLQMILNGINNYKNPKLTRMLTFKFYMPKKA |
| | N88D V69A, | TELKHLQCLEEELKPLEEALNLAPSKNFHLRPRDLISDINVIVLELKGSE |
| | Q74P, C125S- | TTFMCEYADETATIVEFINRWITFSQSIISTLTGGGGSGGGGSGGGGSGG |
| | G4Sx4-Fc | GGSDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 57 | IL-2 N88D | APTSSSTKKTQLQLEHLLLDLQMILNGINNYKNPKLTRMLTFKFYMPKKA |
| | V69A, Q74P- | TELKHLQCLEEELKPLEEALNLAPSKNFHLRPRDLISDINVIVLELKGSE |
| | G4Sx4-Fc | TTFMCEYADETATIVEFLNRWITFSQSIISTLTGGGGSGGGGSGGGGSGG |
| | | GGSDKTHTCPPCPAPEAAGAPSVELFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 58 | Fc-G4S-IL-2 | DKTHTCPPCPAPELLGGPSVFLFPPKPKDTLMISRTPEVTCVVVDVSHED |
| | N88D V69A, | PEVKFNWYVDGVEVHNAKTKPREEQYGSTYRVVSVLTVLHQDWLNGKEYK |
| | Q74P | CKVSNKALPAPIEKTISKAKGQPREPQVYTLPPSREEMTKNQVSLTCLVK |
| | | GFYPSDIAVEWESNGQPENNYKTTPPVLDSDGSFFLYSKLTVDKSRWQQG |
| | | NVFSCSVMHEALHNHYTQKSLSLSPGGGGGSAPTSSSTKKTQLQLEHLLL |
| | | DLQMILNGINNYKNPKLTRMLTFKFYMPKKATELKHLQCLEEELKPLEEA |
| | | LNLAPSKNFHLRPRDLISDINVIVLELKGSETTFMCEYADETATIVEFLN |
| | | RWITFAQSIISTLT |
| 59 | IL-2 N88D | APTSSSTKKTQLQLEHLLLDLQMILNGINNYKNPKLTRMLTFKFYMPKKA |
| | V69A, Q74P, | TEX₁KHX₂QCLEEELKPLEEALNLAPSKNFHX₃RPRDLISDINVIVLELKG |
| | C125S-G4Sx4- | SETTFMCEYADETATIVEFX₄NRWITFSQSIISTLTGGGGSGGGGSGGGGS |
| | Fc, wherein | GGGGSDKTHTCPPCPAPEAAGAPSVFLFPPKPKDTLMISRTPEVTCVVVD |
| | at least one | VSHEDPEVKFNWYVDGVEVHNAKTKPREEQYNSTYRVVSVLTVLHQDWLN |
| | of X₁, X₂, X₃, | GKEYKCKVSNKALPAPIEKTISKAKGQPREPQVYTLPPSREEMTKNQVSL |
| | and X₄ is I | TCLVKGFYPSDIAVEWESNGQPENNYKTTPPVLDSDGSFFLYSKLTVDKS |
| | and the | RWQQGNVFSCSVMHEALHNHYTQKSLSLSPG |
| | remainder are | |
| | L or I. | |
| 60 | IL-2 N88D | APTSSSTKKTQLQLEHLLLDLQMILNGINNYKNPKLTRMLTFKFYMPKKA |
| | V69A, Q74P, | TEX₁KHX₂QCLEEELKPLEEALNLAPSKNFHX₃RPRDLISDINVIVLELKG |
| | C125S, | SETTFMCEYADETATIVEFX₄NRWITFSQSIISTLT |
| | wherein at | |
| | least one of | |
| | X₁, X₂, X₃, | |
| | and X₄ is I | |
| | and the | |
| | remainder are | |
| | L or I. | |

In some embodiments, the sequences shown in the table or throughout comprise or do not comprise one or more mutations that correspond to positions L53, L56, L80, and L118. In some embodiments, the sequences shown in the table or throughout the present application comprise or do not comprise one or more mutations that correspond to positions L59I, L631, I24L, L94I, L96I or L132I or other substitutions at the same positions. In some embodiments, the mutation is leucine to isoleucine. In some embodiments, the mutein does not comprise another mutation other than as shown or described herein. In some embodiments, the peptide comprises a sequence of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 56, SEQ ID NO: 57, SEQ ID NO: 58, SEQ ID NO: 59, or SEQ ID NO: 60.

In some embodiments, the protein comprises a TL-2 mutein as provided for herein. In some embodiments, a polypeptide is provided comprising SEQ ID NO: 59 or SEQ ID NO: 60, wherein at least one of X₁, X₂, X₃, and X₄ is I and the remainder X₁, X₂, X₃, and X₄ are L or I. As used herein, and in reference to SEQ ID NO: 59 or SEQ ID NO: 60, X₁ indicates an amino acid at position 53 as compared to SEQ ID NO: 59 or SEQ ID NO: 60. As used herein, and in reference to SEQ ID NO: 59 or SEQ ID NO: 60, X₂ indicates an amino acid at position 56 as compared to SEQ ID NO: 59 or SEQ ID NO: 60. As used herein, and in reference to SEQ ID NO: 59 or SEQ ID NO: 60, X₃ indicates an amino acid at position 80 as compared to SEQ ID NO: 59 or SEQ ID NO: 60. As used herein, and in reference to SEQ ID NO: 59 or SEQ ID NO: 60, X₄ indicates an amino acid at position 118 as compared to SEQ ID NO: 59 or SEQ ID NO: 60. In some embodiments, X₁, X₂, and X₃ are L and X₄ is I. In some embodiments, X₁, X₂, and X₄ are L and X₃ is I. In some embodiments, X₂, X₃, and X₄ are L and X₁ is I. In some embodiments, X₁, X₃, and X₄ are L and X₂ is I. In some embodiments, X₁ and X₂ are L and X₃ and X₄ are I. In some embodiments, X₁ and X₃ are L and X₂ and X₄ are I. In some embodiments, X₁ and X₄ are L and X₂ and X₃ are I. In some embodiments, X₂ and X₃ are L and X₁ and X₄ are I. In some embodiments, X₂ and X₄ are L and X₁ and X₃ are I. In some embodiments, X₃ and X₄ are L and X₁ and X₂ are I. In some embodiments, X₁, X₂, and X₃ are L and X₄ is I. In some embodiments, X₂, X₃, and X₄ are L and X₁ is I. In some embodiments, X₁, X₃, and X₄ are L and X₂ is I. In some embodiments, X₁, X₂, and X₄ are L and X₃ is I.

In some embodiments, the Fc portion of the fusion is not included. In some embodiments, the peptide consists essentially of a IL-2 mutein provided for herein. In some embodiments, the protein is free of a Fc portion.

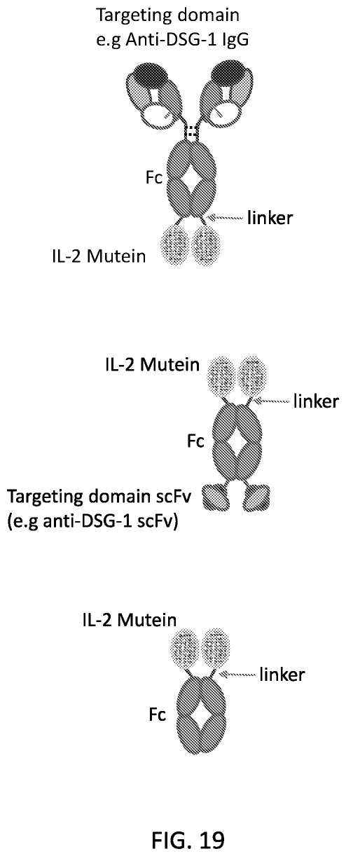

For illustrative purposes only, embodiments of IL-2 mutein fused with a Fc and with a targeting moiety are illustrated in FIG. 19. The targeting moiety is illustrated as an anti-desmoglein antibody, but that is for illustration purposes only and it can be replaced with another targeting moiety, such as an anti-desmoglein 1, 2, 3, and/or 4 antibody. Similarly, the IL-2 mutein can be replaced with a PD-1 agonist or other type of effector molecule, such as CD39 or related family members of the ENTPD gene family.

In some embodiments, the IL-2 mutein is linked directly, or indirectly, to a PD-1 agonist.

The sequences are for illustrative purposes only and are not intended to be limiting. In some embodiments, the compound comprises an amino acid sequence of SEQ ID NO: 53, 54, 55, or 56. In some embodiments, the compound comprises an amino acid sequence of SEQ ID NO: 53, 54, 55, or 56 with or without a C125A or C125S mutation. In some embodiments, the residue at position 125 is C, S, or A. In some embodiments, the compound comprises an amino acid sequence of SEQ ID NO: 59 or SEQ ID NO: 60, wherein at least one of X₁, X₂, X₃, and X₄ is I and the remainder are L or I. In some embodiments, the protein comprises a IL-2 mutein as provided for herein. In some embodiments, a polypeptide is provided comprising SEQ ID NO: 59 or SEQ ID NO: 60, wherein at least one of X₁, X₂, X₃, and X₄ is I and the remainder are L or I. In some embodiments, X₁, X₂, and X₃ are L and X₄ is I. In some embodiments, X₁, X₂, and X₄ are L and X₃ is I. In some embodiments, X₂, X₃, and X₄ are L and X₁ is I. In some embodiments, X₁, X₃, and X₄ are L and X₂ is I. In some embodiments, X₁ and X₂ are L and X₃ and X₄ are I. In some embodiments, X₁ and X₃ are L and X₂ and X₄ are I. In some embodiments, X₁ and X₄ are L and X₂ and X₃ are I. In some embodiments, X₂ and X₃ are L and X₁ and X₄ are I. In some embodiments, X₂ and X₄ are L and X₁ and X₃ are I. In some embodiments, X₃ and X₄ are L and X₁ and X₂ are I. In some embodiments, X₁, X₂, and X₃ are L and X₄ is I. In some embodiments, X₂, X₃, and X₄ are L and X₁ is I. In some embodiments, X₁, X₃, and X₄ are L and X₂ is I. In some embodiments, X₁, X₂, and X₄ are L and X₃ is I.

Each of the proteins may also be considered to have the C125S and the LALA and/or G237A mutations as provided for herein. The C125 substitution can also be C125A as described throughout the present application.

In an embodiment, an IL-2 mutein molecule comprises at least 60, 70, 80, 85, 90, 95, or 97% sequence identity or homology with a naturally occurring human IL-2 molecule, e.g., a naturally occurring IL-2 sequence disclosed herein or those that incorporated by reference.

As described herein the IL-2 muteins can be part of a bispecific molecule with a tethering moiety, such as an anti-desmoglein 1 antibody, an anti-desmoglein 2 antibody, an anti-desmoglein 3 antibody, or an anti-desmoglein 4 antibody that will target the IL-2 mutein to a desmoglein 1, 2, 3, and/or 4 expressing cell. As described herein, the bispecific molecule can be produced from two polypeptide chains. In some embodiments, the anti-desmoglein 1 antibody, an anti-desmoglein 2 antibody, an anti-desmoglein 3 antibody, or an anti-desmoglein 4 antibody, or any antibody binding fragments is linked to an IL-2 mutein effector. In some embodiments, the IL-2 mutein effector is as provided herein.

In some embodiments, an anti-desmoglein 1 antibody, an anti-desmoglein-2 antibody, an anti-demosglein 3 antibody, or an anti-demoglein 4 antibody is linked to a PD-1 effector. In some embodiments, an anti-desmoglein 1 antibody, an anti-desmoglein-2 antibody, an anti-demosglein 3 antibody, or an anti-demoglein 4 antibody is linked to a IL-2 mutein effector. In some embodiments, an anti-desmoglein 1 antibody, an anti-desmoglein-2 antibody, an anti-demosglein 3 antibody, or an anti-demoglein 4 antibody is linked to a CD39 Effector Domain.

In some embodiments, the following sequences or an anti-desmoglein 1 antibody, an anti-desmoglein 2 antibody, an anti-desmoglein 3 antibody, or an anti-desmoglein 4 antibody, or any antibody binding fragments thereof can comprise one or more of the following sequences:

(Anti-DSG1 variable domain, SEQ ID NO: 61)
QVQLQESGPGPVKPSGTLSLTCGVSGGSISSNHWWTWVRQPPGKGLEWIG
EIYHNGSTFLNPSLKSRVTISVDKSNNQFSLKLTSVTAADTAVYYCARGW
HRTGFRGYPSHWYFDLWGRGTLVSVSS,
or
(Anti-DSG1 variable domain, SEQ ID NO: 62)
QSVLTQPPSVSGTPGQRVTISCSGSSSHIGSNYVYWYQQLPGTAPKILIY
SNDQRPAGVPDRESASKSGTSASLAISGLRSEDEADYYCAAWDDGQGGVF
GGGTKLTVL,
or
(SEQ ID NO: 63)
QVQLVQSGAEVKKPGASVKVSCKASGYSFTTYYMHWVRQAPGQGLEWMGI
INPSGGSTSYAQKFQGRVTMTRDTSTSTVYMELSSLRSEDTAVYYCASGW
VYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNH
KPSNTKVDKKVEPKSCDKTHTCPPCPAPEAAGAPSVFLFPPKPKDTLMIS
RTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSF
FLYSKLTVDKSRWQQGNVFSCSVMHEALHNHYTQKSLSLSPGGGGGSGGG
GSGGGGSQSVLTQPPSVSGTPGQRVTISCSGSSSHIGSNYVYWYQQLPGT
APKILIYSNDQRPAGVPDRESASKSGTSASLAISGLRSEDEADYYCAAWD
DGQGGVFGGGTKLTVLGGGGSGGGGSGGGGSGGGGSQVQLQESGPGPVKP
SGTLSLTCGVSGGSISSNHWWTWVRQPPGKGLEWIGEIYHNGSTFLNPSL
KSRVTISVDKSNNQFSLKLTSVTAADTAVYYCARGWHRTGFRGYPSHWYF
DLWGRGTLVSVSS,
or
(SEQ ID NO: 64)
QVQLQESGPGPVKPSGTLSLTCGVSGGSISSNHWWTWVRQPPGKGLEWIG
EIYHNGSTFLNPSLKSRVTISVDKSNNQFSLKLTSVTAADTAVYYCARGW
HRTGFRGYPSHWYFDLWGRGTLVSVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPEAAGAPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSP,
or
(SEQ ID NO: 65)
QSVLTQPPSVSGTPGQRVTISCSGSSSHIGSNYVYWYQQLPGTAPKILIY
SNDQRPAGVPDRFSASKSGTSASLAISGLRSEDEADYYCAAWDDGQGGVF
GGGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVTH
EGSTVEKTVAPTECS,
or
(Constant Lambda, SEQ ID NO: 66)
GQPKAAPSVTLFPPSSEELQANKATLVCLISDFYPGAVTVAWKADSSPVK
AGVETTTPSKQSNNKYAASSYLSLTPEQWKSHRSYSCQVTHEGSTVEKTV
APTECS

In some embodiments, the anti-desmoglein 1 antibody, an anti-desmoglein 2 antibody, an anti-desmoglein 3 antibody, or an anti-desmoglein 4 antibody, or any antibody binding fragments is linked to a CD39 effector domain. In some embodiments, the effector domain has a CD39 sequence as provided herein. In some embodiments, the anti-desmoglein 1 antibody, an anti-desmoglein 2 antibody, an anti-desmoglein 3 antibody, or an anti-desmoglein 4 antibody, or any antibody binding fragments linked to a CD39 effector domain comprises one or more of the following sequences, such as a heavy and light chain:

>Variable Heavy Chain 1
(SEQ ID NO: 67)
QVQLQESGPGPVKPSGTLSLTCGVSGGSISSNHWWTWVRQPPGKGLEWIG
EIYHNGSTFLNPSLKSRVTISVDKSNNQFSLKLTSVTAADTAVYYCARGW
HRTGFRGYPSHWYFDLWGRGTLVSVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPEAAGAPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGGGGGSGGGGSGGGGSTQNKPLPENVKYGIVLDAGSSHTNLYIYK
WPAEKENDTGVVQQLEECQVKGPGISKYAQKTDEIGAYLAECMELSTELI
PTSKHHQTPVYLGATAGMRLLRMESEQSADEVLAAVSTSLKGYPFDFQGA
KIITGQEEGAYGWITINYLLGRFTQEQSWLSLISDSQKQETFGALDLGGA
STQITFVPQNSTIESPENSLQFRLYGEDYTVYTHSFLCYGKDQALWQKLA
KDIQVSSGGVLKDPCFNPGYEKVVNVSELYGTPCTERFEKKLPFDQFRIQ
GTGDYEQCHQSILELFNNSHCPYSQCAFNGVFLPPLHGSFGAFSAFYFVM
DFFKKVAKNSVISQEKMTEITKNFCSKSWEETKTSYPSVKEKYLSEYCFS
GAYILSLLQGYNFTDSSWEQIHFMGKIKDSNAGWTLGYMLNLTNMIPAEQ
PLSPPLPHSTYI
>Variable Light Chain 1
(SEQ ID NO: 68)
QSVLTQPPSVSGTPGQRVTISCSGSSSHIGSNYVYWYQQLPGTAPKILIY
SNDQRPAGVPDRFSASKSGTSASLAISGLRSEDEADYYCAAWDDGQGGVF
GGGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVTH
EGSTVEKTVAPTECS
>Variable Heavy Chain 2
(SEQ ID NO: 69)
QVQLQESGPGPVKPSGTLSLTCGVSGGSISSNHWWTWVRQPPGKGLEWIG
EIYHNGSTFLNPSLKSRVTISVDKSNNQFSLKLTSVTAADTAVYYCARGW
HRTGFRGYPSHWYFDLWGRGTLVSVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPEAAGAPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGGGGGSGGGGSGGGGSTQNKALPENVKYGIVLDAGSSHTSLYIYK
WPAEKENDTGVVHQVEECRVKGPGISKFVQKVNEIGIYLTDCMERAREVI
PRSQHQETPVYLGATAGMRLLRMESEELADRVLDVVERSLSNYPFDFQGA
RIITGQEEGAYGWITINYLLGKFSQKTRWFSIVPYETNNQETFGALDLGG
ASTQVTFVPQNQTIESPDNALQFRLYGKDYNVYTHSFLCYGKDQALWQKL
AKDIQVASNEILRDPCFHPGYKKVVNVSDLYKTPCTKRFEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLLQGYHFTADSWEHIHFIGKIQGSDAGWTLGYMLNLTNMIPAEQ
PLSTPLSHSTYV
>Variable Light Chain 2
(SEQ ID NO: 70)
QSVLTQPPSVSGTPGQRVTISCSGSSSHIGSNYVYWYQQLPGTAPKILIY
SNDQRPAGVPDRESASKSGTSASLAISGLRSEDEADYYCAAWDDGQGGVF
GGGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVTH
EGSTVEKTVAPTECS

The variable heavy and light chains can be mixed with one another. These are illustrative examples only and other CD39 effector domains provided in these examples can be replaced with other effector domains, such as other members of the ENTPD gene family, which are described herein. The linkers illustrated in the above sequences can be replaced with other linkers as described herein or known to one of skill in the art. Where the an anti-desmoglein 1 antibody, an anti-desmoglein 2 antibody, an anti-desmoglein 3 antibody, or an anti-desmoglein 4 antibody are linked to an IL-2 mutein, the IL-2 muteins can be produced with or without a C125A or C125S mutation in the IL-2 mutein. Examples of IL-2 muteins that can be included are illustrated herein, such as, but not limited to, a sequence of SEQ ID NO: 59 or SEQ ID NO: 60.

In some embodiments, the constant kappa domain in any of the light chains can be replaced with a constant lambda domain.

### Auto-Immune Disorders

Therapeutic compounds and methods described herein can be used to treat a subject having, or at risk for having, an unwanted autoimmune response, e.g., an autoimmune response in Type 1 diabetes, multiple sclerosis, cardiomyositis, vitiligo, alopecia, inflammatory bowel disease (IBD, e.g., Crohn's disease or ulcerative colitis), Sjogren's syndrome, focal segmented glomerular sclerosis (FSGS), scleroderma/systemic sclerosis (SSc) or rheumatoid arthritis. In some embodiments, the treatment minimizes rejection of, minimizes immune effector cell mediated damage to, prolongs the survival of subject tissue undergoing, or a risk for, autoimmune attack.

Other examples of autoimmune disorders and diseases that can be treated with the compounds described herein include, but are not limited to, myocarditis, postmyocardial infarction syndrome, postpericardiotomy syndrome, subacute bacterial endocarditis, anti-glomerular basement membrane nephritis, interstitial cystitis, lupus nephritis, membranous glomerulonephropathy, chronic kidney disease (“CKD”), autoimmune hepatitis, primary biliary cirrhosis, primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease (lad), morphea, pemphigus vulgaris, pityriasis lichenoides et varioliformis acuta, Mucha-Habermann disease, psoriasis, systemic scleroderma, vitiligo, Addison's disease, autoimmune polyendocrine syndrome (APS) type 1, autoimmune polyendocrine syndrome (APS) type 2, autoimmune polyendocrine syndrome (APS) type 3, autoimmune pancreatitis (AIP), diabetes mellitus type 1, autoimmune thyroiditis, Ord's thyroiditis, Graves' disease, autoimmune oophoritis, endometriosis, autoimmune orchitis, Sjogren's syndrome, autoimmune enteropathy, coeliac disease, Crohn's disease, microscopic colitis, ulcerative colitis, thrombocytopenia, adiposis, dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, rheumatoid arthritis, sarcoidosis, Schnitzler syndrome, systemic lupus erythematosus (SLE), undifferentiated connective tissue disease (UCTD), dermatomyositis, fibromyalgia, inclusion body myositis, myositis, myasthenia gravis, neuromyotonia, paraneoplastic cerebellar degeneration, polymyositis, acute disseminated encephalomyelitis (ADEM), acute motor axonal neuropathy, anti-N-methyl-D-aspartate (anti-NMDA) receptor encephalitis, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, Oshtoran syndrome, pediatric autoimmune neuropsychiatric disorder associated with streptococcus (PANDAS), progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Meniere's disease, Behcet's disease, eosinophilic granulomatosis with polyangiitis (EGPA), giant cell arteritis, granulmatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumaticia, vasculitis, primary immune deficiency, and the like.

Other examples of potential autoimmune disorders and diseases, as well as autoimmune comorbidities that can be treated with the compounds described herein include, but are not limited to, chronic fatigue syndrome, complex regional pain syndrome, eosinophilic esophagitis, gastirtis, interstitial lung disease, POEMS syndrome, Raynaud's phenomenon, primary immunodeficiency, pyoderma gangrenosum, agammaglobulinemia, anyloidosis, amyotrophic lateral sclerosis, anti-tubular basement membrane nephritis, atopic allergy, atopic dermatitis, autoimmune peripheral neuropathy, Blau syndrome, Castleman's disease, Chagas disease, chronic obstructive pulmonary disease, chronic recurrent multifocal osteomyelitis, complement component 2 deficiency, contact dermatitis, Cushing's syndrome, cutaneous leukocytoclastic angiitis, Dego' disease, eczema, eosinophilic gastroenteritis, eosinophilic pneumonia, erythroblastosis fetalsis, fibrodysplasia ossificans progressive, gastrointestinal pemphigoid, hypogammaglobulinemia, idiopathic giant cell myocarditis, idiopathic pulmonary fibrosis, IgA nephropathy, immunregulatory lipoproteins, IPEX syndrome, ligenous conjunctivitis, Majeed syndrome, narcolepsy, Rasmussen's encephalitis, schizophrenia, serum sickness, spondyloathropathy, Sweet's syndrome, Takayasu's arteritis, and the like.

In some embodiments, the autoimmune disorder does not comprise pemphigus vulgaris, pemphigus. In some embodiments, the autoimmune disorder does not comprise pemphigus foliaceus. In some embodiments, the autoimmune disorder does not comprise bullous pemphigoid. In some embodiments, the autoimmune disorder does not comprise Goodpasture's disease. In some embodiments, the autoimmune disorder does not comprise psoriasis. In some embodiments, the autoimmune disorder does not comprise a skin disorder. In some embodiments, the disorder does not comprise a neoplastic disorder, e.g., cancer.

In some embodiments, the polypeptides and compositions provided for herein can be used to reduce T-cells at a particular target site, such as skin, when the active moiety is tethered to a targeting moiety that binds to a target that is expressed exclusively or predominantly at the target site. As provided herein, skin expresses certain proteins that other organs or tissues do not express, such as the desmoglein proteins. Accordingly, in some embodiments, the polypeptides can be used to reduce melanocyte-specific T cells and/or epidermis resident memory T cells. In some embodiments, the reduction of the melanocyte-specific T cells and/or epidermis resident memory T cells is selective or specific to these tissue types. As used in this context, the term “specific” or “selective” means that the melanocyte T-cells or epidermis resident memory T cells are reduced more than other types of T-cells that are expressed in other tissue types that are not skin, such as the intestine (gut). In some embodiments, the melanocyte-specific T cells are reduced. In some embodiments, the resident memory T cells are reduced in the epidermis. In some embodiments, methods of reducing melanocyte-specific T cells and/or resident memory T cells in a subject are provided. In some embodiments, the subject is a subject in need thereof. In some embodiments, the methods comprise administering a polypeptide, antibody, therapeutic compound, or pharmaceutical composition as provided for herein.

### Therapeutic Compounds

A therapeutic compound, which can be a polypeptide, comprises a specific targeting moiety functionally associated with an effector binding/modulating moiety. In some embodiments, the specific targeting moiety (e.g., an anti-desmoglein 1 antibody, an anti-desmoglein 2 antibody, an anti-desmoglein 3 antibody, or an anti-desmoglein 4 antibody) and effector binding/modulating moiety are linked to one another by a covalent or noncovalent bond, e.g., a covalent or non-covalent bond directly linking the one to the other. In other embodiments, a specific targeting moiety and effector binding/modulating moiety are linked, e.g., covalently or noncovalently, through a linker moiety. E.g., in the case of a fusion polypeptide, a polypeptide sequence comprising the specific targeting moiety and a polypeptide sequence can be directly linked to one another or linked through one or more linker sequences. In some embodiments, the linker moiety comprises a polypeptide. Linkers are not, however, limited to polypeptides. In some embodiments, a linker moiety comprises other backbones, e.g., a non-peptide polymer, e.g., a PEG polymer. In some embodiments, a linker moiety can comprise a particle, e.g., a nanoparticle, e.g., a polymeric nanoparticle. In some embodiments, a linker moiety can comprise a branched molecule, or a dendrimer.

In some embodiments, a therapeutic compound comprises a polypeptide comprising a specific targeting moiety covalently or non-covalently conjugated to an effector binding/modulating moiety. In some embodiments, a therapeutic molecule comprises a fusion protein having comprising a specific targeting moiety fused, e.g., directly or through a linking moiety comprising one or more amino acid residues, to an effector binding/modulating moiety.

In some embodiments, a therapeutic molecule comprises a polypeptide comprising a specific targeting moiety linked by a non-covalent bond or a covalent bond, e.g., a covalent bond other than a peptide bond, e.g., a sulfhydryl bond, to an effector binding/modulating moiety.

In some embodiments, a therapeutic compound comprises polypeptide, e.g., a fusion polypeptide, comprising:
1.a) a specific targeting moiety comprising a target specific binding polypeptide;
1.b) a specific targeting moiety comprising a target ligand binding molecule;
1.c) a specific targeting moiety comprising an antibody molecule;
1.d) a specific targeting moiety comprising a single chain antibody molecule, e.g., a scFv domain; or
1.e) a specific targeting moiety comprising a first of the light or heavy chain variable region of an antibody molecule, and wherein the other variable region is covalently or non-covalently associated with the first;
and
2.a) an effector binding/modulating moiety comprising an effector specific binding polypeptide;
2.b) an effector binding/modulating moiety comprising an effector ligand binding molecule;
2.c) an effector binding/modulating moiety comprising an antibody molecule;
2.d) an effector binding/modulating moiety comprising a single chain antibody molecule, e.g., a scFv domain; or
2.e) an effector binding/modulating moiety comprising a first of the light or heavy chain variable region of an antibody molecule, and wherein the other variable region is covalently or non-covalently associated with the first.

In some embodiments, a therapeutic compound comprises 1.a and 2.a.

In some embodiments, a therapeutic compound comprises 1.a and 2.b.

In some embodiments, a therapeutic compound comprises 1.a and 2.c.

In some embodiments, a therapeutic compound comprises 1.a and 2.d.

In some embodiments, a therapeutic compound comprises 1.a and 2.e.

In some embodiments, a therapeutic compound comprises 1.b and 2.a.

In some embodiments, a therapeutic compound comprises 1.b and 2.b.

In some embodiments, a therapeutic compound comprises 1.b and 2.c.

In some embodiments, a therapeutic compound comprises 1.b and 2.d.

In some embodiments, a therapeutic compound comprises 1.b and 2.e.

In some embodiments, a therapeutic compound comprises 1.c and 2.a.

In some embodiments, a therapeutic compound comprises 1.c and 2.b.

In some embodiments, a therapeutic compound comprises 1.c and 2.c.

In some embodiments, a therapeutic compound comprises 1.c and 2.d.

In some embodiments, a therapeutic compound comprises 1.c and 2.e.

In some embodiments, a therapeutic compound comprises 1.d and 2.a.

In some embodiments, a therapeutic compound comprises 1.d and 2.b.

In some embodiments, a therapeutic compound comprises 1.d and 2.c.

In some embodiments, a therapeutic compound comprises 1.d and 2.d.

In some embodiments, a therapeutic compound comprises 1.d and 2.e.

In some embodiments, a therapeutic compound comprises 1.e and 2.a.

In some embodiments, a therapeutic compound comprises 1.e and 2.b.

In some embodiments, a therapeutic compound comprises 1.e and 2.c.

In some embodiments, a therapeutic compound comprises 1.e and 2.d.

In some embodiments, a therapeutic compound comprises 1.e and 2.e.

Therapeutic compounds disclosed herein can, for example, comprise a plurality of effector binding/modulating and specific targeting moieties. Any suitable linker or platform can be used to present the plurality of moieties. The linker is typically coupled or fused to one or more effector binding/modulating and targeting moieties.

In some embodiments, two (or more) linkers associate, either covalently or non-covalently, e.g., to form a hetero- or homodimeric therapeutic compound. For example, the linker can comprise an Fc region and two Fc regions associate with one another. In some embodiments of a therapeutic compound comprising two linker regions, the linker regions can self associate, e.g., as two identical Fc regions. In some embodiments of a therapeutic compound comprising two linker regions, the linker regions are not capable of, or not capable of substantial, self association, e.g., the two Fc regions can be members of a knob and hole pair.

Non-limiting exemplary configurations of therapeutic compounds comprise the following (e.g., in N to C terminal order):
R1-Linker Region A-R2 or
R3-Linker Region B-R4,
   wherein,
R1, R2, R3, and R4, each independently comprises an effector binding/modulating moiety, e.g., anti-PD-1 molecule, IL-2 mutein, CD39, or is absent, provided that at least one of R1 and R2 is not absent, and at least one of R3 and R4 is not absent;
Linker Region A and Linker Region B comprise moieties that can associate with one another, e.g., Linker A and Linker B each comprises an Fc moiety provided that an effector binding/modulating moiety and a specific targeting moiety are present.

In some embodiments, the polypeptide having the formula of R1-Linker Region A-R2 and the polypeptide having the formula of R3-Linker Region B-R4 interact with one another to form a polypeptide complex.

In some embodiments, the polypeptide having the formula of R1-Linker Region A-R2 and the polypeptide having the formula of R3-Linker Region B-R4 do not interact with one another to form a polypeptide complex.

In some embodiments:
R1 comprises an effector binding/modulating moiety, e.g., anti-PD-1 molecule, IL-2 mutein, CD39, or is absent;
R2 comprises a specific targeting moiety, or is absent;
R3 comprises an effector binding/modulating moiety, e.g., anti-PD-1 molecule, IL-2 mutein, CD39, or is absent;
R4 comprises a specific targeting moiety, or is absent;
Linker Region A and Linker Region B comprise moieties that can associate with one another, e.g., Linker A and Linker B each comprises an Fc moiety, provided that one of R1 or R3 is present and one of R2 or R4 is present.

In some embodiments:
R1 comprises a specific targeting moiety, or is absent;
R2 comprises an effector binding/modulating moiety, e.g., anti-PD-1 molecule, IL-2 mutein, CD39, or is absent;
R3 comprises a specific targeting moiety, or is absent;
R4 comprises an effector binding/modulating moiety, e.g., anti-PD-1 molecule, IL-2 mutein, CD39, or is absent;
Linker Region A and Linker Region B comprise moieties that can associate with one another, e.g., Linker A and Linker B each comprises an Fc moiety, provided that one of R1 or R3 is present and one of R2 or R4 is present.
   Non-limiting examples include, but are not limited to:

| | Linker | | | | | |
|---|---|---|---|---|---|---|
| | Region | | | Linker | | |
| R1 | A | R2 | R3 | Region | R4 | Other |
| HCVR | Fc | fcFv | HCVR | Fc | scFv | Self Pairing |
| and | Region | | and | Region | | Linker Regions |
| LCVR | | | LCVR | | | |
| HCVR | Fc | fcFv | HCVR | Fc | scFv | Non-Self |
| and | Region | | and | Region | | Pairing linker |
| LCVR | | | LCVR | | | regions |
| HCVR | Fc | fcFv | HCVR | Fc | scFv | Self Pairing |
| and | Region | | and | Region | | Linker Regions |
| LCVR | | | LCVR | | | One of R1 or |
| (or | | | (or | | | R3 is absent. |
| absent) | | | absent) | | | |
| HCVR | Fc | fcFv | HCVR | Fc | scFv | Non-Self |
| and | Region | | and | Region | | Pairing Linker |
| LCVR | | | LCVR | | | Regions |
| (or | | | (or | | | One of R1 or |
| absent) | | | absent) | | | R3 is absent. |
| HCVR | Fc | fcFv | HCVR | Fc | scFv | Self Pairing |
| and | Region | (or | and | Region | (or | linker regions |
| LCVR | | absent) | LCVR | | absent) | One of R2 or |
| | | | | | | R4 is absent. |
| HCVR | Fc | fcFv | HCVR | Fc | scFv | Non-Self |
| and | Region | (or | and | Region | (or | Pairing linker |
| LCVR | | absent) | LCVR | | absent) | regions |
| | | | | | | One of R2 or |
| | | | | | | R4 is absent. |
| HCVR | Fc | fcFv | HCVR | Fc | scFv | Self Pairing |
| and | Region | | and | Region | | Linker Regions |
| LCVR | | | LCVR | | | R1 and R3 are |
| | | | | | | the same |
| HCVR | Fc | fcFv | HCVR | Fc | scFv | Non-Self |
| and | Region | | and | Region | | Pairing linker |
| LCVR | | | LCVR | | | regions |
| | | | | | | R1 and R3 are |
| | | | | | | different |
| HCVR | Fc | fcFv | HCVR | Fc | scFv | Self Pairing |
| and | Region | | and | Region | | Linker Regions |
| LCVR | | | LCVR | | | R2 and R4 are |
| | | | | | | the same |
| HCVR | Fc | fcFv | HCVR | Fc | scFv | Non-Self |
| and | Region | | and | Region | | Pairing linker |
| LCVR | | | LCVR | | | regions |
| | | | | | | R2 and R4 are |
| | | | | | | different |

HCVR and LCVR: refers to an moiety comprising an antigen binding portion of a heavy and light chain variable region, typically with the heavy chain fused to the Linker region.
Self pairing: wherein a liker region can pair with itself, e.g., an Fc region that can pair a copy of itself.
Non-self pairing: wherein a Linker Region does not pair with itself, or does not substantially pair with itself, e.g., an Fc region does not, or does not significantly pair with itself, e.g., wherein Linker Region A and Linker Region B are members of a knob and hole pair.

In some embodiments:
R1, R2, R3 and R4 each independently comprise: an effector binding modulating moiety that activates an inhibitory receptor on an immune cell, e.g., a T cell or a B cell, e.g., a PD-L1 molecule or a functional anti-PD-1 antibody molecule (an agonist of PD-1), a specific targeting moiety, or is absent; provided that an effector binding moiety and a specific targeting moiety are present.

In some embodiments, Linker A and Linker B comprise Fc moieties (e.g., self pairing Fc moieties).

In some embodiments:
R1 and R3 independently comprise an effector binding modulating moiety that activates an inhibitory receptor on an immune cell, e.g., a T cell or a B cell, e.g., a PD-L1 molecule or an functional anti-PD-1 antibody molecule (an agonist of PD-1); and
R2 and R4 independently comprise specific targeting moieties, e.g., scFv molecules against a tissue antigen.

In some embodiments, Linker A and Linker B comprise Fc moieties (e.g., self pairing Fc moieties).

In some embodiments:
R1 and R3 independently comprise a functional anti-PD-1 antibody molecule (an agonist of PD-1); and
R2 and R4 independently comprise specific targeting moieties, e.g., scFv molecules against a tissue antigen.

In some embodiments, Linker A and Linker B comprise Fc moieties (e.g., self pairing Fc moieties).

In some embodiments:
R1 and R3 independently comprise specific targeting moieties, e.g., an anti-tissue antigen antibody; and
R2 and R4 independently comprise a functional anti-PD-1 antibody molecule (an agonist of PD-1), e.g., an scFv molecule.

In some embodiments, Linker A and Linker B comprise Fc moieties (e.g., self pairing Fc moieties).

In some embodiments:
R1 and R3 independently comprise a PD-L1 molecule (an agonist of PD-1); and
R2 and R4 independently comprise specific targeting moieties, e.g., scFv molecules against a tissue antigen; and in some embodiments, Linker A and Linker B comprise Fc moieties (e.g., self pairing Fc moieties).

In some embodiments:
R1 and R3 independently comprise specific targeting moieties, e.g., an anti-tissue antigen antibody; and
R2 and R4 independently comprise a PD-L1 molecule (an agonist of PD-1).

In some embodiments, Linker A and Linker B comprise Fc moieties (e.g., self pairing Fc moieties).

In some embodiments:
R1 and R3 independently comprise a CD39 molecule or a CD73 molecule; and
R2 and R4 independently comprise specific targeting moieties, e.g., scFv molecules against a tissue antigen.

In some embodiments, Linker A and Linker B comprise Fc moieties (e.g., self pairing Fc moieties or Fc moieties that do not, or do not substantially self pair).

In some embodiments:
R1 and R3 each comprises a CD39 molecule; and
R2 and R4 independently comprise specific targeting moieties, e.g., scFv molecules against a tissue antigen; and in some embodiments, Linker A and Linker B comprise Fc moieties (e.g., self pairing Fc moieties or Fc moieties that do not, or do not substantially self pair).

In some embodiments:

In an embodiment:
R1, R2, R3 and R4 each independently comprise: an IL-2 mutein molecule; a specific targeting moiety, or is absent;
provided that an IL-2 mutein molecule and a specific targeting moiety are present.

In an embodiment, Linker A and Linker B comprise Fc moieties (e.g., self pairing Fc moieties or Fc moieties that do not, or do not substantially self pair).

In an embodiment:
R1 and R3 each comprise an IL-2 mutein molecule; and
R2 and R4 independently comprise specific targeting moieties, e.g., scFv molecules against a tissue antigen.

In an embodiment Linker A and Linker B comprise Fc moieties (e.g., self pairing Fc moieties or Fc moieties that do, or do not substantially self pair).

In some embodiments, one of R1, R2, R3, and R4 comprises an SM binding/modulating moiety, e.g., a CD39 molecule. In some embodiments, one of R1, R2, R3, and R4 comprises an entity that binds, activates, or maintains, a regulatory immune cell, e.g., a Treg cell or a Breg cell, for example, an IL-2 mutein molecule.

In some embodiments, one of R1, R2, R3, and R4 comprises an agonistic anti-PD-1 antibody. In some embodiments, the PD-1 antibody is replaced with a IL-2 mutein molecule. In some embodiments, one of R1, R2, R3, and R4 comprises an agonistic anti-PD-1 antibody, one comprises an HLA-G molecule, and one comprises CD39 molecule. In some embodiments, the PD-1 antibody is replaced with a IL-2 mutein molecule.

In some embodiments, one of R1 and R2 is an IL-2 mutein and one of R1 and R2 is an anti-desmoglein-1 antibody, an anti-desmoglein-2 antibody, anti-desmoglein-3 antibody, or an anti-desmoglein 4 antibody. In some embodiments, R1 is an IL-2 mutein and R2 is an anti-desmoglein-1 antibody, an anti-desmoglein-2 antibody, anti-desmoglein-3 antibody, or an anti-desmoglein 4 antibody. In some embodiments, R1 is an anti-desmoglein-1 antibody, an anti-desmoglein-2 antibody, anti-desmoglein-3 antibody, or an anti-desmoglein 4 antibody and R2 is an IL-2 mutein. In some embodiments, one of R3 and R4 is an IL-2 mutein and one of R3 and R4 is an anti-desmoglein-1 antibody, an anti-desmoglein-2 antibody, anti-desmoglein-3 antibody, or an anti-desmoglein 4 antibody. In some embodiments, R3 is an IL-2 mutein and R4 is an anti-desmoglein-1 antibody, an anti-desmoglein-2 antibody, anti-desmoglein-3 antibody, or an anti-desmoglein 4 antibody. In some embodiments, R3 is an anti-desmoglein-1 antibody, an anti-desmoglein-2 antibody, anti-desmoglein-3 antibody, or an anti-desmoglein 4 antibody and one R4 is an IL-2 mutein.

In some embodiments, one of R1 and R2 is an anti-PD-1 antibody and one of R1 and R2 is an anti-desmoglein-1 antibody, an anti-desmoglein-2 antibody, anti-desmoglein-3 antibody, or an anti-desmoglein 4 antibody. In some embodiments, R1 is an anti-PD-1 antibody and R2 is an anti-desmoglein-1 antibody, an anti-desmoglein-2 antibody, anti-desmoglein-3 antibody, or an anti-desmoglein 4 antibody. In some embodiments, R1 is an anti-desmoglein-1 antibody, an anti-desmoglein-2 antibody, anti-desmoglein-3 antibody, or an anti-desmoglein 4 antibody and R2 is an anti-PD-1 antibody. In some embodiments, one of R3 and R4 is an anti-PD-1 antibody and one of R3 and R4 is an anti-desmoglein-1 antibody, an anti-desmoglein-2 antibody, anti-desmoglein-3 antibody, or an anti-desmoglein 4 antibody. In some embodiments, R3 is an anti-PD-1 antibody and R4 is an anti-desmoglein-1 antibody, an anti-desmoglein-2 antibody, anti-desmoglein-3 antibody, or an anti-desmoglein 4 antibody. In some embodiments, R3 is an anti-desmoglein-1 antibody, an anti-desmoglein-2 antibody, anti-desmoglein-3 antibody, or an anti-desmoglein 4 antibody and one R4 is an anti-PD-1 antibody.

In some embodiments, one of R1 and R2 is a CD39 Effector Domain and one of R1 and R2 is an anti-desmoglein-1 antibody, an anti-desmoglein-2 antibody, anti-desmoglein-3 antibody, or an anti-desmoglein 4 antibody. In some embodiments, R1 is a CD39 Effector Domain and R2 is an anti-desmoglein-1 antibody, an anti-desmoglein-2 antibody, anti-desmoglein-3 antibody, or an anti-desmoglein 4 antibody. In some embodiments, R1 is an anti-desmoglein-1 antibody, an anti-desmoglein-2 antibody, anti-desmoglein-3 antibody, or an anti-desmoglein 4 antibody and R2 is a CD39 Effector Domain. In some embodiments, one of R3 and R4 is a CD39 Effector Domain and one of R3 and R4 is an anti-desmoglein-1 antibody, an anti-desmoglein-2 antibody, anti-desmoglein-3 antibody, or an anti-desmoglein 4 antibody. In some embodiments, R3 is a CD39 Effector Domain and R4 is an anti-desmoglein-1 antibody, an anti-desmoglein-2 antibody, anti-desmoglein-3 antibody, or an anti-desmoglein 4 antibody. In some embodiments, R3 is an anti-desmoglein-1 antibody, an anti-desmoglein-2 antibody, anti-desmoglein-3 antibody, or an anti-desmoglein 4 antibody and one R4 is a CD39 Effector Domain.

### Linker Regions

As discussed elsewhere herein specific targeting and effector binding/modulating moieties can be linked by linker regions. Any linker region described herein can be used as a linker. For example, Linker Regions A and B can comprise Fc regions. In some embodiments, a therapeutic compound comprises a Linker Region that can self-associate. In some embodiments, a therapeutic compound comprises a Linker Region that has a moiety that minimizes self association, and typically Linker Region A and Linker Region B are heterodimers. Linkers also include glycine/serine linkers. In some embodiments, the linker can comprise one or more repeats of GGGGS (SEQ ID NO: 23). In some embodiments, the linker comprises 1, 2, 3, 4, or 5 repeats of SEQ ID NO: 23. In some embodiments, the linker comprises of GGGGS (SEQ ID NO: 23), GGGGSGGGGSGGGGSGGGGS (SEQ ID NO: 22), or GGGGSGGGGSGGGGS (SEQ ID NO: 30). In some embodiments, the linker comprises of GGGGSEGGGSEGGGSEGGGSE (SEQ ID NO: 71), GGGSEGGGSEGGGSEGGGSE (SEQ ID NO: 72), GGGSEGGGSEGGGSE (SEQ ID NO: 73), AEEEKAEEEKAEEEKAEEEK (SEQ ID NO: 74), GGGSKGGGSKGGGSK (SEQ ID NO: 75), GSAGKGSAGKGSAGK (SEQ ID NO: 76), GGGSKGGGSKGGGSK (SEQ ID NO: 77), GSAGK (SEQ ID NO: 78), GS, GAGGGSKGGGSKGGGSK (SEQ ID NO: 79), GSAGKGSAGKGSAGK (SEQ ID NO: 80), GGGSK (SEQ ID NO: 81), AEEEK (SEQ ID NO: 82), GGSSGSGSGSTGTSSSGTGTSAGTTGTSASTSGSGSGGGGGSGGGGSAGGTATAGASSG S (SEQ ID NO: 83), These linkers can be used in any of the therapeutic compounds or compositions provided herein.

The linker region can comprise a Fc region that has been modified (e.g., mutated) to produce a heterodimer. In some embodiments, the CH3 domain of the Fc region can be mutated. Examples of such Fc regions can be found in, for example, U.S. Pat. No. 9,574,010, which is hereby incorporated by reference in its entirety. The Fc region as defined herein comprises a CH3 domain or fragment thereof, and may additionally comprise one or more addition constant region domains, or fragments thereof, including hinge, CH1, or CH2. It will be understood that the numbering of the Fc amino acid residues is that of the EU index as in Kabat et al 1991, NIH Publication 91-3242, National Technical Information Service, Springfield, Va. The “EU index as set forth in Kabat” refers to the EU index numbering of the human IgG1 Kabat antibody. For convenience, Table B of U.S. Pat. No. 9,574,010 provides the amino acids numbered according to the EU index as set forth in Kabat of the CH2 and CH3 domain from human IgG1, which is hereby incorporated by reference. Table 1.1 of U.S. Pat. No. 9,574,010 provides mutations of variant Fc heterodimers that can be used as linker regions. Table 1.1 of U.S. Pat. No. 9,574,010 is hereby incorporated by reference.

In some embodiments, the Linker Region A comprises a first CH3 domain polypeptide and a the Linker Region B comprises a second CH3 domain polypeptide, the first and second CH3 domain polypeptides independently comprising amino acid modifications as compared to a wild-type CH3 domain polypeptide, wherein the first CH3 domain polypeptide comprises amino acid modifications at positions T350, L351, F405, and Y407, and the second CH3 domain polypeptide comprises amino acid modifications at positions T350, T366, K392 and T394, wherein the amino acid modification at position T350 is T350V, T3501, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V, or T366M; the amino acid modification at position K392 is K392F, K392L or K392M; and the amino acid modification at position T394 is T394W, and wherein the numbering of amino acid residues is according to the EU index as set forth in Kabat.

In some embodiments, the amino acid modification at position K392 is K392M or K392L. In some embodiments, the amino acid modification at position T350 is T350V. In some embodiments, the first CH3 domain polypeptide further comprises one or more amino acid modifications selected from Q347R and one of S400R or S400E. In some embodiments, the second CH3 domain polypeptide further comprises one or more amino acid modifications selected from L351Y, K360E, and one of N390R, N390D or N390E. In some embodiments, the first CH3 domain polypeptide further comprises one or more amino acid modifications selected from Q347R and one of S400R or S400E, and the second CH3 domain polypeptide further comprises one or more amino acid modifications selected from L351Y, K360E, and one of N390R, N390D or N390E. In some embodiments, the amino acid modification at position T350 is T350V. In some embodiments, the amino acid modification at position F405 is F405A. In some embodiments, the amino acid modification at position Y407 is Y407V. In some embodiments, the amino acid modification at position T366 is T366L or T366I. In some embodiments, the amino acid modification at position F405 is F405A, the amino acid modification at position Y407 is and Y407V, the amino acid modification at position T366 is T366L or T366I, and the amino acid modification at position K392 is K392M or K392L. In some embodiments, the first CH3 domain polypeptide comprises the amino acid modifications T350V, L351Y, S400E, F405V and Y407V, and the second CH3 domain polypeptide comprises the amino acid modifications T350V, T366L, N390R, K392M and T394W. In some embodiments, the first CH3 domain polypeptide comprises the amino acid modifications T350V, L351Y, S400E, F405T and Y407V, and the second CH3 domain polypeptide comprises the amino acid modifications T350V, T366L, N390R, K392M and T394W. In some embodiments, the first CH3 domain polypeptide comprises the amino acid modifications T350V, L351Y, S400E, F405S and Y407V, and the second CH3 domain polypeptide comprises the amino acid modifications T350V, T366L, N390R, K392M and T394W. In some embodiments, the first CH3 domain polypeptide comprises the amino acid modifications T350V, L351Y, S400E, F405A and Y407V, and the second CH3 domain polypeptide comprises the amino acid modifications T350V, L351Y, T366L, N390R, K392M and T394W. In some embodiments, the first CH3 domain polypeptide comprises the amino acid modifications Q347R, T350V, L351Y, S400E, F405A and Y407V, and the second CH3 domain polypeptide comprises the amino acid modifications T350V, K360E, T366L, N390R, K392M and T394W. In some embodiments, the first CH3 domain polypeptide comprises the amino acid modifications T350V, L351Y, S400R, F405A and Y407V, and the second CH3 domain polypeptide comprises the amino acid modifications T350V, T366L, N390D, K392M and T394W. In some embodiments, the first CH3 domain polypeptide comprises the amino acid modifications T350V, L351Y, S400R, F405A and Y407V, and the second CH3 domain polypeptide comprises the amino acid modifications T350V, T366L, N390E, K392M and T394W. In some embodiments, the first CH3 domain polypeptide comprises the amino acid modifications T350V, L351Y, S400E, F405A and Y407V, and the second CH3 domain polypeptide comprises the amino acid modifications T350V, T366L, N390R, K392L and T394W. In some embodiments, the first CH3 domain polypeptide comprises the amino acid modifications T350V, L351Y, S400E, F405A and Y407V, and the second CH3 domain polypeptide comprises the amino acid modifications T350V, T366L, N390R, K392F and T394W.

In some embodiments, an isolated heteromultimer comprising a heterodimeric CH3 domain comprising a first CH3 domain polypeptide and a second CH3 domain polypeptide, the first CH3 domain polypeptide comprising amino acid modifications at positions F405 and Y407, and the second CH3 domain polypeptide comprising amino acid modifications at positions T366 and T394, wherein: (i) the first CH3 domain polypeptide further comprises an amino acid modification at position L351, and (ii) the second CH3 domain polypeptide further comprises an amino acid modification at position K392, wherein the amino acid modification at position F405 is F405A, F405T, F405S or F405V; and the amino acid modification at position Y407 is Y407V, Y407A, Y407L or Y407I; the amino acid modification at position T394 is T394W; the amino acid modification at position L351 is L351Y; the amino acid modification at position K392 is K392L, K392M, K392V or K392F, and the amino acid modification at position T366 is T366I, T366L, T366M or T366V, wherein the heterodimeric CH3 domain has a melting temperature (Tm) of about 70° C. or greater and a purity greater than about 90%, and wherein the numbering of amino acid residues is according to the EU index as set forth in Kabat.

In some embodiments, the Linker Region A comprises a first CH3 domain polypeptide and a t Linker Region B comprises a second CH3 domain polypeptide, wherein the first CH3 domain polypeptide comprising amino acid modifications at positions F405 and Y407, and the second CH3 domain polypeptide comprising amino acid modifications at positions T366 and T394, wherein: (i) the first CH3 domain polypeptide further comprises an amino acid modification at position L351, and (ii) the second CH3 domain polypeptide further comprises an amino acid modification at position K392, wherein the amino acid modification at position F405 is F405A, F405T, F405S or F405V; and the amino acid modification at position Y407 is Y407V, Y407A, Y407L or Y407I; the amino acid modification at position T394 is T394W; the amino acid modification at position L351 is L351Y; the amino acid modification at position K392 is K392L, K392M, K392V or K392F, and the amino acid modification at position T366 is T366I, T366L, T366M or T366V, wherein the heterodimeric CH3 domain has a melting temperature (Tm) of about 70 C. or greater and a purity greater than about 90%, and wherein the numbering of amino acid residues is according to the EU index as set forth in Kabat. In some embodiments, the amino acid modification at position F405 is F405A. In some embodiments, the amino acid modification at position T366 is T366I or T366L. In some embodiments, the amino acid modification at position Y407 is Y407V. In some embodiments, the amino acid modification at position F405 is F405A, the amino acid modification at position Y407 is Y407V, the amino acid modification at position T366 is T366I or T366L, and the amino acid modification at position K392 is K392L or K392M. In some embodiments, the amino acid modification at position F405 is F405A, the amino acid modification at position Y407 is Y407V, the amino acid modification at position T366 is T366L, and the amino acid modification at position K392 is K392M. In some embodiments, the amino acid modification at position F405 is F405A, the amino acid modification at position Y407 is Y407V, the amino acid modification at position T366 is T366L, and the amino acid modification at position K392 is K392L. In some embodiments, the amino acid modification at position F405 is F405A, the amino acid modification at position Y407 is Y407V, the amino acid modification at position T366 is T366I, and the amino acid modification at position K392 is K392M. In some embodiments, the amino acid modification at position F405 is F405A, the amino acid modification at position Y407 is Y407V, the amino acid modification at position T366 is T366I, and the amino acid modification at position K392 is K392L. In some embodiments, the first CH3 domain polypeptide further comprises an amino acid modification at position 5400 selected from S400D and S400E, and the second CH3 domain polypeptide further comprises the amino acid modification N390R. In some embodiments, the amino acid modification at position F405 is F405A, the amino acid modification at position Y407 is Y405V, the amino acid modification at position 5400 is S400E, the amino acid modification at position T366 is T366L, and the amino acid modification at position K392 is K392M.

In some embodiments, the modified first and second CH3 domains are comprised by an Fc construct based on a type G immunoglobulin (IgG). The IgG can be an IgG1, IgG2, IgG3, or IgG4.

Other Linker Region A and Linger Region B comprising variant CH3 domains are described in U.S. Pat. Nos. 9,499,634 and 9,562,109, each of which is incorporated by reference in its entirety.

A Linker Region A and Linker Region B can be complementary fragments of a protein, e.g., a naturally occurring protein such as human serum albumin. In embodiments, one of Linker Region A and Linker Region B comprises a first, e.g., an N-terminal fragment of the protein, e.g., hSA, and the other comprises a second, e.g., a C-terminal fragment of the protein, e.g., has. In an embodiment the fragments comprise an N-terminal and a C-terminal fragment. In an embodiment the fragments comprise two internal fragments. Typically the fragments do not overlap. In an embodiment the first and second fragment, together, provide the entire sequence of the original protein, e.g., hSA. The first fragment provides a N-terminus and a C-terminus for linking, e.g., fusing, to other sequences, e.g., sequences of R1, R2, R3, or R4 (as defined herein).

The Linker Region A and the Linker Region B can be derived from albumin polypeptide. In some embodiments, the albumin polypeptide is selected from native human serum albumin polypeptide and human alloalbumin polypeptide. The albumin polypeptide can be modified such that the Linker Region A and Linker Region B interact with one another to form heterodimers. Examples of modified albumin polypeptides are described in U.S. Pat. Nos. 9,388,231 and 9,499,605, each of which is hereby incorporated by reference in its entirety.

Accordingly, provided herein are multifunctional heteromultimer proteins of the formula R1 Linker Region A-R2 and R3-Linker Region B-R4, wherein the Linker Region A and Linker Region B form a heteromultimer. In some embodiments, the Linker Region A comprises a first polypeptide and the Linker Region B comprises a second polypeptide; wherein each of said first and second polypeptides comprises an amino acid sequence comprising a segment of an albumin polypeptide selected from native human serum albumin polypeptide and human alloalbumin polypeptide; wherein said first and second polypeptides are obtained by segmentation of said albumin polypeptide at a segmentation site, such that the segmentation results in a deletion of zero to 3 amino acid residues at the segmentation site; wherein said first polypeptide comprises at least one mutation selected from A194C, L198C, W214C, A217C, L331C and A335C, and said second polypeptide comprises at least one mutation selected from L331C, A335C, V343C, L346C, A350C, V455C, and N458C; and wherein said first and second polypeptides self-assemble to form a quasi-native structure of the monomeric form of the albumin polypeptide.

In some embodiments, the segmentation site resides on a loop of the albumin polypeptide that has a high solvent accessible surface area (SASA) and limited contact with the rest of the albumin structure. In some embodiments, the segmentation results in a complementary interface between the transporter polypeptides. These segmentation sites are described, for example, in U.S. Pat. No. 9,388,231, which is hereby incorporated by reference in its entirety.

In some embodiments, the first polypeptide comprises residues 1-337 or residues 1-293 of the albumin polypeptide with one or more of the mutations described herein. In some embodiments, the second polypeptide comprises residues of 342-585 or 304-585 of the albumin polypeptide with one or more of the mutations described herein. In some embodiments, the first polypeptide comprises residues 1-339, 1-300, 1-364, 1-441, 1-83, 1-171, 1-281, 1-293, 1-114, 1-337, or 1-336 of the albumin protein. In some embodiments, the second polypeptide comprises residues 301-585, 365-585, 442-585, 85-585, 172-585, 282-585, or 115-585, 304-585, 340-585, or 342-585 of the albumin protein.

In some embodiments, the first and second polypeptide comprise the residues of the albumin protein as shown in the table below. The sequence of the albumin protein is described below.

| First Polypeptide Residues | Second Polypeptide Residues |
|---|---|
| 1-300 | 301-585 |
| 1-364 | 365-585 |
| 1-441 | 442-585 |
| 1-83 | 85-585 |
| 1-171 | 172-585 |
| 1-281 | 282-585 |
| 1-114 | 115-585 |
| 1-339 | 340-585 |
| 1-337 | 342-585 |
| 1-293 | 304-585 |
| 1-336 | 342-585 |

In some embodiments, the first and second polypeptides comprise a linker that can form a covalent bond with one another, such as a disulfide bond. A non-limiting example of the linker is a peptide linker. In some embodiments, the peptide linker comprises GGGGS (SEQ ID NO: 23). The linker can be fused to the C-terminus of the first polypeptide and the N-terminus of the second polypeptide. The linker can also be used to attach the moieties described herein without abrogating the ability of the linkers to form a disulfide bond. In some embodiments, the first and second polypeptides do not comprise a linker that can form a covalent bond. In some embodiments, the first and second polypeptides have the following substitutions.

| First Polypeptide Substitution | Second Polypeptide Substitution |
|---|---|
| A217C | V343C |
| L331C | A350C |
| A217C | L346C |
| W214C | V343C |
| A335C | L346C |
| L198C | V455C |
| A217C | A335C |
| A217C | L331C |
| L198C | N458C |
| A194C | V455C |

The sequence of the albumin polypeptide can be the sequence of human albumin as shown, in the post-protein form with the N-terminal signaling residues removed

(MKWVTFISLLFLFSSAYSRGVFRR, SEQ ID NO: 84)
(human albumin, SEQ ID NO: 85)
DAHKSEVAHRFKDLGEENFKALVLIAFAQYLQQCPFEDHV
KLVNEVTEFAKTCVADESAENCDKSLHTLFGDKLCTVATL
RETYGEMADCCAKQEPERNECFLQHKDDNPNLPRLVRPEV
DVMCTAFHDNEETFLKKYLYEIARRHPYFYAPELLFFAKRY
KAAFTECCQAADKAACLLPKLDELRDEGKASSAKQRLKCA
SLQKFGERAFKAWAVARLSQRFPKAEFAEVSKLVTDLTKV
HTECCHGDLLECADDRADLAKYICENQDSISSKLKECCEKP
LLEKSHCIAEVENDEMPADLPSLAADFVESKDVCKNYAEA
KDVFLGMFLYEYARRHPDYSVVLLLRLAKTYETTLEKCCA
AADPHECYAKVFDEFKPLVEEPQNLIKQNCELFEQLGEYKF
QNALLVRYTKKVPQVSTPTLVEVSRNLGKVGSKCCKHPEA
KRMPCAEDYLSVVLNQLCVLHEKTPVSDRVTKCCTESLVN
RRPCFSALEVDETYVPKEFNAETFTFHADICTLSEKERQIKK
QTALVELVKHKPKATKEQLKAVMDDFAAFVEKCCKADDK
ETCFAEEGKKLVAASQAALGL

In some embodiments, the Linker Region A and the Linker Region B form a heterodimer as described herein.

In some embodiments, the polypeptide comprises at the N-terminus an antibody comprised of F(ab′)2 on an IgG1 Fc backbone fused with scFvs on the C-terminus of the IgG Fc backbone. In some embodiments, the IgG Fc backbone is a IgG1 Fc backbone. In some embodiments, the IgG1 backbone is replaced with a IgG4 backbone, IgG2 backbone, or other similar IgG backbone. The IgG backbones described in this paragraph can be used throughout this application where a Fc region is referred to as part of the therapeutic compound. Thus, in some embodiments, the antibody comprised of F(ab′)2 on an IgG1 Fc backbone can be an anti- an anti-desmoglein 1 antibody, an anti-desmoglein 2 antibody, an anti-desmoglein 3 antibody, or an anti-desmoglein 4 antibody or an anti-PD-1 antibody on an IgG1 Fc or any other targeting moiety or effector binding/modulating moiety provided herein. In some embodiments, the scFV segments fused to the C-terminus could be an anti-PD-1 antibody, if the N-terminus region is an anti-desmoglein 1 antibody, an anti-desmoglein 2 antibody, an anti-desmoglein 3 antibody, or an anti-desmoglein 4 antibody, if the N-terminus region is an anti-PD-1 antibody. In this non-limiting example, the N-terminus can be the targeting moiety, such as any one of the ones provided for herein, and the C-terminus can be the effector binding/modulating moiety, such as any of the ones provided for herein. Alternatively, in some embodiments, the N-terminus can be the effector binding/modulating moiety, such as any one of the ones provided for herein, and the C-terminus can be the targeting moiety, such as any of the ones provided for herein.

In some embodiments, the N-terminus can be the targeting moiety, such as any one of the ones provided for herein, and the C-terminus can be the effector binding/modulating moiety, such as any of the ones provided for herein.

In some embodiments, the therapeutic compound comprises two polypeptides that homodimerize. In some embodiments, the N-terminus of the polypeptide comprises an effector binding/modulating moiety that is fused to a human IgG1 Fc domain (e.g., CH2 and/or CH3 domains). In some embodiments, the C-terminus of the Fc domain is another linker that is fused to the targeting moiety. Thus, in some embodiments, the molecule could be represented using the formula of R1-Linker A-Fc Region-Linker B-R2, wherein R1 can be an effector binding/modulating moiety, R2 is a targeting moiety, Linker A and Linker B are independently linkers as provided for herein. In some embodiments, Linker 1 and Linker 2 are different.

In some embodiments, the molecule could be represented using the formula of R1-Linker A-Fc Region-Linker B-R2, wherein R1 can be a targeting moiety, R2 is an effector binding/modulating moiety, Linker A and Linker B are independently linkers as provided for herein. In some embodiments, Linker A and Linker B are different. The linkers can be chosen from the non-limiting examples provided for herein. In some embodiments, R1 and R2 are independently selected from F(ab′)2 and scFV antibody domains. In some embodiments, R1 and R2 are different antibody domains. In some embodiments, the scFV is in the VL-VH domain orientation.

In some embodiments, the therapeutic compound is a bispecific antibody. In some embodiments, the bispecific antibodies are comprised of four polypeptide chains comprising the following:
Chain 1: nt-VH1-CH1-CH2-CH3-Linker A-scFv[VL2-Linker B-VH2]-ct
Chain 2: nt-VH1-CH1-CH2-CH3-Linker A-scFv[VL2-Linker B-VH2]-ct
Chain 3: nt-VL1-CL-ct
Chain 4: nt-VL1-CL-ct,
wherein chains 1 and 2 are identical to each other, and chains 3 and 4 are identical to each other,
wherein chain 1 forms a homodimer with chain 2; and chain 3 and 4 associate with chain 1 and chain 2. That is, when each light chain associates with each heavy chain, VL1 associates with VH1 and CL associates with CH1 to form two functional Fab units. Without being bound to any particular theory, each scFv unit is intrinsically functional since VL2 and VH2 are covalently linked in tandem with a linker as provided herein (e.g., GGGGSG (SEQ ID NO: 23), GGGGSGGGGS (SEQ ID NO: 531), GGGGSGGGGSGGGGSGGGGS (SEQ ID NO: 22), or GGGGSGGGGSGGGGS (SEQ ID NO: 30). The sequences of Linker A and Linker B, which are independent of one another can be the same or different and as otherwise described throughout the present application. Thus, in some embodiments, Linker A comprises GGGGS (SEQ ID NO: 23), or two repeats thereof, GGGGSGGGGSGGGGS (SEQ ID NO: 30), or GGGGSGGGGSGGGGSGGGGS (SEQ ID NO: 22). In some embodiments, Linker B comprises GGGGS (SEQ ID NO: 23), or two repeats thereof, GGGGSGGGGS (SEQ ID NO: 531), GGGGSGGGGSGGGGS (SEQ ID NO: 30), or GGGGSGGGGSGGGGSGGGGS (SEQ ID NO: 22). The scFv may be arranged in the NT-VH2-VL2-CT or NT-VL2-VH2-CT orientation. NT or nt stands for N-terminus and CT or ct stands for C-terminus of the protein. CH1, CH2, and CH3 are the domains from the IgG Fc region, and CL stands for Constant Light chain, which can be either kappa or lambda family light chains. The other definitions stand for the way they are normally used in the art.

In some embodiments, the VH1 and VL1 domains are derived from the effector molecule and the VH2 and VL2 domains are derived from the targeting moiety. In some embodiments the VH1 and VL1 domains are derived from a targeting moiety and the VH2 and VL2 domains are derived from an effector binding/modulating moiety.

In some embodiments, the VH1 and VL1 domains are derived from an anti-PD-1 antibody, and the VH2 and VL2 domains are derived from an anti-desmoglein 1 antibody, an anti-desmoglein 2 antibody, an anti-desmoglein 3 antibody, or an anti-desmoglein 4 antibody. In some embodiments the VH1 and VL1 domains are derived from an anti-desmoglein 1 antibody, an anti-desmoglein 2 antibody, an anti-desmoglein 3 antibody, or an anti-desmoglein 4 antibody and the VH2 and VL2 domains are derived from an anti-PD-1 antibody.

In some embodiments, Linker A comprises 1, 2, 3, 4, or 5 GGGGS (SEQ ID NO: 23) repeats. In some embodiments, Linker B comprises 1, 2, 3, 4, or 5 GGGGS (SEQ ID NO: 23) repeats. For the avoidance of doubt, the sequences of Linker A and Linker B, which are used throughout this application, are independent of one another. Therefore, in some embodiments, Linker A and Linker B can be the same or different. In some embodiments, Linker A comprises GGGGS (SEQ ID NO: 23), or two repeats thereof, GGGGSGGGGS (SEQ ID NO: 531), GGGGSGGGGSGGGGS (SEQ ID NO: 30), or GGGGSGGGGSGGGGSGGGGS (SEQ ID NO: 22). In some embodiments, Linker B comprises GGGGS (SEQ ID NO: 23), or two repeats thereof, GGGGSGGGGS (SEQ ID NO: 531), GGGGSGGGGSGGGGS (SEQ ID NO: 30), or GGGGSGGGGSGGGGSGGGGS (SEQ ID NO: 22).

In some embodiments, the therapeutic compound comprises a light chain and a heavy chain. In some embodiments, the light and heavy chain begin at the N-terminus with the VH domain of a targeting moiety followed by the CH1 domain of a human IgG1, which is fused to a Fc region (e.g., CH2-CH3) of human IgG1. In some embodiments, at the C-terminus of the Fc region is fused to a linker as provided herein, such as but not limited to, GGGGS (SEQ ID NO: 23), or two or three repeats thereof, GGGGSGGGGS (SEQ ID NO: 531), or GGGGSGGGGSGGGGS (SEQ ID NO: 22). The linker can then be fused to an effector binding/modulating moiety, such as any one of the effector moieties provided for herein. The polypeptides can homodimerize because through the heavy chain homodimerization, which results in a therapeutic compound having two effector moieties, such as two anti-PD-1 antibodies. In this orientation, the targeting moiety is an IgG format, there are two Fab arms that each recognize binding partner of the targeting moiety, for example, desmoglein 1, 2, 3, and/or 4 being bound by the desmoglein 1, 2, 3, and/or 4 targeting moiety.

In some embodiments, the targeting moiety is an anti-desmoglein 1 antibody, an anti-desmoglein 2 antibody, an anti-desmoglein 3 antibody, or an anti-desmoglein 4 antibody.

In some embodiments, the an anti-desmoglein 1 antibody, an anti-desmoglein 2 antibody, an anti-desmoglein 3 antibody, or an anti-desmoglein 4 antibody comprises a sequence as provided for herein. In some embodiments, the desmoglein moiety is a single chain scFv as provided for herein. In some embodiments, the desmoglein moiety is a single chain Fab as provided for herein.

TABLE 1
Desmoglein Antibody

| Ab | VH | VK | | | | | | |
|---|---|---|---|---|---|---|---|---|
| (scFab) | Seq | Seq | CDR1 | CDR2 | CDR3 | LCDR1 | LCDR2 | LCDR3 |
| SD1 | QVQLQ | QSVL | SSNH | GEIY | ARGW | SGSSS | SNDQR | AAWDD |
| | ESGPG | TQPP | W | HNGS | HRTG | HIGSN | PA | GQGGV |
| | PVKPS | SVSG | (SEQ | TFLN | FRGY | YVY | (SEQ | (SEQ |
| | GTLSL | TPGQ | ID | PSLK | PSHW | (SEQ | ID | ID |
| | TCGVS | RVTI | NO: | (SEQ | YFDL | ID | NO: | NO: |
| | GGSIS | SCSG | 88) | ID | (SEQ | NO: | 92) | 93) |
| | SNHWW | SSSH | | NO: | ID | 91) | | |
| | TWVRQ | IGSN | | 89) | NO: | | | |
| | PPGKG | YVYW | | | 90) | | | |
| | LEWIG | YQQL | | | | | | |
| | EIYHN | PGTA | | | | | | |
| | GSTFL | PKIL | | | | | | |
| | NPSLK | IYSN | | | | | | |
| | SRVTI | DQRP | | | | | | |
| | SVDKS | AGVP | | | | | | |
| | NNQFS | DRFS | | | | | | |
| | LKLTS | ASKS | | | | | | |
| | VTAAD | GTSA | | | | | | |
| | TAVYY | SLAI | | | | | | |
| | CARGW | SGLR | | | | | | |
| | HRTGF | SEDE | | | | | | |
| | RGYPS | ADYY | | | | | | |
| | HWYFD | CAAW | | | | | | |
| | LWGRG | DDGQ | | | | | | |
| | TLVSV | GGVF | | | | | | |
| | SSAST | GGGT | | | | | | |
| | KGPSV | KLTV | | | | | | |
| | FPLAP | LGQP | | | | | | |
| | SSKST | KAAP | | | | | | |
| | SGGTA | SVTL | | | | | | |
| | ALGCL | FPPS | | | | | | |
| | VKDYF | SEEL | | | | | | |
| | PEPVT | QANK | | | | | | |
| | VSWNS | ATLV | | | | | | |
| | GALTS | CLIS | | | | | | |
| | GVHTF | DFYP | | | | | | |
| | PAVLQ | GAVT | | | | | | |
| | SSGLY | VAWK | | | | | | |
| | SLSSV | ADSS | | | | | | |
| | VTVPS | PVKA | | | | | | |
| | SSLGT | GVET | | | | | | |
| | QTYIC | TTPS | | | | | | |
| | NVNHK | KQSN | | | | | | |
| | PSNTK | NKYA | | | | | | |
| | VDKKV | ASSY | | | | | | |
| | EPKSC | LSLT | | | | | | |
| | DKT | PEQW | | | | | | |
| | (SEQ | KSHR | | | | | | |
| | ID | SYSC | | | | | | |
| | NO: | QVTH | | | | | | |
| | 86) | EGST | | | | | | |
| | | VEKT | | | | | | |
| | | VAPT | | | | | | |
| | | ECS | | | | | | |
| | | (SEQ | | | | | | |
| | | ID | | | | | | |
| | | NO: | | | | | | |
| | | 87) | | | | | | |

TABLE 2
Desmoglein Antibody

| Clone | VH | VK | | | | | | |
|---|---|---|---|---|---|---|---|---|
| (scFv) | Seq | Seq | CDR1 | CDR2 | CDR3 | LCDR1 | LCDR2 | LCDR3 |
| SD1 | QVQLQ | QSVL | SSNH | GEIY | ARGW | SGSSS | SNDQR | AAWDD |
| | ESGPG | TQPP | W | HNGS | HRTG | HIGSN | PA | GQGGV |
| | PVKPS | SVSG | (SEQ | TFLN | FRGY | YVY | (SEQ | (SEQ |
| | GTLSL | TPGQ | ID | PSLK | PSHW | (SEQ | ID | ID |
| | TCGVS | RVTI | NO: | (SEQ | YFDL | ID | NO: | NO: |
| | GGSIS | SCSG | 88) | ID | (SEQ | NO: | 92) | 93) |
| | SNHWW | SSSH | | NO: | ID | 91) | | |
| | TWVRQ | IGSN | | 89) | NO: | | | |
| | PPGKG | YVYW | | | 90) | | | |
| | LEWIG | YQQL | | | | | | |
| | EIYHN | PGTA | | | | | | |
| | GSTFL | PKIL | | | | | | |
| | NPSLK | IYSN | | | | | | |
| | SRVTI | DQRP | | | | | | |
| | SVDKS | AGVP | | | | | | |
| | NNQFS | DRFS | | | | | | |
| | LKLTS | ASKS | | | | | | |
| | VTAAD | GTSA | | | | | | |
| | TAVYY | SLAI | | | | | | |
| | CARGW | SGLR | | | | | | |
| | HRTGF | SEDE | | | | | | |
| | RGYPS | ADYY | | | | | | |
| | HWYFD | CAAW | | | | | | |
| | LWGRG | DDGQ | | | | | | |
| | TLVSV | GGVF | | | | | | |
| | SS | GGGT | | | | | | |
| | (SEQ | KLTV | | | | | | |
| | ID | L | | | | | | |
| | NO: | (SEQ | | | | | | |
| | 94) | ID | | | | | | |
| | | NO: | | | | | | |
| | | 95) | | | | | | |
| SD2 | QVQLV | DIQM | TTYD | MNPT | ARGL | RASQS | AASSL | QQSYS |
| | QSGAE | TQSP | IN | SGNT | FFGV | ISYLS | QS | TPRT |
| | VRKPG | SSLS | (SEQ | AYAQ | VTKP | N | (SEQ | (SEQ |
| | ASVRV | ASVG | ID | KFQG | NYYY | (SEQ | ID | ID |
| | SCKAS | DRVT | NO: | (SEQ | YAMD | ID | NO: | NO: |
| | GYTLT | ITCR | 98) | ID | V | NO: | 102) | 103) |
| | TYDIN | ASQS | | NO: | (SEQ | 101) | | |
| | WVRQA | ISSY | | 99) | ID | | | |
| | TGQGL | LNWY | | | NO: | | | |
| | EWMGW | QQKP | | | 100) | | | |
| | MNPTS | GKAP | | | | | | |
| | GNTAY | KLLI | | | | | | |
| | AQKFQ | YAAS | | | | | | |
| | GRVTM | SLQS | | | | | | |
| | TRNTS | GVPS | | | | | | |
| | ISTAY | RFSG | | | | | | |
| | MELSS | SGSG | | | | | | |
| | LRSED | TDFT | | | | | | |
| | TAVYY | LTIS | | | | | | |
| | CARGL | SLQP | | | | | | |
| | FFGVV | EDFA | | | | | | |
| | TKPNY | TYYC | | | | | | |
| | YYYAM | QQSY | | | | | | |
| | DVWGQ | STPR | | | | | | |
| | GTTVT | TFGG | | | | | | |
| | VSS | GTKL | | | | | | |
| | (SEQ | EIK | | | | | | |
| | ID | (SEQ | | | | | | |
| | NO: | ID | | | | | | |
| | 96) | NO: | | | | | | |
| | | 97) | | | | | | |
| SD3 | EVQLV | ELQM | DDYA | GISW | AKDG | QASQD | DASYL | QQYDN |
| | ESGGG | TQSP | MH | NSGS | SRVF | IGNYL | ET | LPFT |
| | LVQPG | SSLS | (SEQ | IDYA | GVGG | N | (SEQ | (SEQ |
| | RSLRL | ASVG | ID | DSVK | GFDF | (SEQ | ID | ID |
| | SCAAS | DRVT | NO: | G | (SEQ | ID | NO: | NO: |
| | GFTFD | ITCQ | 106) | (SEQ | ID | NO: | 110) | 111) |
| | DYAMH | ASQD | | ID | NO: | 109) | | |
| | WVRQA | IGNY | | NO: | 108) | | | |
| | PGKGL | LNWY | | 107) | | | | |
| | EWVSG | QQKP | | | | | | |
| | ISWNS | GKAP | | | | | | |
| | GSIDY | KLLI | | | | | | |
| | ADSVK | YDAS | | | | | | |
| | GRFTI | YLET | | | | | | |
| | SRDNA | GVPS | | | | | | |
| | KNSLY | RFSG | | | | | | |
| | LQMNS | SGSG | | | | | | |
| | LRVED | TDFT | | | | | | |
| | TALYY | FTIS | | | | | | |
| | CAKDG | SLQP | | | | | | |
| | SRVFG | EDIA | | | | | | |
| | VGGGF | TYYC | | | | | | |
| | DFWGQ | QQYD | | | | | | |
| | GTMVT | NLPF | | | | | | |
| | VSS | TFGP | | | | | | |
| | (SEQ | GTKV | | | | | | |
| | ID | DIK | | | | | | |
| | NO: | (SEQ | | | | | | |
| | 104) | ID | | | | | | |
| | | NO: | | | | | | |
| | | 105) | | | | | | |
| SD4 | QVQLV | ELVL | SDYA | VISH | ARDR | GGNNI | DDSDR | QVWDS |
| | QSGGG | TQPP | MH | GGTK | VEGY | GRQTV | P | SSDHV |
| | VVQSG | SVPV | (SEQ | KYTGD | V | H | (SEQ | V |
| | RSLRL | APGQ | ID | SVKG | (SEQ | (SEQ | ID | (SEQ |
| | SCAAS | TANI | NO: | (SEQ | ID | ID | NO: | ID |
| | GFTFS | SCGG | 114) | ID | NO: | NO: | 118) | NO: |
| | DYAMH | NNIG | | NO: | 116) | 117) | | 119) |
| | WVRQA | RQTV | | 115) | | | | |
| | PGKGL | HWYQ | | | | | | |
| | EWVAV | QKPG | | | | | | |
| | ISHGG | QAPV | | | | | | |
| | TKKYT | LVVF | | | | | | |
| | GDSVK | DDSD | | | | | | |
| | GRFII | RPAG | | | | | | |
| | SRDNS | IPER | | | | | | |
| | KNTVF | FSGS | | | | | | |
| | LQMNS | NSGN | | | | | | |
| | LRVED | TATL | | | | | | |
| | TAVYY | TISR | | | | | | |
| | CARDR | VEAG | | | | | | |
| | VEGYV | DEAD | | | | | | |
| | WGGTF | YYCQ | | | | | | |
| | DHWGQ | VWDS | | | | | | |
| | GTPVT | SSDH | | | | | | |
| | VSS | VVFG | | | | | | |
| | (SEQ | GGTQ | | | | | | |
| | ID | LTVL | | | | | | |
| | NO: | (SEQID | | | | | | |
| | 112) | NO: | | | | | | |
| | | 113) | | | | | | |

In some embodiments, the CDR sequences of the anti-desmoglein antibodies are provided using Kabat, JMGT, or CHOTHIA numbering as shown in the DSG CDR Table.

DSG CDR Table

| | CDR | | | | | | |
|---|---|---|---|---|---|---|---|
| | Anno- | | | | | | |
| Anti- | tation | | | | | | |
| body | System | HCDR1 | HCDR2 | HCDR3 | LCDR1 | LCDR2 | LCDR3 |
| SD1 | Kabat | SSNHW | GEIYH | GWHRT | SGSSS | SNDQR | AAWDD |
| | | WT | NGSTF | GFRGY | HIGSN | PA | GQGGV |
| | | (SEQ | LNPSL | PSHWY | YVY | (SEQ | (SEQ |
| | | ID | K | FDL | (SEQ | ID | IDNO: |
| | | NO: | (SEQ | (SEQ | ID | NO: | |
| | | 484) | ID | ID | NO: | 92) | 93) |
| | | | NO: | NO: | 91) | | |
| | | | 485) | 486) | | | |
| | IMGT | GSISS | IYHNG | ARGWH | SSHIG | SNDQR | AAWDD |
| | | NHW | STFLN | RTGFR | SNY | PAGVP | GQGGV |
| | | (SEQ | (SEQ | GYPSH | (SEQ | (SEQ | (SEQ |
| | | ID | ID | WYFDL | ID | ID | ID |
| | | NO: | NO: | (SEQ | NO: | NO: | NO: |
| | | 487) | 488) | ID | 490) | 491) | 492) |
| | | | | NO: | | | |
| | | | | 489) | | | |
| | CHOTHIA | GGSIS | IYHNG | GWHRT | SSSHI | SND | WDDGQ |
| | | SNH | (SEQ | GFRGY | GSNY | | GGV |
| | | (SEQ | ID | PSHWY | (SEQ | | (SEQ |
| | | ID | NO: | FDL | ID | | ID |
| | | NO: | 494) | (SEQ | NO: | | NO: |
| | | 493) | | ID | 496) | | 497) |
| | | | | NO: | | | |
| | | | | 495) | | | |
| SD2 | Kabat | TTYDI | WMNPT | GLFFG | RASQS | AASSL | QQSYS |
| | | N | SGNTA | VVTKP | ISSYL | QS | TPRT |
| | | (SEQ | YAQKF | NYYYY | N | (SEQ | (SEQ |
| | | ID | QG | AMDV | (SEQ | ID | ID |
| | | NO: | (SEQ | (SEQ | ID | NO: | NO: |
| | | 98) | ID | ID | NO: | 102) | 103) |
| | | | NO: | NO: | 101) | | |
| | | | 498) | 499) | | | |
| | IMGT | YTLTT | MNPTS | ARGLF | QSISS | AASSL | QQSYS |
| | | YD | GNTAY | FGVVT | Y | QSGVP | TPRT |
| | | (SEQ | (SEQ | KPNYY | (SEQ | (SEQ | (SEQ |
| | | ID | ID | YYAMD | ID | ID | ID |
| | | NO: | NO: | V | NO: | NO: | NO: |
| | | 500) | 501) | (SEQ | 503) | 504) | 103) |
| | | | | ID | | | |
| | | | | NO: | | | |
| | | | | 502) | | | |
| | CHOTHIA | GYTLT | NPTSG | GLFFG | SQSIS | AAS | SYSTP |
| | | TY | (SEQ | VVTKP | SY | | RT |
| | | (SEQ | ID | NYYYY | (SEQ | | (SEQ |
| | | ID | NO: | AMDV | ID | | ID |
| | | NO: | 506) | (SEQ | NO: | | NO: |
| | | 505) | | ID | 508) | | 509) |
| | | | | NO: | | | |
| | | | | 507) | | | |
| SD3 | Kabat | DDYAM | GISWN | DGSRV | QASQD | DASYL | QQYDN |
| | | H | SGSID | FGVGG | IGNYL | ET | LPFT |
| | | (SEQ | YADSV | GFDF | N | (SEQ | (SEQ |
| | | ID | KG | (SEQ | (SEQ | ID | ID |
| | | NO: | (SEQ | ID | ID | NO: | NO: |
| | | 106) | ID | NO: | NO: | 110) | 111) |
| | | | NO: | 510) | 109) | | |
| | | | 107) | | | | |
| | IMGT | FTFDD | ISWNS | AKDGS | QDIGN | DASYL | QQYDN |
| | | YA | GSIDY | RVFGV | Y | ETGVP | LPFT |
| | | (SEQ | (SEQ | GGGFD | (SEQ | (SEQ | (SEQ |
| | | ID | ID | F( | ID | ID | ID |
| | | NO: | NO: | SEQ | NO: | NO: | NO: |
| | | 511) | 512) | ID: | 514) | 110) | 111) |
| | | | | NO | | | |
| | | | | 513) | | | |
| | CHOTHIA | GFTFD | SWNSG | DGSRV | SQDIG | DAS | YDNLP |
| | | DY | (SEQ | FGVGG | NY | | FT |
| | | (SEQ | ID | GFDF | (SEQ | | (SEQ |
| | | ID | NO: | (SEQ | ID | | ID |
| | | NO: | 516) | ID | NO: | | NO: |
| | | 515) | | NO: | 517) | | 518) |
| | | | | 510) | | | |
| SD4 | Kabat | SDYAM | VISHG | DRVEG | GGNNI | DDSDR | QVWDS |
| | | H | GTKKY | YVWGG | GRQTV | PA | SSDHV |
| | | (SEQ | TGDSV | TFDH | H | (SEQ | V |
| | | ID | K | (SEQ | (SEQ | ID | (SEQ |
| | | NO: | (SEQ | ID | ID | NO: | ID |
| | | 114) | ID | NO: | NO: | 521) | NO: |
| | | | NO: | 520) | 117) | | 119) |
| | | | 519) | | | | |
| | IMGT | FTFSD | ISHGG | ARDRV | NIGRQ | DDSDR | QVWDS |
| | | YA | TKKYT | EGYVW | T | PAGIP | SSDHV |
| | | (SEQ | (SEQ | GGTFD | (SEQ | (SEQ | V |
| | | ID | ID | H | ID | ID | (SEQ |
| | | NO: | NO: | (SEQ | NO: | NO: | ID |
| | | 522) | 523) | ID | 525) | 526) | NO: |
| | | | | NO: | | | 119) |
| | | | | 524) | | | |
| | CHOTHIA | GFTFS | SHGGT | DRVEG | NNIGR | DDS | WDSSS |
| | | DY | (SEQ | YVWGG | QT | | DHVV |
| | | (SEQ | ID | TFDH | (SEQ | | (SEQ |
| | | ID | NO: | (SEQ | ID | | ID |
| | | NO: | 528) | ID | NO: | | NO: |
| | | 527) | | NO: | 529) | | 530) |
| | | | | 520) | | | |

In some embodiments, the antibody is linked to another antibody or therapeutic. In some embodiments, the anti-desmoglein 1 antibody, anti-desmoglein 2 antibody, anti-desmoglein 3 antibody, or anti-desmoglein 4 antibody is linked to a PD-1 antibody, a IL-2 mutein, or CD39 as provided herein or that is incorporated by reference.

In some embodiment, the anti-desmoglein 1 antibody, anti-desmoglein 2 antibody, anti-desmoglein 3 antibody, or anti-desmoglein 4 antibody, as provided herein, is linked to a IL-2 mutein comprising L118I, N88D, V69A, Q74P, and C125S mutations, and having the following sequence:

(SEQ ID NO: 120)
APTSSSTKKTQLQLEHLLLDLQMILNGINNYKNPKLTRMLTFKFYMPKKA
TELKHLQCLEEELKPLEEALNLAPSKNFHLRPRDLISDINVIVLELKGSE
TTFMCEYADETATIVEFINRWITFSQSIISTLT

In some embodiments, the anti-desmoglein antibody linked to IL-2 mutein has the following heavy and light chain sequence:

DSG-IL-2 Table

| Ab# | Heavy chain Seq | Light chain Seq |
|---|---|---|
| IL2DSGAB1 | QVQLQESGPGPVKPSGTLSLTCGVSGGSISSNHWWTW | QSVLTQPPSVSGTPGQRVTISCSGSSSHIGSN |
| | VRQPPGKGLEWIGEIYHNGSTFLNPSLKSRVTISVDK | YVYWYQQLPGTAPKILIYSNDQRPAGVPDRFS |
| | SNNQFSLKLTSVTAADTAVYYCARGWHRTGFRGYPSH | ASKSGTSASLAISGLRSEDEADYYCAAWDDGQ |
| | WYFDLWGRGTLVSVSSASTKGPSVFPLAPSSKSTSGG | GGVFGGGTKLTVLGQPKAAPSVTLFPPSSEEL |
| | TAALGCLVKDYFPEPVTVSWNSGALTSGVHTFPAVLQ | QANKATLVCLISDFYPGAVTVAWKADSSPVKA |
| | SSGLYSLSSVVTVPSSSLGTQTYICNVNHKPSNTKVD | GVETTTPSKQSNNKYAASSYLSLTPEQWKSHR |
| | KKVEPKSCDKTHTCPPCPAPEAAGAPSVFLFPPKPKD | SYSCQVTHEGSTVEKTVAPTECS (SEQ ID |
| | TLMISRTPEVTCVVVDVSHEDPEVKFNWYVDGVEVHN | NO: 122) |
| | AKTKPREEQYNSTYRVVSVLTVLHQDWLNGKEYKCKV | |
| | SNKALPAPIEKTISKAKGQPREPQVYTLPPSREEMTK | |
| | NQVSLTCLVKGFYPSDIAVEWESNGQPENNYKTTPPV | |
| | LDSDGSFFLYSKLTVDKSRWQQGNVFSCSVMHEALHN | |
| | HYTQKSLSLSPGGGGGSAPTSSSTKKTQLQLEHLLLD | |
| | LQMILNGINNYKNPKLTRMLTFKFYMPKKATELKHLQ | |
| | CLEEELKPLEEALNLAPSKNFHLRPRDLISDINVIVL | |
| | ELKGSETTFMCEYADETATIVEFINRWITFSQSIIST | |
| | LT (SEQ ID NO: 121) | |
| IL2DSGAB2 | QVQLVQSGAEVRKPGASVRVSCKASGYTLTTYDINWV | DIQMTQSPSSLSASVGDRVTITCRASQSISSY |
| | RQATGQGLEWMGWMNPTSGNTAYAQKFQGRVTMTRNT | LNWYQQKPGKAPKLLIYAASSLQSGVPSRFSG |
| | SISTAYMELSSLRSEDTAVYYCARGLFFGVVTKPNYY | SGSGTDFTLTISSLQPEDFATYYCQQSYSTPR |
| | YYAMDVWGQGTTVTVSSASTKGPSVFPLAPSSKSTSG | TFGGGTKLEIKRTVAAPSVFIFPPSDEQLKSG |
| | GTAALGCLVKDYFPEPVTVSWNSGALTSGVHTFPAVL | TASVVCLLNNFYPREAKVQWKVDNALQSGNSQ |
| | QSSGLYSLSSVVTVPSSSLGTQTYICNVNHKPSNTKV | ESVTEQDSKDSTYSLSSTLTLSKADYEKHKVY |
| | DKKVEPKSCDKTHTCPPCPAPEAAGAPSVFLFPPKPK | ACEVTHQGLSSPVTKSFNRGEC (SEQ ID |
| | DTLMISRTPEVTCVVVDVSHEDPEVKFNWYVDGVEVH | NO: 124) |
| | NAKTKPREEQYNSTYRVVSVLTVLHQDWLNGKEYKCK | |
| | VSNKALPAPIEKTISKAKGQPREPQVYTLPPSREEMT | |
| | KNQVSLTCLVKGFYPSDIAVEWESNGQPENNYKTTPP | |
| | VLDSDGSFFLYSKLTVDKSRWQQGNVFSCSVMHEALH | |
| | NHYTQKSLSLSPGGGGGSAPTSSSTKKTQLQLEHLLL | |
| | DLQMILNGINNYKNPKLTRMLTFKFYMPKKATELKHL | |
| | QCLEEELKPLEEALNLAPSKNFHLRPRDLISDINVIV | |
| | LELKGSETTFMCEYADETATIVEFINRWITFSQSIIS | |
| | TLT (SEQ ID NO: 123) | |
| IL2DSGAB3 | EVQLVESGGGLVQPGRSLRLSCAASGFTFDDYAMHWV | ELQMTQSPSSLSASVGDRVTITCQASQDIGNY |
| | RQAPGKGLEWVSGISWNSGSIDYADSVKGRFTISRDN | LNWYQQKPGKAPKLLIYDASYLETGVPSRFSG |
| | AKNSLYLQMNSLRVEDTALYYCAKDGSRVFGVGGGFD | SGSGTDFTFTISSLQPEDIATYYCQQYDNLPF |
| | FWGQGTMVTVSSASTKGPSVFPLAPSSKSTSGGTAAL | TFGPGTKVDIKRTVAAPSVFIFPPSDEQLKSG |
| | GCLVKDYFPEPVTVSWNSGALTSGVHTFPAVLQSSGL | TASVVCLLNNFYPREAKVQWKVDNALQSGNSQ |
| | YSLSSVVTVPSSSLGTQTYICNVNHKPSNTKVDKKVE | ESVTEQDSKDSTYSLSSTLTLSKADYEKHKVY |
| | PKSCDKTHTCPPCPAPEAAGAPSVFLFPPKPKDTLMI | ACEVTHQGLSSPVTKSFNRGEC (SEQ ID |
| | SRTPEVTCVVVDVSHEDPEVKFNWYVDGVEVHNAKTK | NO: 126) |
| | PREEQYNSTYRVVSVLTVLHQDWLNGKEYKCKVSNKA | |
| | LPAPIEKTISKAKGQPREPQVYTLPPSREEMTKNQVS | |
| | LTCLVKGFYPSDIAVEWESNGQPENNYKTTPPVLDSD | |
| | GSFFLYSKLTVDKSRWQQGNVFSCSVMHEALHNHYTQ | |
| | KSLSLSPGGGGGSAPTSSSTKKTQLQLEHLLLDLQMI | |
| | LNGINNYKNPKLTRMLTFKFYMPKKATELKHLQCLEE | |
| | ELKPLEEALNLAPSKNFHLRPRDLISDINVIVLELKG | |
| | SETTFMCEYADETATIVEFINRWITFSQSIISTLT | |
| | (SEQ ID NO: 125) | |
| IL2DSGAB4 | QVQLVQSGGGVVQSGRSLRLSCAASGFTFSDYAMHWV | ELVLTQPPSVPVAPGQTANISCGGNNIGRQTV |
| | RQAPGKGLEWVAVISHGGTKKYTGDSVKGRFIISRDN | HWYQQKPGQAPVLVVFDDSDRPAGIPERFSGS |
| | SKNTVFLQMNSLRVEDTAVYYCARDRVEGYVWGGTFD | NSGNTATLTISRVEAGDEADYYCQVWDSSSDH |
| | HWGQGTPVTVSSASTKGPSVFPLAPSSKSTSGGTAAL | VVFGGGTQLTVLGQPKAAPSVTLFPPSSEELQ |
| | GCLVKDYFPEPVTVSWNSGALTSGVHTFPAVLQSSGL | ANKATLVCLISDFYPGAVTVAWKADSSPVKAG |
| | YSLSSVVTVPSSSLGTQTYICNVNHKPSNTKVDKKVE | VETTTPSKQSNNKYAASSYLSLTPEQWKSHRS |
| | PKSCDKTHTCPPCPAPEAAGAPSVFLFPPKPKDTLMI | YSCQVTHEGSTVEKTVAPTECS (SEQ ID |
| | SRTPEVTCVVVDVSHEDPEVKFNWYVDGVEVHNAKTK | NO: 128) |
| | PREEQYNSTYRVVSVLTVLHQDWLNGKEYKCKVSNKA | |
| | LPAPIEKTISKAKGQPREPQVYTLPPSREEMTKNQVS | |
| | LTCLVKGFYPSDIAVEWESNGQPENNYKTTPPVLDSD | |
| | GSFFLYSKLTVDKSRWQQGNVFSCSVMHEALHNHYTQ | |
| | KSLSLSPGGGGGSAPTSSSTKKTQLQLEHLLLDLQMI | |
| | LNGINNYKNPKLTRMLTFKFYMPKKATELKHLQCLEE | |
| | ELKPLEEALNLAPSKNFHLRPRDLISDINVIVLELKG | |
| | SETTFMCEYADETATIVEFINRWITFSQSIISTLT | |
| | (SEQ ID NO: 127) | |
| IL2DSGAB5 | QVQLQESGPGPVKPSGTLSLTCGVSGGSISSNHWWTW | QSVLTQPPSVSGTPGQRVTISCSGSSSHIGSN |
| | VRQPPGKGLEWIGEIYHNGSTFLNPSLKSRVTISVDK | YVYWYQQLPGTAPKILIYSNDQRPAGVPDRFS |
| | SNNQFSLKLTSVTAADTAVYYCARGWHRTGFRGYPSH | ASKSGTSASLAISGLRSEDEADYYCAAWDDGQ |
| | WYFDLWGRGTLVSVSSASTKGPSVFPLAPSSKSTSGG | GGVFGGGTKLTVLGQPKAAPSVTLFPPSSEEL |
| | TAALGCLVKDYFPEPVTVSWNSGALTSGVHTFPAVLQ | QANKATLVCLISDFYPGAVTVAWKADSSPVKA |
| | SSGLYSLSSVVTVPSSSLGTQTYICNVNHKPSNTKVD | GVETTTPSKQSNNKYAASSYLSLTPEQWKSHR |
| | KKVEPKSCDKTHTCPPCPAPEAAGAPSVFLFPPKPKD | SYSCQVTHEGSTVEKTVAPTECSGGGGSGGGG |
| | TLMISRTPEVTCVVVDVSHEDPEVKFNWYVDGVEVHN | SGGGGSAPTSSSTKKTQLQLEHLLLDLQMILN |
| | AKTKPREEQYNSTYRVVSVLTVLHQDWLNGKEYKCKV | GINNYKNPKLTRMLTFKFYMPKKATELKHLQC |
| | SNKALPAPIEKTISKAKGQPREPQVYTLPPSREEMTK | LEEELKPLEEALNLAPSKNFHLRPRDLISDIN |
| | NQVSLTCLVKGFYPSDIAVEWESNGQPENNYKTTPPV | VIVLELKGSETTFMCEYADETATIVEFINRWI |
| | LDSDGSFFLYSKLTVDKSRWQQGNVFSCSVMHEALHN | TFSQSIISTLT (SEQ ID NO: 130) |
| | HYTQKSLSLSPG (SEQ ID NO: 129) | |
| IL2DSGAB6 | QVQLVQSGAEVRKPGASVRVSCKASGYTLTTYDINWV | DIQMTQSPSSLSASVGDRVTITCRASQSISSY |
| | RQATGQGLEWMGWMNPTSGNTAYAQKFQGRVTMTRNT | LNWYQQKPGKAPKLLIYAASSLQSGVPSRFSG |
| | SISTAYMELSSLRSEDTAVYYCARGLFFGVVTKPNYY | SGSGTDFTLTISSLQPEDFATYYCQQSYSTPR |
| | YYAMDVWGQGTTVTVSSASTKGPSVFPLAPSSKSTSG | TFGGGTKLEIKRTVAAPSVFIFPPSDEQLKSG |
| | GTAALGCLVKDYFPEPVTVSWNSGALTSGVHTFPAVL | TASVVCLLNNFYPREAKVQWKVDNALQSGNSQ |
| | QSSGLYSLSSVVTVPSSSLGTQTYICNVNHKPSNTKV | ESVTEQDSKDSTYSLSSTLTLSKADYEKHKVY |
| | DKKVEPKSCDKTHTCPPCPAPEAAGAPSVFLFPPKPK | ACEVTHQGLSSPVTKSFNRGECGGGSEGGGSE |
| | DTLMISRTPEVTCVVVDVSHEDPEVKFNWYVDGVEVH | GGGSEAPTSSSTKKTQLQLEHLLLDLQMILNG |
| | NAKTKPREEQYNSTYRVVSVLTVLHQDWLNGKEYKCK | INNYKNPKLTRMLTFKFYMPKKATELKHLQCL |
| | VSNKALPAPIEKTISKAKGQPREPQVYTLPPSREEMT | EEELKPLEEALNLAPSKNFHLRPRDLISDINV |
| | KNQVSLTCLVKGFYPSDIAVEWESNGQPENNYKTTPP | IVLELKGSETTFMCEYADETATIVEFINRWIT |
| | VLDSDGSFFLYSKLTVDKSRWQQGNVFSCSVMHEALH | FSQSIISTLT (SEQ ID NO: 132) |
| | NHYTQKSLSLSPG (SEQ ID NO: 131) | |
| IL2DSGAB7 | EVQLVESGGGLVQPGRSLRLSCAASGFTFDDYAMHWV | ELQMTQSPSSLSASVGDRVTITCQASQDIGNY |
| | RQAPGKGLEWVSGISWNSGSIDYADSVKGRFTISRDN | LNWYQQKPGKAPKLLIYDASYLETGVPSRFSG |
| | AKNSLYLQMNSLRVEDTALYYCAKDGSRVFGVGGGFD | SGSGTDFTFTISSLQPEDIATYYCQQYDNLPF |
| | FWGQGTMVTVSSASTKGPSVFPLAPSSKSTSGGTAAL | TFGPGTKVDIKRTVAAPSVFIFPPSDEQLKSG |
| | GCLVKDYFPEPVTVSWNSGALTSGVHTFPAVLQSSGL | TASVVCLLNNFYPREAKVQWKVDNALQSGNSQ |
| | YSLSSVVTVPSSSLGTQTYICNVNHKPSNTKVDKKVE | ESVTEQDSKDSTYSLSSTLTLSKADYEKHKVY |
| | PKSCDKTHTCPPCPAPEAAGAPSVFLFPPKPKDTLMI | ACEVTHQGLSSPVTKSFNRGECGGGSEGGGSE |
| | SRTPEVTCVVVDVSHEDPEVKFNWYVDGVEVHNAKTK | GGGSEGGGSEAPTSSSTKKTQLQLEHLLLDLQ |
| | PREEQYNSTYRVVSVLTVLHQDWLNGKEYKCKVSNKA | MILNGINNYKNPKLTRMLTFKFYMPKKATELK |
| | LPAPIEKTISKAKGQPREPQVYTLPPSREEMTKNQVS | HLQCLEEELKPLEEALNLAPSKNFHLRPRDLI |
| | LTCLVKGFYPSDIAVEWESNGQPENNYKTTPPVLDSD | SDINVIVLELKGSETTFMCEYADETATIVEFI |
| | GSFFLYSKLTVDKSRWQQGNVFSCSVMHEALHNHYTQ | NRWITFSQSIISTLT (SEQ ID NO: 134) |
| | KSLSLSPG (SEQ ID NO: 133) | |
| IL2DSGAB8 | QVQLVQSGGGVVQSGRSLRLSCAASGFTFSDYAMHWV | ELVLTQPPSVPVAPGQTANISCGGNNIGRQTV |
| | RQAPGKGLEWVAVISHGGTKKYTGDSVKGRFIISRDN | HWYQQKPGQAPVLVVFDDSDRPAGIPERFSGS |
| | SKNTVFLQMNSLRVEDTAVYYCARDRVEGYVWGGTFD | NSGNTATLTISRVEAGDEADYYCQVWDSSSDH |
| | HWGQGTPVTVSSASTKGPSVFPLAPSSKSTSGGTAAL | VVFGGGTQLTVLGQPKAAPSVTLFPPSSEELQ |
| | GCLVKDYFPEPVTVSWNSGALTSGVHTFPAVLQSSGL | ANKATLVCLISDFYPGAVTVAWKADSSPVKAG |
| | YSLSSVVTVPSSSLGTQTYICNVNHKPSNTKVDKKVE | VETTTPSKQSNNKYAASSYLSLTPEQWKSHRS |
| | PKSCDKTHTCPPCPAPEAAGAPSVFLFPPKPKDTLMI | YSCQVTHEGSTVEKTVAPTECSAEEEKAEEEK |
| | SRTPEVTCVVVDVSHEDPEVKFNWYVDGVEVHNAKTK | AEEEKAEEEKAPTSSSTKKTQLQLEHLLLDLQ |
| | PREEQYNSTYRVVSVLTVLHQDWLNGKEYKCKVSNKA | MILNGINNYKNPKLTRMLTFKFYMPKKATELK |
| | LPAPIEKTISKAKGQPREPQVYTLPPSREEMTKNQVS | HLQCLEEELKPLEEALNLAPSKNFHLRPRDLI |
| | LTCLVKGFYPSDIAVEWESNGQPENNYKTTPPVLDSD | SDINVIVLELKGSETTFMCEYADETATIVEFI |
| | GSFFLYSKLTVDKSRWQQGNVFSCSVMHEALHNHYTQ | NRWITFSQSIISTLT (SEQ ID NO: 136) |
| | KSLSLSPG (SEQ ID NO: 135) | |

In some embodiments, the anti-desmoglein antibody, anti-desmoglein antibody, anti-desmoglein 3 antibody, or anti-desmoglein 4 antibody comprises a sequence as provided for herein. In some embodiments, the antibody is in a scFV format as illustrated herein. In some embodiments, the antibody comprises a CDR of any one of the sequences provided for herein. In some embodiments, the amino acid residues of the CDRs provided herein contain mutations. In some embodiments, the CDRs contain 1, 2, 3, 4, 5, 6, 7, 8, 9, or 10 substitutions or mutations. In some embodiments, the substitution is a conservative substitution.

In some embodiments, as provided for herein, the anti-desmoglein 1 antibody, anti-desmoglein 2 antibody, anti-desmoglein 3 antibody, or anti-desmoglein 4 antibody, or binding fragment thereof, is linked directly or indirectly to a PD-1 antibody or binding fragment thereof.

In some embodiments, the PD-1 antibody is selected from the following table:

PD-1 Antibody Table

| Clone | | | | | | | | |
|---|---|---|---|---|---|---|---|---|
| (scFv) | VH Seq | VK Seq | CDR1 | CDR2 | CDR3 | LCDR1 | LCDR2 | LCDR3 |
| PD1AB1 | QVQLVQSGAE | DIQMTQS | GSFTGYY | GWINPN | CARDT | QASHDID | SSLQS | QQANSLPL |
| | VKKPGASVKV | PSSLSAS | MH (SEQ | DGAIHY | VTGDF | KYLN | (SEQ | T (SEQ |
| | SCKASGGSFT | VGDRVTI | ID NO: | A (SEQ | DYW | (SEQ ID | ID | ID NO: |
| | GYYMHWVRQA | TCQASHD | 139) | ID NO: | (SEQ | NO: | NO: | 144) |
| | PGQGLEWMGW | IDKYLNW | | 140) | ID | 142) | 143) | |
| | INPNDGAIHY | YQQKPGK | | | NO: | | | |
| | AQNFQGRVTM | APKLLIY | | | 141) | | | |
| | TRDTSTSTVY | AASSLQS | | | | | | |
| | MELSSLRSED | GVPSRFS | | | | | | |
| | TAVYYCARDT | GSGSGTD | | | | | | |
| | VTGDFDYWGQ | FTLTISS | | | | | | |
| | GTLVTVSS | LQPEDFA | | | | | | |
| | (SEQ ID | TYYCQQA | | | | | | |
| | NO: 137) | NSLPLTF | | | | | | |
| | | GGGTKVE | | | | | | |
| | | IK (SEQ | | | | | | |
| | | ID NO: | | | | | | |
| | | 138) | | | | | | |
| PD1AB2 | QVQLVQSGAE | DIQMTQS | GTFSRYA | GWINPN | CAKQG | RASQSIS | STLES | QQSYSTPF |
| | VKKPGASVKV | PSSLSAS | VS (SEQ | SGGTSY | DYGGG | SWLA | (SEQ | T (SEQ |
| | SCKASGGTFS | VGDRVTI | ID NO: | A (SEQ | YYFDY | (SEQ ID | ID | ID NO: |
| | RYAVSWVRQA | TCRASQS | 147) | ID NO: | W | NO: | NO: | 152) |
| | PGQGLEWMGW | ISSWLAW | | 148) | (SEQ | 150) | 151) | |
| | INPNSGGTSY | YQQKPGK | | | ID | | | |
| | AQRFQGRVTM | APKLLIY | | | NO: | | | |
| | TRDTSTSTVY | KTSTLES | | | 149) | | | |
| | MELSSLRSED | GVPSRFS | | | | | | |
| | TAVYYCAKQG | GSGSGTD | | | | | | |
| | DYGGGYYFDY | FTLTISS | | | | | | |
| | WGQGTLVTVS | LQPEDFA | | | | | | |
| | S (SEQ ID | TYYCQQS | | | | | | |
| | NO: 145) | YSTPFTF | | | | | | |
| | | GQGTKVE | | | | | | |
| | | IK (SEQ | | | | | | |
| | | ID NO: | | | | | | |
| | | 146) | | | | | | |
| PD1AB3 | QVQLVQSGAE | DIQMTQS | GTFSSYA | GWMNPN | CARVG | RASQSIN | SSLQS | QQSYSTPF |
| | VKKPGASVKV | PSSLSAS | IS (SEQ | SGNTGY | YSYGY | NWLA | (SEQ | T (SEQ |
| | SCKASGGTFS | VGDRVTI | ID NO: | A (SEQ | GMDVW | (SEQ ID | ID | ID NO: |
| | SYAISWVRQA | TCRASQS | 155) | ID NO: | (SEQ | NO: | NO: | 152) |
| | PGQGLEWMGW | INNWLAW | | 156) | ID | 158) | 143) | |
| | MNPNSGNTGY | YQQKPGK | | | NO: | | | |
| | AQKFQGRVTM | APKLLIY | | | 157) | | | |
| | TRDTSTSTVY | AASSLQS | | | | | | |
| | MELSSLRSED | GVPSRFS | | | | | | |
| | TAVYYCARVG | GSGSGTD | | | | | | |
| | YSYGYGMDVW | FTLTISS | | | | | | |
| | GQGTTVTVSS | LQPEDFA | | | | | | |
| | (SEQ ID | TYYCQQS | | | | | | |
| | NO: 153) | YSTPFTF | | | | | | |
| | | GPGTKVD | | | | | | |
| | | IK (SEQ | | | | | | |
| | | ID NO: | | | | | | |
| | | 154) | | | | | | |
| PD1AB4 | QVQLVQSGAE | DIQMTQS | YSFTTYY | GIINPS | CASGW | QASRDIK | SSLQS | QQSYSTPP |
| | VKKPGASVKV | PSSLSAS | MH (SEQ | GGSTSY | VYW | NYLA | (SEQ | T (SEQ |
| | SCKASGYSFT | VGDRVTI | ID NO: | A (SEQ | (SEQ | (SEQ ID | ID | ID NO: |
| | TYYMHWVRQA | TCQASRD | 161) | ID NO: | ID | NO: | NO: | 165) |
| | PGQGLEWMGI | IKNYLAW | | 162) | NO: | 164) | 143) | |
| | INPSGGSTSY | YQQKPGK | | | 163) | | | |
| | AQKFQGRVTM | APKLLIY | | | | | | |
| | TRDTSTSTVY | AASSLQS | | | | | | |
| | MELSSLRSED | GVPSRES | | | | | | |
| | TAVYYCASGW | GSGSGTD | | | | | | |
| | VYWGQGTLVT | FTLTISS | | | | | | |
| | VSS (SEQ | LQPEDFA | | | | | | |
| | ID NO: | TYYCQQS | | | | | | |
| | 159) | YSTPPTF | | | | | | |
| | | GPGTKVD | | | | | | |
| | | IK (SEQ | | | | | | |
| | | ID NO: | | | | | | |
| | | 160) | | | | | | |
| PD1AB5 | EVQLLESGGG | DIQMTQS | FTFSSYA | AAIWSD | CARGL | RASQSIS | STLQS | QQSYSTPL |
| | LVQPGGSLRL | PSSLSAS | MS (SEQ | GSHQYY | GVERG | SWLA | (SEQ | T (SEQ |
| | SCAASGFTFS | VGDRVTI | ID NO: | A (SEQ | LDYW | (SEQ ID | ID | ID NO: |
| | SYAMSWVRQA | TCRASQS | 168) | ID NO: | (SEQ | NO: | NO: | 172) |
| | PGKGLEWVAA | ISSWLAW | | 169) | ID | 150) | 171) | |
| | IWSDGSHQYY | YQQKPGK | | | NO: | | | |
| | ADSVKGRFTI | APKLLIY | | | 170) | | | |
| | SRDNSKNTLY | AASTLQS | | | | | | |
| | LQMNSLRAED | GVPSRFS | | | | | | |
| | TAVYYCARGL | GSGSGTD | | | | | | |
| | GVERGLDYWG | FTLTISS | | | | | | |
| | QGTLVTVSS | LQPEDFA | | | | | | |
| | (SEQ ID | TYYCQQS | | | | | | |
| | NO: 166) | YSTPLTF | | | | | | |
| | | GQGTKVE | | | | | | |
| | | IK (SEQ | | | | | | |
| | | ID NO: | | | | | | |
| | | 167) | | | | | | |
| PD1AB6 | EVQLLESGGG | DIQMTQS | FTFSNYP | ALISDD | CARDS | RASQSIN | SNLET | QQSYSTPL |
| | LVQPGGSLRL | PSSLSAS | MH (SEQ | GTNEHY | KFANY | NYLS | (SEQ | T (SEQ |
| | SCAASGFTFS | VGDRVTI | ID NO: | A (SEQ | YYYYD | (SEQ ID | ID | ID NO: |
| | NYPMHWVRQA | TCRASQS | 175) | ID NO: | MDVW | NO: | NO: | 172) |
| | PGKGLEWVAL | INNYLSW | | 176) | (SEQ | 178) | 179) | |
| | ISDDGTNEHY | YQQKPGK | | | ID | | | |
| | ADSVKGRFTI | APKLLIY | | | NO: | | | |
| | SRDNSKNTLY | DASNLET | | | 177) | | | |
| | LQMNSLRAED | GVPSRFS | | | | | | |
| | TAVYYCARDS | GSGSGTD | | | | | | |
| | KFANYYYYYD | FTLTISS | | | | | | |
| | MDVWGQGTTV | LQPEDFA | | | | | | |
| | TVSS (SEQ | TYYCQQS | | | | | | |
| | ID NO: | YSTPLTF | | | | | | |
| | 173) | GPGTKVD | | | | | | |
| | | IK (SEQ | | | | | | |
| | | ID NO: | | | | | | |
| | | 174) | | | | | | |
| PD1AB7 | QVQLVQSGAE | DIVMTQS | YSFTGHY | GIINPN | CARGK | RSSQSIL | STRQS | QQYYSIPV |
| | VKKPGASVKV | PDSLAVS | IH (SEQ | GGSTTY | FDFYG | YSSNNRD | (SEQ | T (SEQ |
| | SCKASGYSFT | LGERATI | ID NO: | A (SEQ | DYVTA | YLA | ID | ID NO: |
| | GHYIHWVRQA | NCRSSQS | 182) | ID NO: | FDIW | (SEQ ID | NO: | 187) |
| | PGQGLEWMGI | ILYSSNN | | 183) | (SEQ | NO: | 186) | |
| | INPNGGSTTY | RDYLAWY | | | ID | 185) | | |
| | AQKLQGRVTM | QQKPGQP | | | NO: | | | |
| | TRDTSTSTVY | PKLLIYW | | | 184) | | | |
| | MELSSLRSED | ASTRQSG | | | | | | |
| | TAVYYCARGK | VPDRFSG | | | | | | |
| | FDFYGDYVTA | SGSGTDF | | | | | | |
| | FDIWGQGTMV | TLTISSL | | | | | | |
| | TVSS (SEQ | QAEDVAV | | | | | | |
| | ID NO: | YYCQQYY | | | | | | |
| | 180) | SIPVTFG | | | | | | |
| | | GGTKVEI | | | | | | |
| | | K (SEQ | | | | | | |
| | | ID NO: | | | | | | |
| | | 181) | | | | | | |
| PD1AB8 | QVQLVQSGAE | DIQMTQS | YTFSNYD | GWMNPN | CARGA | RASQSIN | SSLQG | QQSYSFPY |
| | VKKPGASVKV | PSSLSAS | MN (SEQ | SGHTGS | FGLHL | NWLA | (SEQ | T (SEQ |
| | SCKASGYTFS | VGDRVTI | ID NO: | A (SEQ | GELSL | (SEQ ID | ID | ID NO: |
| | NYDMNWVRQA | TCRASQS | 190) | ID NO: | HYYGM | NO: | NO: | 194) |
| | PGQGLEWMGW | INNWLAW | | 191) | DVW | 158) | 193) | |
| | MNPNSGHTGS | YQQKPGK | | | (SEQ | | | |
| | APKFQGRVTM | APKLLIY | | | ID | | | |
| | TRDTSTSTVY | AASSLQG | | | | | | |
| | MELSSLRSED | GVPSRFS | | | NO: | | | |
| | TAVYYCARGA | GSGSGTD | | | | | | |
| | FGLHLGELSL | FTLTISS | | | 192) | | | |
| | HYYGMDVWGQ | LQPEDFA | | | | | | |
| | GTTVTVSS | TYYCQQS | | | | | | |
| | (SEQ ID | YSFPYTF | | | | | | |
| | NO: 188) | GQGTKLE | | | | | | |
| | | IK (SEQ | | | | | | |
| | | ID NO: | | | | | | |
| | | 189) | | | | | | |
| PD1AB9 | QVQLVQSGAE | DIQMTQS | YTFTGYY | GKIVPM | CARGP | RASQSIS | SSLQS | QQANSFPV |
| | VKKPGSSVKV | PSSLSAS | MH (SEQ | FDAANY | KWELD | RWLA | (SEQ | T (SEQ |
| | SCKASGYTFT | VGDRVTI | ID NO: | A (SEQ | TW | (SEQ ID | ID | ID NO: |
| | GYYMHWVRQA | TCRASQS | 197) | ID NO: | (SEQ | NO: | NO: | 201) |
| | PGQGLEWMGK | ISRWLAW | | 198) | ID | 200) | 143) | |
| | IVPMFDAANY | YQQKPGK | | | NO: | | | |
| | APKFQGRVTI | APKLLIY | | | 199) | | | |
| | TADESTSTAY | GASSLQS | | | | | | |
| | MELSSLRSED | GVPSRFS | | | | | | |
| | TAVYYCARGP | GSGSGTD | | | | | | |
| | KWELDTWGQG | FTLTISS | | | | | | |
| | TLVTVSS | LQPEDFA | | | | | | |
| | (SEQ ID | TYYCQQA | | | | | | |
| | NO: 195) | NSFPVTF | | | | | | |
| | | GGGTKVE | | | | | | |
| | | IK (SEQ | | | | | | |
| | | ID NO: | | | | | | |
| | | 196) | | | | | | |
| PD1AB10 | QVQLVQSGAE | DIQMTQS | YTFTGYY | GIINPS | CAKTA | RASQSIN | SSLQS | QQGYSVPL |
| | VKKPGASVKV | PSSLSAS | MH (SEQ | GGSTSY | GYDWL | SWLA | (SEQ | S (SEQ |
| | SCKASGYTFT | VGDRVTI | ID NO: | A (SEQ | PSGLG | (SEQ ID | ID | ID NO: |
| | GYYMHWVRQA | TCRASQS | 197) | ID NO: | MDVW | NO: | NO: | 206) |
| | PGQGLEWMGI | INSWLAW | | 162) | (SEQ | 205) | 143) | |
| | INPSGGSTSY | YQQKPGK | | | ID | | | |
| | AQKFQGRVTM | APKLLIY | | | NO: | | | |
| | TRDTSTSTVY | YASSLQS | | | 204) | | | |
| | MELSSLRSED | GVPSRFS | | | | | | |
| | TAVYYCAKTA | GSGSGTD | | | | | | |
| | GYDWLPSGLG | FTLTISS | | | | | | |
| | MDVWGQGTTV | LQPEDFA | | | | | | |
| | TVSS (SEQ | TYYCQQG | | | | | | |
| | ID NO: | YSVPLSF | | | | | | |
| | 202) | GQGTKLE | | | | | | |
| | | IK (SEQ | | | | | | |
| | | ID NO: | | | | | | |
| | | 203) | | | | | | |
| PD1AB11 | QVQLVQSGAE | DIQMTQS | YTFSNYG | GGIIPI | CARWR | RASQGIS | SNLET | QQSYSTPL |
| | VKKPGSSVKV | PSSLSAS | IT (SEQ | FGSTAS | SDAFD | NWLA | (SEQ | T (SEQ |
| | SCKASGYTFS | VGDRVTI | ID NO: | YA | IW | (SEQ ID | ID | ID NO: |
| | NYGITWVRQA | TCRASQG | 209) | (SEQ | (SEQ | NO : | NO : | 172) |
| | PGQGLEWMGG | ISNWLAW | | ID NO: | ID | 212) | 179) | |
| | IIPIFGSTAS | YQQKPGK | | 210) | NO: | | | |
| | YAQKFQGRVT | APKLLIY | | | 211) | | | |
| | ITADESTSTA | DASNLET | | | | | | |
| | YMELSSLRSE | GVPSRES | | | | | | |
| | DTAVYYCARW | GSGSGTD | | | | | | |
| | RSDAFDIWGQ | FTLTISS | | | | | | |
| | GTMVTVSS | LQPEDFA | | | | | | |
| | (SEQ ID | TYYCQQS | | | | | | |
| | NO: 207) | YSTPLTF | | | | | | |
| | | GGGTKVE | | | | | | |
| | | IK (SEQ | | | | | | |
| | | ID NO: | | | | | | |
| | | 208) | | | | | | |
| PD1AB12 | QVQLVQSGAE | DIQMTQS | GTFSTYA | GWINPN | CARVN | RASQGIR | STLNS | QQSYSTPF |
| | VKKPGASVKV | PSSLSAS | IS (SEQ | SGGTNY | YDFYY | NDLG | (SEQ | T (SEQ |
| | SCKASGGTFS | VGDRVTI | ID NO: | A (SEQ | GMDVW | (SEQ ID | ID | ID NO: |
| | TYAISWVRQA | TCRASQG | 215) | ID NO: | (SEQ | NO: | NO: | 152) |
| | PGQGLEWMGW | IRNDLGW | | 216) | ID | 218) | 219) | |
| | INPNSGGTNY | YQQKPGK | | | NO: | | | |
| | AQKFQGRVTM | APKLLIY | | | 217) | | | |
| | TRDTSTSIVY | RASTLNS | | | | | | |
| | MELSSLRSED | GVPSRFS | | | | | | |
| | TAVYYCARVN | GSGSGTD | | | | | | |
| | YDFYYGMDVW | FTLTISS | | | | | | |
| | GQGTTVTVSS | LQPEDFA | | | | | | |
| | (SEQ ID | TYYCQQS | | | | | | |
| | NO: 213) | YSTPFTF | | | | | | |
| | | GPGTKVD | | | | | | |
| | | IK (SEQ | | | | | | |
| | | ID NO: | | | | | | |
| | | 214) | | | | | | |
| PD1AB13 | QVQLVQSGAE | DIQMTQS | GTFSTYA | GWINPN | CARVN | RASQGIR | STLNS | QQSYSTPF |
| | VKKPGASVKV | PSSLSAS | IS (SEQ | SGGTNY | YDFYY | NDLG | (SEQ | T (SEQ |
| | SCKASGGTFS | VGDRVTI | ID NO: | A (SEQ | GMDVW | (SEQ ID | ID | ID NO: |
| | TYAISWVRQA | TCRASQG | 215) | ID NO: | (SEQ | NO: | NO: | 152) |
| | PGQGLEWMGW | IRNDLGW | | 216) | ID | 218) | 219) | |
| | INPNSGGTNY | YQQKPGK | | | NO: | | | |
| | AQKFQGRVTM | APKLLIY | | | 217) | | | |
| | TRDTSTSTVY | RASTLNS | | | | | | |
| | MELSSLRSED | GVPSRFS | | | | | | |
| | TAVYYCARVN | GSGSGTD | | | | | | |
| | YDFYYGMDVW | FTLTISS | | | | | | |
| | GQGTTVTVSS | LQPEDFA | | | | | | |
| | (SEQ ID | TYYCQQS | | | | | | |
| | NO: 220) | YSTPFTF | | | | | | |
| | | GPGTKVD | | | | | | |
| | | IK (SEQ | | | | | | |
| | | ID NO: | | | | | | |
| | | 214) | | | | | | |
| PD1AB14 | EVQLLESGGG | DIQMTQS | FSFSSYD | SGISGS | CASPY | RASQDIA | SSVQT | QQSYTTPY |
| | LVQPGGSLRL | PSSLSAS | MS (SEQ | GSSTYY | GMGYM | NYLA | (SEQ | T (SEQ |
| | SCAASGFSFS | VGDRVTI | ID NO: | A (SEQ | DVW | (SEQ ID | ID | ID NO: |
| | SYDMSWVRQA | TCRASQD | 223) | ID NO: | (SEQ | NO : | NO: | 228) |
| | PGKGLEWVSG | IANYLAW | | 224) | ID | 226) | 227) | |
| | ISGSGSSTYY | YQQKPGK | | | NO: | | | |
| | ADSVKGRFTI | APKLLIY | | | 225) | | | |
| | SRDNSKNTLY | GASSVQT | | | | | | |
| | LQMNSLRAED | GVPSRFS | | | | | | |
| | TAVYYCASPY | GSGSGTD | | | | | | |
| | GMGYMDVWGK | FTLTISS | | | | | | |
| | GTTVTVSS | LQPEDFA | | | | | | |
| | (SEQ ID | TYYCQQS | | | | | | |
| | NO: 221) | YTTPYTF | | | | | | |
| | | GQGTRLE | | | | | | |
| | | IK (SEQ | | | | | | |
| | | ID NO: | | | | | | |
| | | 222) | | | | | | |
| PD1AB15 | QVQLVQSGAE | DIQMTQS | GSFNNYA | GWINPN | CARVS | QASQDIS | SNLQS | QQSYSTPF |
| | VKKPGASVKV | PSSLSAS | IS (SEQ | TGGTSY | YGVGY | RYLN | (SEQ | T (SEQ |
| | SCKASGGSFN | VGDRVTI | ID NO: | A (SEQ | YMDVW | (SEQ ID | ID | ID NO: |
| | NYAISWVRQA | TCQASQD | 231) | ID NO: | (SEQ | NO: | NO: | 152) |
| | PGQGLEWMGW | ISRYLNW | | 232) | ID | 234) | 235) | |
| | INPNTGGTSY | YQQKPGK | | | NO: | | | |
| | AQKFQGRVTM | APKLLIY | | | 233) | | | |
| | TRDTSTSTVY | AASNLQS | | | | | | |
| | MELSSLRSED | GVPSRFS | | | | | | |
| | TAVYYCARVS | GSGSGTD | | | | | | |
| | YGVGYYMDVW | FTLTISS | | | | | | |
| | GKGTTVTVSS | LQPEDFA | | | | | | |
| | (SEQ ID | TYYCQQS | | | | | | |
| | NO: 229) | YSTPFTF | | | | | | |
| | | GPGTKVD | | | | | | |
| | | IK (SEQ | | | | | | |
| | | ID NO: | | | | | | |
| | | 230) | | | | | | |
| PD1AB16 | QVQLVQSGAE | DIQMTQS | GSFNNYA | GWINPN | CARVS | QASQDIS | SNLQS | QQSYSTPF |
| | VKKPGASVKV | PSSLSAS | IS (SEQ | TGGTSY | YGVGY | RYLN | (SEQ | T (SEQ |
| | SCKASGGSFN | VGDRVTI | ID NO: | A (SEQ | YMDVW | (SEQ ID | ID | ID NO: |
| | NYAISWVRQA | TCQASQD | 231) | ID NO: | (SEQ | NO: | NO: | 152) |
| | PGQGLEWMGW | ISRYLNW | | 232) | ID | 234) | 235) | |
| | INPNTGGTSY | YQQKPGK | | | NO: | | | |
| | AQKFQGRVTM | APKLLIY | | | 233) | | | |
| | TRDTSTSTVY | AASNLQS | | | | | | |
| | MELSSLRSED | GVPSRFS | | | | | | |
| | TAVYYCARVS | GSGSGTD | | | | | | |
| | YGVGYYMDVW | STLTISS | | | | | | |
| | GKGTTVTVSS | LQPEDFA | | | | | | |
| | (SEQ ID | TYYCQQS | | | | | | |
| | NO: 229) | YSTPFTF | | | | | | |
| | | GPGTKVD | | | | | | |
| | | IK (SEQ | | | | | | |
| | | ID NO: | | | | | | |
| | | 236) | | | | | | |
| PD1AB17 | QVQLVQSGAE | DIQMTQS | YTFTDDY | GWMNTN | CARGG | RASQGVG | SSLQS | QQAYSFPW |
| | VKKPGASVKV | PSSLSAS | IH (SEQ | SGNTGY | SYSSG | NALG | (SEQ | T (SEQ |
| | SCKASGYTFT | VGDRVTI | ID NO: | A (SEQ | WYGRL | (SEQ ID | ID | ID NO: |
| | DDYIHWVRQA | TCRASQG | 239) | ID NO: | DYYYG | NO: | NO: | 243) |
| | PGQGLEWMGW | VGNALGW | | 240) | MDVW | 242) | | |
| | MNTNSGNTGY | YQQKPGK | | | (SEQ | | 143) | |
| | AQKFQGRVTM | APKLLIY | | | ID | | | |
| | TRDTSTSTVY | AASSLQS | | | NO: | | | |
| | MELSSLRSED | GVPSRFS | | | 241) | | | |
| | TAVYYCARGG | GSGSGTD | | | | | | |
| | SYSSGWYGRL | FTLTISS | | | | | | |
| | DYYYGMDVWG | LQPEDFA | | | | | | |
| | QGTTVTVSS | TYYCQQA | | | | | | |
| | (SEQ ID | YSFPWTF | | | | | | |
| | NO: 237) | GQGTKLE | | | | | | |
| | | IK (SEQ | | | | | | |
| | | ID NO: | | | | | | |
| | | 238) | | | | | | |
| PD1AB18 | QVQLVQSGAE | DIQMTQS | YTFTDYA | GWLNPN | CAAGL | RASQSIN | SSLES | QQSYSIPI |
| | VKKPGASVKV | PSSLSAS | MH (SEQ | SGNTGY | FIW | RWLA | (SEQ | T (SEQ |
| | SCKASGYTFT | VGDRVTI | ID NO: | A (SEQ | (SEQ | (SEQ ID | ID | ID NO: |
| | DYAMHWVRQA | TCRASQS | 246) | ID NO: | ID | NO: | NO: | 251) |
| | PGQGLEWMGW | INRWLAW | | 247) | NO: | 249) | 250) | |
| | LNPNSGNTGY | YQQKPGK | | | 248) | | | |
| | APKFQGRVTM | APKLLIY | | | | | | |
| | TRDTSTSTVY | DASSLES | | | | | | |
| | MELSSLRSED | GVPSRFS | | | | | | |
| | TAVYYCAAGL | GSGSGTD | | | | | | |
| | FIWGQGTMVT | FTLTISS | | | | | | |
| | VSS (SEQ | LQPEDFA | | | | | | |
| | ID NO: | TYYCQQS | | | | | | |
| | 244) | YSIPITF | | | | | | |
| | | GQGTRLE | | | | | | |
| | | IK (SEQ | | | | | | |
| | | ID NO: | | | | | | |
| | | 245) | | | | | | |
| PD1AB19 | QVQLVQSGAE | DIVMTQS | GTFSSYA | GGIIPG | CTTEY | RSSQSLL | SNRAP | MQALQTPL |
| | VKKPGSSVKV | PLSLPVT | IS (SEQ | FGSPNY | CSSTS | HSNGYNY | (SEQ | T (SEQ |
| | SCKASGGTFS | PGEPASI | ID NO: | A (SEQ | CSDYW | LD (SEQ | ID | ID NO: |
| | SYAISWVRQA | SCRSSQS | 155) | ID NO: | (SEQ | ID NO: | NO : | 258) |
| | PGQGLEWMGG | LLHSNGY | | 254) | ID | 256) | 257) | |
| | IIPGFGSPNY | NYLDWYL | | | NO: | | | |
| | APNFQGRVTI | QKPGQSP | | | 255) | | | |
| | TADESTSTAY | QLLIYQG | | | | | | |
| | MELSSLRSED | SNRAPGV | | | | | | |
| | TAVYYCTTEY | PDRFSGS | | | | | | |
| | CSSTSCSDYW | GSGTDFT | | | | | | |
| | GQGTLVTVSS | LKISRVE | | | | | | |
| | (SEQ ID | AEDVGVY | | | | | | |
| | NO: 252) | YCMQALQ | | | | | | |
| | | TPLTFGQ | | | | | | |
| | | GTKVEIK | | | | | | |
| | | (SEQ ID | | | | | | |
| | | NO: | | | | | | |
| | | 253) | | | | | | |
| PD1AB20 | QVQLVQSGAE | DIQMTQS | YTFSDHY | GTINPS | CAADN | RASQSIS | STLQS | QQSHSLPL |
| | VKKPGASVKV | PSSLSAS | MH (SEQ | GGRTSY | GHASG | NWVA | (SEQ | T (SEQ |
| | SCKASGYTFS | VGDRVTI | ID NO: | A (SEQ | WLYYY | (SEQ ID | | ID NO: |
| | DHYMHWVRQA | TCRASQS | 261) | ID NO: | GMDVW | NO: | ID | 265) |
| | PGQGLEWMGT | ISNWVAW | | 262) | (SEQ | | NO: | |
| | INPSGGRTSY | YQQKPGK | | | ID | 264) | 171) | |
| | AQKFQGRVTM | APKLLIY | | | NO: | | | |
| | TRDTSTSTVY | RASTLQS | | | 263) | | | |
| | MELSSLRSED | GVPSRFS | | | | | | |
| | TAVYYCAADN | GSGSGTD | | | | | | |
| | GHASGWLYYY | FTLTISS | | | | | | |
| | GMDVWGQGTT | LQPEDFA | | | | | | |
| | VTVSS (SEQ | TYYCQQS | | | | | | |
| | ID NO: | HSLPLTF | | | | | | |
| | 259) | GPGTKVD | | | | | | |
| | | IK (SEQ | | | | | | |
| | | ID NO: | | | | | | |
| | | 260) | | | | | | |
| PD1AB21 | EVQLLESGGG | DIQMTQS | FTFSSYA | SGISGG | CASEY | RASQSIS | SSLQS | QQYRNFPY |
| | LVQPGGSLRL | PSSLSAS | MS (SEQ | GGTTYY | YGMDV | GWLA | (SEQ | T (SEQ |
| | SCAASGFTFS | VGDRVTI | ID NO: | A (SEQ | W | (SEQ ID | ID | ID NO: |
| | SYAMSWVRQA | TCRASQS | 168) | ID NO: | (SEQ | NO: | NO: | 271) |
| | PGKGLEWVSG | ISGWLAW | | 268) | ID | 270) | 143) | |
| | ISGGGGTTYY | YQQKPGK | | | NO: | | | |
| | ADSVKGRFTI | APKLLIY | | | 269) | | | |
| | SRDNSKNTLY | AASSLQS | | | | | | |
| | LQMNSLRAED | GVPSRFS | | | | | | |
| | TAVYYCASEY | GSGSGTD | | | | | | |
| | YGMDVWGQGT | FTLTISS | | | | | | |
| | TVTVSS | LQPEDFA | | | | | | |
| | (SEQ ID | TYYCQQY | | | | | | |
| | NO: 266) | RNFPYTF | | | | | | |
| | | GQGTKLE | | | | | | |
| | | IK (SEQ | | | | | | |
| | | ID NO: | | | | | | |
| | | 267) | | | | | | |
| PD1AB22 | QVQLVQSGAE | EIVMTQS | YTFSGYY | GVINPS | CAEGF | RASQGVG | STRAT | QQYYTTPI |
| | VKKPGASVKV | PATLSVS | MH (SEQ | GGSTSY | DYW | RSLA | (SEQ | T (SEQ |
| | SCKASGYTFS | PGERATL | ID NO: | A (SEQ | (SEQ | (SEQ ID | ID | ID NO: |
| | GYYMHWVRQA | SCRASQG | 274) | ID NO: | ID | NO: | NO: | 279) |
| | PGQGLEWMGV | VGRSLAW | | 275) | NO: | 277) | 278) | |
| | INPSGGSTSY | YQQKPGQ | | | 276) | | | |
| | AQKFQGRVTM | APRLLIY | | | | | | |
| | TRDTSTSTVY | GASTRAT | | | | | | |
| | MELSSLRSED | GIPARFS | | | | | | |
| | TAVYYCAEGF | GSGSGTE | | | | | | |
| | DYWGQGTLVT | FTLTISS | | | | | | |
| | VSS (SEQ | LQSEDFA | | | | | | |
| | ID NO: | VYYCQQY | | | | | | |
| | 272) | YTTPITF | | | | | | |
| | | GQGTRLE | | | | | | |
| | | IK (SEQ | | | | | | |
| | | ID NO: | | | | | | |
| | | 273) | | | | | | |
| PD1AB23 | QVQLVQSGAE | DIQMTQS | GTFSNYA | GWMNPN | CARVN | QASQDIS | STLKS | QQADNLPF |
| | VKKPGASVKV | PSSLSAS | IS (SEQ | SGNTGY | YYYYY | NYLN | (SEQ | T (SEQ |
| | SCKASGGTFS | VGDRVTI | ID NO: | A (SEQ | GMDVW | (SEQ ID | ID | ID NO: |
| | NYAISWVRQA | TCQASQD | 282) | ID NO: | (SEQ | NO: | NO: | 286) |
| | PGQGLEWMGW | ISNYLNW | | 156) | ID | 284) | 285) | |
| | MNPNSGNTGY | YQQKPGK | | | NO: | | | |
| | AQKFQGRVTM | APKLLIY | | | 283) | | | |
| | TRDTSTSTVY | KASTLKS | | | | | | |
| | MELSSLRSED | GVPSRFS | | | | | | |
| | TAVYYCARVN | GSGSGTD | | | | | | |
| | YYYYYGMDVW | FTLTISS | | | | | | |
| | GQGTTVTVSS | LQPEDFA | | | | | | |
| | (SEQ ID | TYYCQQA | | | | | | |
| | NO: 280) | DNLPFTF | | | | | | |
| | | GPGTKVD | | | | | | |
| | | IK (SEQ | | | | | | |
| | | ID NO: | | | | | | |
| | | 281) | | | | | | |
| PD1AB24 | QVQLVQSGAE | DIQMTQS | YTFTNYY | GIINPS | CARDW | QASRDIS | SSLQS | QQANSFPP |
| | VKKPGASVKV | PSSLSAS | MH (SEQ | GGSTSY | GWDYY | NYLN | (SEQ | T (SEQ |
| | SCKASGYTFT | VGDRVTI | ID NO: | A (SEQ | YYGMD | (SEQ ID | ID | ID NO: |
| | NYYMHWVRQA | TCQASRD | 289) | ID NO: | VW | NO: | NO: | 292) |
| | PGQGLEWMGI | ISNYLNW | | 162) | (SEQ | 291) | 143) | |
| | INPSGGSTSY | YQQKPGK | | | ID | | | |
| | AQRFQGRVTM | APKLLIY | | | NO: | | | |
| | TRDTSTSTVY | AASSLQS | | | 290) | | | |
| | MELSSLRSED | GVPSRFS | | | | | | |
| | TAVYYCARDW | GSGSGTD | | | | | | |
| | GWDYYYYGMD | FTLTISS | | | | | | |
| | VWGQGTTVTV | LQPEDFA | | | | | | |
| | SS (SEQ ID | TYYCQQA | | | | | | |
| | NO: 287) | NSFPPTF | | | | | | |
| | | GQGTKLE | | | | | | |
| | | IK (SEQ | | | | | | |
| | | ID NO: | | | | | | |
| | | 288) | | | | | | |
| PD1AB25 | EVQLLESGGG | DIVMTQS | FTFSNSD | SGITIS | CARGR | KSSQSVL | STRES | QQYYTTPP |
| | LVQPGGSLRL | PDSLAVS | MS (SEQ | GGSTYY | GGSGW | YSPNNKN | (SEQ | T (SEQ |
| | SCAASGFTFS | LGERATI | ID NO: | A (SEQ | LDYW | YLA | ID | ID NO: |
| | NSDMSWVRQA | NCKSSQS | 295) | ID NO: | (SEQ | (SEQ ID | NO: | 300) |
| | PGKGLEWVSG | VLYSPNN | | 296) | ID | NO: | 299) | |
| | ITISGGSTYY | KNYLAWY | | | NO: | 298) | | |
| | ADSVRGRFTI | QQKPGQP | | | 297) | | | |
| | SRDNSKNTLY | PKLLIYW | | | | | | |
| | LQMSSLRAED | ASTRESG | | | | | | |
| | TAVYYCARGR | VPDRFSG | | | | | | |
| | GGSGWLDYWG | SGSGTDF | | | | | | |
| | QGTLVTVSS | TLTISSL | | | | | | |
| | (SEQ ID | QAEDVAV | | | | | | |
| | NO: 293) | YYCQQYY | | | | | | |
| | | TTPPTFG | | | | | | |
| | | QGTRLEI | | | | | | |
| | | K (SEQ | | | | | | |
| | | ID NO: | | | | | | |
| | | 294) | | | | | | |
| PD1AB26 | QVQLVQSGAE | DIQMTQS | YTFTGYY | GKIVPM | CARGP | RASQSIS | SSLQS | QQANSFPV |
| | VKKPGSSVKV | PSSLSAS | MH (SEQ | FDAANY | KWELD | RWLA | (SEQ | T (SEQ |
| | SCKASGYTFT | VGDRVTI | ID NO: | A (SEQ | TW | SEQ ID | ID | ID NO: |
| | GYYMHWVRQA | TCRASQS | 197) | ID NO: | (SEQ | NO: | NO: | 201) |
| | PGQGLEWMGK | ISRWLAW | | 198) | ID | 200) | 143) | |
| | IVPMFDAANY | YQQKPGK | | | NO: | | | |
| | APKFQGRVTI | APKLLIY | | | 199) | | | |
| | TADESTSTAY | GASSLQS | | | | | | |
| | MELSSLRSED | GVPSRFS | | | | | | |
| | TAVYYCARGP | GSGSGTD | | | | | | |
| | KWELDTWGQG | FTLTISS | | | | | | |
| | TLVTVSS | LQPEDFA | | | | | | |
| | (SEQ ID | TYYCQQA | | | | | | |
| | NO: 195) | NSFPVTF | | | | | | |
| | | GGGTKVD | | | | | | |
| | | IK (SEQ | | | | | | |
| | | ID NO: | | | | | | |
| | | 301) | | | | | | |
| PD1AB27 | QVQLVQSGAE | DIQMTQS | YTFTGYY | GKIVPM | CARGP | RASQSIS | SSLQS | QQANSFPV |
| | VKKPGSSVKV | PSSLSAS | MH (SEQ | FDAANY | KWELD | RWLA | (SEQ | T (SEQ |
| | SCKASGYTFT | VGDRVTI | ID NO: | A (SEQ | TW | (SEQ ID | ID | ID NO: |
| | GYYMHWVRQA | TCRASQS | 197) | ID NO: | (SEQ | NO: | NO: | 201) |
| | PGQGLEWMGK | ISRWLAW | | 198) | ID | 200) | 143) | |
| | IVPMFDAANY | YQQKPGK | | | NO : | | | |
| | APKFQGRVTI | APKLLIY | | | 199) | | | |
| | TADESTSTAY | GASSLQS | | | | | | |
| | MELSSLRSED | GVPSRFS | | | | | | |
| | TAVYYCARGP | GSGSGTD | | | | | | |
| | KWELDTWGQG | FTFTISS | | | | | | |
| | TLVTVSS | LQPEDFA | | | | | | |
| | (SEQ ID | TYYCQQA | | | | | | |
| | NO: 195) | NSFPVTF | | | | | | |
| | | GGGTKVD | | | | | | |
| | | IK (SEQ | | | | | | |
| | | ID NO: | | | | | | |
| | | 302) | | | | | | |
| PD1AB28 | QVQLVQSGAE | DIQMTQS | GDFSNYF | GWINPH | CARGG | RASQSIS | STLQS | QQSYSTPF |
| | VKKPGSSVKV | PSSLSAS | VS (SEQ | NGDTMY | YSYGY | TWLA | (SEQ | T (SEQ |
| | SCKASGGDFS | VGDRVTI | ID NO: | A (SEQ | TFDIW | (SEQ ID | ID | ID NO: |
| | NYFVSWVRQA | TCRASQS | 305) | ID NO : | (SEQ | NO: | NO: | 152) |
| | PGQGLEWMGW | ISTWLAW | | 306) | ID | 308) | 171) | |
| | INPHNGDTMY | YQQKPGK | | | NO: | | | |
| | AQKFQGRVTI | APKLLIY | | | 307) | | | |
| | TADESTSTAY | AASTLQS | | | | | | |
| | MELSSLRSED | GVPSRFS | | | | | | |
| | TAVYYCARGG | GSGSGTD | | | | | | |
| | YSYGYTFDIW | FTLTISS | | | | | | |
| | GQGTMVTVSS | LQPEDFA | | | | | | |
| | (SEQ ID | TYYCQQS | | | | | | |
| | NO: 303) | YSTPFTF | | | | | | |
| | | GGGTKVE | | | | | | |
| | | IK (SEQ | | | | | | |
| | | ID NO: | | | | | | |
| | | 304) | | | | | | |
| PD1AB29 | EVQLLESGGG | DIVMTQS | FTFSNSD | SGITIS | CARGR | KSSQSVL | STRES | QQYYITPP |
| | LVQPGGSLRL | PDSLAVS | MS (SEQ | GGSTYY | GGSGW | YSPNNKN | (SEQ | T (SEQ |
| | SCAASGFTFS | LGERATI | ID NO: | A (SEQ | LDYW | YLA | ID | ID NO: |
| | NSDMSWVRQA | NCKSSQS | 295) | ID NO: | (SEQ | (SEQ ID | NO : | 311) |
| | PGKGLEWVSG | VLYSPNN | | 296) | ID | NO: | 299) | |
| | ITISGGSTYY | KNYLAWY | | | NO: | 298) | | |
| | ADSVKGRFTI | QQKPGQP | | | 297) | | | |
| | SRDNSKNTLY | PKLLIYW | | | | | | |
| | LQMNSLRAED | ASTRESG | | | | | | |
| | TAVYYCARGR | VPDRFSG | | | | | | |
| | GGSGWLDYWG | SGSGTDF | | | | | | |
| | QGTLVTVSS | TLTISSL | | | | | | |
| | (SEQ ID | QAEDVAV | | | | | | |
| | NO: 309) | YYCQQYY | | | | | | |
| | | ITPPTFG | | | | | | |
| | | QGTRLEI | | | | | | |
| | | K (SEQ | | | | | | |
| | | ID NO: | | | | | | |
| | | 310) | | | | | | |
| PD1AB30 | EVQLLESGGG | DIVMTQS | FTFSNSD | SGITIS | CARGR | KSSQSVL | STRES | QQYYTTPP |
| | LVQPGGSLRL | PDSLAVS | MS (SEQ | GGSTYY | GGSGW | YSPNNKN | (SEQ | T (SEQ |
| | SCAASGFTFS | LGERATI | ID NO: | A (SEQ | LDYW | YLA | ID | ID NO: |
| | NSDMSWVRQA | NCKSSQS | 295) | ID NO: | (SEQ | (SEQ ID | NO: | 300) |
| | PGKGLEWVSG | VLYSPNN | | 296) | ID | NO: | 299) | |
| | ITISGGSTYY | KNYLAWY | | | NO: | 298) | | |
| | ADSVKGRFTI | QQKPGQP | | | 297) | | | |
| | SRDNSKNTLY | PKLLIYW | | | | | | |
| | LQMNSLRAED | ASTRESG | | | | | | |
| | TAVYYCARGR | VPDRFSG | | | | | | |
| | GGSGWLDYWG | SGSGTDF | | | | | | |
| | QGTLVTVSS | TLTISSL | | | | | | |
| | (SEQ ID | QAEDVAV | | | | | | |
| | NO: 309) | YYCQQYY | | | | | | |
| | | TTPPTFG | | | | | | |
| | | QGTRLEI | | | | | | |
| | | K (SEQ | | | | | | |
| | | ID NO: | | | | | | |
| | | 294) | | | | | | |
| PD1AB31 | QVQLVQSGAE | EIVMTQS | HTFTDYY | GIINPS | CASGW | RASQSVS | SSRAT | QQYTTSPI |
| | VKKPGSSVKV | PATLSVS | MH (SEQ | GGSTSY | TDW | SYLA | (SEQ | T (SEQ |
| | SCKASGHTFT | PGERATL | ID NO: | A (SEQ | (SEQ | (SEQ ID | ID | ID NO: |
| | DYYMHWVRQA | SCRASQS | 314) | ID NO: | ID | NO: | NO: | 318) |
| | PGQGLEWMGI | VSSYLAW | | 162) | NO: | 316) | 317) | |
| | INPSGGSTSY | YQQKPGQ | | | 315) | | | |
| | AQKFQGRVTI | APRLLIY | | | | | | |
| | TADESTSTAY | GTSSRAT | | | | | | |
| | MELSSLRSED | GIPARFS | | | | | | |
| | TAVYYCASGW | GSGSGTE | | | | | | |
| | TDWGQGTLVT | FTLTISS | | | | | | |
| | VSS (SEQ | LQSEDFA | | | | | | |
| | ID NO: | VYYCQQY | | | | | | |
| | 312) | TTSPITF | | | | | | |
| | | GQGTRLE | | | | | | |
| | | IKR | | | | | | |
| | | (SEQ ID | | | | | | |
| | | NO: | | | | | | |
| | | 313) | | | | | | |
| PD1AB32 | QVQLVQSGAE | DIQMTQS | YTFTDYY | GGIFPV | CARDH | QASQDIS | KDLHP | QESFSTLT |
| | VKKPGASVKV | PSSLSAS | MH (SEQ | FGSSTY | GSGLD | NYLN | (SEQ | (SEQ ID |
| | SCKASGYTFT | VGDRVTI | ID NO: | A (SEQ | VW | (SEQ ID | ID | NO: 325) |
| | DYYMHWVRQA | TCQASQD | 321) | ID NO: | (SEQ | NO: | NO: | |
| | PGQGLEWMGG | ISNYLNW | | 322) | ID | 284) | 324) | |
| | IFPVFGSSTY | YQQKPGK | | | NO: | | | |
| | AQKFQGRVTM | APKLLIY | | | 323) | | | |
| | TRDTSTSTVY | DAKDLHP | | | | | | |
| | MELSSLRSED | GVPSRFS | | | | | | |
| | TAVYYCARDH | GSGSGTD | | | | | | |
| | GSGLDVWGQG | FTLTISS | | | | | | |
| | TTVTVSS | LQPEDFA | | | | | | |
| | (SEQ ID | TYYCQES | | | | | | |
| | NO: 319) | FSTLTFG | | | | | | |
| | | QGTKVEI | | | | | | |
| | | KR (SEQ | | | | | | |
| | | ID NO: | | | | | | |
| | | 320) | | | | | | |
| PD1AB33 | QVQLVQSGAE | DIQMTQS | YSFTTYY | GIIAPS | CASGW | QASRDIK | SSLQS | QQSYSTPP |
| | VKKPGASVKV | PSSLSAS | MH (SEQ | GGSTSY | VYW | NYLA | (SEQ | T (SEQ |
| | SCKASGYSFT | VGDRVTI | ID NO: | A (SEQ | (SEQ | (SEQ ID | ID | ID NO: |
| | TYYMHWVRQA | TCQASRD | 161) | ID NO: | ID | NO: | NO: | 165) |
| | PGQGLEWMGI | IKNYLAW | | 327) | NO: | 164) | 143) | |
| | IAPSGGSTSY | YQQKPGK | | | 163) | | | |
| | AQKFQGRVTM | APKLLIY | | | | | | |
| | TRDTSTSTVY | AASSLQS | | | | | | |
| | MELSSLRSED | GVPSRFS | | | | | | |
| | TAVYYCASGW | GSGSGTD | | | | | | |
| | VYWGQGTLVT | FTLTISS | | | | | | |
| | VSS (SEQ | LQPEDFA | | | | | | |
| | ID NO: | TYYCQQS | | | | | | |
| | 326) | YSTPPTF | | | | | | |
| | | GPGTKVD | | | | | | |
| | | IK (SEQ | | | | | | |
| | | ID NO: | | | | | | |
| | | 160) | | | | | | |
| PD1AB34 | QVQLVQSGAE | DIQMTQS | YSFTTYY | GIIAPS | CASGW | QASRDIK | SSLQS | QQSYSTPP |
| | VKKPGASVKV | PSSLSAS | MH (SEQ | GGSTSY | VYW | NYLA | (SEQ | T (SEQ |
| | SCKASGYSFT | VGDRVTI | ID NO: | A (SEQ | (SEQ | (SEQ ID | ID | ID NO: |
| | TYYMHWVRQA | TCQASRD | 161) | ID NO: | ID | NO: | NO: | 165) |
| | PGQGLEWMGI | IKNYLAW | | 327) | NO: | 164) | 143) | |
| | IGPSGGSTSY | YQQKPGK | | | 163) | | | |
| | AQKFQGRVTM | APKLLIY | | | | | | |
| | TRDTSTSTVY | AASSLQS | | | | | | |
| | MELSSLRSED | GVPSRFS | | | | | | |
| | TAVYYCASGW | GSGSGTD | | | | | | |
| | VYWGQGTLVT | FTLTISS | | | | | | |
| | VSS (SEQ | LQPEDFA | | | | | | |
| | ID NO: | TYYCQQS | | | | | | |
| | 328) | YSTPPTF | | | | | | |
| | | GPGTKVD | | | | | | |
| | | IK (SEQ | | | | | | |
| | | ID NO: | | | | | | |
| | | 160) | | | | | | |
| PD1AB35 | QVQLVQSGAE | DIQMTQS | YTFSDHY | GTIAPS | CAADN | RASQSIS | STLQS | QQSHSLPL |
| | VKKPGASVKV | PSSLSAS | MH (SEQ | GGRTSY | GHASG | NWVA | (SEQ | T (SEQ |
| | SCKASGYTFS | VGDRVTI | ID NO: | A (SEQ | WLYYY | (SEQ ID | ID | ID NO: |
| | DHYMHWVRQA | TCRASQS | 261) | ID NO: | GMDVW | NO: | NO : | 265) |
| | PGQGLEWMGT | ISNWVAW | | 330) | (SEQ | 264) | 171) | |
| | IAPSGGRTSY | YQQKPGK | | | ID | | | |
| | AQKFQGRVTM | APKLLIY | | | NO: | | | |
| | TRDTSTSTVY | RASTLQS | | | 263) | | | |
| | MELSSLRSED | GVPSRFS | | | | | | |
| | TAVYYCAADN | GSGSGTD | | | | | | |
| | GHASGWLYYY | FTLTISS | | | | | | |
| | GMDVWGQGTT | LQPEDFA | | | | | | |
| | VTVSS (SEQ | TYYCQQS | | | | | | |
| | ID NO: | HSLPLTF | | | | | | |
| | 329) | GPGTKVD | | | | | | |
| | | IK (SEQ | | | | | | |
| | | ID NO: | | | | | | |
| | | 260) | | | | | | |
| PD1AB36 | QVQLVQSGAE | DIQMTQS | YTFSDHY | GTIAPS | CAADN | RASQSIS | STLQS | QQSHSLPL |
| | VKKPGASVKV | PSSLSAS | MH (SEQ | GGRTSY | GHASG | NWVA | (SEQ | T (SEQ |
| | SCKASGYTFS | VGDRVTI | ID NO: | A (SEQ | WLYYY | (SEQ ID | ID | ID NO: |
| | DHYMHWVRQA | TCRASQS | 261) | ID NO : | GMDVW | NO: | NO: | 265) |
| | PGQGLEWMGT | ISNWVAW | | 330) | (SEQ | 264) | 171) | |
| | IGPSGGRTSY | YQQKPGK | | | ID | | | |
| | AQKFQGRVTM | APKLLIY | | | NO: | | | |
| | TRDTSTSTVY | RASTLQS | | | 263) | | | |
| | MELSSLRSED | GVPSRFS | | | | | | |
| | TAVYYCAADN | GSGSGTD | | | | | | |
| | GHASGWLYYY | FTLTISS | | | | | | |
| | GMDVWGQGTT | LQPEDFA | | | | | | |
| | VTVSS (SEQ | TYYCQQS | | | | | | |
| | ID NO: | HSLPLTF | | | | | | |
| | 331) | GPGTKVD | | | | | | |
| | | IK (SEQ | | | | | | |
| | | ID NO: | | | | | | |
| | | 260) | | | | | | |
| PD1AB37 | QVQLVQSGAE | DIQMTQS | TYYMH | IINPSG | ASGWV | QASRDIK | AASSL | QQSYSTPP |
| | VKKPGASVKV | PSSLSAS | (SEQ ID | GSTSYA | Y | NYLA | QS | T (SEQ |
| | SCKASGYSFT | VGDRVTI | NO: | QKFQG | (SEQ | (SEQ ID | (SEQ | ID NO: |
| | TYYMHWVRQA | TCQASRD | 332) | (SEQ | ID | NO: | ID | 165) |
| | PGQGLEWMGI | IKNYLAW | | ID NO: | NO: | 164) | NO : | |
| | INPSGGSTSY | YQQKPGK | | 333) | 334) | | 335) | |
| | AQKFQGRVTM | APKLLIY | | | | | | |
| | TRDTSTSTVY | AASSLQS | | | | | | |
| | MELSSLRSED | GVPSRFS | | | | | | |
| | TAVYYCASGW | GSGSGTD | | | | | | |
| | VYWGQGTLVT | FTLTISS | | | | | | |
| | VSS (SEQ | LQPEDFA | | | | | | |
| | ID NO: | TYYCQQS | | | | | | |
| | 159) | YSTPPTF | | | | | | |
| | | GPGTKVD | | | | | | |
| | | IK (SEQ | | | | | | |
| | | ID NO: | | | | | | |
| | | 160) | | | | | | |
| PD1AB38 | EVQLLESGGG | DIVMTQS | SNSDMS | GITISG | ARGRG | KSSQSVL | WASTR | QQYYTTPP |
| | LVQPGGSLRL | PDSLAVS | (SEQ ID | GSTYYA | GSGWL | YSPNNKN | ES | T (SEQ |
| | SCAASGFTFS | LGERATI | NO : | DSVK | DY | YLA | (SEQ | ID NO: |
| | NSDMSWVRQA | NCKSSQS | 336) | (SEQ | (SEQ | (SEQ ID | ID | 300) |
| | PGKGLEWVSG | VLYSPNN | | ID NO: | ID | NO: | NO : | |
| | ITISGGSTYY | KNYLAWY | | 337) | NO: | 298) | 339) | |
| | ADSVKGRFTI | QQKPGQP | | | 338) | | | |
| | SRDNSKNTLY | PKLLIYW | | | | | | |
| | LQMNSLRAED | ASTRESG | | | | | | |
| | TAVYYCARGR | VPDRFSG | | | | | | |
| | GGSGWLDYWG | SGSGTDF | | | | | | |
| | QGTLVTVSS | TLTISSL | | | | | | |
| | (SEQ ID | QAEDVAV | | | | | | |
| | NO: 309) | YYCQQYY | | | | | | |
| | | TTPPTFG | | | | | | |
| | | QGTRLEI | | | | | | |
| | | K (SEQ | | | | | | |
| | | ID NO: | | | | | | |
| | | 294) | | | | | | |

In some embodiments, the antibody is linked to another antibody or therapeutic. In some embodiments, the PD-1 antibody is linked to an anti-desmoglein 1 antibody, an anti-desmoglein 2 antibody, an anti-desmoglein 3 antibody, or an anti-desmoglein 4 antibody or a IL-2 mutein as provided herein or that is incorporated by reference.

In some embodiments, the anti-desmoglein antibody linked to the PD-1 antibody has a sequence as set forth in DSG-PD-1 Table.

DSG-PD-1 Table

| Ab# | Heavy chain Seq | Light chain Seq |
|---|---|---|
| PD1DSGAB1 | QVQLVQSGAEVKKPGASVKVSCKASGYSFTTYYMHWV | DIQMTQSPSSLSASVGDRVTITCQASRDIKNY |
| | RQAPGQGLEWMGIINPSGGSTSYAQKFQGRVTMTRDT | LAWYQQKPGKAPKLLIYAASSLQSGVPSRFSG |
| | STSTVYMELSSLRSEDTAVYYCASGWVYWGQGTLVTV | SGSGTDFTLTISSLQPEDFATYYCQQSYSTPP |
| | SSASTKGPSVFPLAPSSKSTSGGTAALGCLVKDYFPE | TFGPGTKVDIKRTVAAPSVFIFPPSDEQLKSG |
| | PVTVSWNSGALTSGVHTFPAVLQSSGLYSLSSVVTVP | TASVVCLLNNFYPREAKVQWKVDNALQSGNSQ |
| | SSSLGTQTYICNVNHKPSNTKVDKKVEPKSCDKTHTC | ESVTEQDSKDSTYSLSSTLTLSKADYEKHKVY |
| | PPCPAPEAAGAPSVFLFPPKPKDTLMISRTPEVTCVV | ACEVTHQGLSSPVTKSFNRGEC (SEQ ID |
| | VDVSHEDPEVKFNWYVDGVEVHNAKTKPREEQYNSTY | NO: 341) |
| | RVVSVLTVLHQDWLNGKEYKCKVSNKALPAPIEKTIS | |
| | KAKGQPREPQVYTLPPSREEMTKNQVSLTCLVKGFYP | |
| | SDIAVEWESNGQPENNYKTTPPVLDSDGSFFLYSKLT | |
| | VDKSRWQQGNVFSCSVMHEALHNHYTQKSLSLSPGGG | |
| | GGSGGGGSGGGGSQSVLTQPPSVSGTPGQRVTISCSG | |
| | SSSHIGSNYVYWYQQLPGTAPKILIYSNDQRPAGVPD | |
| | RESASKSGTSASLAISGLRSEDEADYYCAAWDDGQGG | |
| | VFGGGTKLTVLGGGGSGGGGSGGGGSGGGGSQVQLQE | |
| | SGPGPVKPSGTLSLTCGVSGGSISSNHWWTWVRQPPG | |
| | KGLEWIGEIYHNGSTFLNPSLKSRVTISVDKSNNQFS | |
| | LKLTSVTAADTAVYYCARGWHRTGFRGYPSHWYFDLW | |
| | GRGTLVSVSS (SEQ ID NO: 340) | |
| PD1DSGAB2 | QVQLVQSGAEVKKPGASVKVSCKASGYSFTTYYMHWV | DIQMTQSPSSLSASVGDRVTITCQASRDIKNY |
| | RQAPGQGLEWMGIINPSGGSTSYAQKFQGRVTMTRDT | LAWYQQKPGKAPKLLIYAASSLQSGVPSRFSG |
| | STSTVYMELSSLRSEDTAVYYCASGWVYWGQGTLVTV | SGSGTDFTLTISSLQPEDFATYYCQQSYSTPP |
| | SSASTKGPSVFPLAPSSKSTSGGTAALGCLVKDYFPE | TFGPGTKVDIKRTVAAPSVFIFPPSDEQLKSG |
| | PVTVSWNSGALTSGVHTFPAVLQSSGLYSLSSVVTVP | TASVVCLLNNFYPREAKVQWKVDNALQSGNSQ |
| | SSSLGTQTYICNVNHKPSNTKVDKKVEPKSCDKTHTC | ESVTEQDSKDSTYSLSSTLTLSKADYEKHKVY |
| | PPCPAPEAAGAPSVFLFPPKPKDTLMISRTPEVTCVV | ACEVTHQGLSSPVTKSFNRGEC (SEQ ID |
| | VDVSHEDPEVKFNWYVDGVEVHNAKTKPREEQYNSTY | NO: 343) |
| | RVVSVLTVLHQDWLNGKEYKCKVSNKALPAPIEKTIS | |
| | KAKGQPREPQVYTLPPSREEMTKNQVSLTCLVKGFYP | |
| | SDIAVEWESNGQPENNYKTTPPVLDSDGSFFLYSKLT | |
| | VDKSRWQQGNVFSCSVMHEALHNHYTQKSLSLSPGGG | |
| | GGSGGGGSGGGGSDIQMTQSPSSLSASVGDRVTITCR | |
| | ASQSISSYLNWYQQKPGKAPKLLIYAASSLQSGVPSR | |
| | FSGSGSGTDFTLTISSLQPEDFATYYCQQSYSTPRTF | |
| | GGGTKLEIKGGGGSGGGGSGGGGSGGGGSQVQLVQSG | |
| | AEVRKPGASVRVSCKASGYTLTTYDINWVRQATGQGL | |
| | EWMGWMNPTSGNTAYAQKFQGRVTMTRNTSISTAYME | |
| | LSSLRSEDTAVYYCARGLFFGVVTKPNYYYYAMDVWG | |
| | QGTTVTVSS (SEQ ID NO: 342) | |
| PD1DSGAB3 | QVQLVQSGAEVKKPGASVKVSCKASGYSFTTYYMHWV | DIQMTQSPSSLSASVGDRVTITCQASRDIKNY |
| | RQAPGQGLEWMGIINPSGGSTSYAQKFQGRVTMTRDT | LAWYQQKPGKAPKLLIYAASSLQSGVPSRFSG |
| | STSTVYMELSSLRSEDTAVYYCASGWVYWGQGTLVTV | SGSGTDFTLTISSLQPEDFATYYCQQSYSTPP |
| | SSASTKGPSVFPLAPSSKSTSGGTAALGCLVKDYFPE | TFGPGTKVDIKRTVAAPSVFIFPPSDEQLKSG |
| | PVTVSWNSGALTSGVHTFPAVLQSSGLYSLSSVVTVP | TASVVCLLNNFYPREAKVQWKVDNALQSGNSQ |
| | SSSLGTQTYICNVNHKPSNTKVDKKVEPKSCDKTHTC | ESVTEQDSKDSTYSLSSTLTLSKADYEKHKVY |
| | PPCPAPEAAGAPSVFLFPPKPKDTLMISRTPEVTCVV | ACEVTHQGLSSPVTKSFNRGEC (SEQ ID |
| | VDVSHEDPEVKFNWYVDGVEVHNAKTKPREEQYNSTY | NO: 345) |
| | RVVSVLTVLHQDWLNGKEYKCKVSNKALPAPIEKTIS | |
| | KAKGQPREPQVYTLPPSREEMTKNQVSLTCLVKGFYP | |
| | SDIAVEWESNGQPENNYKTTPPVLDSDGSFFLYSKLT | |
| | VDKSRWQQGNVFSCSVMHEALHNHYTQKSLSLSPGGG | |
| | GGSGGGGSGGGGSELQMTQSPSSLSASVGDRVTITCQ | |
| | ASQDIGNYLNWYQQKPGKAPKLLIYDASYLETGVPSR | |
| | FSGSGSGTDFTFTISSLQPEDIATYYCQQYDNLPFTF | |
| | GPGTKVDIKGGGGSGGGGSGGGGSGGGGSEVQLVESG | |
| | GGLVQPGRSLRLSCAASGFTFDDYAMHWVRQAPGKGL | |
| | EWVSGISWNSGSIDYADSVKGRFTISRDNAKNSLYLQ | |
| | MNSLRVEDTALYYCAKDGSRVFGVGGGFDFWGQGTMV | |
| | TVSS (SEQ ID NO: 344) | |
| PD1DSGAB4 | QVQLVQSGAEVKKPGASVKVSCKASGYSFTTYYMHWV | DIQMTQSPSSLSASVGDRVTITCQASRDIKNY |
| | RQAPGQGLEWMGIINPSGGSTSYAQKFQGRVTMTRDT | LAWYQQKPGKAPKLLIYAASSLQSGVPSRFSG |
| | STSTVYMELSSLRSEDTAVYYCASGWVYWGQGTLVTV | SGSGTDFTLTISSLQPEDFATYYCQQSYSTPP |
| | SSASTKGPSVFPLAPSSKSTSGGTAALGCLVKDYFPE | TFGPGTKVDIKRTVAAPSVFIFPPSDEQLKSG |
| | PVTVSWNSGALTSGVHTFPAVLQSSGLYSLSSVVTVP | TASVVCLLNNFYPREAKVQWKVDNALQSGNSQ |
| | SSSLGTQTYICNVNHKPSNTKVDKKVEPKSCDKTHTC | ESVTEQDSKDSTYSLSSTLTLSKADYEKHKVY |
| | PPCPAPEAAGAPSVFLFPPKPKDTLMISRTPEVTCVV | ACEVTHQGLSSPVTKSFNRGEC (SEQ ID |
| | VDVSHEDPEVKFNWYVDGVEVHNAKTKPREEQYNSTY | NO: 347) |
| | RVVSVLTVLHQDWLNGKEYKCKVSNKALPAPIEKTIS | |
| | KAKGQPREPQVYTLPPSREEMTKNQVSLTCLVKGFYP | |
| | SDIAVEWESNGQPENNYKTTPPVLDSDGSFFLYSKLT | |
| | VDKSRWQQGNVFSCSVMHEALHNHYTQKSLSLSPGGG | |
| | GGSGGGGSGGGGSELVLTQPPSVPVAPGQTANISCGG | |
| | NNIGRQTVHWYQQKPGQAPVLVVFDDSDRPAGIPERF | |
| | SGSNSGNTATLTISRVEAGDEADYYCQVWDSSSDHVV | |
| | FGGGTQLTVLGGGGSGGGGSGGGGSGGGGSQVQLVQS | |
| | GGGVVQSGRSLRLSCAASGFTFSDYAMHWVRQAPGKG | |
| | LEWVAVISHGGTKKYTGDSVKGRFIISRDNSKNTVFL | |
| | QMNSLRVEDTAVYYCARDRVEGYVWGGTFDHWGQGTP | |
| | VTVSS (SEQ ID NO: 346) | |
| PD1DSGAB5 | QVQLVQSGAEVKKPGASVKVSCKASGYSFTTYYMHWV | DIQMTQSPSSLSASVGDRVTITCQASRDIKNY |
| | RQAPGQGLEWMGIINPSGGSTSYAQKFQGRVTMTRDT | LAWYQQKPGKAPKLLIYAASSLQSGVPSRFSG |
| | STSTVYMELSSLRSEDTAVYYCASGWVYWGQGTLVTV | SGSGTDFTLTISSLQPEDFATYYCQQSYSTPP |
| | SSASTKGPSVFPLAPSSKSTSGGTAALGCLVKDYFPE | TFGPGTKVDIKRTVAAPSVFIFPPSDEQLKSG |
| | PVTVSWNSGALTSGVHTFPAVLQSSGLYSLSSVVTVP | TASVVCLLNNFYPREAKVQWKVDNALQSGNSQ |
| | SSSLGTQTYICNVNHKPSNTKVDKKVEPKSCDKTHTC | ESVTEQDSKDSTYSLSSTLTLSKADYEKHKVY |
| | PPCPAPEAAGAPSVFLFPPKPKDTLMISRTPEVTCVV | ACEVTHQGLSSPVTKSFNRGEC (SEQ ID |
| | VDVSHEDPEVKFNWYVDGVEVHNAKTKPREEQYNSTY | NO: 349) |
| | RVVSVLTVLHQDWLNGKEYKCKVSNKALPAPIEKTIS | |
| | KAKGQPREPQVYTLPPSREEMTKNQVSLTCLVKGFYP | |
| | SDIAVEWESNGQPENNYKTTPPVLDSDGSFFLYSKLT | |
| | VDKSRWQQGNVFSCSVMHEALHNHYTQKSLSLSPGGG | |
| | GGSGGGGSGGGGSGGGGSQVQLQESGPGPVKPSGTLS | |
| | LTCGVSGGSISSNHWWTWVRQPPGKGLEWIGEIYHNG | |
| | STFLNPSLKSRVTISVDKSNNQFSLKLTSVTAADTAV | |
| | YYCARGWHRTGFRGYPSHWYFDLWGRGTLVSVSSAST | |
| | KGPSVFPLAPSSKSTSGGTAALGCLVKDYFPEPVTVS | |
| | WNSGALTSGVHTFPAVLQSSGLYSLSSVVTVPSSSLG | |
| | TQTYICNVNHKPSNTKVDKKVEPKSCDKTGGSSGSGS | |
| | GSTGTSSSGTGTSAGTTGTSASTSGSGSGGGGGSGGG | |
| | GSAGGTATAGASSGSQSVLTQPPSVSGTPGQRVTISC | |
| | SGSSSHIGSNYVYWYQQLPGTAPKILIYSNDQRPAGV | |
| | PDRFSASKSGTSASLAISGLRSEDEADYYCAAWDDGQ | |
| | GGVFGGGTKLTVLGQPKAAPSVTLFPPSSEELQANKA | |
| | TLVCLISDFYPGAVTVAWKADSSPVKAGVETTTPSKQ | |
| | SNNKYAASSYLSLTPEQWKSHRSYSCQVTHEGSTVEK | |
| | TVAPTECS (SEQ ID NO: 348) | |
| PD1DSGAB6 | QVQLVQSGAEVKKPGASVKVSCKASGYSFTTYYMHWV | DIQMTQSPSSLSASVGDRVTITCQASRDIKNY |
| | RQAPGQGLEWMGIINPSGGSTSYAQKFQGRVTMTRDT | LAWYQQKPGKAPKLLIYAASSLQSGVPSRFSG |
| | STSTVYMELSSLRSEDTAVYYCASGWVYWGQGTLVTV | SGSGTDFTLTISSLQPEDFATYYCQQSYSTPP |
| | SSASTKGPSVFPLAPSSKSTSGGTAALGCLVKDYFPE | TFGPGTKVDIKRTVAAPSVFIFPPSDEQLKSG |
| | PVTVSWNSGALTSGVHTFPAVLQSSGLYSLSSVVTVP | TASVVCLLNNFYPREAKVQWKVDNALQSGNSQ |
| | SSSLGTQTYICNVNHKPSNTKVDKKVEPKSCDKTHTC | ESVTEQDSKDSTYSLSSTLTLSKADYEKHKVY |
| | PPCPAPEAAGAPSVFLFPPKPKDTLMISRTPEVTCVV | ACEVTHQGLSSPVTKSFNRGEC (SEQ ID |
| | VDVSHEDPEVKFNWYVDGVEVHNAKTKPREEQYNSTY | NO: 351) |
| | RVVSVLTVLHQDWLNGKEYKCKVSNKALPAPIEKTIS | |
| | KAKGQPREPQVYTLPPSREEMTKNQVSLTCLVKGFYP | |
| | SDIAVEWESNGQPENNYKTTPPVLDSDGSFFLYSKLT | |
| | VDKSRWQQGNVFSCSVMHEALHNHYTQKSLSLSPGGG | |
| | GGSEGGGSEGGGSEGGGSEQVQLQESGPGPVKPSGTL | |
| | SLTCGVSGGSISSNHWWTWVRQPPGKGLEWIGEIYHN | |
| | GSTFLNPSLKSRVTISVDKSNNQFSLKLTSVTAADTA | |
| | VYYCARGWHRTGFRGYPSHWYFDLWGRGTLVSVSSAS | |
| | TKGPSVFPLAPSSKSTSGGTAALGCLVKDYFPEPVTV | |
| | SWNSGALTSGVHTFPAVLQSSGLYSLSSVVTVPSSSL | |
| | GTQTYICNVNHKPSNTKVDKKVEPKSCDKTGGSSGSG | |
| | SGSTGTSSSGTGTSAGTTGTSASTSGSGSGGGGGSGG | |
| | GGSAGGTATAGASSGSQSVLTQPPSVSGTPGQRVTIS | |
| | CSGSSSHIGSNYVYWYQQLPGTAPKILIYSNDQRPAG | |
| | VPDRESASKSGTSASLAISGLRSEDEADYYCAAWDDG | |
| | QGGVFGGGTKLTVLGQPKAAPSVTLFPPSSEELQANK | |
| | ATLVCLISDFYPGAVTVAWKADSSPVKAGVETTTPSK | |
| | QSNNKYAASSYLSLTPEQWKSHRSYSCQVTHEGSTVE | |
| | KTVAPTECS (SEQ ID NO: 350) | |
| PD1DSGAB7 | QVQLVQSGAEVKKPGASVKVSCKASGYSFTTYYMHWV | DIQMTQSPSSLSASVGDRVTITCQASRDIKNY |
| | RQAPGQGLEWMGIINPSGGSTSYAQKFQGRVTMTRDT | LAWYQQKPGKAPKLLIYAASSLQSGVPSRFSG |
| | STSTVYMELSSLRSEDTAVYYCASGWVYWGQGTLVTV | SGSGTDFTLTISSLQPEDFATYYCQQSYSTPP |
| | SSASTKGPSVFPLAPSSKSTSGGTAALGCLVKDYFPE | TFGPGTKVDIKRTVAAPSVFIFPPSDEQLKSG |
| | PVTVSWNSGALTSGVHTFPAVLQSSGLYSLSSVVTVP | TASVVCLLNNFYPREAKVQWKVDNALQSGNSQ |
| | SSSLGTQTYICNVNHKPSNTKVDKKVEPKSCDKTHTC | ESVTEQDSKDSTYSLSSTLTLSKADYEKHKVY |
| | PPCPAPEAAGAPSVFLFPPKPKDTLMISRTPEVTCVV | ACEVTHQGLSSPVTKSFNRGECGGGSEGGGSE |
| | VDVSHEDPEVKFNWYVDGVEVHNAKTKPREEQYNSTY | GGGSEGGGSEQSVLTQPPSVSGTPGQRVTISC |
| | RVVSVLTVLHQDWLNGKEYKCKVSNKALPAPIEKTIS | SGSSSHIGSNYVYWYQQLPGTAPKILIYSNDQ |
| | KAKGQPREPQVYTLPPSREEMTKNQVSLTCLVKGFYP | RPAGVPDRESASKSGTSASLAISGLRSEDEAD |
| | SDIAVEWESNGQPENNYKTTPPVLDSDGSFFLYSKLT | YYCAAWDDGQGGVFGGGTKLTVLGQPKAAPSV |
| | VDKSRWQQGNVFSCSVMHEALHNHYTQKSLSLSPG | TLFPPSSEELQANKATLVCLISDFYPGAVTVA |
| | (SEQ ID NO: 352) | WKADSSPVKAGVETTTPSKQSNNKYAASSYLS |
| | | LTPEQWKSHRSYSCQVTHEGSTVEKTVAPTEC |
| | | SGGSSGSGSGSTGTSSSGTGTSAGTTGTSAST |
| | | SGSGSGGGGGSGGGGSAGGTATAGASSGSQVQ |
| | | LQESGPGPVKPSGTLSLTCGVSGGSISSNHWW |
| | | TWVRQPPGKGLEWIGEIYHNGSTFLNPSLKSR |
| | | VTISVDKSNNQFSLKLTSVTAADTAVYYCARG |
| | | WHRTGFRGYPSHWYFDLWGRGTLVSVSSASTK |
| | | GPSVFPLAPSSKSTSGGTAALGCLVKDYFPEP |
| | | VTVSWNSGALTSGVHTFPAVLQSSGLYSLSSV |
| | | VTVPSSSLGTQTYICNVNHKPSNTKVDKKVEP |
| | | KSCDKT (SEQ ID NO: 353) |
| PD1DSGAB8 | QVQLVQSGAEVKKPGASVKVSCKASGYSFTTYYMHWV | DIQMTQSPSSLSASVGDRVTITCQASRDIKNY |
| | RQAPGQGLEWMGIINPSGGSTSYAQKFQGRVTMTRDT | LAWYQQKPGKAPKLLIYAASSLQSGVPSRFSG |
| | STSTVYMELSSLRSEDTAVYYCASGWVYWGQGTLVTV | SGSGTDFTLTISSLQPEDFATYYCQQSYSTPP |
| | SSASTKGPSVFPLAPSSKSTSGGTAALGCLVKDYFPE | TFGPGTKVDIKRTVAAPSVFIFPPSDEQLKSG |
| | PVTVSWNSGALTSGVHTFPAVLQSSGLYSLSSVVTVP | TASVVCLLNNFYPREAKVQWKVDNALQSGNSQ |
| | SSSLGTQTYICNVNHKPSNTKVDKKVEPKSCDKTHTC | ESVTEQDSKDSTYSLSSTLTLSKADYEKHKVY |
| | PPCPAPEAAGAPSVFLFPPKPKDTLMISRTPEVTCVV | ACEVTHQGLSSPVTKSFNRGECGGGGSGGGGS |
| | VDVSHEDPEVKFNWYVDGVEVHNAKTKPREEQYNSTY | GGGGSGGGGSQSVLTQPPSVSGTPGQRVTISC |
| | RVVSVLTVLHQDWLNGKEYKCKVSNKALPAPIEKTIS | SGSSSHIGSNYVYWYQQLPGTAPKILIYSNDQ |
| | KAKGQPREPQVYTLPPSREEMTKNQVSLTCLVKGFYP | RPAGVPDRFSASKSGTSASLAISGLRSEDEAD |
| | SDIAVEWESNGQPENNYKTTPPVLDSDGSFFLYSKLT | YYCAAWDDGQGGVFGGGTKLTVLGQPKAAPSV |
| | VDKSRWQQGNVFSCSVMHEALHNHYTQKSLSLSPG | TLFPPSSEELQANKATLVCLISDFYPGAVTVA |
| | (SEQ ID NO: 354) | WKADSSPVKAGVETTTPSKQSNNKYAASSYLS |
| | | LTPEQWKSHRSYSCQVTHEGSTVEKTVAPTEC |
| | | SGGSSGSGSGSTGTSSSGTGTSAGTTGTSAST |
| | | SGSGSGGGGGSGGGGSAGGTATAGASSGSQVQ |
| | | LQESGPGPVKPSGTLSLTCGVSGGSISSNHWW |
| | | TWVRQPPGKGLEWIGEIYHNGSTFLNPSLKSR |
| | | VTISVDKSNNQFSLKLTSVTAADTAVYYCARG |
| | | WHRTGFRGYPSHWYFDLWGRGTLVSVSSASTK |
| | | GPSVFPLAPSSKSTSGGTAALGCLVKDYFPEP |
| | | VTVSWNSGALTSGVHTFPAVLQSSGLYSLSSV |
| | | VTVPSSSLGTQTYICNVNHKPSNTKVDKKVEP |
| | | KSCDKT (SEQ ID NO: 355) |
| PD1DSGAB9 | EVQLLESGGGLVQPGGSLRLSCAASGFTFSNSDMSWV | DIVMTQSPDSLAVSLGERATINCKSSQSVLYS |
| | RQAPGKGLEWVSGITISGGSTYYADSVKGRFTISRDN | PNNKNYLAWYQQKPGQPPKLLIYWASTRESGV |
| | SKNTLYLQMNSLRAEDTAVYYCARGRGGSGWLDYWGQ | PDRFSGSGSGTDFTLTISSLQAEDVAVYYCQQ |
| | GTLVTVSSASTKGPSVFPLAPSSKSTSGGTAALGCLV | YYTTPPTFGQGTRLEIKRTVAAPSVFIFPPSD |
| | KDYFPEPVTVSWNSGALTSGVHTFPAVLQSSGLYSLS | EQLKSGTASVVCLLNNFYPREAKVQWKVDNAL |
| | SVVTVPSSSLGTQTYICNVNHKPSNTKVDKKVEPKSC | QSGNSQESVTEQDSKDSTYSLSSTLTLSKADY |
| | DKTHTCPPCPAPEAAGAPSVFLFPPKPKDTLMISRTP | EKHKVYACEVTHQGLSSPVTKSFNRGEC |
| | EVTCVVVDVSHEDPEVKFNWYVDGVEVHNAKTKPREE | (SEQ ID NO: 357) |
| | QYNSTYRVVSVLTVLHQDWLNGKEYKCKVSNKALPAP | |
| | IEKTISKAKGQPREPQVYTLPPSREEMTKNQVSLTCL | |
| | VKGFYPSDIAVEWESNGQPENNYKTTPPVLDSDGSFF | |
| | LYSKLTVDKSRWQQGNVFSCSVMHEALHNHYTQKSLS | |
| | LSPGGGGGSGGGGSGGGGSQSVLTQPPSVSGTPGQRV | |
| | TISCSGSSSHIGSNYVYWYQQLPGTAPKILIYSNDQR | |
| | PAGVPDRFSASKSGTSASLAISGLRSEDEADYYCAAW | |
| | DDGQGGVFGGGTKLTVLGGGGSGGGGSGGGGSGGGGS | |
| | QVQLQESGPGPVKPSGTLSLTCGVSGGSISSNHWWTW | |
| | VRQPPGKGLEWIGEIYHNGSTFLNPSLKSRVTISVDK | |
| | SNNQFSLKLTSVTAADTAVYYCARGWHRTGFRGYPSH | |
| | WYFDLWGRGTLVSVSS (SEQ ID NO: 356) | |

In some embodiments, the PD-1 antibody comprises a sequence as shown in PD-1 Antibody Table. In some embodiments, the antibody is in a scFV format as illustrated in the PD-1 Antibody Table. In some embodiments, the antibody comprises a CDR1 from any one of clones of the PD-1 Antibody Table, a CDR2 from any one of clones of the PD-1 Antibody Table, and a CDR3 from any one of clones of the PD-1 Antibody Table. In some embodiments, the antibody comprises a LCDR1 from any one of clones of the PD-1 Antibody Table, a LCDR2 from any one of clones of the PD-1 Antibody Table, and a LCDR3 from any one of clones of the PD-1 Antibody Table. In some embodiments, the amino acid residues of the CDRs shown above contain mutations. In some embodiments, the CDRs contain 1, 2, 3, 4, 5, 6, 7, 8, 9, or 10 substitutions or mutations. In some embodiments, the substitution is a conservative substitution.

In some embodiments, the PD-1 antibody has a VH region selected from any one of clones of the PD-1 Antibody Table and a VL region selected from any one of clones as set forth in the PD-1 Antibody Table.

In some embodiments, as provided for herein, the PD-1 antibody, or binding fragment thereof, is linked directly or indirectly to an anti-desmoglein 1 antibody, an anti-desmoglein 2 antibody, an anti-desmoglein 3 antibody, or an anti-desmoglein 4 antibody or binding fragment thereof. Examples of the anti-desmoglein 1 antibody, anti-desmoglein 2 antibody, anti-desmoglein 3 antibody, or anti-desmoglein 4 antibody are provided herein, but these are non-limiting examples and they can linked to other antibodies as well.

In some embodiments, as provided for herein, the anti-desmoglein 1 antibody, anti-desmoglein 2 antibody, anti-desmoglein 3 antibody, or anti-desmoglein 4 antibody, or binding fragment thereof, is linked directly or indirectly to a IL-2 mutein or binding fragment thereof. The IL-2 mutein can be any mutein as provided for herein or other IL-2 muteins known to one of skill in the art. In some embodiments, as provided herein, the anti-desmoglein 1 antibody, anti-desmoglein 2 antibody, anti-desmoglein 3 antibody, or anti-desmoglein 4 antibody, or binding fragment thereof, is linked directly or indirectly to a PD-1 antibody, such as those described herein.

In some embodiments, as provided herein, the PD-1 antibody, or binding fragment thereof, is linked directly or indirectly to an anti-desmoglein 1 antibody, an anti-desmoglein 2 antibody, an anti-desmoglein 3 antibody, or an anti-desmoglein 4 antibody, such as those described herein.

In some embodiments, the PD-1 antibody comprises a sequence as shown in PD-1 Antibody Table 1. In some embodiments, the antibody is in a scFV format. In some embodiments, the antibody comprises a VH sequence from any one of clones of PD-1 Antibody Table 1. In some embodiments, the antibody comprises a VK sequence from any one of clones of the PD-1 Antibody Table 1. In some embodiments, the amino acid residues of the VH or VK shown above contain mutations. In some embodiments, the VH or VK contain 1, 2, 3, 4, 5, 6, 7, 8, 9, or 10 substitutions or mutations. In some embodiments, the substitution is a conservative substitution.

In some embodiments, as provided for herein, the anti-desmoglein 1 antibody, anti-desmoglein 2 antibody, anti-desmoglein 3 antibody, or anti-desmoglein 4 antibody, or binding fragment thereof, is linked directly or indirectly to a CD39 molecule.

In some embodiments, the anti-desmoglein antibody linked to CD39 molecule has a sequence as set forth in DSG-CD39 Table.

DSG-CD39 Table 1

| Ab# | Heavy chain Seq | Light chain Seq |
|---|---|---|
| CD39DSGAB1 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPSREEMT | NO: 359) |
| | KNQVSLTCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGGSTQNKPLPENVKYGIVLDAGSSHTNLYIYKWP | |
| | AEKENDTGVVQQLEECQVKGPGISKYAQKTDEIGAYLAECM | |
| | ELSTELIPTSKHHQTPVYLGATAGMRLLRMESEQSADEVLA | |
| | AVSTSLKGYPFDFQGAKIITGQEEGAYGWITINYLLGRFTQ | |
| | EQSWLSLISDSQKQETFGALDLGGASTQITFVPQNSTIESP | |
| | ENSLQFRLYGEDYTVYTHSFLCYGKDQALWQKLAKDIQVSS | |
| | GGVLKDPCFNPGYEKVVNVSELYGTPCTERFEKKLPFDQFR | |
| | IQGTGDYEQCHQSILELFNNSHCPYSQCAFNGVFLPPLHGS | |
| | FGAFSAFYFVMDFFKKVAKNSVISQEKMTEITKNFCSKSWE | |
| | ETKTSYPSVKEKYLSEYCFSGAYILSLLQGYNFTDSSWEQI | |
| | HFMGKIKDSNAGWTLGYMLNLTNMIPAEQPLSPPLPHSTYI | |
| | (SEQ ID NO: 358) | |
| CD39DSGAB2 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPSREEMT | NO: 361) |
| | KNQVSLTCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGGSGGGGSTQNKPLPENVKYGIVLDAGSSHTNLY | |
| | IYKWPAEKENDTGVVQQLEECQVKGPGISKYAQKTDEIGAY | |
| | LAECMELSTELIPTSKHHQTPVYLGATAGMRLLRMESEQSA | |
| | DEVLAAVSTSLKGYPFDFQGAKIITGQEEGAYGWITINYLL | |
| | GRFTQEQSWLSLISDSQKQETFGALDLGGASTQITFVPQNS | |
| | TIESPENSLQFRLYGEDYTVYTHSFLCYGKDQALWQKLAKD | |
| | IQVSSGGVLKDPCFNPGYEKVVNVSELYGTPCTERFEKKLP | |
| | FDQFRIQGTGDYEQCHQSILELFNNSHCPYSQCAFNGVFLP | |
| | PLHGSFGAFSAFYFVMDFFKKVAKNSVISQEKMTEITKNFC | |
| | SKSWEETKTSYPSVKEKYLSEYCFSGAYILSLLQGYNFTDS | |
| | SWEQIHFMGKIKDSNAGWTLGYMLNLTNMIPAEQPLSPPLP | |
| | HSTYI (SEQ ID NO: 360) | |
| CD39DSGAB3 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPSREEMT | NO: 363) |
| | KNQVSLTCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGGSGGGGSGGGGSTQNKPLPENVKYGIVLDAGSS | |
| | HTNLYIYKWPAEKENDTGVVQQLEECQVKGPGISKYAQKTD | |
| | EIGAYLAECMELSTELIPTSKHHQTPVYLGATAGMRLLRME | |
| | SEQSADEVLAAVSTSLKGYPFDFQGAKIITGQEEGAYGWIT | |
| | INYLLGRFTQEQSWLSLISDSQKQETFGALDLGGASTQITF | |
| | VPQNSTIESPENSLQFRLYGEDYTVYTHSFLCYGKDQALWQ | |
| | KLAKDIQVSSGGVLKDPCFNPGYEKVVNVSELYGTPCTERF | |
| | EKKLPFDQFRIQGTGDYEQCHQSILELFNNSHCPYSQCAFN | |
| | GVFLPPLHGSFGAFSAFYFVMDFFKKVAKNSVISQEKMTEI | |
| | TKNFCSKSWEETKTSYPSVKEKYLSEYCFSGAYILSLLQGY | |
| | NFTDSSWEQIHFMGKIKDSNAGWTLGYMLNLTNMIPAEQPL | |
| | SPPLPHSTYI (SEQ ID NO: 362) | |
| CD39DSGAB4 | QVQLVQSGAEVRKPGASVRVSCKASGYTLTTYDINWVRQAT | DIQMTQSPSSLSASVGDRVTITCRASQS |
| | GQGLEWMGWMNPTSGNTAYAQKFQGRVTMTRNTSISTAYME | ISSYLNWYQQKPGKAPKLLIYAASSLQS |
| | LSSLRSEDTAVYYCARGLFFGVVTKPNYYYYAMDVWGQGTT | GVPSRFSGSGSGTDFTLTISSLQPEDFA |
| | VTVSSASTKGPSVFPLAPSSKSTSGGTAALGCLVKDYFPEP | TYYCQQSYSTPRTFGGGTKLEIKRTVAA |
| | VTVSWNSGALTSGVHTFPAVLQSSGLYSLSSVVTVPSSSLG | PSVFIFPPSDEQLKSGTASVVCLLNNFY |
| | TQTYICNVNHKPSNTKVDKKVEPKSCDKTHTCPPCPAPEAA | PREAKVQWKVDNALQSGNSQESVTEQDS |
| | GAPSVFLFPPKPKDTLMISRTPEVTCVVVDVSHEDPEVKFN | KDSTYSLSSTLTLSKADYEKHKVYACEV |
| | WYVDGVEVHNAKTKPREEQYNSTYRVVSVLTVLHQDWLNGK | THQGLSSPVTKSFNRGEC (SEQ ID |
| | EYKCKVSNKALPAPIEKTISKAKGQPREPQVYTLPPSREEM | NO: 365) |
| | TKNQVSLTCLVKGFYPSDIAVEWESNGQPENNYKTTPPVLD | |
| | SDGSFFLYSKLTVDKSRWQQGNVFSCSVMHEALHNHYTQKS | |
| | LSLSPGGGGGSTQNKPLPENVKYGIVLDAGSSHTNLYIYKW | |
| | PAEKENDTGVVQQLEECQVKGPGISKYAQKTDEIGAYLAEC | |
| | MELSTELIPTSKHHQTPVYLGATAGMRLLRMESEQSADEVL | |
| | AAVSTSLKGYPFDFQGAKIITGQEEGAYGWITINYLLGRFT | |
| | QEQSWLSLISDSQKQETFGALDLGGASTQITFVPQNSTIES | |
| | PENSLQFRLYGEDYTVYTHSFLCYGKDQALWQKLAKDIQVS | |
| | SGGVLKDPCFNPGYEKVVNVSELYGTPCTERFEKKLPFDQF | |
| | RIQGTGDYEQCHQSILELFNNSHCPYSQCAFNGVFLPPLHG | |
| | SFGAFSAFYFVMDFFKKVAKNSVISQEKMTEITKNFCSKSW | |
| | EETKTSYPSVKEKYLSEYCFSGAYILSLLQGYNFTDSSWEQ | |
| | IHFMGKIKDSNAGWTLGYMLNLTNMIPAEQPLSPPLPHSTY | |
| | I (SEQ ID NO: 364) | |
| CD39DSGAB5 | QVQLVQSGAEVRKPGASVRVSCKASGYTLTTYDINWVRQAT | DIQMTQSPSSLSASVGDRVTITCRASQS |
| | GQGLEWMGWMNPTSGNTAYAQKFQGRVTMTRNTSISTAYME | ISSYLNWYQQKPGKAPKLLIYAASSLQS |
| | LSSLRSEDTAVYYCARGLFFGVVTKPNYYYYAMDVWGQGTT | GVPSRFSGSGSGTDFTLTISSLQPEDFA |
| | VTVSSASTKGPSVFPLAPSSKSTSGGTAALGCLVKDYFPEP | TYYCQQSYSTPRTFGGGTKLEIKRTVAA |
| | VTVSWNSGALTSGVHTFPAVLQSSGLYSLSSVVTVPSSSLG | PSVFIFPPSDEQLKSGTASVVCLLNNFY |
| | TQTYICNVNHKPSNTKVDKKVEPKSCDKTHTCPPCPAPEAA | PREAKVQWKVDNALQSGNSQESVTEQDS |
| | GAPSVFLFPPKPKDTLMISRTPEVTCVVVDVSHEDPEVKFN | KDSTYSLSSTLTLSKADYEKHKVYACEV |
| | WYVDGVEVHNAKTKPREEQYNSTYRVVSVLTVLHQDWLNGK | THQGLSSPVTKSFNRGEC (SEQ ID |
| | EYKCKVSNKALPAPIEKTISKAKGQPREPQVYTLPPSREEM | NO: 367) |
| | TKNQVSLTCLVKGFYPSDIAVEWESNGQPENNYKTTPPVLD | |
| | SDGSFFLYSKLTVDKSRWQQGNVFSCSVMHEALHNHYTQKS | |
| | LSLSPGGGGGSGGGGSTQNKPLPENVKYGIVLDAGSSHTNL | |
| | YIYKWPAEKENDTGVVQQLEECQVKGPGISKYAQKTDEIGA | |
| | YLAECMELSTELIPTSKHHQTPVYLGATAGMRLLRMESEQS | |
| | ADEVLAAVSTSLKGYPFDFQGAKIITGQEEGAYGWITINYL | |
| | LGRFTQEQSWLSLISDSQKQETFGALDLGGASTQITFVPQN | |
| | STIESPENSLQFRLYGEDYTVYTHSFLCYGKDQALWQKLAK | |
| | DIQVSSGGVLKDPCFNPGYEKVVNVSELYGTPCTERFEKKL | |
| | PFDQFRIQGTGDYEQCHQSILELFNNSHCPYSQCAFNGVFL | |
| | PPLHGSFGAFSAFYFVMDFFKKVAKNSVISQEKMTEITKNF | |
| | CSKSWEETKTSYPSVKEKYLSEYCFSGAYILSLLQGYNFTD | |
| | SSWEQIHFMGKIKDSNAGWTLGYMLNLTNMIPAEQPLSPPL | |
| | PHSTYI (SEQ ID NO: 366) | |
| CD39DSGAB6 | QVQLVQSGAEVRKPGASVRVSCKASGYTLTTYDINWVRQAT | DIQMTQSPSSLSASVGDRVTITCRASQS |
| | GQGLEWMGWMNPTSGNTAYAQKFQGRVTMTRNTSISTAYME | ISSYLNWYQQKPGKAPKLLIYAASSLQS |
| | LSSLRSEDTAVYYCARGLFFGVVTKPNYYYYAMDVWGQGTT | GVPSRFSGSGSGTDFTLTISSLQPEDFA |
| | VTVSSASTKGPSVFPLAPSSKSTSGGTAALGCLVKDYFPEP | TYYCQQSYSTPRTFGGGTKLEIKRTVAA |
| | VTVSWNSGALTSGVHTFPAVLQSSGLYSLSSVVTVPSSSLG | PSVFIFPPSDEQLKSGTASVVCLLNNFY |
| | TQTYICNVNHKPSNTKVDKKVEPKSCDKTHTCPPCPAPEAA | PREAKVQWKVDNALQSGNSQESVTEQDS |
| | GAPSVFLFPPKPKDTLMISRTPEVTCVVVDVSHEDPEVKFN | KDSTYSLSSTLTLSKADYEKHKVYACEV |
| | WYVDGVEVHNAKTKPREEQYNSTYRVVSVLTVLHQDWLNGK | THQGLSSPVTKSFNRGEC (SEQ ID |
| | EYKCKVSNKALPAPIEKTISKAKGQPREPQVYTLPPSREEM | NO: 369) |
| | TKNQVSLTCLVKGFYPSDIAVEWESNGQPENNYKTTPPVLD | |
| | SDGSFFLYSKLTVDKSRWQQGNVFSCSVMHEALHNHYTQKS | |
| | LSLSPGGGGGSGGGGSGGGGSTQNKPLPENVKYGIVLDAGS | |
| | SHTNLYIYKWPAEKENDTGVVQQLEECQVKGPGISKYAQKT | |
| | DEIGAYLAECMELSTELIPTSKHHQTPVYLGATAGMRLLRM | |
| | ESEQSADEVLAAVSTSLKGYPFDFQGAKIITGQEEGAYGWI | |
| | TINYLLGRFTQEQSWLSLISDSQKQETFGALDLGGASTQIT | |
| | FVPQNSTIESPENSLQFRLYGEDYTVYTHSFLCYGKDQALW | |
| | QKLAKDIQVSSGGVLKDPCFNPGYEKVVNVSELYGTPCTER | |
| | FEKKLPFDQFRIOGTGDYEQCHQSILELFNNSHCPYSQCAF | |
| | NGVFLPPLHGSFGAFSAFYFVMDFFKKVAKNSVISQEKMTE | |
| | ITKNFCSKSWEETKTSYPSVKEKYLSEYCFSGAYILSLLQG | |
| | YNFTDSSWEQIHFMGKIKDSNAGWTLGYMLNLTNMIPAEQP | |
| | LSPPLPHSTYI (SEQ ID NO: 368) | |
| CD39DSGAB7 | EVQLVESGGGLVQPGRSLRLSCAASGFTFDDYAMHWVRQAP | ELQMTQSPSSLSASVGDRVTITCQASQD |
| | GKGLEWVSGISWNSGSIDYADSVKGRFTISRDNAKNSLYLQ | IGNYLNWYQQKPGKAPKLLIYDASYLET |
| | MNSLRVEDTALYYCAKDGSRVFGVGGGFDFWGQGTMVTVSS | GVPSRFSGSGSGTDFTFTISSLQPEDIA |
| | ASTKGPSVFPLAPSSKSTSGGTAALGCLVKDYFPEPVTVSW | TYYCQQYDNLPFTFGPGTKVDIKRTVAA |
| | NSGALTSGVHTFPAVLQSSGLYSLSSVVTVPSSSLGTQTYI | PSVFIFPPSDEQLKSGTASVVCLLNNFY |
| | CNVNHKPSNTKVDKKVEPKSCDKTHTCPPCPAPEAAGAPSV | PREAKVQWKVDNALQSGNSQESVTEQDS |
| | FLFPPKPKDTLMISRTPEVTCVVVDVSHEDPEVKFNWYVDG | KDSTYSLSSTLTLSKADYEKHKVYACEV |
| | VEVHNAKTKPREEQYNSTYRVVSVLTVLHQDWLNGKEYKCK | THQGLSSPVTKSFNRGEC (SEQ ID |
| | VSNKALPAPIEKTISKAKGQPREPQVYTLPPSREEMTKNQV | NO: 371) |
| | SLTCLVKGFYPSDIAVEWESNGQPENNYKTTPPVLDSDGSF | |
| | FLYSKLTVDKSRWQQGNVFSCSVMHEALHNHYTQKSLSLSP | |
| | GGGGGSTQNKPLPENVKYGIVLDAGSSHTNLYIYKWPAEKE | |
| | NDTGVVQQLEECQVKGPGISKYAQKTDEIGAYLAECMELST | |
| | ELIPTSKHHQTPVYLGATAGMRLLRMESEQSADEVLAAVST | |
| | SLKGYPFDFQGAKIITGQEEGAYGWITINYLLGRFTQEQSW | |
| | LSLISDSQKQETFGALDLGGASTQITFVPQNSTIESPENSL | |
| | QFRLYGEDYTVYTHSFLCYGKDQALWQKLAKDIQVSSGGVL | |
| | KDPCFNPGYEKVVNVSELYGTPCTERFEKKLPFDQFRIQGT | |
| | GDYEQCHQSILELFNNSHCPYSQCAFNGVFLPPLHGSFGAF | |
| | SAFYFVMDFFKKVAKNSVISQEKMTEITKNFCSKSWEETKT | |
| | SYPSVKEKYLSEYCFSGAYILSLLQGYNFTDSSWEQIHFMG | |
| | KIKDSNAGWTLGYMLNLTNMIPAEQPLSPPLPHSTYI | |
| | (SEQ ID NO: 370) | |
| CD39DSGAB8 | EVQLVESGGGLVQPGRSLRLSCAASGFTFDDYAMHWVRQAP | ELQMTQSPSSLSASVGDRVTITCQASQD |
| | GKGLEWVSGISWNSGSIDYADSVKGRFTISRDNAKNSLYLQ | IGNYLNWYQQKPGKAPKLLIYDASYLET |
| | MNSLRVEDTALYYCAKDGSRVFGVGGGFDFWGQGTMVTVSS | GVPSRFSGSGSGTDFTFTISSLQPEDIA |
| | ASTKGPSVFPLAPSSKSTSGGTAALGCLVKDYFPEPVTVSW | TYYCQQYDNLPFTFGPGTKVDIKRTVAA |
| | NSGALTSGVHTFPAVLQSSGLYSLSSVVTVPSSSLGTQTYI | PSVFIFPPSDEQLKSGTASVVCLLNNFY |
| | CNVNHKPSNTKVDKKVEPKSCDKTHTCPPCPAPEAAGAPSV | PREAKVQWKVDNALQSGNSQESVTEQDS |
| | FLFPPKPKDTLMISRTPEVTCVVVDVSHEDPEVKFNWYVDG | KDSTYSLSSTLTLSKADYEKHKVYACEV |
| | VEVHNAKTKPREEQYNSTYRVVSVLTVLHQDWLNGKEYKCK | THQGLSSPVTKSFNRGEC (SEQ ID |
| | VSNKALPAPIEKTISKAKGQPREPQVYTLPPSREEMTKNQV | NO: 373) |
| | SLTCLVKGFYPSDIAVEWESNGQPENNYKTTPPVLDSDGSF | |
| | FLYSKLTVDKSRWQQGNVFSCSVMHEALHNHYTQKSLSLSP | |
| | GGGGGSGGGGSTQNKPLPENVKYGIVLDAGSSHTNLYIYKW | |
| | PAEKENDTGVVQQLEECQVKGPGISKYAQKTDEIGAYLAEC | |
| | MELSTELIPTSKHHQTPVYLGATAGMRLLRMESEQSADEVL | |
| | AAVSTSLKGYPFDFQGAKIITGQEEGAYGWITINYLLGRFT | |
| | QEQSWLSLISDSQKQETFGALDLGGASTQITFVPQNSTIES | |
| | PENSLQFRLYGEDYTVYTHSFLCYGKDQALWQKLAKDIQVS | |
| | SGGVLKDPCFNPGYEKVVNVSELYGTPCTERFEKKLPFDQF | |
| | RIQGTGDYEQCHQSILELFNNSHCPYSQCAFNGVFLPPLHG | |
| | SFGAFSAFYFVMDFFKKVAKNSVISQEKMTEITKNFCSKSW | |
| | EETKTSYPSVKEKYLSEYCFSGAYILSLLQGYNFTDSSWEQ | |
| | IHFMGKIKDSNAGWTLGYMLNLTNMIPAEQPLSPPLPHSTY | |
| | I (SEQ ID NO: 372) | |
| CD39DSGAB9 | EVQLVESGGGLVQPGRSLRLSCAASGFTFDDYAMHWVRQAP | ELQMTQSPSSLSASVGDRVTITCQASQD |
| | GKGLEWVSGISWNSGSIDYADSVKGRFTISRDNAKNSLYLQ | IGNYLNWYQQKPGKAPKLLIYDASYLET |
| | MNSLRVEDTALYYCAKDGSRVFGVGGGFDFWGQGTMVTVSS | GVPSRFSGSGSGTDFTFTISSLQPEDIA |
| | ASTKGPSVFPLAPSSKSTSGGTAALGCLVKDYFPEPVTVSW | TYYCQQYDNLPFTFGPGTKVDIKRTVAA |
| | NSGALTSGVHTFPAVLQSSGLYSLSSVVTVPSSSLGTQTYI | PSVFIFPPSDEQLKSGTASVVCLLNNFY |
| | CNVNHKPSNTKVDKKVEPKSCDKTHTCPPCPAPEAAGAPSV | PREAKVQWKVDNALQSGNSQESVTEQDS |
| | FLFPPKPKDTLMISRTPEVTCVVVDVSHEDPEVKFNWYVDG | KDSTYSLSSTLTLSKADYEKHKVYACEV |
| | VEVHNAKTKPREEQYNSTYRVVSVLTVLHQDWLNGKEYKCK | THQGLSSPVTKSFNRGEC (SEQ ID |
| | VSNKALPAPIEKTISKAKGQPREPQVYTLPPSREEMTKNQV | NO: 375) |
| | SLTCLVKGFYPSDIAVEWESNGQPENNYKTTPPVLDSDGSF | |
| | FLYSKLTVDKSRWQQGNVFSCSVMHEALHNHYTQKSLSLSP | |
| | GGGGGSGGGGSGGGGSTQNKPLPENVKYGIVLDAGSSHTNL | |
| | YIYKWPAEKENDTGVVQQLEECQVKGPGISKYAQKTDEIGA | |
| | YLAECMELSTELIPTSKHHQTPVYLGATAGMRLLRMESEQS | |
| | ADEVLAAVSTSLKGYPFDFQGAKIITGQEEGAYGWITINYL | |
| | LGRFTQEQSWLSLISDSQKQETFGALDLGGASTQITFVPQN | |
| | STIESPENSLQFRLYGEDYTVYTHSFLCYGKDQALWQKLAK | |
| | DIQVSSGGVLKDPCFNPGYEKVVNVSELYGTPCTERFEKKL | |
| | PFDQFRIQGTGDYEQCHQSILELFNNSHCPYSQCAFNGVFL | |
| | PPLHGSFGAFSAFYFVMDFFKKVAKNSVISQEKMTEITKNF | |
| | CSKSWEETKTSYPSVKEKYLSEYCFSGAYILSLLQGYNFTD | |
| | SSWEQIHFMGKIKDSNAGWTLGYMLNLTNMIPAEQPLSPPL | |
| | PHSTYI (SEQ ID NO: 374) | |
| CD39DSGAB10 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPSREEMT | NO: 377) |
| | KNQVSLTCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGGSGGGSGGGGSTQNKALPENVKYGIVLDAGSSH | |
| | TSLYIYKWPAEKENDTGVVHQVEECRVKGPGISKFVQKVNE | |
| | IGIYLTDCMERAREVIPRSQHQETPVYLGATAGMRLLRMES | |
| | EELADRVLDVVERSLSNYPFDFQGARIITGQEEGAYGWITI | |
| | NYLLGKFSQKTRWFSIVPYETNNQETFGALDLGGASTQVTF | |
| | VPQNQTIESPDNALQFRLYGKDYNVYTHSFLCYGKDQALWQ | |
| | KLAKDIQVASNEILRDPCFHPGYKKVVNVSDLYKTPCTKRF | |
| | EMTLPFQQFEIQGIGNYQQCHQSILELFNTSYCPYSQCAFN | |
| | GIFLPPLQGDFGAFSAFYFVMKFLNLTSEKVSQEKVTEMMK | |
| | KFCAQPWEEIKTSYAGVKEKYLSEYCFSGTYILSLLLQGYH | |
| | FTADSWEHIHFIGKIQGSDAGWTLGYMLNLTNMIPAEQPLS | |
| | TPLSHSTYV (SEQ ID NO: 376) | |
| CD39DSGAB11 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPSREEMT | NO: 379) |
| | KNQVSLTCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGGSGGGSGGGGSNVKYGIVLDAGSSHTSLYIYKW | |
| | PAEKENDTGVVHQVECRVKGPGISKFVQKVNEIGIYLTDCM | |
| | ERAREVIPRSQHQETPVYLGATAGMRLLRMESEELADRVLD | |
| | VVERSLSNYPFDFQGARIITGQEEGAYGWITINYLLGKFSQ | |
| | KTRWFSIVPYETNNQETFGALDLGGASTQVTFVPQNQTIHS | |
| | FLCYGKDQALWQKLAKDIQVASNEILRDPCFHPGYKKVVNV | |
| | SDLYKTPCTKRFEMTLPFQQFEIQGIGNYQQCHQSILELEN | |
| | TSYCPYSQCAFNGIFLPPLQGDFGAFSAFYFVMKFLNLTSE | |
| | KVSQEKVTEMMKKFCAQPWEEIKTSYAGVKEKYLSEYCFSG | |
| | TYILSLLLQGYHFTADSWEHIHFIGKIQGSDAGWTLGYMLN | |
| | LTNMIPAEQPLSTPLSHSTYV (SEQ ID NO: 378) | |
| CD39DSGAB12 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPSREEMT | NO: 381) |
| | KNQVSLTCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGSGGGGSGGGGSNVKYGIVLDAGSSHTSLYIYKW | |
| | PAEKENDTGVVHQVECRVKGPGISKFVQKVNEIGIYLTDCM | |
| | ERAREVIPRSQHQETPVYLGATAGMRLLRMESEELADRVLD | |
| | VVERSLSNYPFDFQGARIITGQEAGAYGWITINYLLGKFSQ | |
| | KTRWFSIVPYETNNQETFGALDLGGASTQVTFVPQNQTIHS | |
| | FLCYGKDQALWQKLAKDIQVASNEILRDPCFHPGYKKVVNV | |
| | SDLYKTPCTKRFEMTLPFQQFEIQGIGNYQQCHQSILELFN | |
| | TSYCPYSQCAFNGIFLPPLQGDFGAFSAFYFVMKFLNLTSE | |
| | KVSQEKVTEMMKKFCAQPWEEIKTSYAGVKEKYLSEYCFSG | |
| | TYILSLLLQGYHFTADSWEHIHFIGKIQGSDAGWTLGYMLN | |
| | LTNMIPAEQPLSTPLSHSTYV (SEQ ID NO: 380) | |
| CD39DSGAB13 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPSREEMT | NO: 383) |
| | KNQVSLTCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGSKGGGSKGGGSKNVKYGIVLDAGSSHTSLYIYK | |
| | WPAEKENDTGVVHQVECRVKGPGISKFVQKVNEIGIYLTDC | |
| | MERAREVIPRSQHQETPVYLGATAGMRLLRMESEELADRVL | |
| | DVVERSLSNYPFDFQGARIITGQEEGAYGWITINYLLGKFS | |
| | QKTRWFSIVPYETNNQETFGALDLGGASTQVTFVPQNQTIH | |
| | SFLCYGKDQALWQKLAKDIQVASNEILRDPCFHPGYKKVVN | |
| | VSDLYKTPCTKRFEMTLPFQQFEIQGIGNYQQCHQSILELF | |
| | NTSYCPYSQCAFNGIFLPPLQGDFGAFSAFYFVMKFLNLTS | |
| | EKVSQEKVTEMMKKFCAQPWEEIKTSYAGVKEKYLSEYCFS | |
| | GTYILSLLLQGYHFTADSWEHIHFIGKIQGSDAGWTLGYML | |
| | NLTNMIPAEQPLSTPLSHSTYV (SEQ ID NO: 382) | |
| CD39DSGAB14 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPSREEMT | NO: 385) |
| | KNQVSLTCLVKGFYPSDIAVEWESNGQPENNYKTTPPVLDS | |
| | DGSFFLYSKLTVDKSRWQQGNVFSCSVMHEALHNHYTQKSL | |
| | SLSPGGSAGKGSAGKGSAGKNVKYGIVLDAGSSHTSLYIYK | |
| | WPAEKENDTGVVHQVECRVKGPGISKFVQKVNEIGIYLTDC | |
| | MERAREVIPRSQHQETPVYLGATAGMRLLRMESEELADRVL | |
| | DVVERSLSNYPFDFQGARIITGQEEGAYGWITINYLLGKFS | |
| | QKTRWFSIVPYETNNQETFGALDLGGASTQVTFVPQNQTIH | |
| | SFLCYGKDQALWQKLAKDIQVASNEILRDPCFHPGYKKVVN | |
| | VSDLYKTPCTKRFEMTLPFQQFEIQGIGNYQQCHQSILELF | |
| | NTSYCPYSQCAFNGIFLPPLQGDFGAFSAFYFVMKELNLTS | |
| | EKVSQEKVTEMMKKFCAQPWEEIKTSYAGVKEKYLSEYCFS | |
| | GTYILSLLLQGYHFTADSWEHIHFIGKIQGSDAGWTLGYML | |
| | NLTNMIPAEQPLSTPLSHSTYV (SEQ ID NO: 384) | |
| CD39DSGAB15 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPSREEMT | NO: 387) |
| | KNQVSLTCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGSKGGGSKGGGSKTQNKALPENVKYGIVLDAGSS | |
| | HTSLYIYKWPAEKENDTGVVHQVEECRVKGPGISKFVQKVN | |
| | EIGIYLTDCMERAREVIPRSQHQETPVYLGATAGMRLLRME | |
| | SEELADRVLDVVERSLSNYPFDFQGARIITGQEEGAYGWIT | |
| | INYLLGKFSQKTRWFSIVPYETNNQETFGALDLGGASTQVT | |
| | FVPQNQTIESPDNALQFRLYGKDYNVYTHSFLCYGKDQALW | |
| | QKLAKDIQVASNEILRDPCFHPGYKKVVNVSDLYKTPCTKR | |
| | FEMTLPFQQFEIQGIGNYQQCHQSILELFNTSYCPYSQCAF | |
| | NGIFLPPLQGDFGAFSAFYFVMKFLNLTSEKVSQEKVTEMM | |
| | KKFCAQPWEEIKTSYAGVKEKYLSEYCFSGTYILSLLLQGY | |
| | HFTADSWEHIHFIGKIQGSDAGWTLGYMLNLTNMIPAEQPL | |
| | STPLSHSTYV (SEQ ID NO: 386) | |
| CD39DSGAB16 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPSREEMT | NO: 389) |
| | KNQVSLTCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGSKGGGSKGGGSKTQNKALPENVKYGIVLDAGSS | |
| | HTSLYIYKWPAEKENDTGVVHQVEECRVKGPGISKFVQKVN | |
| | EIGIYLTDCMERAREVIPRSQHQETPVYLGATAGMRLLRME | |
| | SEELADRVLDVVERSLSNYPFDFQGARIITGQEEGAYGWIT | |
| | INYLLGKFSQKTRWFSIVPYETNNQETFGALDLGGASTQVT | |
| | FVPQNQTIESPDNALQFRLYGKDYNVYTHSFLCYGKDQALW | |
| | QKLAKDIQVASNEILRDPCFHPGYKKVVNVSDLYKTPCTKR | |
| | FEMTLPFQQFEIQGIGNYQQCHQSILELFNTSYCPYSQCAF | |
| | NGIFLPPLQGDFGAFSAFYFVMKFLNLTSEKVSQEKVTEMM | |
| | KKFCAQPWEEIKTSYAGVKEKYLSEYCFSGTYILSLLLQGY | |
| | HFTADSWEHIHFIGKIQGSDAGWTLGYMLNLTNMIPAEQPL | |
| | STPLSHSTYV (SEQ ID NO: 388) | |
| CD39DSGAB17 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVELFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPSREEMT | NO: 391) |
| | KNQVSLTCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGSKGGGSKGGGSKTQNKALPENVKYGIVLDAGSS | |
| | HTSLYIYKWPAEKENDTGVVHQVEECRVKGPGISKFVQKVN | |
| | EIGIYLTDCMERAREVIPRSQHQETPVYLGATAGMRLLRME | |
| | SEELADRVLDVVERSLSNYPFDFQGARIITGQEEGAYGWIT | |
| | INYLLGKFSQKTRWFSIVPYETNNQETFGALDLGGASTQVT | |
| | FVPQNQTIESPDNALQFRLYGKDYNVYTHSFLCYGKDQALW | |
| | QKLAKDIQVASNEILRDPCFHPGYKKVVNVSDLYKTPCTKR | |
| | FEMTLPFQQFEIQGIGNYQQCHQSILELFNTSYCPYSQCAF | |
| | NGIFLPPLQGDFGAFSAFYFVMKFLNLTSEKVSQEKVTEMM | |
| | KKFCAQPWEEIKTSYAGVKEKYLSEYCFSGTYILSLLLQGY | |
| | HFTADSWEHIHFIGKIQGSDAGWTLGYMLNLTNMIPAEQPL | |
| | STPLSHSTYV (SEQ ID NO: 390) | |
| CD39DSGAB18 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPSREEMT | NO: 393) |
| | KNQVSLTCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGSKGGGSKGGGSKTRDVREPPALKYGIVLDAGSS | |
| | HTSMFIYKWPADKENDTGIVGQHSSCDVPGGGISSYADNPS | |
| | GASQSLVGCLEQALQDVPKERHAGTPLYLGATAGMRLLNLT | |
| | NPEASTSVLMAVTHTLTQYPFDFRGARILSGQEEGVFGWVT | |
| | ANYLLENFIKYGWVGRWFRPRKGTLGAMDLGGASTQITFET | |
| | TSPAEDRASEVQLHLYGQHYRVYTHSFLCYGRDQVLQRLLA | |
| | SALQTHGFHPCWPRGFSTQVLLGDVYQSPCTMAQRPQNFNS | |
| | SARVSLSGSSDPHLCRDLVSGLFSFSSCPFSRCSFNGVFQP | |
| | PVAGNFVAFSAFFYTVDFLRTSMGLPVATLQQLEAAAVNVC | |
| | NQTWAQLQARVPGQRARLADYCAGAMFVQQLLSRGYGFDER | |
| | AFGGVIFQKKAADTAVGWALGYMLNLTNLIPADPPGLRKGT | |
| | DFS (SEQ ID NO: 392) | |
| CD39DSGAB19 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPSREEMT | NO: 395) |
| | KNQVSLTCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGSKGGGSKGGGSKQIHKQEVLPPGLKYGIVLDAG | |
| | SSRTTVYVYQWPAEKENNTGVVSQTFKCSVKGSGISSYGNN | |
| | PQDVPRAFEECMQKVKGQVPSHLHGSTPIHLGATAGMRLLR | |
| | LQNETAANEVLESIQSYFKSQPFDFRGAQIISGQEEGVYGW | |
| | ITANYLMGNFLEKNLWHMWVHPHGVETTGALDLGGASTQIS | |
| | FVAGEKMDLNTSDIMQVSLYGYVYTLYTHSFQCYGRNEAEK | |
| | KFLAMLLQNSPTKNHLTNPCYPRDYSISFTMGHVFDSLCTV | |
| | DQRPESYNPNDVITFEGTGDPSLCKEKVASIFDFKACHDQE | |
| | TCSFDGVYQPKIKGPFVAFAGFYYTASALNLSGSFSLDTFN | |
| | SSTWNFCSQNWSQLPLLLPKFDEVYARSYCFSANYIYHLFV | |
| | NGYKFTEETWPQIHFEKEVGNSSIAWSLGYMLSLTNQIPAE | |
| | SPLIRLPIEPP (SEQ ID NO: 394) | |
| CD39DSGAB20 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPSREEMT | NO: 397) |
| | KNQVSLTCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGSKGGGSKGGGSKKVEATSVLLPTDIKFGIVFDA | |
| | GSSHTSLFLYQWLANKENGTGVVSQALACQVEGPGISSYTS | |
| | NAAQAGESLQGCLEEALVLIPEAQHRKTPTFLGATAGMRLL | |
| | SRKNSSQARDIFAAVTQVLGRSPVDFWGAELLAGQAEGAFG | |
| | WITVNYGLGTLVKYSFTGEWIQPPEEMLVGALDMGGASTQI | |
| | TFVPGGPILDKSTQADFRLYGSDYSVYTHSYLCFGRDQMLS | |
| | RLLVGLVQSRPAALLRHPCYLSGYQTTLALGPLYESPCVHA | |
| | TPPLSLPQNLTVEGTGNPGACVSAIRELFNFSSCQGQEDCA | |
| | FDGVYQPPLRGQFYAFSNFYYTFHFLNLTSRQPLSTVNATI | |
| | WEFCQRPWKLVEASYPGQDRWLRDYCASGLYILTLLHEGYG | |
| | FSEETWPSLEFRKQAGGVDIGWTLGYMLNLTGMIPADAPAQ | |
| | WRAESYGVWVAK (SEQ ID NO: 396) | |
| CD39DSGAB21 | EVQLQESGPGPVKPSGTLSLTCGVSGGSISSNHWWTWVRQP | QSVLTQPPSVSGTPGQRVTISCSGSSSH |
| | PGKGLEWIGEIYHSGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPSREEMT | NO: 399) |
| | KNQVSLTCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGSKGGGSKGGGSKTQNKALPENVKYGIVLDAGSS | |
| | HTSLYIYKWPAEKENDTGVVHQVEECRVKGPGISKFVQKVN | |
| | EIGIYLTDCMERAREVIPRSQHQETPVYLGATAGMRLLRME | |
| | SEELADRVLDVVERSLSNYPFDFQGARIITGQEEGAYGWIT | |
| | INYLLGKFSQKTRWFSIVPYETNNQETFGALDLGGASTQVT | |
| | FVPQNQTIESPDNALQFRLYGKDYNVYTHSFLCYGKDQALW | |
| | QKLAKDIQVASNEILRDPCFHPGYKKVVNVSDLYKTPCTKR | |
| | FEMTLPFQQFEIQGIGNYQQCHQSILELFNTSYCPYSQCAF | |
| | NGIFLPPLQGDFGAFSAFYFVMKFLNLTSEKVSQEKVTEMM | |
| | KKFCAQPWEEIKTSYAGVKEKYLSEYCFSGTYILSLLLQGY | |
| | HFTADSWEHIHFIGKIQGSDAGWTLGYMLNLTNMIPAEQPL | |
| | STPLSHSTYV (SEQ ID NO: 398) | |
| CD39DSGAB | QVQLQESGPGPVKPSGTLSLTCGVSGGSISSNHWWTWVRQP | QSVLTQPPSVSGTPGQRVTISCSGSSSH |
| 22 | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECSGSAGKNVKY |
| | YKCKVSNKALPAPIEKTISKAKGQPREPQVYTLPPSREEMT | GIVLDAGSSHTSLYIYKWPAEKENDTGV |
| | KNQVSLTCLVKGFYPSDIAVEWESNGQPENNYKTTPPVLDS | VHQVECRVKGPGISKFVQKVNEIGIYLT |
| | DGSFFLYSKLTVDKSRWQQGNVFSCSVMHEALHNHYTQKSL | DCMERAREVIPRSQHQETPVYLGATAGM |
| | SLSPG (SEQ ID NO: 400) | RLLRMESEELADRVLDVVERSLSNYPFD |
| | | FQGARIITGQEEGAYGWITINYLLGKFS |
| | | QKTRWFSIVPYETNNQETFGALDLGGAS |
| | | TQVTFVPQNQTIHSFLCYGKDQALWQKL |
| | | AKDIQVASNEILRDPCFHPGYKKVVNVS |
| | | DLYKTPCTKRFEMTLPFQQFEIQGIGNY |
| | | QQCHQSILELFNTSYCPYSQCAFNGIFL |
| | | PPLQGDFGAFSAFYFVMKFLNLTSEKVS |
| | | QEKVTEMMKKFCAQPWEEIKTSYAGVKE |
| | | KYLSEYCFSGTYILSLLLQGYHFTADSW |
| | | EHIHFIGKIQGSDAGWTLGYMLNLTNMI |
| | | PAEQPLSTPLSHSTYV (SEQ ID NO: |
| | | 401) |
| CD39DSGAB23 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECSGSNVKYGIV |
| | YKCKVSNKALPAPIEKTISKAKGQPREPQVYTLPPSREEMT | LDAGSSHTSLYIYKWPAEKENDTGVVHQ |
| | KNQVSLTCLVKGFYPSDIAVEWESNGQPENNYKTTPPVLDS | VECRVKGPGISKFVQKVNEIGIYLTDCM |
| | DGSFFLYSKLTVDKSRWQQGNVFSCSVMHEALHNHYTQKSL | ERAREVIPRSQHQETPVYLGATAGMRLL |
| | SLSPG (SEQ ID NO: 402) | RMESEELADRVLDVVERSLSNYPFDFQG |
| | | ARIITGQEEGAYGWITINYLLGKFSQKT |
| | | RWFSIVPYETNNQETFGALDLGGASTQV |
| | | TFVPQNQTIHSFLCYGKDQALWQKLAKD |
| | | IQVASNEILRDPCFHPGYKKVVNVSDLY |
| | | KTPCTKRFEMTLPFQQFEIQGIGNYQQC |
| | | HQSILELFNTSYCPYSQCAFNGIFLPPL |
| | | QGDFGAFSAFYFVMKFLNLTSEKVSQEK |
| | | VTEMMKKFCAQPWEEIKTSYAGVKEKYL |
| | | SEYCFSGTYILSLLLQGYHFTADSWEHI |
| | | HFIGKIQGSDAGWTLGYMLNLTNMIPAE |
| | | QPLSTPLSHSTYV (SEQ ID NO: |
| | | 403) |
| CD39DSGAB24 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECSGSAGKGSAG |
| | YKCKVSNKALPAPIEKTISKAKGQPREPQVYTLPPSREEMT | KGSAGKNVKYGIVLDAGSSHTSLYIYKW |
| | KNQVSLTCLVKGFYPSDIAVEWESNGQPENNYKTTPPVLDS | PAEKENDTGVVHQVECRVKGPGISKFVQ |
| | DGSFFLYSKLTVDKSRWQQGNVFSCSVMHEALHNHYTQKSL | KVNEIGIYLTDCMERAREVIPRSQHQET |
| | SLSPG (SEQ ID NO: 404) | PVYLGATAGMRLLRMESEELADRVLDVV |
| | | ERSLSNYPFDFQGARIITGQEEGAYGWI |
| | | TINYLLGKFSQKTRWFSIVPYETNNQET |
| | | FGALDLGGASTQVTFVPQNQTIHSFLCY |
| | | GKDQALWQKLAKDIQVASNEILRDPCFH |
| | | PGYKKVVNVSDLYKTPCTKRFEMTLPFQ |
| | | QFEIQGIGNYQQCHQSILELFNTSYCPY |
| | | SQCAFNGIFLPPLQGDFGAFSAFYFVMK |
| | | FLNLTSEKVSQEKVTEMMKKFCAQPWEE |
| | | IKTSYAGVKEKYLSEYCFSGTYILSLLL |
| | | QGYHFTADSWEHIHFIGKIQGSDAGWTL |
| | | GYMLNLTNMIPAEQPLSTPLSHSTYV |
| | | (SEQ ID NO: 405) |
| CD39DSGAB25 | QVQLQESGPGPVKPSGTLSLTCGVSGGSISSNHWWTWVRQP | QSVLTQPPSVSGTPGQRVTISCSGSSSH |
| | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCCVECPPCPAPPAAAGA | DFYPGAVTVAWKADSSPVKAGVETTTPS |
| | TQNKALPENVKYGIVLDAGSSHTSLYIYKWPAEKENDTGVV | KQSNNKYAASSYLSLTPEQWKSHRSYSC |
| | HQVEECRVKGPGISKFVQKVNEIGIYLTDCMERAREVIPRS | QVTHEGSTVEKTVAPTECS (SEQ ID |
| | QHQETPVYLGATAGMRLLRMESEELADRVLDVVERSLSNYP | NO: 407) |
| | FDFQGARIITGQEEGAYGWITINYLLGKFSQKTRWFSIVPY | |
| | ETNNQETFGALDLGGASTQVTFVPQNQTIESPDNALQFRLY | |
| | GKDYNVYTHSFLCYGKDQALWQKLAKDIQVASNEILRDPCF | |
| | HPGYKKVVNVSDLYKTPCTKRFEMTLPFQQFEIQGIGNYQQ | |
| | CHQSILELFNTSYCPYSQCAFNGIFLPPLQGDFGAFSAFYF | |
| | VMKFLNLTSEKVSQEKVTEMMKKFCAQPWEEIKTSYAGVKE | |
| | KYLSEYCFSGTYILSLLLQGYHFTADSWEHIHFIGKIQGSD | |
| | AGWTLGYMLNLTNMIPAEQPLSTPLSHSTYV (SEQ ID | |
| | NO: 406) | |
| CD39DSGAB26 | TQNKALPENVKYGIVLDAGSSHTSLYIYKWPAEKENDTGVV | QSVLTQPPSVSGTPGQRVTISCSGSSSH |
| | HQVEECRVKGPGISKFVQKVNEIGIYLTDCMERAREVIPRS | IGSNYVYWYQQLPGTAPKILIYSNDQRP |
| | QHQETPVYLGATAGMRLLRMESEELADRVLDVVERSLSNYP | AGVPDRFSASKSGTSASLAISGLRSEDE |
| | FDFQGARIITGQEEGAYGWITINYLLGKFSQKTRWFSIVPY | ADYYCAAWDDGQGGVFGGGTKLTVLGQP |
| | ETNNQETFGALDLGGASTQVTFVPQNQTIESPDNALQFRLY | KAAPSVTLFPPSSEELQANKATLVCLIS |
| | GKDYNVYTHSFLCYGKDQALWQKLAKDIQVASNEILRDPCF | DFYPGAVTVAWKADSSPVKAGVETTTPS |
| | HPGYKKVVNVSDLYKTPCTKRFEMTLPFQQFEIQGIGNYQQ | KQSNNKYAASSYLSLTPEQWKSHRSYSC |
| | CHQSILELFNTSYCPYSQCAFNGIFLPPLQGDFGAFSAFYF | QVTHEGSTVEKTVAPTECS (SEQ ID |
| | VMKFLNLTSEKVSQEKVTEMMKKFCAQPWEEIKTSYAGVKE | NO: 409) |
| | KYLSEYCFSGTYILSLLLQGYHFTADSWEHIHFIGKIQGSD | |
| | AGWTLGYMLNLTNMIPAEQPLSTPLSHSTYVGGGSKGGGSK | |
| | GGGSKQVQLQESGPGPVKPSGTLSLTCGVSGGSISSNHWWT | |
| | WVRQPPGKGLEWIGEIYHNGSTFLNPSLKSRVTISVDKSNN | |
| | QFSLKLTSVTAADTAVYYCARGWHRTGFRGYPSHWYFDLWG | |
| | RGTLVSVSSASTKGPSVFPLAPSSKSTSGGTAALGCLVKDY | |
| | FPEPVTVSWNSGALTSGVHTFPAVLQSSGLYSLSSVVTVPS | |
| | SSLGTQTYICNVNHKPSNTKVDKKVEPKSCDKTHTCPPCPA | |
| | PEAAGAPSVFLFPPKPKDTLMISRTPEVTCVVVDVSHEDPE | |
| | VKFNWYVDGVEVHNAKTKPREEQYNSTYRVVSVLTVLHQDW | |
| | LNGKEYKCKVSNKALPAPIEKTISKAKGQPREPQVYTLPPS | |
| | REEMTKNQVSLTCLVKGFYPSDIAVEWESNGQPENNYKTTP | |
| | PVLDSDGSFFLYSKLTVDKSRWQQGNVFSCSVMHEALHNHY | |
| | TQKSLSLSPG (SEQ ID NO: 408) | |
| CD39DSGAB27 | TQNKALPENVKYGIVLDAGSSHTSLYIYKWPAEKENDTGVV | QSVLTQPPSVSGTPGQRVTISCSGSSSH |
| | HQVEECRVKGPGISKFVQKVNEIGIYLTDCMERAREVIPRS | IGSNYVYWYQQLPGTAPKILIYSNDQRP |
| | QHQETPVYLGATAGMRLLRMESEELADRVLDVVERSLSNYP | AGVPDRFSASKSGTSASLAISGLRSEDE |
| | FDFQGARIITGQEEGAYGWITINYLLGKFSQKTRWFSIVPY | ADYYCAAWDDGQGGVFGGGTKLTVLGQP |
| | ETNNQETFGALDLGGASTQVTFVPQNQTIESPDNALQFRLY | KAAPSVTLFPPSSEELQANKATLVCLIS |
| | GKDYNVYTHSFLCYGKDQALWQKLAKDIQVASNEILRDPCF | DFYPGAVTVAWKADSSPVKAGVETTTPS |
| | HPGYKKVVNVSDLYKTPCTKRFEMTLPFQQFEIQGIGNYQQ | KQSNNKYAASSYLSLTPEQWKSHRSYSC |
| | CHQSILELFNTSYCPYSQCAFNGIFLPPLQGDFGAFSAFYF | QVTHEGSTVEKTVAPTECS (SEQ ID |
| | VMKFLNLTSEKVSQEKVTEMMKKFCAQPWEEIKTSYAGVKE | NO: 411) |
| | KYLSEYCFSGTYILSLLLQGYHFTADSWEHIHFIGKIQGSD | |
| | AGWTLGYMLNLTNMIPAEQPLSTPLSHSTYVGSQVQLQESG | |
| | PGPVKPSGTLSLTCGVSGGSISSNHWWTWVRQPPGKGLEWI | |
| | GEIYHNGSTFLNPSLKSRVTISVDKSNNQFSLKLTSVTAAD | |
| | TAVYYCARGWHRTGFRGYPSHWYFDLWGRGTLVSVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVN | |
| | HKPSNTKVDKKVEPKSCDKTHTCPPCPAPEAAGAPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSNK | |
| | ALPAPIEKTISKAKGQPREPQVYTLPPSREEMTKNQVSLTC | |
| | LVKGFYPSDIAVEWESNGQPENNYKTTPPVLDSDGSFFLYS | |
| | KLTVDKSRWQQGNVFSCSVMHEALHNHYTQKSLSLSPG | |
| | (SEQ ID NO: 410) | |
| CD39DSGAB28 | TQNKALPENVKYGIVLDAGSSHTSLYIYKWPAEKENDTGVV | QSVLTQPPSVSGTPGQRVTISCSGSSSH |
| | HQVEECRVKGPGISKFVQKVNEIGIYLTDCMERAREVIPRS | IGSNYVYWYQQLPGTAPKILIYSNDQRP |
| | QHQETPVYLGATAGMRLLRMESEELADRVLDVVERSLSNYP | AGVPDRFSASKSGTSASLAISGLRSEDE |
| | FDFQGARIITGQEEGAYGWITINYLLGKFSQKTRWFSIVPY | ADYYCAAWDDGQGGVFGGGTKLTVLGQP |
| | ETNNQETFGALDLGGASTQVTFVPQNQTIESPDNALQFRLY | KAAPSVTLFPPSSEELQANKATLVCLIS |
| | GKDYNVYTHSFLCYGKDQALWQKLAKDIQVASNEILRDPCF | DFYPGAVTVAWKADSSPVKAGVETTTPS |
| | HPGYKKVVNVSDLYKTPCTKRFEMTLPFQQFEIQGIGNYQQ | KQSNNKYAASSYLSLTPEQWKSHRSYSC |
| | CHQSILELFNTSYCPYSQCAFNGIFLPPLQGDFGAFSAFYF | QVTHEGSTVEKTVAPTECS (SEQ ID |
| | VMKFLNLTSEKVSQEKVTEMMKKFCAQPWEEIKTSYAGVKE | NO: 413) |
| | KYLSEYCFSGTYILSLLLQGYHFTADSWEHIHFIGKIQGSD | |
| | AGWTLGYMLNLTNMIPAEQPLSTPLSHSTYVGGGSKQVQLQ | |
| | ESGPGPVKPSGTLSLTCGVSGGSISSNHWWTWVRQPPGKGL | |
| | EWIGEIYHNGSTFLNPSLKSRVTISVDKSNNQFSLKLTSVT | |
| | AADTAVYYCARGWHRTGFRGYPSHWYFDLWGRGTLVSVSSA | |
| | STKGPSVFPLAPSSKSTSGGTAALGCLVKDYFPEPVTVSWN | |
| | SGALTSGVHTFPAVLQSSGLYSLSSVVTVPSSSLGTQTYIC | |
| | NVNHKPSNTKVDKKVEPKSCDKTHTCPPCPAPEAAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKCKV | |
| | SNKALPAPIEKTISKAKGQPREPQVYTLPPSREEMTKNQVS | |
| | LTCLVKGFYPSDIAVEWESNGQPENNYKTTPPVLDSDGSFF | |
| | LYSKLTVDKSRWQQGNVFSCSVMHEALHNHYTQKSLSLSPG | |
| | (SEQ ID NO: 412) | |
| CD39DSGAB29 | QVQLQESGPGPVKPSGTLSLTCGVSGGSISSNHWWTWVRQP | NVKYGIVLDAGSSHTSLYIYKWPAEKEN |
| | PGKGLEWIGEIYHNGSTFLNPSLKSRVTISVDKSNNQFSLK | DTGVVHQVECRVKGPGISKFVQKVNEIG |
| | LTSVTAADTAVYYCARGWHRTGFRGYPSHWYFDLWGRGTLV | IYLTDCMERAREVIPRSQHQETPVYLGA |
| | SVSSASTKGPSVFPLAPSSKSTSGGTAALGCLVKDYFPEPV | TAGMRLLRMESEELADRVLDVVERSLSN |
| | TVSWNSGALTSGVHTFPAVLQSSGLYSLSSVVTVPSSSLGT | YPFDFQGARIITGQEEGAYGWITINYLL |
| | QTYICNVNHKPSNTKVDKKVEPKSCDKTHTCPPCPAPEAAG | GKFSQKTRWFSIVPYETNNQETFGALDL |
| | APSVFLFPPKPKDTLMISRTPEVTCVVVDVSHEDPEVKFNW | GGASTQVTFVPQNQTIHSFLCYGKDQAL |
| | YVDGVEVHNAKTKPREEQYNSTYRVVSVLTVLHQDWLNGKE | WQKLAKDIQVASNEILRDPCFHPGYKKV |
| | YKCKVSNKALPAPIEKTISKAKGQPREPQVYTLPPSREEMT | VNVSDLYKTPCTKRFEMTLPFQQFEIQG |
| | KNQVSLTCLVKGFYPSDIAVEWESNGQPENNYKTTPPVLDS | IGNYQQCHQSILELFNTSYCPYSQCAFN |
| | DGSFFLYSKLTVDKSRWQQGNVFSCSVMHEALHNHYTQKSL | GIFLPPLQGDFGAFSAFYFVMKFLNLTS |
| | SLSPG (SEQ ID NO: 414) | EKVSQEKVTEMMKKFCAQPWEEIKTSYA |
| | | GVKEKYLSEYCFSGTYILSLLLQGYHFT |
| | | ADSWEHIHFIGKIQGSDAGWTLGYMLNL |
| | | TNMIPAEQPLSTPLSHSTYVAEEEKQSV |
| | | LTQPPSVSGTPGQRVTISCSGSSSHIGS |
| | | NYVYWYQQLPGTAPKILIYSNDQRPAGV |
| | | PDRFSASKSGTSASLAISGLRSEDEADY |
| | | YCAAWDDGQGGVFGGGTKLTVLGQPKAA |
| | | PSVTLFPPSSEELQANKATLVCLISDFY |
| | | PGAVTVAWKADSSPVKAGVETTTPSKQS |
| | | NNKYAASSYLSLTPEQWKSHRSYSCQVT |
| | | HEGSTVEKTVAPTECS (SEQ ID NO: |
| | | 415) |
| CD39DSGAB30 | QVQLQESGPGPVKPSGTLSLTCGVSGGSISSNHWWTWVRQP | NVKYGIVLDAGSSHTSLYIYKWPAEKEN |
| | PGKGLEWIGEIYHNGSTFLNPSLKSRVTISVDKSNNQFSLK | DTGVVHQVECRVKGPGISKFVQKVNEIG |
| | LTSVTAADTAVYYCARGWHRTGFRGYPSHWYFDLWGRGTLV | IYLTDCMERAREVIPRSQHQETPVYLGA |
| | SVSSASTKGPSVFPLAPSSKSTSGGTAALGCLVKDYFPEPV | TAGMRLLRMESEELADRVLDVVERSLSN |
| | TVSWNSGALTSGVHTFPAVLQSSGLYSLSSVVTVPSSSLGT | YPFDFQGARIITGQEEGAYGWITINYLL |
| | QTYICNVNHKPSNTKVDKKVEPKSCDKTHTCPPCPAPEAAG | GKFSQKTRWFSIVPYETNNQETFGALDL |
| | APSVFLFPPKPKDTLMISRTPEVTCVVVDVSHEDPEVKFNW | GGASTQVTFVPQNQTIHSFLCYGKDQAL |
| | YVDGVEVHNAKTKPREEQYNSTYRVVSVLTVLHQDWLNGKE | WQKLAKDIQVASNEILRDPCFHPGYKKV |
| | YKCKVSNKALPAPIEKTISKAKGQPREPQVYTLPPSREEMT | VNVSDLYKTPCTKRFEMTLPFQQFEIQG |
| | KNQVSLTCLVKGFYPSDIAVEWESNGQPENNYKTTPPVLDS | IGNYQQCHQSILELFNTSYCPYSQCAFN |
| | DGSFFLYSKLTVDKSRWQQGNVFSCSVMHEALHNHYTQKSL | GIFLPPLQGDFGAFSAFYFVMKFLNLTS |
| | SLSPG (SEQ ID NO: 416) | EKVSQEKVTEMMKKFCAQPWEEIKTSYA |
| | | GVKEKYLSEYCFSGTYILSLLLQGYHFT |
| | | ADSWEHIHFIGKIQGSDAGWTLGYMLNL |
| | | TNMIPAEQPLSTPLSHSTYVGSQSVLTQ |
| | | PPSVSGTPGQRVTISCSGSSSHIGSNYV |
| | | YWYQQLPGTAPKILIYSNDQRPAGVPDR |
| | | FSASKSGTSASLAISGLRSEDEADYYCA |
| | | AWDDGQGGVFGGGTKLTVLGQPKAAPSV |
| | | TLFPPSSEELQANKATLVCLISDFYPGA |
| | | VTVAWKADSSPVKAGVETTTPSKQSNNK |
| | | YAASSYLSLTPEQWKSHRSYSCQVTHEG |
| | | STVEKTVAPTECS (SEQ ID NO: |
| | | 417) |
| CD39DSGAB31 | QVQLQESGPGPVKPSGTLSLTCGVSGGSISSNHWWTWVRQP | NVKYGIVLDAGSSHTSLYIYKWPAEKEN |
| | PGKGLEWIGEIYHNGSTFLNPSLKSRVTISVDKSNNQFSLK | DTGVVHQVECRVKGPGISKFVQKVNEIG |
| | LTSVTAADTAVYYCARGWHRTGFRGYPSHWYFDLWGRGTLV | IYLTDCMERAREVIPRSQHQETPVYLGA |
| | SVSSASTKGPSVFPLAPSSKSTSGGTAALGCLVKDYFPEPV | TAGMRLLRMESEELADRVLDVVERSLSN |
| | TVSWNSGALTSGVHTFPAVLQSSGLYSLSSVVTVPSSSLGT | YPFDFQGARIITGQEEGAYGWITINYLL |
| | QTYICNVNHKPSNTKVDKKVEPKSCDKTHTCPPCPAPEAAG | GKFSQKTRWFSIVPYETNNQETFGALDL |
| | APSVFLFPPKPKDTLMISRTPEVTCVVVDVSHEDPEVKFNW | GGASTQVTFVPQNQTIHSFLCYGKDQAL |
| | YVDGVEVHNAKTKPREEQYNSTYRVVSVLTVLHQDWLNGKE | WQKLAKDIQVASNEILRDPCFHPGYKKV |
| | YKCKVSNKALPAPIEKTISKAKGQPREPQVYTLPPSREEMT | VNVSDLYKTPCTKRFEMTLPFQQFEIQG |
| | KNQVSLTCLVKGFYPSDIAVEWESNGQPENNYKTTPPVLDS | IGNYQQCHQSILELFNTSYCPYSQCAFN |
| | DGSFFLYSKLTVDKSRWQQGNVFSCSVMHEALHNHYTQKSL | GIFLPPLQGDFGAFSAFYFVMKFLNLTS |
| | SLSPG (SEQ ID NO: 418) | EKVSQEKVTEMMKKFCAQPWEEIKTSYA |
| | | GVKEKYLSEYCFSGTYILSLLLQGYHFT |
| | | ADSWEHIHFIGKIQGSDAGWTLGYMLNL |
| | | TNMIPAEQPLSTPLSHSTYVGGGGSGGG |
| | | GSGGGGSQSVLTQPPSVSGTPGQRVTIS |
| | | CSGSSSHIGSNYVYWYQQLPGTAPKILI |
| | | YSNDQRPAGVPDRFSASKSGTSASLAIS |
| | | GLRSEDEADYYCAAWDDGQGGVFGGGTK |
| | | LTVLGQPKAAPSVTLFPPSSEELQANKA |
| | | TLVCLISDFYPGAVTVAWKADSSPVKAG |
| | | VETTTPSKQSNNKYAASSYLSLTPEQWK |
| | | SHRSYSCQVTHEGSTVEKTVAPTECS |
| | | (SEQ ID NO: 419) |
| CD39DSGAB32 | QVQLQESGPGPVKPSGTLSLTCGVSGGSISSNHWWTWVRQP | TQNKALPENVKYGIVLDAGSSHTSLYIY |
| | PGKGLEWIGEIYHNGSTFLNPSLKSRVTISVDKSNNQFSLK | KWPAEKENDTGVVHQVEECRVKGPGISK |
| | LTSVTAADTAVYYCARGWHRTGFRGYPSHWYFDLWGRGTLV | FVQKVNEIGIYLTDCMERAREVIPRSQH |
| | SVSSASTKGPSVFPLAPSSKSTSGGTAALGCLVKDYFPEPV | QETPVYLGATAGMRLLRMESEELADRVL |
| | TVSWNSGALTSGVHTFPAVLQSSGLYSLSSVVTVPSSSLGT | DVVERSLSNYPFDFQGARIITGQEEGAY |
| | QTYICNVNHKPSNTKVDKKVEPKSCDKTHTCPPCPAPEAAG | GWITINYLLGKFSQKTRWFSIVPYETNN |
| | APSVFLFPPKPKDTLMISRTPEVTCVVVDVSHEDPEVKFNW | QETFGALDLGGASTQVTFVPQNQTIESP |
| | YVDGVEVHNAKTKPREEQYNSTYRVVSVLTVLHQDWLNGKE | DNALQFRLYGKDYNVYTHSFLCYGKDQA |
| | YKCKVSNKALPAPIEKTISKAKGQPREPQVYTLPPSREEMT | LWQKLAKDIQVASNEILRDPCFHPGYKK |
| | KNQVSLTCLVKGFYPSDIAVEWESNGQPENNYKTTPPVLDS | VVNVSDLYKTPCTKRFEMTLPFQQFEIQ |
| | DGSFFLYSKLTVDKSRWQQGNVFSCSVMHEALHNHYTQKSL | GIGNYQQCHQSILELFNTSYCPYSQCAF |
| | SLSPG (SEQ ID NO: 420) | NGIFLPPLQGDFGAFSAFYFVMKFLNLT |
| | | SEKVSQEKVTEMMKKFCAQPWEEIKTSY |
| | | AGVKEKYLSEYCFSGTYILSLLLQGYHF |
| | | TADSWEHIHFIGKIQGSDAGWTLGYMLN |
| | | LTNMIPAEQPLSTPLSHSTYVGGGSKGG |
| | | GSKGGGSKQSVLTQPPSVSGTPGQRVTI |
| | | SCSGSSSHIGSNYVYWYQQLPGTAPKIL |
| | | IYSNDQRPAGVPDRFSASKSGTSASLAI |
| | | SGLRSEDEADYYCAAWDDGQGGVFGGGT |
| | | KLTVLGQPKAAPSVTLFPPSSEELQANK |
| | | ATLVCLISDFYPGAVTVAWKADSSPVKA |
| | | GVETTTPSKQSNNKYAASSYLSLTPEQW |
| | | KSHRSYSCQVTHEGSTVEKTVAPTECS |
| | | (SEQ ID NO: 421) |
| CD39DSGAB33 | QVQLQESGPGPVKPSGTLSLTCGVSGGSISSNHWWTWVRQP | TQNKALPENVKYGIVLDAGSSHTSLYIY |
| | PGKGLEWIGEIYHNGSTFLNPSLKSRVTISVDKSNNQFSLK | KWPAEKENDTGVVHQVEECRVKGPGISK |
| | LTSVTAADTAVYYCARGWHRTGFRGYPSHWYFDLWGRGTLV | FVQKVNEIGIYLTDCMERAREVIPRSQH |
| | SVSSASTKGPSVFPLAPSSKSTSGGTAALGCLVKDYFPEPV | QETPVYLGATAGMRLLRMESEELADRVL |
| | TVSWNSGALTSGVHTFPAVLQSSGLYSLSSVVTVPSSSLGT | DVVERSLSNYPFDFQGARIITGQEEGAY |
| | QTYICNVNHKPSNTKVDKKVEPKSCDKTHTCPPCPAPEAAG | GWITINYLLGKFSQKTRWFSIVPYETNN |
| | APSVELFPPKPKDTLMISRTPEVTCVVVDVSHEDPEVKFNW | QETFGALDLGGASTQVTFVPQNQTIESP |
| | YVDGVEVHNAKTKPREEQYNSTYRVVSVLTVLHQDWLNGKE | DNALQFRLYGKDYNVYTHSFLCYGKDQA |
| | YKCKVSNKALPAPIEKTISKAKGQPREPQVYTLPPSREEMT | LWQKLAKDIQVASNEILRDPCFHPGYKK |
| | KNQVSLTCLVKGFYPSDIAVEWESNGQPENNYKTTPPVLDS | VVNVSDLYKTPCTKRFEMTLPFQQFEIQ |
| | DGSFFLYSKLTVDKSRWQQGNVFSCSVMHEALHNHYTQKSL | GIGNYQQCHQSILELFNTSYCPYSQCAF |
| | SLSPG (SEQ ID NO: 422) | NGIFLPPLQGDFGAFSAFYFVMKFLNLT |
| | | SEKVSQEKVTEMMKKFCAQPWEEIKTSY |
| | | AGVKEKYLSEYCFSGTYILSLLLQGYHF |
| | | TADSWEHIHFIGKIQGSDAGWTLGYMLN |
| | | LTNMIPAEQPLSTPLSHSTYVGSQSVLT |
| | | QPPSVSGTPGQRVTISCSGSSSHIGSNY |
| | | VYWYQQLPGTAPKILIYSNDQRPAGVPD |
| | | RFSASKSGTSASLAISGLRSEDEADYYC |
| | | AAWDDGQGGVFGGGTKLTVLGQPKAAPS |
| | | VTLFPPSSEELQANKATLVCLISDFYPG |
| | | AVTVAWKADSSPVKAGVETTTPSKQSNN |
| | | KYAASSYLSLTPEQWKSHRSYSCQVTHE |
| | | GSTVEKTVAPTECS (SEQ ID NO: |
| | | 423) |
| CD39DSGAB34 | QVQLQESGPGPVKPSGTLSLTCGVSGGSISSNHWWTWVRQP | TQNKALPENVKYGIVLDAGSSHTSLYIY |
| | PGKGLEWIGEIYHNGSTFLNPSLKSRVTISVDKSNNQFSLK | KWPAEKENDTGVVHQVEECRVKGPGISK |
| | LTSVTAADTAVYYCARGWHRTGFRGYPSHWYFDLWGRGTLV | FVQKVNEIGIYLTDCMERAREVIPRSQH |
| | SVSSASTKGPSVFPLAPSSKSTSGGTAALGCLVKDYFPEPV | QETPVYLGATAGMRLLRMESEELADRVL |
| | TVSWNSGALTSGVHTFPAVLQSSGLYSLSSVVTVPSSSLGT | DVVERSLSNYPFDFQGARIITGQEEGAY |
| | QTYICNVNHKPSNTKVDKKVEPKSCDKTHTCPPCPAPEAAG | GWITINYLLGKFSQKTRWFSIVPYETNN |
| | APSVFLFPPKPKDTLMISRTPEVTCVVVDVSHEDPEVKFNW | QETFGALDLGGASTQVTFVPQNQTIESP |
| | YVDGVEVHNAKTKPREEQYNSTYRVVSVLTVLHQDWLNGKE | DNALQFRLYGKDYNVYTHSFLCYGKDQA |
| | YKCKVSNKALPAPIEKTISKAKGQPREPQVYTLPPSREEMT | LWQKLAKDIQVASNEILRDPCFHPGYKK |
| | KNQVSLTCLVKGFYPSDIAVEWESNGQPENNYKTTPPVLDS | VVNVSDLYKTPCTKRFEMTLPFQQFEIQ |
| | DGSFFLYSKLTVDKSRWQQGNVFSCSVMHEALHNHYTQKSL | GIGNYQQCHQSILELFNTSYCPYSQCAF |
| | SLSPG (SEQ ID NO: 424) | NGIFLPPLQGDFGAFSAFYFVMKFLNLT |
| | | SEKVSQEKVTEMMKKFCAQPWEEIKTSY |
| | | AGVKEKYLSEYCFSGTYILSLLLQGYHF |
| | | TADSWEHIHFIGKIQGSDAGWTLGYMLN |
| | | LTNMIPAEQPLSTPLSHSTYVGGGSKQS |
| | | VLTQPPSVSGTPGQRVTISCSGSSSHIG |
| | | SNYVYWYQQLPGTAPKILIYSNDQRPAG |
| | | VPDRFSASKSGTSASLAISGLRSEDEAD |
| | | YYCAAWDDGQGGVFGGGTKLTVLGQPKA |
| | | APSVTLFPPSSEELQANKATLVCLISDF |
| | | YPGAVTVAWKADSSPVKAGVETTTPSKQ |
| | | SNNKYAASSYLSLTPEQWKSHRSYSCQV |
| | | THEGSTVEKTVAPTECS (SEQ ID |
| | | NO: 425) |
| CD39DSGAB35 | QVQLQESGPGPVKPSGTLSLTCGVSGGSISSNHWWTWVRQP | QSVLTQPPSVSGTPGQRVTISCSGSSSH |
| HC1 | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVCTLPPSREEMT | NO: 427) |
| | KNQVSLWCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGGSGGGGSGGGGSTQNKALPENVKYGIVLDAGSS | |
| | HTSLYIYKWPAEKENDTGVVHQVEECRVKGPGISKFVQKVN | |
| | EIGIYLTDCMERAREVIPRSQHQETPVYLGATAGMRLLRME | |
| | SEELADRVLDVVERSLSNYPFDFQGARIITGQEEGAYGWIT | |
| | INYLLGKFSQKTRWFSIVPYETNNQETFGALDLGGASTQVT | |
| | FVPQNQTIESPDNALQFRLYGKDYNVYTHSFLCYGKDQALW | |
| | QKLAKDIQVASNEILRDPCFHPGYKKVVNVSDLYKTPCTKR | |
| | FEMTLPFQQFEIQGIGNYQQCHQSILELFNTSYCPYSQCAF | |
| | NGIFLPPLQGDFGAFSAFYFVMKFLNLTSEKVSQEKVTEMM | |
| | KKFCAQPWEEIKTSYAGVKEKYLSEYCFSGTYILSLLLQGY | |
| | HFTADSWEHIHFIGKIQGSDAGWTLGYMLNLTNMIPAEQPL | |
| | STPLSHSTYV (SEQ ID NO: 426) | |
| CD39DSGAB35 | QVQLQESGPGPVKPSGTLSLTCGVSGGSISSNHWWTWVRQP | QSVLTQPPSVSGTPGQRVTISCSGSSSH |
| HC2 | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPCREEMT | NO: 427) |
| | KNQVSLSCAVKGFYPSDIAVEWESNGQPENNYKTTPPVLDS | |
| | DGSFFLVSKLTVDKSRWQQGNVFSCSVMHEALHNHYTQKSL | |
| | SLSPG (SEQ ID NO: 428) | |
| CD39DSGAB36 | QVQLQESGPGPVKPSGTLSLTCGVSGGSISSNHWWTWVRQP | QSVLTQPPSVSGTPGQRVTISCSGSSSH |
| HC1 | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPCREEMT | NO: 427) |
| | KNQVSLSCAVKGFYPSDIAVEWESNGQPENNYKTTPPVLDS | |
| | DGSFFLVSKLTVDKSRWQQGNVFSCSVMHEALHNHYTQKSL | |
| | SLSPGGGGSKGGGSKGGGSKTQNKALPENVKYGIVLDAGSS | |
| | HTSLYIYKWPAEKENDTGVVHQVEECRVKGPGISKFVQKVN | |
| | EIGIYLTDCMERAREVIPRSQHQETPVYLGATAGMRLLRME | |
| | SEELADRVLDVVERSLSNYPFDFQGARIITGQEEGAYGWIT | |
| | INYLLGKFSQKTRWFSIVPYETNNQETFGALDLGGASTQVT | |
| | FVPQNQTIESPDNALQFRLYGKDYNVYTHSFLCYGKDQALW | |
| | QKLAKDIQVASNEILRDPCFHPGYKKVVNVSDLYKTPCTKR | |
| | FEMTLPFQQFEIQGIGNYQQCHQSILELFNTSYCPYSQCAF | |
| | NGIFLPPLQGDFGAFSAFYFVMKFLNLTSEKVSQEKVTEMM | |
| | KKFCAQPWEEIKTSYAGVKEKYLSEYCFSGTYILSLLLQGY | |
| | HFTADSWEHIHFIGKIQGSDAGWTLGYMLNLTNMIPAEQPL | |
| | STPLSHSTYV (SEQ ID NO: 429) | |
| CD39DSGAB36 | QVQLQESGPGPVKPSGTLSLTCGVSGGSISSNHWWTWVRQP | QSVLTQPPSVSGTPGQRVTISCSGSSSH |
| HC2 | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVCTLPPSREEMT | NO: 427) |
| | KNQVSLWCLVKGFYPSDIAVEWESNGQPENNYKTTPPVLDS | |
| | DGSFFLYSKLTVDKSRWQQGNVFSCSVMHEALHNHYTQKSL | |
| | SLSPG (SEQ ID NO: 430) | |
| CD39DSGAB37 | QVQLQESGPGPVKPSGTLSLTCGVSGGSISSNHWWTWVRQP | QSVLTQPPSVSGTPGQRVTISCSGSSSH |
| HC1 | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPCREEMT | NO: 427) |
| | KNQVSLWCLVKGFYPSDIAVEWESNGQPENNYKTTPPVLDS | |
| | DGSFFLYSKLTVDKSRWQQGNVFSCSVMHEALHNHYTQKSL | |
| | SLSPGGGGSKGGGSKGGGSKTQNKALPENVKYGIVLDAGSS | |
| | HTSLYIYKWPAEKENDTGVVHQVEECRVKGPGISKFVQKVN | |
| | EIGIYLTDCMERAREVIPRSQHQETPVYLGATAGMRLLRME | |
| | SEELADRVLDVVERSLSNYPFDFQGARIITGQEEGAYGWIT | |
| | INYLLGKFSQKTRWFSIVPYETNNQETFGALDLGGASTQVT | |
| | FVPQNQTIESPDNALQFRLYGKDYNVYTHSFLCYGKDQALW | |
| | QKLAKDIQVASNEILRDPCFHPGYKKVVNVSDLYKTPCTKR | |
| | FEMTLPFQQFEIQGIGNYQQCHQSILELFNTSYCPYSQCAF | |
| | NGIFLPPLQGDFGAFSAFYFVMKFLNLTSEKVSQEKVTEMM | |
| | KKFCAQPWEEIKTSYAGVKEKYLSEYCFSGTYILSLLLQGY | |
| | HFTADSWEHIHFIGKIQGSDAGWTLGYMLNLTNMIPAEQPL | |
| | STPLSHSTYV (SEQ ID NO: 431) | |
| CD39DSGAB37 | QVQLQESGPGPVKPSGTLSLTCGVSGGSISSNHWWTWVRQP | QSVLTQPPSVSGTPGQRVTISCSGSSSH |
| HC2 | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVCTLPPSREEMT | NO: 427) |
| | KNQVSLSCAVKGFYPSDIAVEWESNGQPENNYKTTPPVLDS | |
| | DGSFFLVSKLTVDKSRWQQGNVFSCSVMHEALHNHYTQKSL | |
| | SLSPG (SEQ ID NO: 432) | |
| CD39DSGAB38 | QVQLQESGPGPVKPSGTLSLTCGVSGGSISSNHWWTWVRQP | QSVLTQPPSVSGTPGQRVTISCSGSSSH |
| HC1 | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVFLFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVCTLPPSREEMT | NO: 427) |
| | KNQVSLSCAVKGFYPSDIAVEWESNGQPENNYKTTPPVLDS | |
| | DGSFFLVSKLTVDKSRWQQGNVFSCSVMHEALHNHYTQKSL | |
| | SLSPGGGGSKGGGSKGGGSKTQNKALPENVKYGIVLDAGSS | |
| | HTSLYIYKWPAEKENDTGVVHQVEECRVKGPGISKFVQKVN | |
| | EIGIYLTDCMERAREVIPRSQHQETPVYLGATAGMRLLRME | |
| | SEELADRVLDVVERSLSNYPFDFQGARIITGQEEGAYGWIT | |
| | INYLLGKFSQKTRWFSIVPYETNNQETFGALDLGGASTQVT | |
| | FVPQNQTIESPDNALQFRLYGKDYNVYTHSFLCYGKDQALW | |
| | QKLAKDIQVASNEILRDPCFHPGYKKVVNVSDLYKTPCTKR | |
| | FEMTLPFQQFEIQGIGNYQQCHQSILELFNTSYCPYSQCAF | |
| | NGIFLPPLQGDFGAFSAFYFVMKFLNLTSEKVSQEKVTEMM | |
| | KKFCAQPWEEIKTSYAGVKEKYLSEYCFSGTYILSLLLQGY | |
| | HFTADSWEHIHFIGKIQGSDAGWTLGYMLNLTNMIPAEQPL | |
| | STPLSHSTYV (SEQ ID NO: 433) | |
| CD39DSGAB38 | QVQLQESGPGPVKPSGTLSLTCGVSGGSISSNHWWTWVRQP | QSVLTQPPSVSGTPGQRVTISCSGSSSH |
| HC2 | PGKGLEWIGEIYHNGSTFLNPSLKSRVTISVDKSNNQFSLK | IGSNYVYWYQQLPGTAPKILIYSNDQRP |
| | LTSVTAADTAVYYCARGWHRTGFRGYPSHWYFDLWGRGTLV | AGVPDRFSASKSGTSASLAISGLRSEDE |
| | SVSSASTKGPSVFPLAPSSKSTSGGTAALGCLVKDYFPEPV | ADYYCAAWDDGQGGVFGGGTKLTVLGQP |
| | TVSWNSGALTSGVHTFPAVLQSSGLYSLSSVVTVPSSSLGT | KAAPSVTLFPPSSEELQANKATLVCLIS |
| | QTYICNVNHKPSNTKVDKKVEPKSCDKTHTCPPCPAPEAAG | DFYPGAVTVAWKADSSPVKAGVETTTPS |
| | APSVELFPPKPKDTLMISRTPEVTCVVVDVSHEDPEVKFNW | KQSNNKYAASSYLSLTPEQWKSHRSYSC |
| | YVDGVEVHNAKTKPREEQYNSTYRVVSVLTVLHQDWLNGKE | QVTHEGSTVEKTVAPTECS (SEQ ID |
| | YKCKVSNKALPAPIEKTISKAKGQPREPQVYTLPPCREEMT | NO: 427) |
| | KNQVSLWCLVKGFYPSDIAVEWESNGQPENNYKTTPPVLDS | |
| | DGSFFLYSKLTVDKSRWQQGNVFSCSVMHEALHNHYTQKSL | |
| | SLSPG (SEQ ID NO: 434) | |

In some embodiments, the CD39 molecule is linked to the anti-desmoglein antibody in the N- to C-terminus direction. In some embodiments, the C-terminus of an anti-desmoglein antibody scFv is linked via a G/S linked to the N-terminus of a CD39 molecule, and has the sequence of:

(CD39DSGAB39, SEQ ID NO: 435)
QSVLTQPPSVSGTPGQRVTISCSGSSSHIGSNYVYWYQQLPGTAPKILIY
SNDQRPAGVPDRFSASKSGTSASLAISGLRSEDEADYYCAAWDDGQGGVF
GGGTKLTVLGGGGSGGGGSGGGGSGGGGSQVQLQESGPGPVKPSGTLSLT
CGVSGGSISSNHWWTWVRQPPGKGLEWIGEIYHNGSTFLNPSLKSRVTIS
VDKSNNQFSLKLTSVTAADTAVYYCARGWHRTGFRGYPSHWYFDLWGRGT
LVSVSSGAGGGSKGGGSKGGGSKTQNKALPENVKYGIVLDAGSSHTSLYI
YKWPAEKENDTGVVHQVEECRVKGPGISKFVQKVNEIGIYLTDCMERARE
VIPRSQHQETPVYLGATAGMRLLRMESEELADRVLDVVERSLSNYPFDFQ
GARIITGQEEGAYGWITINYLLGKFSQKTRWFSIVPYETNNQETFGALDL
GGASTQVTFVPQNQTIESPDNALQFRLYGKDYNVYTHSFLCYGKDQALWQ
KLAKDIQVASNEILRDPCFHPGYKKVVNVSDLYKTPCTKRFEMTLPFQQF
EIQGIGNYQQCHQSILELFNTSYCPYSQCAFNGIFLPPLQGDFGAFSAFY
FVMKFLNLTSEKVSQEKVTEMMKKFCAQPWEEIKTSYAGVKEKYLSEYCF
SGTYILSLLLQGYHFTADSWEHIHFIGKIQGSDAGWTLGYMLNLINMIPA
EQPLSTPLSHSTYV;

or

In some embodiments, the CD39 molecule is linked to the anti-desmoglein antibody in the C- to N-terminus direction. In some embodiments, the C-terminus of CD39 molecule is linked via a G/S linker to the N-terminus of an Fc molecule further linked at the N-terminus of the Fc molecule via a G/S linker to the N-terminus of an anti-desmoglein antibody scFv, and has the sequence of:

(CD39DSGAB40, SEQ ID NO: 436)
TQNKALPENVKYGIVLDAGSSHTSLYIYKWPAEKENDTGVVHQVEECRVKG
PGISKFVQKVNEIGIYLTDCMERAREVIPRSQHQETPVYLGATAGMRLLRM
ESEELADRVLDVVERSLSNYPFDFQGARIITGQEEGAYGWITINYLLGKFS
QKTRWFSIVPYETNNQETFGALDLGGASTQVTFVPQNQTIESPDNALQFRL
YGKDYNVYTHSFLCYGKDQALWQKLAKDIQVASNEILRDPCFHPGYKKVVN
VSDLYKTPCTKRFEMTLPFQQFEIQGIGNYQQCHQSILELFNTSYCPYSQC
AFNGIFLPPLQGDFGAFSAFYFVMKFLNLTSEKVSQEKVTEMMKKFCAQPW
EEIKTSYAGVKEKYLSEYCFSGTYILSLLLQGYHFTADSWEHIHFIGKIQG
SDAGWTLGYMLNLTNMIPAEQPLSTPLSHSTYVGGGGSGGGGSGGGGSDKT
HTCPPCPAPEAAGAPSVFLFPPKPKDTLMISRTPEVTCVVVDVSHEDPEVK
FNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCSV
MHEALHNHYTQKSLSLSPGGGGGSGGGGSGGGGSQSVLTQPPSVSGTPGQR
VTISCSGSSSHIGSNYVYWYQQLPGTAPKILIYSNDQRPAGVPDRFSASKS
GTSASLAISGLRSEDEADYYCAAWDDGQGGVFGGGTKLTVLGGGGSGGGGS
GGGGSGGGGSQVQLQESGPGPVKPSGTLSLTCGVSGGSISSNHWWTWVRQP
PGKGLEWIGEIYHNGSTFLNPSLKSRVTISVDKSNNQFSLKLTSVTAADTA
VYYCARGWHRTGFRGYPSHWYFDLWGRGTLVSVSS

In some embodiments, the anti-desmoglein antibody linked to the CD39 molecule comprises a heterodimeric molecule, further comprising a bivalent anti-desmoglein antibody and monovalent human CD39 molecules. In some embodiments, the heterodimeric molecule comprises a heavy chain 1 as set forth in SEQ ID NO: 426, a heavy chain 2 as set forth in SEQ ID NO: 428, and a light chain as set forth in SEQ ID NO: 427. In some embodiments, the heterodimeric molecule comprises a heavy chain 1 as set forth in SEQ ID NO: 429, a heavy chain 2 as set forth in SEQ ID NO: 430, and a light chain as set forth in SEQ ID NO: 427. In some embodiments, the heterodimeric molecule comprises a heavy chain 1 as set forth in SEQ ID NO 431, a heavy chain 2 as set forth in SEQ ID NO: 432, and a light chain as set forth in SEQ ID NO: 427. In some embodiments, the heterodimeric molecule comprises a heavy chain 1 as set forth in SEQ ID NO: 433, a heavy chain 2 as set forth in SEQ ID NO: 434, and a light chain as set forth in SEQ ID NO: 427.

In some embodiments, the anti-desmoglein antibody linked to the CD39 molecule comprises a heterodimeric molecule, further comprising a monovalent anti-desmoglein antibody and monovalent human CD39 molecules. In some embodiments, the heterodimeric molecule comprises a heavy chain 1 as set forth in SEQ ID NO: 437, an Fc as set forth in SEQ ID NO: 438, and a light chain as set forth in SEQ ID NO: 427. In some embodiments, the heterodimeric molecule comprises a heavy chain 1 as set forth in SEQ ID NO: 439, an Fc as set forth in SEQ ID NO: 440, and a light chain as set forth in SEQ ID NO: 427. In some embodiments, the heterodimeric molecule comprises a heavy chain 1 as set forth in SEQ ID NO: 441, an Fc as set forth in SEQ ID NO: 442, and a light chain as set forth in SEQ ID NO: 427. In some embodiments, the heterodimeric molecule comprises a heavy chain 1 as set forth in SEQ ID NO: 443, an Fc as set forth in SEQ ID NO: 444, and a light chain as set forth in SEQ ID NO: 427.

TABLE 2
DSG-CD39

| | Heavy | | Light |
|---|---|---|---|
| Ab# | chain Seq | Fc Seq | chain Seq |
| CD39DSGAB | QVQLQESGP | DKTHTCPPCPAPEA | QSVLTQPPS |
| 41 | GPVKPSGTL | AGAPSVFLFPPKPK | VSGTPGQRV |
| | SLTCGVSGG | DTLMISRTPEVTCV | TISCSGSSS |
| | SISSNHWWT | VVDVSHEDPEVKFN | HIGSNYVYW |
| | WVRQPPGKG | WYVDGVEVHNAKTK | YQQLPGTAP |
| | LEWIGEIYH | PREEQYNSTYRVVS | KILIYSNDQ |
| | NGSTFLNPS | VLTVLHQDWLNGKE | RPAGVPDRF |
| | LKSRVTISV | YKCKVSNKALPAPI | SASKSGTSA |
| | DKSNNQFSL | EKTISKAKGQPREP | SLAISGLRS |
| | KLTSVTAAD | QVYTLPPCREEMTK | EDEADYYCA |
| | TAVYYCARG | NQVSLSCAVKGFYP | AWDDGQGGV |
| | WHRTGFRGY | SDIAVEWESNGQPE | FGGGTKLTV |
| | PSHWYFDLW | NNYKTTPPVLDSDG | LGQPKAAPS |
| | GRGTLVSVS | SFFLVSKLTVDKSR | VTLFPPSSE |
| | SASTKGPSV | WQQGNVFSCSVMHE | ELQANKATL |
| | FPLAPSSKS | ALHNHYTQKSLSLS | VCLISDFYP |
| | TSGGTAALG | PGGGGSKGGGSKGG | GAVTVAWKA |
| | CLVKDYFPE | GSKTQNKALPENVK | DSSPVKAGV |
| | PVTVSWNSG | YGIVLDAGSSHTSL | ETTTPSKQS |
| | ALTSGVHTF | YIYKWPAEKENDTG | NNKYAASSY |
| | PAVLQSSGL | VVHQVEECRVKGPG | LSLTPEQWK |
| | YSLSSVVTV | ISKFVQKVNEIGIY | SHRSYSCQV |
| | PSSSLGTQT | LTDCMERAREVIPR | THEGSTVEK |
| | YICNVNHKP | SQHQETPVYLGATA | TVAPTECS |
| | SNTKVDKKV | GMRLLRMESEELAD | (SEQ ID |
| | EPKSCDKTH | RVLDVVERSLSNYP | NO: 427) |
| | TCPPCPAPE | FDFQGARIITGQEE | |
| | AAGAPSVFL | GAYGWITINYLLGK | |
| | FPPKPKDTL | FSQKTRWFSIVPYE | |
| | MISRTPEVT | TNNQETFGALDLGG | |
| | CVVVDVSHE | ASTQVTFVPQNQTI | |
| | DPEVKFNWY | ESPDNALQFRLYGK | |
| | VDGVEVHNA | DYNVYTHSFLCYGK | |
| | KTKPREEQY | DQALWQKLAKDIQV | |
| | NSTYRVVSV | ASNEILRDPCFHPG | |
| | LTVLHQDWL | YKKVVNVSDLYKTP | |
| | NGKEYKCKV | CTKRFEMTLPFQQF | |
| | SNKALPAPI | EIQGIGNYQQCHQS | |
| | EKTISKAKG | ILELFNTSYCPYSQ | |
| | QPREPQVCT | CAFNGIFLPPLQGD | |
| | LPPSREEMT | FGAFSAFYFVMKFL | |
| | KNQVSLWCL | NLTSEKVSQEKVTE | |
| | VKGFYPSDI | MMKKFCAQPWEEIK | |
| | AVEWESNGQ | TSYAGVKEKYLSEY | |
| | PENNYKTTP | CFSGTYILSLLLQG | |
| | PVLDSDGSF | YHFTADSWEHIHFI | |
| | FLYSKLTVD | GKIQGSDAGWTLGY | |
| | KSRWQQGNV | MLNLTNMIPAEQPL | |
| | FSCSVMHEA | STPLSHSTYV | |
| | LHNHYTQKS | (SEQ ID | |
| | LSLSPG | NO: 438) | |
| | (SEQ ID | | |
| | NO: 437) | | |
| CD39DSGAB | QVQLQESGP | DKTHTCPPCPAPEA | QSVLTQPPS |
| 42 | GPVKPSGTL | AGAPSVFLFPPKPK | VSGTPGQRV |
| | SLTCGVSGG | DTLMISRTPEVTCV | TISCSGSSS |
| | SISSNHWWT | VVDVSHEDPEVKFN | HIGSNYVYW |
| | WVRQPPGKG | WYVDGVEVHNAKTK | YQQLPGTAP |
| | LEWIGEIYH | PREEQYNSTYRVVS | KILIYSNDQ |
| | NGSTFLNPS | VLTVLHQDWLNGKE | RPAGVPDRF |
| | LKSRVTISV | YKCKVSNKALPAPI | SASKSGTSA |
| | DKSNNQFSL | EKTISKAKGQPREP | SLAISGLRS |
| | KLTSVTAAD | QVCTLPPSREEMTK | EDEADYYCA |
| | TAVYYCARG | NQVSLWCLVKGFYP | AWDDGQGGV |
| | WHRTGFRGY | SDIAVEWESNGQPE | FGGGTKLTV |
| | PSHWYFDLW | NNYKTTPPVLDSDG | LGQPKAAPS |
| | GRGTLVSVS | SFFLYSKLTVDKSR | VTLFPPSSE |
| | SASTKGPSV | WQQGNVFSCSVMHE | ELQANKATL |
| | FPLAPSSKS | ALHNHYTQKSLSLS | VCLISDFYP |
| | TSGGTAALG | PGGGGSKGGGSKGG | GAVTVAWKA |
| | CLVKDYFPE | GSKTQNKALPENVK | DSSPVKAGV |
| | PVTVSWNSG | YGIVLDAGSSHTSL | ETTTPSKQS |
| | ALTSGVHTF | YIYKWPAEKENDTG | NNKYAASSY |
| | PAVLQSSGL | VVHQVEECRVKGPG | LSLTPEQWK |
| | YSLSSVVTV | ISKFVQKVNEIGIY | SHRSYSCQV |
| | PSSSLGTQT | LTDCMERAREVIPR | THEGSTVEK |
| | YICNVNHKP | SQHQETPVYLGATA | TVAPTECS |
| | SNTKVDKKV | GMRLLRMESEELAD | (SEQ ID |
| | EPKSCDKTH | RVLDVVERSLSNYP | NO: 427) |
| | TCPPCPAPE | FDFQGARIITGQEE | |
| | AAGAPSVFL | GAYGWITINYLLGK | |
| | FPPKPKDTL | FSQKTRWFSIVPYE | |
| | MISRTPEVT | TNNQETFGALDLGG | |
| | CVVVDVSHE | ASTQVTFVPQNQTI | |
| | DPEVKFNWY | ESPDNALQFRLYGK | |
| | VDGVEVHNA | DYNVYTHSFLCYGK | |
| | KTKPREEQY | DQALWQKLAKDIQV | |
| | NSTYRVVSV | ASNEILRDPCFHPG | |
| | LTVLHQDWL | YKKVVNVSDLYKTP | |
| | NGKEYKCKV | CTKRFEMTLPFQQF | |
| | SNKALPAPI | EIQGIGNYQQCHQS | |
| | EKTISKAKG | ILELFNTSYCPYSQ | |
| | QPREPQVYT | CAFNGIFLPPLQGD | |
| | LPPCREEMT | FGAFSAFYFVMKFL | |
| | KNQVSLSCA | NLTSEKVSQEKVTE | |
| | VKGFYPSDI | MMKKFCAQPWEEIK | |
| | AVEWESNGQ | TSYAGVKEKYLSEY | |
| | PENNYKTTP | CFSGTYILSLLLQG | |
| | PVLDSDGSF | YHFTADSWEHIHFI | |
| | FLVSKLTVD | GKIQGSDAGWTLGY | |
| | KSRWQQGNV | MLNLTNMIPAEQPL | |
| | FSCSVMHEA | STPLSHSTYV | |
| | LHNHYTQKS | (SEQ ID | |
| | LSLSPG | NO: 440) | |
| | (SEQ ID | | |
| | NO: 439) | | |
| CD39DSGAB | QVQLQESGP | DKTHTCPPCPAPEA | QSVLTQPPS |
| 43 | GPVKPSGTL | AGAPSVFLFPPKPK | VSGTPGQRV |
| | SLTCGVSGG | DTLMISRTPEVTCV | TISCSGSSS |
| | SISSNHWWT | VVDVSHEDPEVKFN | HIGSNYVYW |
| | WVRQPPGKG | WYVDGVEVHNAKTK | YQQLPGTAP |
| | LEWIGEIYH | PREEQYNSTYRVVS | KILIYSNDQ |
| | NGSTFLNPS | VLTVLHQDWLNGKE | RPAGVPDRF |
| | LKSRVTISV | YKCKVSNKALPAPI | SASKSGTSA |
| | DKSNNQFSL | EKTISKAKGQPREP | SLAISGLRS |
| | KLTSVTAAD | QVYTLPPCREEMTK | EDEADYYCA |
| | TAVYYCARG | NQVSLWCLVKGFYP | AWDDGQGGV |
| | WHRTGFRGY | SDIAVEWESNGQPE | FGGGTKLTV |
| | PSHWYFDLW | NNYKTTPPVLDSDG | LGQPKAAPS |
| | GRGTLVSVS | SFFLYSKLTVDKSR | VTLFPPSSE |
| | SASTKGPSV | WQQGNVFSCSVMHE | ELQANKATL |
| | FPLAPSSKS | ALHNHYTQKSLSLS | VCLISDFYP |
| | TSGGTAALG | PGGGGSKGGGSKGG | GAVTVAWKA |
| | CLVKDYFPE | GSKTQNKALPENVK | DSSPVKAGV |
| | PVTVSWNSG | YGIVLDAGSSHTSL | ETTTPSKQS |
| | ALTSGVHTF | YIYKWPAEKENDTG | NNKYAASSY |
| | PAVLQSSGL | VVHQVEECRVKGPG | LSLTPEQWK |
| | YSLSSVVTV | ISKFVQKVNEIGIY | SHRSYSCQV |
| | PSSSLGTQT | LTDCMERAREVIPR | THEGSTVEK |
| | YICNVNHKP | SQHQETPVYLGATA | TVAPTECS |
| | SNTKVDKKV | GMRLLRMESEELAD | (SEQ ID |
| | EPKSCDKTH | RVLDVVERSLSNYP | NO: 427) |
| | TCPPCPAPE | FDFQGARIITGQEE | |
| | AAGAPSVFL | GAYGWITINYLLGK | |
| | FPPKPKDTL | FSQKTRWFSIVPYE | |
| | MISRTPEVT | TNNQETFGALDLGG | |
| | CVVVDVSHE | ASTQVTFVPQNQTI | |
| | DPEVKFNWY | ESPDNALQFRLYGK | |
| | VDGVEVHNA | DYNVYTHSFLCYGK | |
| | KTKPREEQY | DQALWQKLAKDIQV | |
| | NSTYRVVSV | ASNEILRDPCFHPG | |
| | LTVLHQDWL | YKKVVNVSDLYKTP | |
| | NGKEYKCKV | CTKRFEMTLPFQQF | |
| | SNKALPAPI | EIQGIGNYQQCHQS | |
| | EKTISKAKG | ILELFNTSYCPYSQ | |
| | QPREPQVCT | CAFNGIFLPPLQGD | |
| | LPPSREEMT | FGAFSAFYFVMKFL | |
| | KNQVSLSCA | NLTSEKVSQEKVTE | |
| | VKGFYPSDI | MMKKFCAQPWEEIK | |
| | AVEWESNGQ | TSYAGVKEKYLSEY | |
| | PENNYKTTP | CFSGTYILSLLLQG | |
| | PVLDSDGSF | YHFTADSWEHIHFI | |
| | FLVSKLTVD | GKIQGSDAGWTLGY | |
| | KSRWQQGNV | MLNLTNMIPAEQPL | |
| | FSCSVMHEA | STPLSHSTYV | |
| | LHNHYTQKS | (SEQ ID | |
| | LSLSPG | NO: 442) | |
| | (SEQ ID | | |
| | NO: 441) | | |
| CD39DSGAB | QVQLQESGP | DKTHTCPPCPAPEA | QSVLTQPPS |
| 44 | GPVKPSGTL | AGAPSVFLFPPKPK | VSGTPGQRV |
| | SLTCGVSGG | DTLMISRTPEVTCV | TISCSGSSS |
| | SISSNHWWT | VVDVSHEDPEVKFN | HIGSNYVYW |
| | WVRQPPGKG | WYVDGVEVHNAKTK | YQQLPGTAP |
| | LEWIGEIYH | PREEQYNSTYRVVS | KILIYSNDQ |
| | NGSTFLNPS | VLTVLHQDWLNGKE | RPAGVPDRF |
| | LKSRVTISV | YKCKVSNKALPAPI | SASKSGTSA |
| | DKSNNQFSL | EKTISKAKGQPREP | SLAISGLRS |
| | KLTSVTAAD | QVCTLPPSREEMTK | EDEADYYCA |
| | TAVYYCARG | NQVSLSCAVKGFYP | AWDDGQGGV |
| | WHRTGFRGY | SDIAVEWESNGQPE | FGGGTKLTV |
| | PSHWYFDLW | NNYKTTPPVLDSDG | LGQPKAAPS |
| | GRGTLVSVS | SFFLVSKLTVDKSR | VTLFPPSSE |
| | SASTKGPSV | WQQGNVFSCSVMHE | ELQANKATL |
| | FPLAPSSKS | ALHNHYTQKSLSLS | CVLISDFYP |
| | TSGGTAALG | PGGGGSKGGGSKGG | GAVTVAWKA |
| | CLVKDYFPE | GSKTQNKALPENVK | DSSPVKAGV |
| | PVTVSWNSG | YGIVLDAGSSHTSL | ETTTPSKQS |
| | ALTSGVHTF | YIYKWPAEKENDTG | NNKYAASSY |
| | PAVLQSSGL | VVHQVEECRVKGPG | LSLTPEQWK |
| | YSLSSVVTV | ISKFVQKVNEIGIY | SHRSYSCQV |
| | PSSSLGTQT | LTDCMERAREVIPR | THEGSTVEK |
| | YICNVNHKP | SQHQETPVYLGATA | TVAPTECS |
| | SNTKVDKKV | GMRLLRMESEELAD | (SEQ ID |
| | EPKSCDKTH | RVLDVVERSLSNYP | NO: 427) |
| | TCPPCPAPE | FDFQGARIITGQEE | |
| | AAGAPSVFL | GAYGWITINYLLGK | |
| | FPPKPKDTL | FSQKTRWFSIVPYE | |
| | MISRTPEVT | TNNQETFGALDLGG | |
| | CVVVDVSHE | ASTQVTFVPQNQTI | |
| | DPEVKFNWY | ESPDNALQFRLYGK | |
| | VDGVEVHNA | DYNVYTHSFLCYGK | |
| | KTKPREEQY | DQALWQKLAKDIQV | |
| | NSTYRVVSV | ASNEILRDPCFHPG | |
| | LTVLHQDWL | YKKVVNVSDLYKTP | |
| | NGKEYKCKV | CTKRFEMTLPFQQF | |
| | SNKALPAPI | EIQGIGNYQQCHQS | |
| | EKTISKAKG | ILELFNTSYCPYSQ | |
| | QPREPQVYT | CAFNGIFLPPLQGD | |
| | LPPCREEMT | FGAFSAFYFVMKFL | |
| | KNQVSLWCL | NLTSEKVSQEKVTE | |
| | VKGFYPSDI | MMKKFCAQPWEEIK | |
| | AVEWESNGQ | TSYAGVKEKYLSEY | |
| | PENNYKTTP | CFSGTYILSLLLQG | |
| | PVLDSDGSF | YHFTADSWEHIHFI | |
| | FLYSKLTVD | GKIQGSDAGWTLGY | |
| | KSRWQQGNV | MLNLTNMIPAEQPL | |
| | FSCSVMHEA | STPLSHSTYV | |
| | LHNHYTQKS | (SEQ ID | |
| | LSLSPG | NO: 444) | |
| | (SEQ ID | | |
| | NO: 443) | | |

In some embodiments, the C-terminus of the CD39 is linked via a G/S or A/E linker to the N-terminus of the Fe. In some embodiments, the Fc-CD39, has the sequence of:

(CD39AB1, SEQ ID NO: 445)
DKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGGGGGSNVKYGIVLDAGSSHTSLYI
YKWPAEKENDTGVVHQVECRVKGPGISKFVQKVNEIGIYLTDCMERAREV
IPRSQHQETPVYLGATAGMRLLRMESEELADRVLDVVERSLSNYPFDFQG
ARIITGQEEGAYGWITINYLLGKFSQKTRWFSIVPYETNNQETFGALDLG
GASTQVTFVPQNQTIHSFLCYGKDQALWQKLAKDIQVASNEILRDPCFHP
GYKKVVNVSDLYKTPCTKRFEMTLPFQQFEIQGIGNYQQCHQSILELFNT
SYCPYSQCAFNGIFLPPLQGDFGAFSAFYFVMKELNLTSEKVSQEKVTEM
MKKFCAQPWEEIKTSYAGVKEKYLSEYCFSGTYILSLLLQGYHFTADSWE
HIHFIGKIQGSDAGWTLGYMLNLTNMIPAEQPLSTPLSHSTYV
(CD39AB2, SEQ ID NO: 446)
DKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGGGGGSNVKYGIVLDAGSSHTSLYI
YKWPAEKENDTGVVHQVECRVKGPGISKFVQKVNEIGIYLTDCMERAREV
IPRSQHQETPVYLGATAGMRLLRMESEELADRVLDVVERSLSNYPFDFQG
ARIITGQEEGAYGWITINYLLGKFSQKTRWFSIVPYETNNQETFGALDLG
GASTQVTFVPQNQTIHSFLCYGKDQALWQKLAKDIQVASNEILRDPCFHP
GYKKVVNVSDLYKTPCTKRSEMTLPFQQFEIQGIGNYQQCHQSILELFNT
SYCPYSQCAFNGIFLPPLQGDFGAFSAFYFVMKFLNLTSEKVSQEKVTEM
MKKFCAQPWEEIKTSYAGVKEKYLSEYCFSGTYILSLLLQGYHFTADSWE
HIHFIGKIQGSDAGWTLGYMLNLINMIPAEQPLSTPLSHSTYV
(CD39AB3, SEQ ID NO: 447)
DKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGGGGGSNVKYGIVLDAGSSHTSLYI
YKWPAEKENDTGVVHQVECRVKGPGISKFVQKVNEIGIYLTDCMERAREV
IPRSQHQETPVYLGATAGMRLLRMESEELADRVLDVVERSLSNYPFDFQG
ARIITGQEEGAYGWITINYLLGKFSQKTRWFSIVPYETNNQETFGALDLG
GASTQVTFVPQNQTIHSFLCYGKDQALWQKLAKDIQVASNEILRDPCFHP
GYKKVVNVSDLYKTPCTKRFEMTEPFQQFEIQGIGNYQQCHQSILELFNT
SYCPYSQCAFNGIFLPPLQGDFGAFSAFYFVMKFLNLTSEKVSQEKVTEM
MKKFCAQPWEEIKTSYAGVKEKYLSEYCFSGTYILSLLLQGYHFTADSWE
HIHFIGKIQGSDAGWTLGYMLNLTNMIPAEQPLSTPLSHSTYV
(CD39AB4, SEQ ID NO: 448)
DKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGGGGGSNVKYGIVLDAGSSHTSLYI
YKWPAEKENDTGVVHQVECRVKGPGISKFVQKVNEIGIYLTDCMERAREV
IPRSQHQETPVYLGATAGMRLLRMESEELADRVLDVVERSLSNYPFDFQG
ARIITGQEEGAYGWITINYLLGKFSQKTRWFSIVPYETNNQETFGALDLG
GASTQVTFVPQNQTIHSFLCYGKDQALWQKLAKDIQVASNEILRDPCFHP
GYKKVVNVSDLYKTPCTKRFEMTLPFQQTEIQGIGNYQQCHQSILELFNT
SYCPYSQCAFNGIFLPPLQGDFGAFSAFYFVMKFLNLTSEKVSQEKVTEM
MKKFCAQPWEEIKTSYAGVKEKYLSEYCFSGTYILSLLLQGYHFTADSWE
HIHFIGKIQGSDAGWTLGYMLNLTNMIPAEQPLSTPLSHSTYV
(CD39AB5, SEQ ID NO: 449)
DKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGGGGGSNVKYGIVLDAGSSHTSLYI
YKWPAEKENDTGVVHQVECRVKGPGISKFVQKVNEIGIYLTDCMERAREV
IPRSQHQETPVYLGATAGMRLLRMESEELADRVLDVVERSLSNYPFDFQG
ARIITGQEEGAYGWITINYLLGKFSQKTRWFSIVPYETNNQETFGALDLG
GASTQVTFVPQNQTIHSFLCYGKDQALWQKLAKDIQVASNEILRDPCFHP
GYKKVVNVSDLYKTPCTKRFEMTLPFQQFEIQGIGNYQQCHQSILELFNT
SYCPYSQCAFNGIFLPPLQGDFGAFSAFYFVMKFLNLTSEKVSQEKVTEM
MKKFCAQPWEEIKTSYAGVKEKYLSEYCRSGTYILSLLLQGYHFTADSWE
HIHFIGKIQGSDAGWTLGYMLNLTNMIPAEQPLSTPLSHSTYV
(CD39AB6, SEQ ID NO: 450)
DKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGGGGGSNVKYGIVLDAGSSHTSLYI
YKWPAEKENDTGVVHQVECRVKGPGISKFVQKVNEIGIYLTDCMERAREV
IPRSQHQETPVYLGATAGMRLLRMESEELADRVLDVVERSLSNYPFDFQG
ARIITGQEEGAYGWITINYLLGKFSQKTRWFSIVPYETNNQETFGALDLG
GASTQVTFVPQNQTIHSFLCYGKDQALWQKLAKDIQVASNEILRDPCFHP
GYKKVVNVSDLYKTPCTKRFEMTLPFQQFEIQGIGNYQQCHQSILELFNT
SYCPYSQCAFNGIFLPPLQGDFGAFSAFYFVMKFLNLTSEKVSQEKVTEM
MKKFCAQPWEEIKTSYAGVKEKYLSEYCFSGTYISSLLLQGYHFTADSWE
HIHFIGKIQGSDAGWTLGYMLNLTNMIPAEQPLSTPLSHSTYV
(CD39AB7, SEQ ID NO: 451)
DKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGGGGGSNVKYGIVLDAGSSHTSLYI
YKWPAEKENDTGVVHQVECRVKGPGISKFVQKVNEIGIYLTDCMERAREV
IPRSQHQETPVYLGATAGMRLLRMESEELADRVLDVVERSLSNYPFDFQG
ARIITGQEEGAYGWITINYLLGKFSQKTRWFSIVPYETNNQETFGALDLG
GASTQVTFVPQNQTIHSFLCYGKDQALWQKLAKDIQVASNEILRDPCFHP
GYKKVVNVSDLYKTPCTKRFEMTLPFQQFEIQGIGNYQQCHQSILELFNT
SYCPYSQCAFNGIFLPPLQGDFGAFSAFYFVMKFLNLTSEKVSQEKVTEM
MKKFCAQPWEEIKTSYAGVKEKYLSEYCFSGTYILSLLSQGYHFTADSWE
HIHFIGKIQGSDAGWTLGYMLNLTNMIPAEQPLSTPLSHSTYV

In some embodiments, the N-terminus of the CD39 is linked via a G/S or A/E linker to the C-terminus of the Fc. In some embodiments, the CD39-Fc, has the sequence of:

(CD39AB8, SEQ ID NO: 452)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLLQGYHFTADSWEHIHFIGKIQGSDAGWTLGYMLNLTNMIPAEQ
PLSTPLSHSTYVGGGGSGGGGSGGGGSGGGGSDKTHTCPPCPAPEAAGAP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQ
KSLSLSPG
(CD39AB9, SEQ ID NO: 453)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLLQGYHFTADSWEHIHFIGKIQGSDAGWTLGYMLNLTNMIPAEQ
PLSTPLSHSTYVGGGSEGGGSEGGGSEGGGSEDKTHTCPPCPAPEAAGAP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQ
KSLSLSPG
(CD39AB10, SEQ ID NO: 454)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLLQGYHFTADSWEHIHFIGKIQGSDAGWTLGYMLNLINMIPAEQ
PLSTPLSHSTYVAEEEKAEEEKAEEEKAEEEKDKTHTCPPCPAPEAAGAP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQ
KSLSLSPG
(CD39AB11, SEQ ID NO: 455)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLLQGYHFTADSWEHIHFIGKIQGSDAGWTLGYMLNLTNMIPAEQ
PLSTPLSHSTYVGGGGSGGGGSGGGGSGGGGSDKTHTCPPCPAPEAAGAP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQ
KSLSLSPG
(CD39AB12, SEQ ID NO: 456)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLLQGYHFTADSWEHIHFIGKIQGSDAGWTLGYMLNLTNMIPAEQ
PLSTPLSHSTYVGGGSEGGGSEGGGSEGGGSEDKTHTCPPCPAPEAAGAP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQ
KSLSLSPG
(CD39AB13, SEQ ID NO: 457)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLLQGYHFTADSWEHIHFIGKIQGSDAGWTLGYMLNLTNMIPAEQ
PLSTPLSHSTYVAEEEKAEEEKAEEEKAEEEKDKTHTCPPCPAPEAAGAP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQ
KSLSLSPG
(CD39AB14, SEQ ID NO: 458)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPEDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLLQGYHFTADSWEHIHFIGKIQGSDAGWTLGYMLNLTNMIPAEQ
PLSTPLSHSTYVGGGGSGGGGSGGGGSGGGGSDKTHTCPPCPAPEAAGAP
SVELFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQ
KSLSLSPG
(CD39AB15, SEQ ID NO: 459)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLLQGYHFTADSWEHIHFIGKIQGSDAGWTLGYMLNLINMIPAEQ
PLSTPLSHSTYVGGGSEGGGSEGGGSEGGGSEDKTHTCPPCPAPEAAGAP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQ
KSLSLSPG
(CD39AB16, SEQ ID NO: 460)
NVKYGIVLDAGSSHTSLYIYKWPAEKENDTGVVHQVECRVKGPGISKFVQ
KVNEIGIYLTDCMERAREVIPRSQHQETPVYLGATAGMRLLRMESEELAD
RVLDVVERSLSNYPFDFQGARIITGQEEGAYGWITINYLLGKFSQKTRWF
SIVPYETNNQETFGALDLGGASTQVTFVPQNQTIHSFLCYGKDQALWQKL
AKDIQVASNEILRDPCFHPGYKKVVNVSDLYKTPCTKRFEMTLPFQQFEI
QGIGNYQQCHQSILELFNTSYCPYSQCAFNGIFLPPLQGDFGAFSAFYFV
MKFLNLTSEKVSQEKVTEMMKKFCAQPWEEIKTSYAGVKEKYLSEYCFSG
TYILSLLLQGYHFTADSWEHIHFIGKIQGSDAGWTLGYMLNLTNMIPAEQ
PLSTPLSHSTYVAEEEKAEEEKAEEEKAEEEKDKTHTCPPCPAPEAAGAP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQ
KSLSLSPG

The molecules comprising an anti-desmoglein 1 antibody, an anti-desmoglein 2 antibody, an anti-desmoglein 3 antibody, or an anti-desmoglein 4 antibody (generically referred to as an “anti-desmoglein antibody”) and a PD-1 antibody, an IL-2 mutein, or a CD39 can be various formats as described herein. For example, they can be in the following formats:

### PD-1 ML-N Format:

Heavy Chain: NT-[VH_PD-1]-[CH1-CH2-CH3]-[LinkerA]-[anti-desmoglein antibodyscFv]-CT
Light Chain: NT-[VK_PD-1]-[CK]-CT

### PD-1 ML-N(2) Format:

Heavy Chain: NT-[VH_PD-1]-[CH1-CH2-CH3]-[LinkerC]-[anti-desmoglein antibodyscFab]-CT
Light Chain: NT-[VK_PD-1]-[CK]-CT

### PD-1 ML-C Format:

Heavy Chain: NT-[VH_anti-desmoglein antibody]-[CH1-CH2-CH3]-[LinkerA]-[PD-1scFv]-CT
Light Chain: NT-[VK_anti-desmoglein antibody]-[CK]-CT

### PD-1 CL-N Format:

Light Chain: NT-[VH_PD-1]-[CK]-[LinkerD]-[anti-desmoglein antibodyscFab]-CT
Heavy Chain: NT-[VK_PD-1]-[CH1-CH2-CH3]-CT

### PD-1 IgG Format:

Heavy Chain: NT-[VH_PD-1]-[CH1-CH2-CH3]
Light Chain: NT-[VK_PD-1]-[CK]-CT

### IL-2 ML-N Format:

Heavy Chain: NT-[VH_anti-desmoglein antibody]-[CH1-CH2-CH3]-[LinkerA]-[IL-2 mutein]-CT
Light Chain: NT-[VK_anti-desmoglein antibody]-[CL]-CT

### IL-2 CL-N Format:

Light Chain: NT-[VK_anti-desmoglein antibody]-[CL]-[LinkerC/D]-[IL-2 mutein]-CT
Heavy Chain: NT-[VH_anti-desmoglein antibody]-[CH1-CH2-CH3]-CT

### CD39 ML-N Format:

Heavy Chain: NT-[VH_anti-desmoglein antibody]-[CH1-CH2-CH3]-[LinkerA/B/C]-[CD39]-CT
Light Chain: NT-[VK_anti-desmoglein antibody]-[CL]-CT

### CD39 ML-N(2) Format:

Heavy Chain: NT-[CD39]-[LinkerA/B/C]-[VH_anti-desmoglein antibody]-[CH1-CH2-CH3]-CT
Light Chain: NT-[VK_anti-desmoglein antibody]-[CL]-CT

### CD39 CL-N Format:

Light Chain: NT-[VK_anti-desmoglein antibody]-[CL]-[LinkerA/B/C]-[CD39]-CT
Heavy Chain: NT-[VH_anti-desmoglein antibody]-[CH1-CH2-CH3]-CT

### CD39 CL-N(2) Format:

Light Chain: NT-[CD39]-[LinkerA/B/C/D]-[VK_anti-desmoglein antibody]-[CL]-CT
Heavy Chain: NT-[VH_anti-desmoglein antibody]-[CH1-CH2-CH3]-CT

### CD39 ML-N(3) Format:

Heavy Chain 1: NT-[VH_anti-desmoglein antibody]-[CH1-CH2-CH3]-[LinkerA/B/C/D]-[CD39]-CT
Heavy Chain 2: NT-[VH_anti-desmoglein antibody]-[CH1-CH2-CH3]-CT
Light Chain: NT-[VK_anti-desmoglein antibody]-[CL]-CT

### CD39 ML-N(4) Format:

Heavy Chain 1: NT-[VH_anti-desmoglein antibody]-[CH1-CH2-CH3]-CT
Heavy Chain 2: NT-[IgG1Fc]-[LinkerA/B/C/D]-[CD39]-CT
Light Chain: NT-[VK_anti-desmoglein antibody]-[CL]-CT

### CD39 ML-N(5) Format:

Heavy Chain 1: NT-[VH_anti-desmoglein antibody]-[CH1]-[IgG2H]-[CD39]-CT
Light Chain: NT-[VK_anti-desmoglein antibody]-[CL]-CT

The abbreviations used above are as follows:

| Component | Description |
|---|---|
| NT | N-terminus |
| CT | C-terminus |
| VH_PD-1 | VH domain of PD-1 antibody |
| | as provided herein. |
| VK_PD-1 | VK domain of PD-1 antibody |
| | as provided herein. |
| PD-1scFv | PD-1 antibody in scFv |
| | comprising the VH and VK |
| | domain. |
| VH_anti- desmoglein antibody | VH domain of- anti- |
| | desmoglein antibody Ab as |
| | provided herein. |
| VK_anti- desmoglein antibody | VK domain of- anti- |
| | desmoglein antibody Ab as |
| | provided herein. This can |
| | also be substituted with a VL |
| | sequences as provided herein. |
| anti- desmoglein antibody scFv | anti- desmoglein antibody |
| | scFV Ab as provided herein. |
| anti- desmoglein antibody scFab | anti- desmoglein antibody |
| | scFab Ab as provided herein. |
| VH_anti- desmoglein antibody_BM1 | Rat anti-mouse anti- |
| | desmoglein antibody |
| | placeholder VH domain |
| VK_anti- desmoglein antibody_BM1 | Rat anti-mouse anti- |
| | desmoglein antibody |
| | placeholder VK domain |
| anti- desmoglein antibody | Rat anti-mouse anti- |
| scFv_BM1 | desmoglein antibody |
| | placeholder scFv |
| VH_PD-1_BM1 | Anti-human PD-1 agonist |
| | placeholder VH domain |
| VK_PD-1_BM1 | Anti-human PD-1 agonist |
| | placeholder VK domain |
| CH1-CH2-CH3 | Human IgG1 Constant Heavy |
| | 1 (CH1), Constant Heavy 2 |
| | (CH2), and Constant Heavy 3 |
| | (CH3) domains |
| IgG1 Fc | Human IgG1 Fc |
| IgG2H | Human IgG2 Hinger region |
| CK | Human constant kappa |
| | domain |
| CL | Constant lambda domain |
| IL-2_Mutein | IL-2 moiety such as those |
| | provided herein. |
| CD39 | CD39 effector domains such |
| | as those provided herein |
| Linker_A | G/S, or G/S/E, or A/E/K |
| | linker (5 amino acid length) |
| Linker_B | G/S, or G/S/E, or A/E/K |
| | linker (10 amino acid length) |
| Linker_C | G/S, or G/S/E, or A/E/K |
| | linker (15 amino acid length) |
| Linker_D | G/S, or G/S/E, or A/E/K |
| | linker (20 amino acid length) |

The sequence of CH1-CH2-CH3 can be, for example,

(SEQ ID NO: 44)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPG;
or
(KIH mutations; SEQ ID NO: 461)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPCREEMTKNQVSLSC
AVKGFYPSDIAVEWESNGQPENNYKTTPPVLDSDGSFFLVSKLTVDKSRW
QQGNVFSCSVMHEALHNHYTQKSLSLSPG;
or
(KIH mutations; SEQ ID NO: 462)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVCTLPPSREEMTKNQVSLWC
LVKGFYPSDIAVEWESNGQPENNYKTTPPVLDSDGSFFLYSKLTVDKSRW
QQGNVFSCSVMHEALHNHYTQKSLSLSPG;
or
(KIH mutations; SEQ ID NO: 463)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVCTLPPSREEMTKNQVSLSC
AVKGFYPSDIAVEWESNGQPENNYKTTPPVLDSDGSFFLVSKLTVDKSRW
QQGNVFSCSVMHEALHNHYTQKSLSLSPG;
or
(KIH mutations; SEQ ID NO: 464)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPCREEMTKNQVSLWC
LVKGFYPSDIAVEWESNGQPENNYKTTPPVLDSDGSFFLYSKLTVDKSRW
QQGNVFSCSVMHEALHNHYTQKSLSLSPG

The sequence of CK/CL can be, for example,

(SEQ ID NO: 45)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

The sequence of IgG2 hinge can be, for example, EPKSCCVECPPCPAPPAAAGA (SEQ ID NO: 465).

In some embodiments, if the therapeutic compound comprises a Fc portion, the Fc domain, (portion) bears mutations to render the Fc region “effectorless” that is unable to bind FcRs. The mutations that render Fc regions effectorless are known. In some embodiments, the mutations in the Fc region, which is according to the known numbering system, are selected from the group consisting of: K322A, L234A, L235A, G237A, L234F, L235E, N297, P331S, or any combination thereof. In some embodiments, the Fc mutations comprises a mutation at L234 and/or L235 and/or G237. In some embodiments, the Fc mutations comprise L234A and/or L235A mutations, which can be referred to as LALA mutations. In some embodiments, the Fc mutations comprise L234A, L235A, and G237A mutations.

In some embodiments, the Fc portion has a sequence of:

(SEQ ID NO: 466)
DKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPCREEMTKNQVSLSCAVK
GFYPSDIAVEWESNGQPENNYKTTPPVLDSDGSFFLVSKLTVDKSRWQQG
NVFSCSVMHEALHNHYTQKSLSLSPG;
or
(SEQ ID NO: 467)
DKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVCTLPPSREEMTKNQVSLWCLVK
GFYPSDIAVEWESNGQPENNYKTTPPVLDSDGSFFLYSKLTVDKSRWQQG
NVFSCSVMHEALHNHYTQKSLSLSPG;
or
(SEQ ID NO: 468)
DKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPCREEMTKNQVSLWCLVK
GFYPSDIAVEWESNGQPENNYKTTPPVLDSDGSFFLYSKLTVDKSRWQQG
NVFSCSVMHEALHNHYTQKSLSLSPG;
or
(SEQ ID NO: 469)
DKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVCTLPPSREEMTKNQVSLSCAVK
GFYPSDIAVEWESNGQPENNYKTTPPVLDSDGSFFLVSKLTVDKSRWQQG
NVFSCSVMHEALHNHYTQKSLSLSPG.

Disclosed herein are Linker Region polypeptides, therapeutic peptides, and nucleic acids encoding the polypeptides (e.g., therapeutic compounds), vectors comprising the nucleic acid sequences, and cells comprising the nucleic acids or vectors.

Therapeutic compounds can comprise a plurality of specific targeting moieties. In some embodiments, the therapeutic compound comprises a plurality one specific targeting moiety, a plurality of copies of a donor specific targeting moiety or a plurality of tissue specific targeting moieties. In some embodiments, a therapeutic compound comprises a first and a second donor specific targeting moiety, e.g., a first donor specific targeting moiety specific for a first donor target and a second donor specific targeting moiety specific for a second donor target, e.g., wherein the first and second target are found on the same donor tissue. In some embodiments, the therapeutic compound comprises e.g., a first specific targeting moiety for a tissue specific target and a second specific targeting moiety for a second target, e.g., wherein the first and second target are found on the same or different target tissue.

Polypeptides Derived from Reference, e.g., Human Polypeptides

In some embodiments, a component of a therapeutic molecule is derived from or based on a reference molecule, e.g., in the case of a therapeutic molecule for use in humans, from a naturally occurring human polypeptide. E.g., in some embodiments, all or a part of a CD39 molecule, a specific targeting moiety, a target ligand binding molecule, or a tissue specific targeting moiety, can be based on or derived from a naturally occurring human polypeptide. E.g., a PD-L1 molecule can be based on or derived from a human PD-L1 sequence.

In some embodiments, a therapeutic compound component, e.g., a PD-L1 molecule:
a) comprises all or a portion of, e.g., an active portion of, a naturally occurring form of the human polypeptide;
b) comprises all or a portion of, e.g., an active portion of, a human polypeptide having a sequence appearing in a database, e.g., GenBank database, on Jan. 11, 2017, a naturally occurring form of the human polypeptide that is not associated with a disease state;
c) comprises a human polypeptide having a sequence that differs by no more than 1, 2, 3, 4, 5, 10, 20, or 30 amino acid residues from a sequence of a) or b);
d) comprises a human polypeptide having a sequence that differs by no more than 1, 2, 3, 4, 5 10, 20, or 30% its amino acids residues from a sequence of a) or b);
e) comprises a human polypeptide having a sequence that does not differ substantially from a sequence of a) or b); or
f) comprises a human polypeptide having a sequence of c), d), or e) that does not differ substantially in biological activity, e.g., ability to enhance or inhibit an immune response, from a human polypeptide having the sequence of a) or b).

### Pharmaceutical Compositions and Kits

In another aspect, the present embodiments provide compositions, e.g., pharmaceutically acceptable compositions, which include a therapeutic compound described herein, formulated together with a pharmaceutically acceptable carrier. As used herein, “pharmaceutically acceptable carrier” includes any and all solvents, dispersion media, isotonic and absorption delaying agents, and the like that are physiologically compatible.

The carrier can be suitable for intravenous, intramuscular, subcutaneous, parenteral, rectal, local, ophthalmic, topical, spinal or epidermal administration (e.g., by injection or infusion). As used herein, the term “carrier” means a diluent, adjuvant, or excipient with which a compound is administered. In some embodiments, pharmaceutical carriers can also be liquids, such as water and oils, including those of petroleum, animal, vegetable or synthetic origin, such as peanut oil, soybean oil, mineral oil, sesame oil, and the like. The pharmaceutical carriers can also be saline, gum acacia, gelatin, starch paste, talc, keratin, colloidal silica, urea, and the like. In addition, auxiliary, stabilizing, thickening, lubricating, and coloring agents can be used. The carriers can be used in pharmaceutical compositions comprising the therapeutic compounds provided for herein.

The compositions and compounds of the embodiments provided herein may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In some embodiments, the mode of administration is parenteral (e.g., intravenous, subcutaneous, intraperitoneal, intramuscular). In some embodiments, the therapeutic molecule is administered by intravenous infusion or injection. In another embodiment, the therapeutic molecule is administered by intramuscular or subcutaneous injection. In another embodiment, the therapeutic molecule is administered locally, e.g., by injection, or topical application, to a target site. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion.

Therapeutic compositions typically should be sterile and stable under the conditions of manufacture and storage. The composition can be formulated as a solution, microemulsion, dispersion, liposome, or other ordered structure suitable to high therapeutic molecule concentration. Sterile injectable solutions can be prepared by incorporating the active compound (i.e., therapeutic molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a therapeutic compound can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a therapeutic compound is 0.1-30 mg/kg, more preferably 1-25 mg/kg. Dosages and therapeutic regimens of the therapeutic compound can be determined by a skilled artisan. In certain embodiments, the therapeutic compound is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the therapeutic compound is administered at a dose from about 10 to 20 mg/kg every other week. The therapeutic compound can be administered by intravenous infusion at a rate of more than 20 mg/min, e.g., 20-40 mg/min, and typically greater than or equal to 40 mg/min to reach a dose of about 35 to 440 mg/m2, typically about 70 to 310 mg/m2, and more typically, about 110 to 130 mg/m2. In embodiments, the infusion rate of about 110 to 130 mg/m2 achieves a level of about 3 mg/kg. In other embodiments, the therapeutic compound can be administered by intravenous infusion at a rate of less than 10 mg/min, e.g., less than or equal to 5 mg/min to reach a dose of about 1 to 100 mg/m2, e.g., about 5 to 50 mg/m2, about 7 to 25 mg/m2, or, about 10 mg/m2. In some embodiments, the therapeutic compound is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. The pharmaceutical compositions may include a “therapeutically effective amount” or a “prophylactically effective amount” of a therapeutic molecule. A “therapeutically effective amount” refers to an amount effective, at dosages and for periods of time necessary, to achieve the desired therapeutic result. A therapeutically effective amount of a therapeutic molecule may vary according to factors such as the disease state, age, sex, and weight of the individual, and the ability of the therapeutic compound to elicit a desired response in the individual. A therapeutically effective amount is also one in which any toxic or detrimental effects of a therapeutic molecule t is outweighed by the therapeutically beneficial effects. A “therapeutically effective dosage” preferably inhibits a measurable parameter, e.g., immune attack at least about 20%, more preferably by at least about 40%, even more preferably by at least about 60%, and still more preferably by at least about 80% relative to untreated subjects. The ability of a compound to inhibit a measurable parameter, e.g., immune attack, can be evaluated in an animal model system predictive of efficacy in transplant rejection or autoimmune disorders. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the embodiments is a kit comprising a therapeutic compound described herein. The kit can include one or more other elements including: instructions for use; other reagents, e.g., a label, a therapeutic agent, or an agent useful for chelating, or otherwise coupling, a therapeutic molecule to a label or other therapeutic agent, or a radioprotective composition; devices or other materials for preparing the a therapeutic molecule for administration; pharmaceutically acceptable carriers; and devices or other materials for administration to a subject.

In some embodiments, embodiments provided herein also include, but are not limited to:
1. A polypeptide comprising a skin targeting moiety that binds to a target cell and an effector binding/modulating moiety, wherein the effector binding/modulating moiety is a PD-1 agonist, CD39 Effector Domain, or an IL-2 mutein polypeptide (IL-2 mutein).
2. The polypeptide of embodiment 1, wherein the skin targeting moiety comprises an antibody that binds to a target protein on the surface of the skin cell.
3. The polypeptide of embodiment 2, wherein the antibody that binds to the target protein on the surface of the skin cell is an antibody that binds to a desmoglein protein.
4. The polypeptide of embodiment 3, wherein the antibody that binds to a desmoglein protein is an anti-desmoglein-1 antibody, an anti-desmoglein-2 antibody, an anti-desmoglein-3 antibody, or an anti-desmoglein-4 antibody.
5. The polypeptide of any one of embodiments 1-4, wherein the effector binding/modulating moiety binds to a receptor expressed by an immune cell.
6. The polypeptide of embodiment 5, wherein the effector binding/modulating moiety is a PD-1 agonist, a CD39 Effector Domain, or an IL-2 mutein.
7. The polypeptide of embodiment 4, wherein the immune cell contributes to an unwanted immune response.
8. The polypeptide of any one of embodiments 5-7, wherein the immune cell causes a disease pathology.
9. The polypeptide of any one of embodiments 1-8, wherein the targeting moiety comprises an anti-desmoglein 1 antibody, an anti-desmoglein 2 antibody, an anti-desmoglein 3 antibody, or an anti-desmoglein 4 antibody.
10. The polypeptide of embodiment 1, wherein the polypeptide has the formula from N-terminus to C-terminus:
   R1-Linker Region A-R2; or
   R3-Linker Region B-R4,
wherein,
   R1, R2, R3, and R4, each independently comprises the effector binding/modulating moiety, the targeting moiety, or is absent, provided that at least one of R1 and R2 is not absent, and at least one of R3 and R4 is not absent.
11. The polypeptide of embodiment 10, wherein the polypeptide having the formula of R1 Linker Region A-R2 and the polypeptide having the formula of R3-Linker Region B-R4 interact with one another to form a polypeptide complex.
12. The polypeptide of embodiment 10, wherein the polypeptide having the formula of R1 Linker Region A-R2 and the polypeptide having the formula of R3-Linker Region B-R4 do not interact with one another to form a polypeptide complex.
13. The polypeptide of any one of embodiments 10-12, wherein each of Linker Region A and Linker Region B comprises an Fc region.
14. The polypeptide of any one of embodiments 9-12, wherein the Linker Region A and Linker Region B each comprise a linker.
15. The polypeptide of embodiment 14, wherein the linker is absent.
16. The polypeptide of embodiment 14, wherein the linker comprises a Fc region.
17. The polypeptide of embodiment 14, wherein the linker comprises a glycine/serine linker.
18. The polypeptide of embodiment 17, wherein the linker is

(SEQ ID NO: 22)
GGGGSGGGGSGGGGSGGGGS,
(SEQ ID NO: 30)
GGGGSGGGGSGGGGS,
(SEQ ID NO: 531)
GGGGSGGGGS,
or
(SEQ ID NO: 23)
GGGGS.

19. The polypeptide of any one of embodiments 10-12, wherein one of R1 and R2 is an IL-2 mutein and one of R1 and R2 is an anti-desmoglein 1 antibody, an anti-desmoglein 2 antibody, an anti-desmoglein 3 antibody, or an anti-desmoglein 4 antibody.
20. The polypeptide of any one of embodiments 10-12 or 19, wherein R1 is an TL-2 mutein and R2 is an anti-desmoglein 1 antibody, an anti-desmoglein 2 antibody, an anti-desmoglein 3 antibody, or an anti-desmoglein 4 antibody.
21. The polypeptide of any one of embodiments 10-12 or 19, wherein R1 is an anti-desmoglein 1 antibody, an anti-desmoglein 2 antibody, an anti-desmoglein 3 antibody, or an anti-desmoglein 4 antibody and R2 is an IL-2 mutein.
22. The polypeptide of any one of embodiments 10-12, wherein one of R3 and R4 is an IL-2 mutein and one of R3 and R4 is an anti-desmoglein 1 antibody, an anti-desmoglein 2 antibody, an anti-desmoglein 3 antibody, or an anti-desmoglein 4 antibody.
23. The polypeptide of any one of embodiments 10-12 or 22, wherein R3 is an TL-2 mutein and R4 is an anti-desmoglein 1 antibody, an anti-desmoglein 2 antibody, an anti-desmoglein 3 antibody, or an anti-desmoglein 4 antibody.
24. The polypeptide of any one of embodiments 10-12 or 22, wherein R3 is an anti-desmoglein 1 antibody, an anti-desmoglein 2 antibody, an anti-desmoglein 3 antibody, or an anti-desmoglein 4 antibody and one R4 is an TL-2 mutein.
25. The polypeptide of any one of embodiments 10-12, wherein one of R1 and R2 is an anti-PD-1 antibody and one of R1 and R2 is an anti-desmoglein 1 antibody, an anti-desmoglein 2 antibody, an anti-desmoglein 3 antibody, or an anti-desmoglein 4 antibody.
26. The polypeptide of any one of embodiments 10-12 or 25, wherein R1 is an anti-PD-1 antibody and R2 is an anti-desmoglein 1 antibody, an anti-desmoglein 2 antibody, an anti-desmoglein 3 antibody, or an anti-desmoglein 4 antibody.
27. The polypeptide of any one of embodiments 10-12 or 25, wherein R1 is an anti-desmoglein 1 antibody, an anti-desmoglein 2 antibody, an anti-desmoglein 3 antibody, or an anti-desmoglein 4 antibody and R2 is an anti-PD-1 antibody.
28. The polypeptide of any one of embodiments 10-12, wherein one of R3 and R4 is an anti-PD-1 antibody and one of R3 and R4 is an anti-desmoglein 1 antibody, an anti-desmoglein 2 antibody, an anti-desmoglein 3 antibody, or an anti-desmoglein 4 antibody.
29. The polypeptide of any one of embodiments 10-12 or 28, wherein R3 is an anti-PD-1 antibody and R4 is an anti-desmoglein 1 antibody, an anti-desmoglein 2 antibody, an anti-desmoglein 3 antibody, or an anti-desmoglein 4 antibody.
30. The polypeptide of any one of embodiments 10-12 or 28, wherein R3 is an anti-desmoglein 1 antibody, an anti-desmoglein 2 antibody, an anti-desmoglein 3 antibody, or an anti-desmoglein 4 antibody and one R4 is an anti-PD-1 antibody.
31. The polypeptide of any one of embodiments 10-12, wherein one of R1 and R2 is a CD39 Effector Domain and one of R1 and R2 is an anti-desmoglein 1 antibody, an anti-desmoglein 2 antibody, an anti-desmoglein 3 antibody, or an anti-desmoglein 4 antibody.
32. The polypeptide of any one of embodiments 10-12 or 31, wherein R1 is a CD39 Effector Domain and R2 is an anti-desmoglein 1 antibody, an anti-desmoglein 2 antibody, an anti-desmoglein 3 antibody, or an anti-desmoglein 4 antibody.
33. The polypeptide of any one of embodiments 10-12 or 31, wherein R1 is an anti-desmoglein 1 antibody, an anti-desmoglein 2 antibody, an anti-desmoglein 3 antibody, or an anti-desmoglein 4 antibody and R2 is a CD39 Effector Domain.
34. The polypeptide of any one of embodiments 10-12, wherein one of R3 and R4 is a CD39 Effector Domain and one of R3 and R4 is an anti-desmoglein 1 antibody, an anti-desmoglein 2 antibody, an anti-desmoglein 3 antibody, or an anti-desmoglein 4 antibody.
35. The polypeptide of any one of embodiments 10-12 or 34, wherein R3 is a CD39 Effector Domain and R4 is an anti-desmoglein 1 antibody, an anti-desmoglein 2 antibody, an anti-desmoglein 3 antibody, or an anti-desmoglein 4 antibody.
36. The polypeptide of any one of embodiments 10-12 or 34, wherein R3 is an anti-desmoglein 1 antibody, an anti-desmoglein 2 antibody, an anti-desmoglein 3 antibody, or an anti-desmoglein 4 antibody and one R4 is a CD39 Effector Domain.
37. The polypeptide of any one of embodiments 1, 6, or 19-24, wherein the IL-2 mutein comprises a IL-2 sequence of SEQ ID NO: 6, and wherein the sequence comprises a mutation at a position that corresponds to position 53, 56, 80, or 118 of SEQ ID NO: 6.
38. The polypeptide of any one of embodiments 1, 6, or 19-24, wherein the IL-2 mutein comprises the sequence of SEQ ID NO: 60, wherein at least one of X₁, X₂, X₃, and X₄ is I and the remainder are L or I.
39. The polypeptide of embodiment 37, wherein the mutation is a L to I substitution at position 53, 56, 80, or 118 of SEQ ID NO: 6.
40. The polypeptide of embodiment 1, 6, 19-24, or 37 or 39, wherein the IL-2 mutein comprises a IL-2 sequence of SEQ ID NO: 6, further comprising a mutation at one or more positions of 29, 31, 35, 37, 48, 69, 71, 74, 88, and 125 of SEQ ID NO: 6.
41. The polypeptide of embodiment 38, wherein the mutation is a L to I substitution at position 53, 56, 80, or 118 of SEQ ID NO: 60.
42. The polypeptide of embodiment 1, 6, 19-24, or 38, wherein the IL-2 mutein comprises a IL-2 sequence of SEQ ID NO: 60, further comprising a mutation at one or more positions of 29, 31, 35, 37, 48, 69, 71, 74, 88, and 125 of SEQ ID NO: 60.
43. The polypeptide of embodiment 1, 6, 19-24, or 37-42, wherein the TL-2 mutein further comprises a mutation at one or more of positions of E15, H16, Q22, D84, E95, or Q126.
44. The polypeptide of embodiment 1, 6, 19-24, or 37-43, wherein the mutation in the IL-2 mutein is a mutation at one or more of E15Q, H16N, Q22E, D84N, E95Q, or Q126E.
45. The polypeptide of embodiment 1, 6, 19-24, or 37-44, wherein the TL-2 mutein comprises a N29S mutation.
46. The polypeptide of embodiment 1, 6, 19-24, or 37-45, wherein the TL-2 mutein comprises a Y31S or a Y51H mutation.
47. The polypeptide of embodiment 1, 6, 19-24, or 37-46, wherein the TL-2 mutein comprises a K35R mutation.
48. The polypeptide of embodiment 1, 6, 19-24, or 37-47, wherein the TL-2 mutein comprises a T37A mutation.
49. The polypeptide of embodiment 1, 6, 19-24, or 37-48, wherein the TL-2 mutein comprises a K48E mutation.
50. The polypeptide of embodiment 1, 6, 19-24, or 37-49, wherein the TL-2 mutein comprises a V69A mutation.
51. The polypeptide of embodiment 1, 6, 19-24, or 37-50, wherein the TL-2 mutein comprises a N71R mutation.
52. The polypeptide of embodiment 1, 6, 19-24, or 37-51, wherein the TL-2 mutein comprises a Q74P mutation.
53. The polypeptide of embodiment 1, 6, 19-24, or 37-52, wherein the TL-2 mutein comprises a N88D or a N88R mutation.
54. The polypeptide of embodiment 1, 6, 19-24, or 37-53, wherein the TL-2 mutein comprises a C125A or C125S mutation.
55. The polypeptide of embodiment 1, 6, 19-24, or 37-54, wherein the TL-2 mutein is fused or linked to a Fc peptide, such as a protein comprising a sequence of SEQ ID NO: 59.
56. The polypeptide of embodiment 55, wherein the Fc peptide comprises a mutation at one or more of positions of L234, L247, L235, L248, G237, and G250.
57. The polypeptide of embodiment 55, wherein the mutation in the Fc polypeptide is L to A or G to A mutation.
58. The polypeptide of embodiment 55, wherein the Fc peptide comprises L247A, L248A, and G250A mutations.
59. The polypeptide of embodiment 55, wherein the Fc peptide comprises a L234A mutation, a L235A mutation, and/or a G237A mutation.
60. The polypeptide of embodiment 1, wherein the polypeptide comprises a first chain and a second chain that form the polypeptide, wherein
   the first chain comprises:
   V_{H}—H_{c}-Linker-C₁, wherein V_{H} is a variable heavy domain that binds to the target skin cell with a V_{L} domain of the second chain; H_{c} is a heavy chain of antibody comprising CH1-CH2-CH3 domain, the Linker is a glycine/serine linker, and C₁ is an IL-2 mutein fused or linked to a Fc protein in either the N-terminal or C-terminal orientation; and
   the second chain comprises:
   V_{L}-L_{c}, wherein V_{L} is a variable light chain domain that binds to the target cell with the V_{H} domain of the first chain, and the Lc domain is a light chain CK domain.
61. The polypeptide of embodiment 60, wherein the VH and VL domain are anti-desmoglein-1, anti-desmoglein-2, anti-desmoglein-3, and/or anti-desmoglein-4 variable domains that bind to desmoglein-1, desmoglein-2, desmoglein-3, and/or desmoglein-4, respectively, expressed on a cell.
62. The polypeptide of embodiment 60, wherein the IL-2 mutein comprises a mutation at a position that corresponds to position 53, 56, 80, or 118 of SEQ ID NO: 6.
63. The polypeptide of embodiment 62, wherein the mutation is a L to I mutation at position 53, 56, 80, or 118 of SEQ ID NO: 6.
64. The polypeptide of embodiment 60, wherein the IL-2 mutein comprises a mutation at a position that corresponds to position 53, 56, 80, or 118 of SEQ ID NO: 60.
65. The polypeptide of embodiment 64, wherein the mutation is a L to I mutation at position 53, 56, 80, or 118 of SEQ ID NO: 60.
66. The polypeptide of any one of embodiments 60 or 62, wherein the mutein further comprises a mutation at a position that corresponds to position 69, 75, 88, or 125, or any combination thereof.
67. The polypeptide of embodiment 60, comprises a mutation selected from the group consisting of: at one of L53I, L56I, L80I, and L118I and the mutations of V69A, Q74P, N88D or N88R, and optionally C125A or C125S.
68. The polypeptide of any one of embodiments 60-67, wherein the IL-2 mutein comprises a L53I mutation.
69. The polypeptide of any one of embodiments 60-67, wherein the IL-2 mutein comprises a L56I mutation.
70. The polypeptide of any one of embodiments 60-67, wherein the IL-2 mutein comprises a L80I mutation.
71. The polypeptide of any one of embodiments 60-67, wherein the IL-2 mutein comprises a L118I mutation.
72. The polypeptide of any one of embodiments 60-67, wherein the IL-2 mutein does not comprises any other mutations.
73. The polypeptide of any one of embodiments 60-67, wherein the Fc protein comprises L247A, L248A, and G250A mutations, or a L234A mutation, a L235A mutation, and/or a G237A mutation according to Kabat numbering.
74. The polypeptide of any one of embodiments 60-67, wherein the Linker comprises a sequence of GGGGSGGGGSGGGGS (SEQ ID NO: 30) or GGGGSGGGGSGGGGSGGGGS (SEQ ID NO: 22).
75. The polypeptide of any of embodiments 1-74, wherein the skin targeting moiety is an antibody that binds to desmoglein-1, desmoglein-2, desmoglein-3, or desmoglein-4.
76. The polypeptide of embodiment 75, wherein the antibody that binds to desmoglein-1, desmoglein-2, desmoglein-3, and/or desmoglein-4 comprises as a sequence as provided for herein.
77. The polypeptide of any of the preceding embodiments, wherein the PD-1 agonist is an antibody selected from the PD-1 Antibody Table.
78. A method of treating a subject with inflammatory bowel disease, GVHID, or a skin auto-immune disorder as provided herein, the method comprising administering a therapeutically effective amount of the polypeptide of any of embodiments 1-77 to the subject to treat the inflammatory bowel disease.
79. The method of embodiment 78, wherein the subject with inflammatory bowel disease has Crohn's disease.
80. The method of embodiment 78, wherein the subject with inflammatory bowel disease has ulcerative colitis.
81. A method of treating a subject with an auto-immune skin disorder, the method comprising administering a therapeutically effective amount of the polypeptide of any of embodiments 1-77 to the subject to treat the disorder.
82. A method of treating GVHD comprising administering a therapeutically effective amount of the polypeptide of any of embodiments 1-77 to the subject to treat the GVHD.
83. A method of treating GVHD of the skin, comprising administering a therapeutically effective amount of the polypeptide of any of embodiments 1-77 to the subject to treat the GVHD of the skin.
84. A method of treating a transplant subject comprising administering a therapeutically effective amount of the polypeptide of any of embodiments 1-77 to the subject, thereby treating a transplant (recipient) subject.
85. A method of treating GVHD in a subject having a transplanted a donor tissue (e.g. skin tissue) comprising administering a therapeutically effective amount of the polypeptide of any of embodiments 1-77 to the subject.
86. A method of treating a subject having, or at risk, or elevated risk, for having, an autoimmune disorder, comprising administering a therapeutically effective amount of the polypeptide of any embodiments 1-77, thereby treating the subject.
87. The method of any of embodiments 78-86, wherein the subject has an autoimmune disorder and the therapeutically effective amount of the polypeptide does not comprise an autoantigenic peptide or polypeptide characteristic of the autoimmune disorder, e.g., does not comprise a peptide or polypeptide against which the subject has autoantibodies.
88. A nucleic acid encoding a therapeutic compound of any of embodiments 1-77.
89. A vector comprising the nucleic acid of embodiment 88.
90. A cell comprising the nucleic acid of embodiment 88 or the vector of embodiment 89.
91. A method of making a polypeptide comprising culturing a cell of embodiment 90 to make the therapeutic compound.
92. A method of making a nucleic acid sequence encoding a polypeptide of embodiment 1-77, comprising
   a) providing a vector comprising sequence encoding a targeting moiety and inserting into the vector sequence encoding an effector binding/modulating moiety to form a sequence encoding a therapeutic compound; or
   b) providing a vector comprising sequence encoding an effector binding/modulating moiety and inserting into the vector sequence encoding a targeting moiety to form a sequence encoding a therapeutic compound,
thereby making a sequence encoding a therapeutic compound.
93. A protein comprising a sequence as provided herein that binds desmoglein-1, desmoglein-2, desmoglein-3, or desmoglein-4.
94. An antibody that binds to desmoglein-1, desmoglein-2, desmoglein-3, or desmoglein-4, wherein the antibody comprises a sequence as provided herein.
95. A pharmaceutical composition comprising a protein of embodiment 93 or an antibody of embodiment 94.
96. A protein comprising a sequence as provided in the Desmoglein Antibody Table 1 and 2.
97. An antibody that binds to desmoglein, wherein the antibody comprises a sequence as provided in the Desmoglein Antibody Table 1 and 2.
98. The antibody of embodiment 97, or antigen binding fragment thereof, wherein the antibody, or antigen binding fragment thereof, comprises:
   (i) a heavy chain variable region comprising heavy chain CDR1, CDR2, and CDR3 sequences, wherein the heavy chain CDR1 sequence has the amino acid sequence of any of the CDR1 sequences set forth in the Desmoglein Antibody Table 1 and 2; the heavy chain CDR2 has the amino acid sequence of any of the CDR2 sequences set forth in the Desmoglein Antibody Table 1 and 2, and the heavy chain CDR3 has the amino acid sequence of any of the CDR3 sequences set forth in the Desmoglein Antibody Table 1 and 2, or variants of any of the foregoing; and
   (ii) a light chain variable region comprising light chain CDR1, CDR2, and CDR3 sequences, wherein the light chain CDR1 sequence has the amino acid sequence of any of the LCDR1 sequences set forth in the Desmoglein Antibody Table 1 and 2; the light chain LCDR2 has the amino acid sequence of any of the LCDR2 sequences set forth in the Desmoglein Antibody Table 1 and 2, and the light chain CDR3 has the amino acid sequence of any of the LCDR3 sequences set forth in the Desmoglein Antibody Table 1 and 2, or variants of any of the foregoing.
99. The antibody of embodiment 97, or antigen binding fragment thereof, wherein the antibody comprises a V_{K} sequence as shown in the Desmoglein Antibody Table 1 and 2.
100. The antibody of embodiment 97, or antigen binding fragment thereof, wherein the antibody comprises a V_{H} sequence as shown in the Desmoglein Antibody Table 1 and 2.
101. The antibody of embodiment 97, or antigen binding fragment thereof, wherein the antibody comprises a V_{K} sequence as shown in the Desmoglein Antibody Table 1 and 2 and a V_{H} sequence as shown in the Desmoglein Antibody Table 1 and 2.
102. A pharmaceutical composition comprising a protein of embodiment 96 or an antibody of any one of embodiments 97-101.
103. A protein comprising a peptide that binds to desmoglein-1, desmoglein-2, desmoglein-3, and/or desmoglein-4 and a peptide that binds to PD-1.
104. The protein of embodiment 103, wherein the peptide that binds to desmoglein-1, desmoglein-2, desmoglein-3, and/or desmoglein-4 is an antibody.
105. The protein of embodiment 103 or 104, wherein the antibody that binds to desmoglein-1, desmoglein-2, desmoglein-3, and/or desmoglein-4 is an antibody of embodiment 75.
106. The protein of embodiment 103, wherein the peptide that binds to PD-1 is an antibody.
107. The protein of embodiment 106, wherein the PD-1 antibody is a PD-1 agonist.
108. The protein of embodiments 106, wherein the PD-1 antibody is an antibody as provided for herein.
109. A protein comprising a peptide that binds to desmoglein-1, desmoglein-2, desmoglein-3, and/or desmoglein-4 and an IL-2 mutein molecule.
110. The protein of embodiment 109, wherein the protein has the sequence as provided in the DSG-TL-2 Table.
111. The protein of embodiment 109, wherein the peptide that binds to desmoglein desmoglein-1, desmoglein-2, desmoglein-3, and/or desmoglein-4 is an antibody.
112. The protein of embodiment 109 or 110, wherein the antibody that binds to desmoglein-1, desmoglein-2, desmoglein-3, and/or desmoglein-4 is an antibody of any one of embodiments 97-101.
113. The protein of embodiments 109 or 110, wherein the IL-2 mutein has the sequence as provided for herein.
114. A protein comprising a peptide that binds to desmoglein-1, desmoglein-2, desmoglein-3, and/or desmoglein-4 and a CD39 molecule.
115. The protein of embodiment 114, wherein the protein has the sequence as provided in the DSG-CD39 Table 1 and 2.
116. The protein of any one of embodiments 114 or 115, wherein the protein has the sequence as set forth in CD39DSGAB1-CD39DSGAB44.
117. The protein of embodiment 114, wherein the peptide that binds to desmoglein-1, desmoglein-2, desmoglein-3, and/or desmoglein-4 is an antibody.
118. The protein of any one of embodiments 114 or 115, wherein the antibody that binds to desmoglein-1, desmoglein-2, desmoglein-3, and/or desmoglein-4 is an antibody of any one of embodiments 97-101.
119. The protein of any one of embodiments 114 or 115, wherein the CD39 molecule has the sequence as provided for herein.
120. A pharmaceutical composition comprising a polypeptide of any one of embodiments 1-77, or a protein of any one of embodiments 103-119.
121. A pharmaceutical composition comprising any peptide comprising a sequence as provided for herein.
122. A method of treating a subject with inflammatory disorder, such as GVHD or a skin disorder, the method comprising administering a therapeutically effective amount of a protein, an antibody, or a pharmaceutical composition of any of embodiments 93-119 to the subject to treat the disorder.
123. The method of embodiment 122, wherein the skin disorder is vitiligo or alopecia.
124. A method of treating a transplant subject, such as a skin transplant, the method comprising administering a therapeutically effective amount of a protein, an antibody, or a pharmaceutical composition of any of embodiments 93-119 to the subject, thereby treating the transplant subject.
125. A method of treating GVHD in a subject having a transplanted a donor tissue comprising administering therapeutically effective amount of a protein, an antibody, or a pharmaceutical composition of any of embodiments 93-119 to the subject.
126. A method of treating a subject having, or at risk, or elevated risk, for having, an autoimmune disorder, comprising administering a protein, antibody, or pharmaceutical composition of any of embodiments 74-103, thereby treating the subject.
127. The method of any of embodiments 122-126, wherein the subject has an autoimmune disorder and the therapeutic compound does not comprise an autoantigenic peptide or polypeptide characteristic of the autoimmune disorder, e.g., does not comprise a peptide or polypeptide against which the subject has autoantibodies.
128. The polypeptide of any one of embodiments 1-77, wherein the polypeptide reduces melanocyte-specific T cells and/or epidermis resident memory T cells.
129. The polypeptide of embodiment 128, wherein the polypeptide reduces melanocyte-specific T cells.
130. The polypeptide of embodiment 128, wherein the polypeptide reduces resident memory T cells.
131. The polypeptide of embodiments 129 or 130, wherein the reduction is selective.
132. A method of reducing melanocyte-specific T cells and/or resident memory T cells in a subject in need thereof, the method comprising administering a therapeutically effective amount of a polypeptide of any one of embodiments 1-77 to the subject.
133. A method of reducing melanocyte-specific T cells and/or resident memory T cells in a subject in need thereof, the method comprising administering therapeutically effective amount of a protein, an antibody, or a pharmaceutical composition of any of embodiments 93-119 to the subject.
134. The method of embodiments 132 or 133, wherein the reduction is selective.

The following examples are illustrative, but not limiting, of the compounds, compositions and methods described herein. Other suitable modifications and adaptations known to those skilled in the art are within the scope of the following embodiments.

### EXAMPLES

### Example 1: CD39 and/or CD73 as Effector Domains Creating a Purinergic Halo Surrounding a Cell Type or Tissue of Interest

A catalytically active fragment of CD39 and/or CD73 is fused to a targeting domain. Upon binding and accumulation at the target site, CD39 phosphohydrolyzes ATP to AMP. Upon binding and accumulation at the target site, CD73 dephosphorylates extracellular AMP to adenosine. A soluble catalytically active form of CD39 suitable for use herein has been found to circulate in human and murine blood, see, e.g., Yegutkin et al FASEB J. 2012 September; 26(9):3875-83. A soluble recombinant CD39 fragment is also described in Inhibition of platelet function by recombinant soluble ecto-ADPase/CD39, Gayle et al J Clin Invest. 1998 May 1; 101(9): 1851-1859. A suitable CD73 molecule comprises a soluble form of CD73 which can be shed from the membrane of endothelial cells by proteolytic cleavage or hydrolysis of the GPI anchor by shear stress see, e.g., reference: Yegutkin G, Bodin P, Burnstock G. Effect of shear stress on the release of soluble ecto-enzymes ATPase and 5′-nucleotidase along with endogenous ATP from vascular endothelial cells. Br J Pharmacol 2000; 129: 921-6.

The local catalysis of ATP to AMP or AMP to adenosine will deplete local energy stores required for fulminant T effector cell function. Treg function should not be impacted by ATP depletion due to their reliance on oxidative phosphorylation for energy needs (which requires less ATP), wherein T memory and other effector cells should be impacted due their reliance on glycolysis (requiring high ATP usage) for fulminant function.

### Example 2: Skin Tethered Targeting Moieties can be Used to Target a PD-1 Agonist or CD39 Effector Domain and Treat Immune Disorders

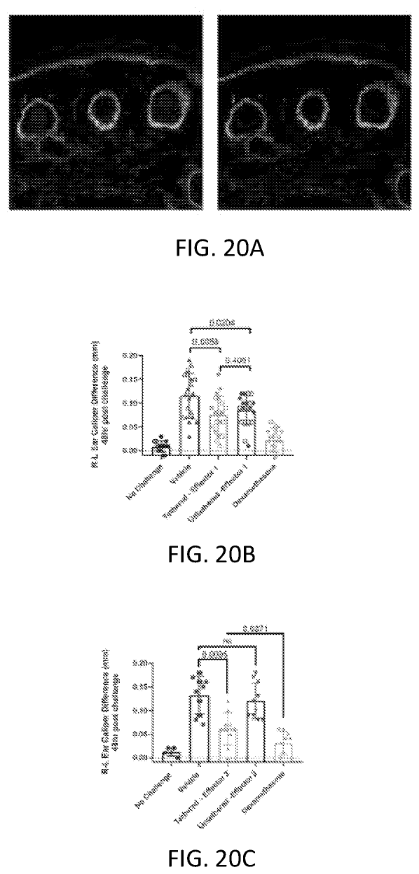

A bi-specific molecule utilizing a skin specific tether was used to localize PD-1 agonist. The targeting moiety was an anti-desmoglein 1 antibody, such as the ones provided herein. FIG. 20A illustrates the specificity of the tether. The reference targeting is shown in the left panel of FIG. 20A and the skin-specific tether utilizing the anti-desmoglein 1 antibody is shown in the right panel of FIG. 20A. The data presented thus far illustrates the specificity of the molecule.

Next, the molecule was tested in BALBc mice. BALBc mice were randomized by weight into test groups. On Day 0 and 1 mice were treated epicutaneously with 20 uL of 0.5% DNFB (4:1 Acetone:Olive Oil) on a shaved area of the abdomen. On day 4 mice were administered test articles (either vehicle, the tethered effector (anti-PD-1 antibody or CD39 Effector Domain tethered to an anti-desmoglein 1 antibody), an untethered PD-1 agonist, or the control dexamethasone) via IV tail vein injection. From day 4-7 mice were treated with Dexamethasone PO at 0.3 mg/kg. On day 5 mice were challenged with 0.2% DNFB on the right ear (10 ul front and back), the left ear was treated with vehicle without DNFB. Caliper measurements of ear thickness were taken using a spring-loaded micrometer caliper (Mitutuyo) on days 5, 6, and 7. On day 7 mice were sacrificed and ears were removed. An 8 mm punch biopsy was taken from each ear and the difference between the weight of the left and right ear biopsies was determined. The results shown in FIG. 20B and FIG. 20C demonstrate that the tethered effector provide a significant effect and reduction in inflammation in a contact hypersensitivity model. These data, illustrated in FIG. 20B and FIG. 20C, demonstrate the efficacy of this tethered molecule.

### Example 3: Desmoglein Tethered PD-1 Agonists Express In Vitro

Proteins were expressed in expi293 (transient expression) following standard protocols. Plasmid vectors carrying the genes for the constructs PD1DSGAB1, PD1DSGAB2, PD1DSGAB3, PD1DSGAB4, PD1DSGAB5, PD1DSGAB6, PD1DSGAB7, PD1DSGAB8, and PD1DSGAB9 were transfected using Gibco ExpiFectamine 293 Transfection Kit (ThermoFisher Scientific) and the cultures were grown at 37 C with shaking (120 rpm), and 8% CO2 for 4 days. Cultures were harvested by centrifugation (3500 rpm, 4 C, 45 minutes) and purified using protein affinity chromatography. Protein concentrations were measured using absorbance at A280 nm. PD1DSGAB1, PD1DSGAB2, PD1DSGAB3, and PD1DSGAB4 showed yields of 70, 78.7, 100, and 72 mg/L (respectively). PD1DSGAB5, PD1DSGAB6, PD1DSGAB7, and PD1DSGAB8 showed yields of 249, 63, 210, and 200 mg/L. PD1DSGAB9 showed yield of 30 mg/L. Accordingly, desmoglein tethered PD-1 agonists are expressed in vitro.

### Example 4: Desmoglein Tethered PD-1 Agonists are Monodisperse

20 μl of 1 mg/ml of the PD1DSGAB1, PD1DSGAB2, PD1DSGAB3, and PD1DSGAB4 was injected into an Agilent AdvanceBio SEC 300 Å 4.6.×150 mm column. The observed elution time was used to extrapolate the molecular weight of the sample from a standard curve generated from proteins of known molecular weight. The percentage of “protein of interest (% POI)” was calculated by integrating the signal of the peak with the expected elution time. The data shows % POI of 99% for PD1DSGAB1, 98% for PD1DSGAB2, 97% for PD1DSGAB3, and 97.8% for PD1DSGAB4. Accordingly, PD1DSGAB1, PD1DSGAB2, PD1DSGAB3, and PD1DSGAB4 are monodisperse.

### Example 5: PD1DSGAB1 Binds Desmoglein-1, -2, -3, and -4

Binding kinetics were measured in order to assess the affinity of constructs to the target: human, mouse, and cyno desmogleins. Anti-human Fc biosensors were equilibrated in assay buffer (PBS, pH 7.4 with 1% BSA, and 0.05% Tween-20) for 30 minutes before the experiment was set-up using an Octet Red QK. Test articles were diluted to 5 ug/mL in assay buffer. A two-fold, seven-point dilution of desmoglein-1, -2, -3, and -4 (starting at 300 nM as the highest concentration) was made. Test articles were captured using anti-human Fc biosensors for 180 s. Biosensors loaded with test articles were equilibrated in assay buffer for 120 s; association was performed for 180 seconds and dissociation was performed for 300 s. Kinetic parameters (kon and kdis) and dissociation constant (K_{D}) were calculated from a 1:1 global fit model using the data analysis software of the Octet96 RED software version 10. PD1DSGAB1 binds all four desmogleins.

### Example 6: PD1DSGAB1 Binds Cyno Desmogleins

Binding of PD1DSGAB1 to cyno desmogleins was measured by ELISA. An immunosorbent plate was coated with cyno desmogleins at a concentration of 2 μg/mL in PBS pH 7.4 and incubated overnight at 4° C. Wells were washed with PBS pH 7.4 containing 0.05% Tween-20 (wash buffer) three times, and then blocked with 1% BSA in PBS pH 7.4 (block buffer) for two hours at room temperature. After 3 washes with wash buffer, a two-fold dilution of PND00377 starting at 300 nM in assay buffer (PBS, pH 7.4 containing 1% BSA and 0.05% Tween-20) was added and incubated for 1 hour at room temperature. After three washes with wash buffer, a donkey anti-human FcY HRP conjugated polyclonal antibody, diluted to 1:5000 in assay buffer, was added to the plate for 1 hr at room temperature. After three washes with wash buffer and three washes with PBS, pH 7.4, the assay was developed with TMB, and stopped with 1N HCL. OD 450 nm was measured. PD1DSGAB1 bound to cyno DSG-1 with the EC50 of 117.1 nM, cyno DSG-2 with the EC50 of 27.3 nM, cyno DSG-3 with the EC50 of 21.63 nM, and cyno DSG-4 with the EC50 of 15.9 nM. The data showed that PD1DSGAB1 binds to cyno desmogleins.

### Example 7: Desmoglein Tethered PD-1 Agonists Bind Human PD-1 with Nanomolar Affinity

Affinity of the PD-1 effector arm to human PD-1 was measured using anti-human Fc biosensors. Said anti-human Fc biosensors were equilibrated in assay buffer (PBS, pH 7.4 with 1% BSA, and 0.05% Tween-20) for 30 minutes before the experiment was set-up. Test articles, PD1DSGAB1, PD1DSGAB2, PD1DSGAB3, and PD1DSGAB4, were diluted to 5 ug/mL in assay buffer. A two-fold, seven-point dilution of human PD-1 (starting at 500 nM as the highest concentration) was made. Test articles were captured using anti-human Fc biosensors for 180 s. Biosensors loaded with test articles were equilibrated in assay buffer for 120 s; association was performed for 180 seconds and dissociation was performed for 300 s. Kinetic parameters (kon and kdis) and dissociation constant (KD) were calculated from a 1:1 global fit model using the data analysis software of the Octet96 RED software version 10. The data showed: Kon of 1.98E+05 l/ms, Koff of 8.25E+03 l/s, and KD of 123 nM for PD1DSGAB1; Kon of 1.46E+05 l/ms, Koff of 5.49E+03 l/s, and KD of 172 nM for PD1DSGAB2; Kon of 7.80E+04 l/ms, Koff of 2.23E+03 l/s, and KD of 328 nM for PD1DSGAB3; and Kon of 8.65E+04 l/ms, Koff of 2.08E+03 l/s, and KD of 243 nM for PD1DSGAB4. Accordingly, PD1DSGAB1, PD1DSGAB2, PD1DSGAB3, and PD1DSGAB4 bind human PD-1 with nanomolar affinity.

### Example 8: Desmoglein Tethered PD-1 Agonists do not Show Nonspecific Binding to DNA or Insulin

Non-specific DNA and Insulin binding (a measure of polyreactivity), which may be predictive of poor PK, was measured. An immunosorbent plate was coated with dsDNA at a concentration of 1 μg/mL or Insulin at 5 μg/mL in PBS pH 7.4 and incubated overnight at 4° C. Wells were washed with PBS pH 7.4 containing 0.05% Tween-20 (wash buffer) three times, and then blocked with 1% BSA in PBS pH 7.4 (block buffer) for two hours at room temperature. After three washes with wash buffer, TAs and controls Lenzilumab and Elotuzumab were diluted to 100 nM in PBS, pH 7.4 containing 1% BSA and 0.05% Tween-20 (assay buffer). The diluted material was added to the DNA/insulin coated plate for 1 hour at room temperature. After three washes with wash buffer, a donkey anti-human FcY HRP conjugated polyclonal antibody, diluted to 1:5000 in assay buffer, was added to the plate for 1 hr at room temperature. After three washes with wash buffer and three washes with wash buffer (with no tween-20), the assay was developed with TMB, and stopped with 1N HCL. OD 450 nm was measured. PD1DSGAB1 showed dsDNA polyreactivity score of 1.4, and Insulin polyreactivity score of 1.3; PD1DSGAB2 showed dsDNA polyreactivity score of 1.7, and Insulin polyreactivity score of 2.2; PD1DSGAB3 showed dsDNA polyreactivity score of 1.1, and Insulin polyreactivity score of 1.7; Elotuzumab control (negative) showed dsDNA polyreactivity score of 1.3, and Insulin polyreactivity score of 2.2; and Lenzilumab control (positive) showed dsDNA polyreactivity score of 56, and Insulin polyreactivity score of 18. PD1DSGAB1, PD1DSGAB2, and PD1DSGAB3 are not polyreactive.

### Example 9: Desmoglein Tethered PD-1 Agonists do not Self Associate

Increased self-association can lead to aggregation and higher viscosity. Accordingly, self association was measured. Gold nanoparticles (15705; Ted Pella Inc.) were coated with a mixture of 80% anti-human goat IgG Fc (109-005-098; Jackson ImmunoResearch) and 20% polyclonal goat non-specific antibody (005-000-003; Jackson ImmunoResearch). The test articles were then incubated with the coated gold particles for 2 h and the wavelength shift was measured using Molecular Devices SpectraMax M2 with SoftMax Pro6 software. The self-interacting antibodies show a higher wavelength shift away from the PBS sample. PD1DSGAB1 showed DIMax of 2 nm, PD1DSGAB2 showed DIMax of 0.1 nm, PD1DSGAB3 showed DIMax of 2 nm, Matuzumab control (negative) showed DIMax of 0 nm, and Infliximab control (positive) showed DIMax of 10 nm. PD1DSGAB1, PD1DSGAB2, and PD1DSGAB3 do not self associate.

### Example 10: Verification of Desmoglein Tethered PD-1 Agonists Identity Via Mass Spectroscopy

PD1DSGAB1, PD2DSG2, and PD1DSGAB3 samples were denatured and reduced by guanidine and DTT. Each sample was analyzed by Waters ACQUITY UPLC coupled to Xevo G2-XS QTOF mass spectrometer using an ACQUITY UPLC Protein BEH SEC column. Mass spectroscopy showed single dominant peak for each sample, which is indicative of accurate molecule weight and expected glysosylation patterns.

### Example 11: Desmoglein Tethered PD-1 Agonists are Stable

PD1DSGAB1 at 5 mg/mL was incubated at 4 and 37 C for up to 28 days to analyze molecule's stability over time. Samples were collected at various time points and then analyzed by size exclusion chromatography (Agilent BioAdvance SEC 300 A column) and dynamic light scattering (UnCLE). PD1DSGAB1 was stable with no loss of peak intensity or accumulation of high or low molecular weight species at 4 C and 37 C for 1 month. Monodispersity of the samples in DLS measurement was constant at 100% and thermal stability (Tm) did not change over time.

### Example 11: PD1DSGAB1 Binds to HaCaT Cells Expressing Desmoglein

HaCaT cells were plated for 24 hours at 37 C. After 24 hours, plates were blocked for 1 hour and test articles were added and incubated with anti-human IgG and binding of test articles to the target was assessed by immunofluorescence imaging. PD1DSGAB1 bound to Desmoglein on HaCaT (Human Keratinocyte) cells as was evident by uniform staining on the cell membrane/borders of the keratinocyte monolayers (data not shown).

### Example 12: PD1DSGAB1 Localizes to Mouse Skin Expressing Desmoglein

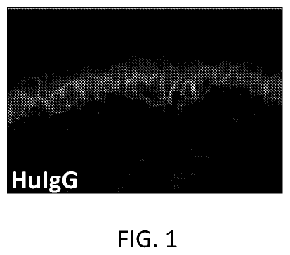

Balb/c mice were intravenously dosed with 10 mg/Kg of bifunctional antibody and skin was harvested 24 hours post dosing. Frozen sections were fixed, blocked and incubated with fluorophore-conjugated anti-human IgG for overnight at 4 C. Tissue was counterstained with DAPI for nuclei staining and mounted with Prolong gold anti-fade mounting agent. Binding of test articles to the skin was assessed by fluorescence imaging microscope. PD1DSGAB1 bound to Desmoglein on mouse tail skin as was evident by staining on the cell surface of keratinocytes throughout the epidermis and as shown in FIG. 1.

### Example 13: PD1DSGAB1 Shows Agonist Activity

Plates were coated with anti-human IgG, blocked, and test articles were added at the indicated concentrations for 1 hour. Plates were washed and PD-1 reporter Jurkat cells were added. SHP-2 recruitment was assessed after 2 hours. PD1DSGAB1 showed EC50 of 106.7 nM at 2 hours, and 11.23 nM at 24 hours, while TTJ2 showed no binding at 2 or 24 hours. PD1DSGAB1 has agonist activity in IgG tethered format.

### Example 14: PD1DSGAB1 Shows No Antagonist Activity

PD-1 reporter Jurkat cells were incubated with the indicated concentrations of test articles for 1 hour. PD-L1 expressing cells were then added and SHP-2 recruitment was assessed after 2 hours. PD1DSGAB1 and TTJ2 showed no detectable antagonist activity, while Pembrolizumab control showed IC50 of 0.02039 nM. Accordingly, PD1DSGAB1 has no antagonist activity.

### Example 15: PD1DSGAB1 Prevents Skin De-Pigmentation

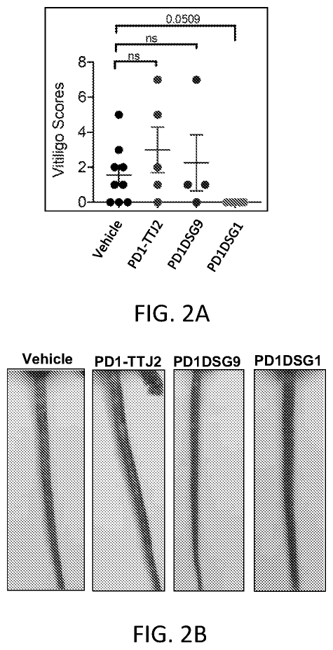

The ability of PD1DSGAB1 to prevent skin de-pigmentation was measured in a mouse model of Vitiligo. Sublethally irradiated Krt14-Kitl* mice were adoptively transferred with PMEL CD8+ T cells isolated from PMEL TCR transgenic mice and treated once per week for five weeks with vehicle or bispecific antibody starting week two post disease induction. Mice were scored for depigmentation during euthanizing at week 7. PD1DSGAB1 prevented skin de-pigmentation as evident from vitiligo de-pigmentation score of 0.1429±0.142 in a mouse model of Vitiligo and as shown in FIG. 2A and FIG. 2B. PD1DSGAB9 contains the does not recognize mouse PD-1 and did not have any activity in the Vitiligo mouse model (depigmentation score of 2.25±1.6). Similarly, vehicle depigmentation score was 1.643±0.509, and PD1-TTJ2 score was 1.8±0.757.

Skin and spleens were subsequently harvested and processed for flow cytometry to assess the immune cell amounts following treatment with the skin-tethered PD-1 agonists. The data showed selective reduction of epidermis melanocyte-specific T cells (PMEL) and resident memory T cells (Trm) in animals treated with PD1DSGAB1 only. Additionally, treatment with PD1DSGAB1 did not result in reduction of host T cells.

Accordingly, PD1DSGAB1 prevents skin de-pigmentation and selectively reduces PMEL and Trm in the skin with no observable systemic effects.

### Example 16: Desmoglein-1 scFab Tethered to the Heavy Chain of the PD-1 Agonists are Monodisperse, while Those Tethered to the Light Chain of the PD-1 Agonists Aggregate

20 μl of 1 mg/ml of the PD1DSGAB5, PD1DSGAB6, PD1DSGAB7, and PD1DSGAB8 was injected into an Agilent AdvanceBio SEC 300 Å 4.6.×150 mm column. The observed elution time was used to extrapolate the molecular weight of the sample from a standard curve generated from proteins of known molecular weight. The percentage of “protein of interest (% POI)” was calculated by integrating the signal of the peak with the expected elution time. The data shows % POI of 96.4% for PD1DSGAB5, 100% for PD1DSGAB6, 50.7% for PD1DSGAB7, and 63.8% for PD1DSGAB8. Accordingly, PD1DSGAB5 and PD1DSGAB6 are monomeric, while PD1DSGAB7 and PD1DSGAB8 have high levels of high molecular weight species or aggregates.

### Example 17: SEC-MALS Measurement of Molecular Weight of PD1DSGAB6

20 μl of 1 mg/ml samples were injected into an Agilent AdvanceBio SEC 300 Å 4.6.×150 mm column connected to a Multi-Angle-Light Scattering instrument and a differential refractometer. Data was analyzed using the Astra software from Wyatt. SEC-MALS showed correct molecular weight of 277 kDa for PD1DSGAB6.

### Example 18: PD1DSGAB9 is Monodisperse

20 μl of 1 mg/ml of the PD1DSGAB9 was injected into an Agilent AdvanceBio SEC 300 Å 4.6.×150 mm column. The observed elution time was used to extrapolate the molecular weight of the sample from a standard curve generated from proteins of known molecular weight. The percentage of “protein of interest (% POI)” was calculated by integrating the signal of the peak with the expected elution time. The data shows % POI of 99%. PD1DSGAB9 is highly monomeric.

### Example 19: PD1DSGAB9 Binds Human DSG-3

Anti-human Fc biosensors were equilibrated in assay buffer (PBS, pH 7.4 with 1% BSA, and 0.05% Tween-20) for 30 minutes before the experiment was set-up. Test articles were diluted to 5 ug/mL in assay buffer. A two-fold, seven-point dilution of human DSG-3 (starting at 300 nM as the highest concentration) was made. Test articles were captured using anti-human Fc biosensors for 180 s. Biosensors loaded with test articles were equilibrated in assay buffer for 120 s; association was performed for 180 seconds and dissociation was performed for 300 s. Kinetic parameters (kon and kdis) and dissociation constant (KD) were calculated from a 1:1 global fit model using the data analysis software of the Octet96 RED software version 10. The data showed Kd value of 29 nM, Kon value of 2.57E0+04 l/ms, and Kdis value of 7.57E-04 l/s. Accordingly, PD1DSGAB9 binds human DSG-3.

### Example 20: SEC-MALS Measurement of Molecular Weight of PD1DSGAB9

20 μl of 1 mg/ml samples were injected into an Agilent AdvanceBio SEC 300 Å 4.6.×150 mm column connected to a Multi-Angle-Light Scattering instrument and a differential refractometer. Data was analyzed using the Astra software from Wyatt. SEC-MALS showed high monodispersity and correct molecular weight of 200 kDa for PD1DSGAB9.

### Example 21: PD1DSGAB9 Binds to HaCaT Cells Expressing Desmoglein

HaCaT cells were plated for 24 hours at 37 C. After 24 hours, plates were blocked for 1 hour and test articles were added and incubated with anti-human IgG and binding of test articles to the target was assessed by immunofluorescence imaging. PD1DS91 bound to Desmoglein on HaCaT (Human Keratinocyte) cells as was evident by uniform staining on the cell membrane/borders of the keratinocyte monolayers (data not shown).

### Example 22: PD1DSGAB9 Shows Agonist Activity

Plates were coated with anti-human IgG, blocked, and test articles were added at the indicated concentrations for 1 hour. Plates were washed and PD-1 reporter Jurkat cells were added. SHP-2 recruitment was assessed after 2 hours. PD1DSGAB9 showed EC50 of 21.22 nM at 2 hours, and 16.58 nM at 24 hours, while TTJ2 showed no binding at 2 or 24 hours. PD1DSGAB9 has agonist activity in IgG tethered format.

### Example 23: PD1DSGAB9 Shows No Antagonist Activity

PD-1 reporter Jurkat cells were incubated with the indicated concentrations of test articles for 1 hour. PD-L1 expressing cells were then added and SHP-2 recruitment was assessed after 2 hours. PD1DSGAB9 and TTJ2 showed no detectable antagonist activity, while Pembrolizumab control showed IC50 of 0.02039 nM. Accordingly, PD1DSGAB9 has no antagonist activity.

### Example 24: PD1DSGAB9 Inhibits Skin Inflammation in Humanized Imiquimod-Induced Psoriasis Model

Mice were engrafted with human CD34+ stem cells, challenged with imiquimod starting day 1 until day 7. Mice were treated on day 1 with vehicle or bispecific antibody at 3 mg/Kg. Mice were scored for PASI (Psoriasis Area and Severity Index) until euthanized at day 7. PD1DSGAB9 showed mean PASI score of 2.375±0.182, PD1-TTJ2 showed mean PASI score of 3.625+0.263, vehicle showed mean PASI score of 4+0.341, while Tacrolimus control showed mean PASI score of 0.769+0.121. PD1DSGAB9 inhibits skin inflammation as evident from mean PASI scores in a humanized imiquimod-induced model of psoriasis.

### Example 25: Desmoglein Tethered IL-2 Muteins Express In Vitro

Proteins were expressed in expi293 (transient expression) following standard protocols. Plasmid vectors carrying the genes for the constructs IL2DSGAB1, IL2DSGAB2, IL2DSGAB3, and IL2DSGAB4 were transfected using Gibco ExpiFectamine 293 Transfection Kit (ThermoFisher Scientific) and the cultures were grown at 37 C with shaking (120 rpm), and 8% CO2 for 4 days. Cultures were harvested by centrifugation (3500 rpm, 4 C, 45 minutes) and purified using protein A affinity chromatography. Protein concentrations were measured using absorbance at A280 nm. IL2DSGAB1, IL2DSGAB2, IL2DSGAB3, and IL2DSGAB4 showed yields of 64, 84.4, 75.2, and 66 mg/L (respectively). IL2DSGAB5, IL2DSGAB6, IL2DSGAB7, and IL2DSGAB8 showed yields of 78.33, 68, 35.33, and 72.67 mg/L (respectively).

Accordingly, desmoglein tethered IL-2 muteins are expressed in vitro.

### Example 26: IL2DSGAB1, IL2DSGAB2, IL2DSGAB3, and IL2DSGAB4 are Monodisperse

20 μl of 1 mg/ml of the PD1DSGAB9 was injected into an Agilent AdvanceBio SEC 300 Å 4.6.×150 mm column. The observed elution time was used to extrapolate the molecular weight of the sample from a standard curve generated from proteins of known molecular weight. The percentage of “protein of interest (% POI)” was calculated by integrating the signal of the peak with the expected elution time. The data shows % POI of 98.8% for IL2DSGAB1, 98.7% for IL2DSGAB2, 100% for IL2DSGAB3, and 98% for IL2DSGAB4. Accordingly, IL2DSGAB1, IL2DSGAB2, IL2DSGAB3, and IL2DSGAB4 are highly monomeric.

### Example 27: IL2DSGAB1 Binds DSG-1, DSG-2, DSG-3, and DSG-4

Anti-human Fc biosensors were equilibrated in assay buffer (PBS, pH 7.4 with 1% BSA, and 0.05% Tween-20) for 30 minutes before the experiment was set-up. Test articles were diluted to 5 ug/mL in assay buffer. A two-fold, seven-point dilution of human DSG-3 (starting at 300 nM as the highest concentration) was made. Test articles were captured using anti-human Fc biosensors for 180 s. Biosensors loaded with test articles were equilibrated in assay buffer for 120 s; association was performed for 180 seconds and dissociation was performed for 300 s. Kinetic parameters (kon and kdis) and dissociation constant (KD) were calculated from a 1:1 global fit model using the data analysis software of the Octet96 RED software version 10. The data showed Kd value of 13 nM for DSG-1, 4.2 nM for DSG-2, 19 nM for DSG-3, and 10 nM for DSG-4. IL2DSGAB1 shows affinity to all four desmogleins.

### Example 28: Bifunctionals with IL-2 Mutein Fused to the Heavy Chain of SD1 Bind Human CD25 and Human DSG-3 Simultaneously

An immunosorbent plate was coated with dsDNA at a concentration of 1 μg/mL hDSG-3 in PBS pH 7.4 and incubated overnight at 4° C. Wells were washed with PBS pH 7.4 containing 0.05% Tween-20 (wash buffer) three times, and then blocked with 6% milk in PBS pH 7.4 (block buffer) for two hours at room temperature. After three washes with wash buffer, 1 nM, 10 nM, and 100 nM of the test articles in assay buffer (PBS, pH 7.4 containing 1% BSA and 0.05% Tween-20) was added and incubated for 1 hr. After three washes with wash buffer 1 μg/mL of his-tagged hCD25 was added and incubated for 1 hr. Detected with anti anti-His-HRP conjugated antibody (1:10,000 dilution). IL2DSGAB1 showed absorbance (OD450 nm) of 1.3 at 100 nM, 0.78 at 10 nM, and 0.31 at 1 nM; IL2DSGAB2 showed absorbance of 1.02 at 100 nM, 0.75 at 10 nM, and 0.26 at 1 nM; IL2DSGAB3 showed absorbance of 0.756 at 100 nM, 0.628 at 10 nM, and 0.508 at 1 nM; and IL2DSGAB4 showed absorbance of 1.16 at 100 nM, 0.545 at 10 nM, and 0.194 at 1 nM. Accordingly, the data shows that IL2DSGAB1, IL2DSGAB2, IL2DSGAB3, and IL2DSGAB4 bind human CD25 and human DSG-3 simultaneously.

### Example 29: IL2DSGAB1, IL2DSGAB2, IL2DSGAB3, and IL2DSGAB4 do not Show Nonspecific Binding to DNA or Insulin

Non-specific DNA and Insulin binding (a measure of polyreactivity), which may be predictive of poor PK, was measured. An immunosorbent plate was coated with dsDNA at a concentration of 1 μg/mL or Insulin at 5 μg/mL in PBS pH 7.4 and incubated overnight at 4° C. Wells were washed with PBS pH 7.4 containing 0.05% Tween-20 (wash buffer) three times, and then blocked with 1% BSA in PBS pH 7.4 (block buffer) for two hours at room temperature. After three washes with wash buffer, TAs and controls Lenzilumab and Elotuzumab were diluted to 100 nM in PBS, pH 7.4 containing 1% BSA and 0.05% Tween-20 (assay buffer). The diluted material was added to the DNA/insulin coated plate for 1 hour at room temperature. After three washes with wash buffer, a donkey anti-human FcY HRP conjugated polyclonal antibody, diluted to 1:5000 in assay buffer, was added to the plate for 1 hr at room temperature. After three washes with wash buffer and three washes with wash buffer (with no tween-20), the assay was developed with TMB, and stopped with 1N HCL. OD 450 nm was measured. IL2DSGAB1 showed dsDNA polyreactivity score of 1.7, and Insulin polyreactivity score of 2.2; IL2DSGAB2 showed dsDNA polyreactivity score of 3.5, and Insulin polyreactivity score of 6.3; IL2DSGAB3 showed dsDNA polyreactivity score of 2.6, and Insulin polyreactivity score of 2.9; IL2DSGAB4 showed dsDNA polyreactivity score of 2.3, and Insulin polyreactivity score of 3.1; Elotuzumab control (negative) showed dsDNA polyreactivity score of 1.3, and Insulin polyreactivity score of 2.2; and Lenzilumab control (positive) showed dsDNA polyreactivity score of 56, and Insulin polyreactivity score of 18. IL2DSGAB1, IL2DSGAB2, IL2DSGAB3, and IL2DSGAB4 are not polyreactive.

### Example 30: IL2DSGAB1, IL2DSGAB2, and IL2DSGAB3 Show Favorable Thermal Stability

Samples at 1 mg/ml concentration were denatured with a thermal ramp from 25-95 C with a step of is. DLS measurements were taken at the beginning and at the end of the step. Tm of each sample was calculated using the first derivative of the barycentric mean (BCM) of fluorescence intensity. The Tagg for each sample was calculated using the intensity of scattered light.

IL2DSGAB1 showed Tm1 of 68.3 C, Tm2 of 76.1 C, Tm3 of 86.9 C, and Tagg of 71.2 C; IL2DSGAB2 showed Tm1 of 66.9 C, Tm2 of 74.1 C, Tm3 of 89 C, and Tagg of 72.7 C; and IL2DSGAB3 showed Tm1 of 71.4 C, Tm2 of 80 C, Tm3 of 89.6 C, and Tagg of 71.1 C. The data shows favorable Tagg and thermal stability.

### Example 31: Identity of IL2DSGAB1 Was Verified Via Mass Spectroscopy

IL2DSGAB1 sample was denatured and reduced by guanidine and DTT and then deglycosylated by PNGase F (MEDNA Bio M3104). The sample was analyzed by Waters ACQUITY UPLC coupled to Xevo G2-XS QTOF mass spectrometer using an ACQUITY UPLC Protein BEH SEC column. Mass spectroscopy showed single dominant peak for the IL2DSGAB1 sample, which is indicative of accurate molecule weight and expected glycosylation patterns.

### Example 32: IL2DSGAB1 Binds to HaCaT Cells Expressing Desmoglein

HaCaT cells were plated for 24 hours at 37 C. After 24 hours, plates were blocked for 1 hour and test articles were added and incubated with anti-human IgG and binding of test articles to the target was assessed by immunofluorescence imaging. IL2DSGAB1 bound to Desmoglein on HaCaT (Human Keratinocyte) cells as was evident by uniform staining on the cell membrane/borders of the keratinocyte monolayers (data not shown).

### Example 33: IL2DSGAB1 Localizes to Mouse Skin Expressing Desmoglein

Balb/c mice were intravenously dosed with 10 mg/Kg of bifunctional antibody and skin was harvested 24 hours post dosing. Frozen sections were fixed, blocked and incubated with fluorophore-conjugated anti-human IgG for overnight at 4 C. Tissue was counterstained with DAPI for nuclei staining and mounted with Prolong gold anti-fade mounting agent. Binding of test articles to the skin was assessed by fluorescence imaging microscope. IL2DSGAB1 bound to Desmoglein on mouse tail skin as was evident by staining on the cell surface of keratinocytes throughout the epidermis (data not shown).

### Example 34: IL2DSGAB1 Selectively Activates T Cells

PBMCs were isolated from fresh human whole blood and incubated with test articles or recombinant IL2 (positive control here) at indicated concentrations for 20 minutes. Cells were washed, fixed and permeabilized and stained with anti-human antibodies against CD3, CD4, CD56, CD25, FOXP3, p-STAT5 for 30 minutes. Samples were washed and acquired on flow cytometer. IL2DSGAB1 selectively activated regulatory T cells (Tregs) with the EC50 of 6.447 pM as indicated by % pSTAT5 positive cells, while IL-2 control activated Tregs with the EC50 of 38.94 pM, and Tconv with the EC50 of 176.1 pM. Accordingly, IL2DSGAB1 can selectively activate Tregs but not the conventional T cells and NK cells in soluble format.

### Example 35: IL2DSGAB5, IL2DSGAB6, IL2DSGAB7, and IL2DSGAB8 Show Favorable Thermal Stability

Samples at 1 mg/ml concentration were denatured with a thermal ramp from 25-95 C with a step of is. DLS measurements were taken at the beginning and at the end of the step. Tm of each sample was calculated using the first derivative of the barycentric mean (BCM) of fluorescence intensity. The Tagg for each sample was calculated using the intensity of scattered light. IL2DSGAB5 showed Tm1 of 68.3 C, Tm2 of 76.1 C; TL2DSGAB6 showed Tm1 of 71.1 C, Tm2 of 78.9 C, and Tagg of 73.7 C; IL2DSGAB7 showed Tm1 of 71.5 C, Tm2 of 78.52 C, and Tagg of 73.6 C; and IL2DSGAB8 showed Tm1 of 71.8 C, Tm2 of 80 C, and Tagg of 73.8 C. The data shows favorable Tm and thermal stability.

### Example 36: IL2DSGAB5, IL2DSGAB6, IL2DSGAB7, and IL2DSGAB8 Bind Human CD25

Binding kinetics were measured in order to assess the affinity of constructs to the target: human, mouse, and cyno desmogleins. Anti-human Fc biosensors were equilibrated in assay buffer (PBS, pH 7.4 with 1% BSA, and 0.05% Tween-20) for 30 minutes before the experiment was set-up using an Octet Red QK. Test articles were diluted to 5 ug/mL in assay buffer. A two-fold, seven-point dilution of desmoglein-1, -2, -3, and -4 (starting at 300 nM as the highest concentration) was made. Test articles were captured using anti-human Fc biosensors for 180 s. Biosensors loaded with test articles were equilibrated in assay buffer for 120 s; association was performed for 180 seconds and dissociation was performed for 300 s. Kinetic parameters (kon and kdis) and dissociation constant (K_{D}) were calculated from a 1:1 global fit model using the data analysis software of the Octet96 RED software version 10. IL2DSGAB5 recorded K_{D} of 3.4 nM, Kon of 4.6E+05 l/ms, and Kdis of 1.5E+03; IL2DSGAB6 recorded K_{D} of 3.7 nM, Kon of 4.06E+05 l/ms, and Kdis of 1.53E+03 l/s; IL2DSGAB7 recorded K_{D} of 3.5 nM, Kon of 4.41E+05 l/ms, and Kdis of 1.55E+03 l/s; and IL2DSGAB8 recorded K_{D} of 3.5 nM, Kon of 4.49E+05 l/ms, and Kdis of 1.59E+03 l/s. IL2DSGAB5, IL2DSGAB6, IL2DSGAB7, and IL2DSGAB8 bind human CD25 with nanomolar affinity.

### Example 37: Bifunctionals with IL-2 Mutein Fused to the Light Chain of SD1 Bind Human CD25 and Human DSG-3 Simultaneously

An immunosorbent plate was coated with dsDNA at a concentration of 1 μg/mL hDSG-3 in PBS pH 7.4 and incubated overnight at 4° C. Wells were washed with PBS pH 7.4 containing 0.05% Tween-20 (wash buffer) three times, and then blocked with 6% milk in PBS pH 7.4 (block buffer) for two hours at room temperature. After three washes with wash buffer, 1 nM, 10 nM, and 100 nM of the test articles in assay buffer (PBS, pH 7.4 containing 1% BSA and 0.05% Tween-20) was added and incubated for 1 hr. After three washes with wash buffer 1 μg/mL of his-tagged hCD25 was added and incubated for 1 hr. Detected with anti anti-His-HRP conjugated antibody (1:10,000 dilution). IL2DSGAB5 showed absorbance (OD450 nm) of 0.965 at 100 nM, 0.483 at 10 nM, and 0.27 at 1 nM; IL2DSGAB6 showed absorbance of 1.04 at 100 nM, 0.887 at 10 nM, and 0.26 at 1 nM; TL2DSGAB7 showed absorbance of 1.2 at 100 nM, 0.55 at 10 nM, and 0.36 at 1 nM; and IL2DSGAB8 showed absorbance of 1.00 at 100 nM, 0.41 at 10 nM, and 0.29 at 1 nM. Accordingly, the data shows that TL2DSGAB5, IL2DSGAB6, TL2DSGAB7, and IL2DSGAB8 bind human CD25 and human DSG-3 simultaneously.

### Example 38: IL2DSGAB5 and IL2DSGAB8 Selectively Activate Tregs

PBMCs were isolated from fresh human whole blood and incubated with test articles or recombinant IL2 (positive control here) at indicated concentrations for 20 minutes. Cells were washed, fixed and permeabilized and stained with anti-human antibodies against CD3, CD4, CD56, CD25, FOXP3, p-STAT5 for 30 minutes. Samples were washed and acquired on flow cytometer. IL2DSGAB5 and IL2DSGAB8 selectively activated regulatory T cells (Tregs) with the EC50 of 6.009 pM and 3.165 pM (respectively) as indicated by % pSTAT5 positive cells. Accordingly, IL2DSGAB5 and IL2DSGAB8 can selectively activate Tregs but not the conventional T cells and NK cells in soluble format.

### Example 39: SD1-mouseCD39 Constructs Express In Vitro

Proteins were expressed in expi293 (transient expression) following standard protocols. Plasmid vectors carrying the genes for the constructs CD39DSGAB1, CD39DSGAB2, CD39DSGAB3, CD39DSGAB4, CD39DSGAB5, CD39DSGAB6, CD39DSGAB7, CD39DSGAB8, and CD39DSGAB9 were transfected using Gibco ExpiFectamine 293 Transfection Kit (ThermoFisher Scientific) and the cultures were grown at 37 C with shaking (120 rpm), and 8% CO2 for 4 days. Cultures were harvested by centrifugation (3500 rpm, 4 C, 45 minutes) and purified using protein A affinity chromatography. Protein concentrations were measured using absorbance at A280 nm. CD39DSGAB1, CD39DSGAB2, CD39DSGAB3, CD39DSGAB4, CD39DSGAB5, CD39DSGAB6, CD39DSGAB7, CD39DSGAB8, and CD39DSGAB9 showed yields of 5.3, 4.9, 5.7, 6.1, 5.2, 6.4, 3.9, 2.3, and 4.2 mg/L (respectively). Accordingly, CD39DSGAB1, CD39DSGAB2, CD39DSGAB3, CD39DSGAB4, CD39DSGAB5, CD39DSGAB6, CD39DSGAB7, CD39DSGAB8, and CD39DSGAB9 are expressed in vitro.

### Example 40: SD1-mouseCD39 Constructs are Monodisperse

20 μl of 1 mg/ml of the CD39DSGAB9 was injected into an Agilent AdvanceBio SEC 300 Å 4.6.×150 mm column. The observed elution time was used to extrapolate the molecular weight of the sample from a standard curve generated from proteins of known molecular weight. The percentage of “protein of interest (% POI)” was calculated by integrating the signal of the peak with the expected elution time. The data shows % POI of 100% for CD39DSGAB1, 98% for CD39DSGAB2, 100% for CD39DSGAB3, 100% for CD39DSGAB4, 97% for CD39DSGAB5, 100% for CD39DSGAB6, 100% for CD39DSGAB7, 98% for CD39DSGAB8, and 100% for CD39DSGAB9. Accordingly, CD39DSGAB1, CD39DSGAB2, CD39DSGAB3, CD39DSGAB4, CD39DSGAB5, CD39DSGAB6, CD39DSGAB7, CD39DSGAB8, and CD39DSGAB9 are highly monomeric.

### Example 41: SD1-mouseCD39 Constructs do not Show Nonspecific Binding to DNA or Insulin

Non-specific DNA and Insulin binding (a measure of polyreactivity), which may be predictive of poor PK, was measured. An immunosorbent plate was coated with dsDNA at a concentration of 1 μg/mL or Insulin at 5 μg/mL in PBS pH 7.4 and incubated overnight at 4° C. Wells were washed with PBS pH 7.4 containing 0.05% Tween-20 (wash buffer) three times, and then blocked with 1% BSA in PBS pH 7.4 (block buffer) for two hours at room temperature. After three washes with wash buffer, TAs and controls Lenzilumab and Elotuzumab were diluted to 100 nM in PBS, pH 7.4 containing 1% BSA and 0.05% Tween-20 (assay buffer). The diluted material was added to the DNA/insulin coated plate for 1 hour at room temperature. After three washes with wash buffer, a donkey anti-human FcY HRP conjugated polyclonal antibody, diluted to 1:5000 in assay buffer, was added to the plate for 1 hr at room temperature. After three washes with wash buffer and three washes with wash buffer (with no tween-20), the assay was developed with TMB, and stopped with 1N HCL. OD 450 nm was measured. CD39DSGAB1 showed dsDNA polyreactivity score of 1.2, and Insulin polyreactivity score of 1.4; CD39DSGAB2 showed dsDNA polyreactivity score of 2.0, and Insulin polyreactivity score of 1.8; CD39DSGAB3 showed dsDNA polyreactivity score of 1.5, and Insulin polyreactivity score of 1.3; CD39DSGAB4 showed dsDNA polyreactivity score of 4.1, and Insulin polyreactivity score of 3.8; CD39DSGAB5 showed dsDNA polyreactivity score of 2.1, and Insulin polyreactivity score of 1.6; CD39DSGAB6 showed dsDNA polyreactivity score of 3.6, and Insulin polyreactivity score of 2.7; CD39DSGAB7 showed dsDNA polyreactivity score of 1.7, and Insulin polyreactivity score of 2.1; CD39DSGAB8 showed dsDNA polyreactivity score of 3.2, and Insulin polyreactivity score of 1.8; CD39DSGAB9 showed dsDNA polyreactivity score of 2.9, and Insulin polyreactivity score of 2.6; Elotuzumab control (negative) showed dsDNA polyreactivity score of 1.6, and Insulin polyreactivity score of 1.4; and Lenzilumab control (positive) showed dsDNA polyreactivity score of 70, and Insulin polyreactivity score of 40. CD39DSGAB1, CD39DSGAB2, CD39DSGAB3, CD39DSGAB4, CD39DSGAB5, CD39DSGAB6, CD39DSGAB7, CD39DSGAB8, and CD39DSGAB9 are not polyreactive.

### Example 42: SEC-MALS Measurement of Molecular Weight of CD39DSGAB3

20 μl of 1 mg/ml samples were injected into an Agilent AdvanceBio SEC 300 Å 4.6.×150 mm column connected to a Multi-Angle-Light Scattering instrument and a differential refractometer. Data was analyzed using the Astra software from Wyatt. SEC-MALS showed monodispersity and correct molecular weight of 242 kDa for CD39DSGAB3.

### Example 43: CD39DSGAB3 Binds to HaCaT Cells Expressing Desmoglein

HaCaT cells were plated for 24 hours at 37 C. After 24 hours, plates were blocked for 1 hour and test articles were added and incubated with anti-human IgG and binding of test articles to the target was assessed by immunofluorescence imaging. CD39DSGAB3 bound to Desmoglein on HaCaT (Human Keratinocyte) cells as was evident by uniform staining on the cell membrane/borders of the keratinocyte monolayers (data not shown).

### Example 44: CD39DSGAB3 Localizes to Mouse Skin Expressing Desmoglein

Balb/c mice were intravenously dosed with 10 mg/Kg of bifunctional antibody and skin was harvested 24 hours post dosing. Frozen sections were fixed, blocked and incubated with fluorophore-conjugated anti-human IgG for overnight at 4 C. Tissue was counterstained with DAPI for nuclei staining and mounted with Prolong gold anti-fade mounting agent. Binding of test articles to the skin was assessed by fluorescence imaging microscope. CD39DSGAB3 bound to Desmoglein on mouse tail skin as was evident by staining on the cell surface of keratinocytes throughout the epidermis (data not shown).

### Example 45: SD1-mouseCD39 Constructs Show ATPase Activity

100 ul of undiluted test articles or 100 ul of rCD39 at 10 ug/ml (initial concentration is 220 ug/ml) was mixed with ATP (100 uM final concentration) and incubate for 30 minutes at 37 C. Next, 100 ul of SN was transferred to a pre-warmed black walled plate and 100 ul of reconstituted Cell Titer Glo reagent was added. Following 10 min incubation at 37 C and the reading was made on EnVision plate reader. CD39DSGAB1 showed RLU (100 nM) of 2508, CD39DSGAB2 showed RLU of 1360, CD39DSGAB3 showed RLU of 1100, CD39DSGAB4 showed RLU of 9312, CD39DSGAB5 showed RLU of 2448, CD39DSGAB6 showed RLU of 4188, CD39DSGAB7 showed RLU of 11964, CD39DSGAB8 showed RLU of 9404, CD39DSGAB9 showed RLU of 624, rCD39 showed RLU of 540, while 100 mM ATP showed RLU of 1362660. Accordingly, SD1-mouseCD39 constructs show ATPase activity in a soluble assay format.

### Example 46: CD39DSGAB3 Attenuates Ear Inflammation in Contact Hypersensitivity Mouse Model

Balb/c mice were sensitized with epicutaneous administration of 0.5% DNFB solution at day 0 and day 1. Mice were dosed with vehicle and 3 mg/kg of bifunctional antibodies on day 4 and challenged on day 5 with 0.2% DNFB on right ear, while left ear with DNFB vehicle. Clinical caliper ear measurements for ear thickness was measured pre-challenge and day 6. CD39DSGAB3 showed mean ear caliper of 0.061±0.01, SD1-TTJ2 isotype control showed mean ear caliper of 0.12±0.012, vehicle showed mean ear caliper of 0.132±0.0125, no challenge showed mean ear caliper of 0.012±0.00374, and Dexamethasone showed mean ear caliper of 0.031±0.007. CD39DSGAB3 attenuates ear inflammation as evident by ear caliper differences in a mouse model of contact hypersensitivity.

### Example 47: SD1-humanCD39 Constructs Express In Vitro

Proteins were expressed in expi293 (transient expression) following standard protocols. Plasmid vectors carrying the genes for the constructs were transfected using Gibco ExpiFectamine 293 Transfection Kit (ThermoFisher Scientific) and the cultures were grown at 37 C with shaking (120 rpm), and 8% CO2 for 4 days. Cultures were harvested by centrifugation (3500 rpm, 4 C, 45 minutes) and purified using protein A affinity chromatography. Protein concentrations were measured using absorbance at A280 nm. CD39DSGAB10 showed a yield of 4.6 mg/L; CD39DSGAB11 showed a yield of 2.3 mg/L; CD39DSGAB12 showed a yield of 3.5 mg/L; CD39DSGAB13 showed a yield of 3.3 mg/L; CD39DSGAB14 showed a yield of 4 mg/L; CD39DSGAB29 showed a yield of 1.2 mg/L; CD39DSGAB22 showed a yield of 1.84 mg/L; CD39DSGAB23 showed a yield of 3.52 mg/L; CD39DSGAB24 showed a yield of 4.96 mg/L; CD39DSGAB30 showed a yield of 2.24 mg/L; CD39DSGAB31 showed a yield of 1.36 mg/L; CD39DSGAB15 showed a yield of 0.62 mg/L; CD39DSGAB16 showed a yield of 2.1 mg/L; CD39DSGAB17 showed a yield of 0.19 mg/L; CD39DSGAB18 showed a yield of 0.4 mg/L; CD39DSGAB19 showed a yield of 0.72 mg/L; CD39DSGAB20 showed a yield of 0.22 mg/L; CD39DSGAB40 showed a yield of 0.1 mg/L; CD39DSGAB25 showed a yield of 3.8 mg/L; CD39DSGAB26 showed a yield of 0.15 mg/L; CD39DSGAB27 showed a yield of 0.2 mg/L; CD39DSGAB28 showed a yield of 0.15 mg/L; CD39DSGAB32 showed a yield of 0.05 mg/L; CD39DSGAB33 showed a yield of 0.15 mg/L; CD39DSGAB34 showed a yield of 0.25 mg/L; CD39DSGAB39 showed a yield of 0.2 mg/L; CD39DSGAB35 showed a yield of 1.29 mg/L; CD39DSGAB36 showed a yield of 3.15 mg/L; CD39DSGAB41 showed a yield of 1.7 mg/L; CD39DSGAB42 showed a yield of 0 mg/L; CD39DSGAB37 showed a yield of 2.13 mg/L; CD39DSGAB43 showed a yield of 2.38 mg/L; CD39DSGAB38 showed a yield of 1.7 mg/L; and CD39DSGAB44 showed a yield of 2.34 mg/L. Accordingly, SD1-humanCD39 constructs are expressed in vitro.

### Example 48: SD1-humanCD39 Constructs Show Heterogeneity

20 μl of 1 mg/ml of the CD39DSGAB10, CD39DSGAB11, CD39DSGAB16, and CD39DSGAB43 was injected into an Agilent AdvanceBio SEC 300 Å 4.6.×150 mm column. The observed elution time was used to extrapolate the molecular weight of the sample from a standard curve generated from proteins of known molecular weight. The percentage of “protein of interest (% POI)” was calculated by integrating the signal of the peak with the expected elution time. The data shows % POI of 50% for CD39DSGAB10, 34% for CD39DSGAB11, 100% for CD39DSGAB16, 100% for CD39DSGAB4, 97% for CD39DSGAB5, 100% for CD39DSGAB6, and 66% for CD39DSGAB43. SD1-humanCD39 constructs show variable levels of heterogeneity based on the format of the molecules. Mutations/Design of CD39DSGAB16 dramatically reduced aggregation and material was 100% monodisperse.

### Example 49: SD1-humanCD39 Constructs Show ATPase Activity

100 ul of undiluted test articles or 100 ul of rCD39 at 10 ug/ml (initial concentration is 220 ug/ml) was mixed with ATP (100 uM final concentration) and incubate for 30 minutes at 37 C. Next, 100 ul of SN was transferred to a pre-warmed black walled plate and 100 ul of reconstituted Cell Titer Glo reagent was added. Following 10 min incubation at 37 C and the reading was made on EnVision plate reader. CD39DSGAB16 showed RLU (100 nM) of 785, CD39DSGAB17 showed RLU of 976, CD39DSGAB18 showed RLU of 1770, CD39DSGAB19 showed RLU of 1220, CD39DSGAB20 showed RLU of 3730, CD39DSGAB15 showed RLU of 1080, CD39DSGAB40 showed RLU of 883, CD39DSGAB10 showed RLU of 529, rCD39 showed RLU of 404, while ATP showed RLU of 1560000. Accordingly, SD1-mouseCD39 constructs show ATPase activity in a soluble assay format.

### Example 50: humanCD39-Fc Fusion Constructs Show Poor Expression In Vitro

Proteins were expressed in expi293 (transient expression) following standard protocols. Plasmid vectors carrying the genes for the constructs were transfected using Gibco ExpiFectamine 293 Transfection Kit (ThermoFisher Scientific) and the cultures were grown at 37 C with shaking (120 rpm), and 8% CO2 for 4 days. Cultures were harvested by centrifugation (3500 rpm, 4 C, 45 minutes) and purified using protein A affinity chromatography. Protein concentrations were measured using absorbance at A280 nm. CD39AB1 showed a yield of 0.63 mg/L; CD39AB8 showed a yield of 0.71 mg/L; CD39AB9 showed a yield of 0.35 mg/L; CD39AB10 showed a yield of 0.88 mg/L; CD39AB11 showed a yield of 0.74 mg/L; CD39AB12 showed a yield of 0.59 mg/L; CD39AB13 showed a yield of 0.64 mg/L; CD39AB14 showed a yield of 0.83 mg/L; CD39AB15 showed a yield of 0.62 mg/L; CD39AB16 showed a yield of 0.58 mg/L; CD39AB2 showed a yield of 0.97 mg/L; CD39AB3 showed a yield of 0.73 mg/L; CD39AB4 showed a yield of 0.94 mg/L; CD39AB5 showed a yield of 0.77 mg/L; CD39AB6 showed a yield of 0.81 mg/L; CD39AB7 showed a yield of 0.73 mg/L; Accordingly, humanCD39-Fc fusion constructs show low yields.

### Example 51: Skin-tethered Bispecific Molecules Prolong Survival and Ameliorate Skin Phenotype in xGVHD in a Tether- and Dose-Specific Manner

Xenogeneic graft versus host disease was induced by the transfer of human PBMC into immunodeficient mice. Beginning 5 days after cell transfer, mice were treated subcutaneously weekly with a vehicle, PD1AB33 at 1 mg/kg, or PD1DSGAB9 at 1 or 3 mg/kg. Skin inflammation was scored according to the following table:

| Criteria | | Score = 0 | Score = 1 | Score = 2 |
|---|---|---|---|---|
| Skin | Fur | Absence | poor grooming/ | No grooming |
| and fur | texture | | ruffling fur | |
| | Dehydration | Absence | Slightly | Important |
| | | | dehydration | dehydration |
| | | | (<2 sec) | (2-3 sec) |
| | Dry Skin | Absence | Lightly dry skin | Important dry |
| | | | | skin with crust |
| | Depilation/ | Absence | Light depilation | Important |
| | Hair loss | | | depilation |

Skin-tethered bispecific molecule, PD1DSGAB9, improved skin phenotype in a dose-dependent manner as compared to soluble PD-1 agonist, PD1AB33, which had no effect on the skin score, and prolonged median survival time to 40 days (1 mg/kg) and 36 days (3 mg/kg) as compared to approximately 29 days for the vehicle and approximately 33 days for the soluble PD-1 agonist, PD1AB33.

Accordingly, skin-tethered bispecific molecule reduced skin disease and improved survival in a concentration and tether dependent manner in a xGVHD mouse model.

The Examples provided herein demonstrate that molecules provided herein can be used to specifically localize therapeutics, such as an IL-2 mutein, PD-1 agonist, or CD39 Effector Domains, and also other therapeutic molecules, such as those described herein.

The disclosures of each and every patent, patent application, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

1. A protein comprising a skin targeting moiety, wherein the skin targeting moiety is an anti-desmoglein-1 antibody, an anti-desmoglein-2 antibody, an anti-desmoglein-3 antibody, or an anti-desmoglein-4 antibody and an effector binding/modulating moiety, wherein the effector binding/modulating moiety is a PD-1 agonist, CD39 Effector Domain, or an IL-2 mutein polypeptide (IL-2 mutein).

2-36. (canceled)

37. The protein of claim 1, wherein the effector binding/modulating moiety is an IL-2 mutein comprising a IL-2 sequence of SEQ ID NO: 6, and wherein the sequence comprises a mutation at a position that corresponds to position 53, 56, 80, or 118 of SEQ ID NO: 6.

38. The protein of claim 1, wherein the effector binding/modulating moiety is an IL-2 mutein comprising the sequence of SEQ ID NO: 60, wherein at least one of X₁, X₂, X₃, and X₄ is I and the remainder are L or I.

39. The protein of claim 37, wherein the mutation is a L to I substitution at position 53, 56, 80, or 118 of SEQ ID NO: 6.

40. The protein of claim 1, wherein the effector binding/modulating moiety is an IL-2 mutein comprising a IL-2 sequence of SEQ ID NO: 6, wherein the sequence comprises a mutation at one or more positions of 29, 31, 35, 37, 48, 69, 71, 74, 88, and 125 of SEQ ID NO: 6.

41. The protein of claim 38, wherein the mutation is a L to I substitution at position 53, 56, 80, or 118 of SEQ ID NO: 60.

42. The protein of claim 1, wherein the effector binding/modulating moiety is an IL-2 mutein comprising a IL-2 sequence of SEQ ID NO: 60, wherein the sequence comprises a mutation at one or more positions of 29, 31, 35, 37, 48, 69, 71, 74, 88, and 125 of SEQ ID NO: 60.

43. The protein of claim 42, wherein the IL-2 mutein further comprises a mutation at one or more of positions of E15, H16, Q22, D84, E95, or Q126 of SEQ ID NO: 60.

44. The protein of claim 43, wherein the mutation in the IL-2 mutein is a substitution selected from one or more of E15Q, H16N, Q22E, D84N, E95Q, or Q126E.

45. The protein of claim 42, wherein the IL-2 mutein comprises a N29S, Y31S, Y31H, K35R, T37A, K48E, V69A, N71R, Q74P, N88D, N88R, C125A or C125S mutation.

46-54. (canceled)

55. The protein of claim 37, wherein the IL-2 mutein is fused or linked to a Fc peptide, wherein the Fc peptide comprises a sequence of SEQ ID NO: 59 or a variant thereof.

56. The protein of claim 55, wherein the Fc peptide comprises a mutation at one or more of positions of L234, L247, L235, L248, G237, and G250 of SEQ ID NO: 59.

57. The protein of claim 55, wherein the mutation in the Fc polypeptide is L to A or G to A mutation.

58. The protein of claim 55, wherein the Fc peptide comprises L247A, L248A, and G250A mutations.

59. The protein of claim 55, wherein the Fc peptide comprises a L234A mutation, a L235A mutation, and/or a G237A mutation.

60. The protein of claim 1, wherein the polypeptide comprises a first chain and a second chain that form the polypeptide, wherein
the first chain comprises:
V_{H}—H_{c}-Linker-C1, wherein V_{H} is a variable heavy domain that binds to the target skin cell with a V_{L} domain of the second chain; H_{c} is a heavy chain of antibody comprising CH1-CH2-CH3 domain, the Linker is a glycine/serine linker, and C₁ is an IL-2 mutein fused or linked to a Fc protein in either the N-terminal or C-terminal orientation; and
the second chain comprises:
V_{L}-L_{c}, wherein V_{L} is a variable light chain domain that binds to the target cell with the V_{H} domain of the first chain, and the Lc domain is a light chain CK domain, and
the V_{H} and V_{L} domain are anti-desmoglein-1, anti-desmoglein-2, anti-desmoglein-3, and/or anti-desmoglein-4 variable domains that bind to desmoglein-1, desmoglein-2, desmoglein-3, and/or desmoglein-4, respectively, expressed on a cell.

61. (canceled)

62. The protein of claim 60, wherein the IL-2 mutein comprises a sequence of SEQ ID NO: 6, which comprises a mutation at a position that corresponds to position 53, 56, 80, or 118 of SEQ ID NO: 6.

63. The protein of claim 62, wherein the mutation is a L to I mutation at position 53, 56, 80, or 118 of SEQ ID NO: 6.

64. The protein of claim 60, wherein the IL-2 mutein comprises a mutation at a position that corresponds to position 53, 56, 80, or 118 of SEQ ID NO: 60.

65. The protein of claim 64, wherein the mutation is a L to I mutation at position 53, 56, 80, or 118 of SEQ ID NO: 60.

66. The protein of claim 62, wherein the mutein further comprises a mutation at a position that corresponds to position 69, 75, 88, or 125, or any combination thereof.

67. The protein of claim 66, further comprising a mutation selected from the group consisting of: L53I, L56I, L80I, and L118I and the mutations of V69A, Q74P, N88D or N88R, and optionally C125A or C125S.

68-72. (canceled)

73. The protein of claim 60, wherein the Fc protein comprises L247A, L248A, and G250A mutations according to Kabat numbering, or a L234A mutation, a L235A mutation, and/or a G237A mutation according to EU numbering.

74. The protein of claim 60, wherein the Linker comprises a sequence of GGGGSGGGGSGGGGS (SEQ ID NO: 30) or GGGGSGGGGSGGGGSGGGGS (SEQ ID NO: 22).

75-134. (canceled)
